SEQUENCE LISTING (554 sequences)

SEQ ID NO: 1 - DNA, 61 bp, Plasmopara viticola
ttatggtgttcaggggtaattatttttattttaatgatggcgactgcatttatgggttatg

SEQ ID NO: 2 - DNA, 61 bp, Rhynchosporium secalis
gtatgaacaataggtacatttatattcatattaatgatcgttacagcattcttgggttatg

SEQ ID NO: 3 - DNA, 61 bp, Pyrenophora teres
gtatgaactattggtactgttatctttatcttaatgatggctacagccttcctgggttacg

SEQ ID NO: 4 - DNA, 101 bp, Pyrenophora teres
cgctatacagataaatttaggttgtagttagccggaacttagacagccttcctgggttaccaacatagcccaaaatggtttaatataagtaataaaaaaag

SEQ ID NO: 5 - DNA, 61 bp, Mycosphaerella gramnicola
acatgaacaatcggtactataatactagttctgatgatggcaaccgcattcttagggtatg

SEQ ID NO: 6 - DNA, 54 bp, Sphaerotheca fulginea
cattaggtgtagttatattcatattaatgatcgttactgcattcctgggttatg

SEQ ID NO: 7 - DNA, 54 bp, Uncinula necator
caattggtacagtaatattcattttaatgatggctacagcattcttgggttatg

SEQ ID NO: 8 - DNA, 101 bp, Colletotrichum graminicola
gtttgagttataggtgctataatacttgtagctatgatgggtataggtttcttagggtatgttttaccttacggacaaatgtcattatgaggtgctacagt

SEQ ID NO: 9 - DNA, 101 bp, Colletotrichum graminicola
gtttgagttataggttgtataatacttgtagctatgatgggtataggtttcttaggatatgttttaccttacggacaaatgtcattatgaggtgctacagt

SEQ ID NO: 10 - DNA, 101 bp, Colletotrichum graminicola
gtttgagttataggtactataatacttgtagctatgatgggtataggtttcctgggttatgttttaccttacggacaaatgtcactatgaggtgcaactgt

SEQ ID NO: 11 - DNA, 101 bp, Colletotrichum graminicola
gtttgagttataggtactataatacttgtagctatgatgggtataggtttcctgggttacttcaacatagcccaaaatgatatgcaattattaggatttca

SEQ ID NO: 12 - DNA, 61 bp, Pythium aphanidermatum
ttatggtgttcaggtgttgttatttttattttaatgatggcaacagctttcatgggttatg

SEQ ID NO: 13 - DNA, 61 bp, Colletotrichum gloeosporiodes chilli
gtttgagttataggtgctataatacttgtagctatgatgggtataggtttcctgggttatg

SEQ ID NO: 14 - DNA, 101 bp, Colletotrichum gloeosporiodes mango
gtttgagtaataggtgctataattcttgtagctatgatgggtataggtttcttgggttatgttttaccttacgggcaaatgtcattatgaggtgcaacagt

SEQ ID NO: 15 - DNA, 61 bp, Oidium lycopersicum
acatgaactataggtacagttatattcatattaatgatggctacagcattcctgggttatg

SEQ ID NO: 16 - DNA, 101 bp, Leveillula taurica
acatgaacaataggtgtggtaatatttatattaatgatggctacagctttcttgggttatgttttaccgtacggtcaaatgagtttatgaggtgcaacagt

SEQ ID NO: 17 - DNA, 101 bp, Leveillula taurica
acatgaacaataggtgttgtaatatttatattaatgatggctacagctttcctaggttacgttttaccatacggacaaatgtcattatgaggtgcaacagt

SEQ ID NO: 18 - DNA, 101 bp, Leveillula taurica
acatgaactattggtgttgttatctttatattaatgatggctactgcctttttaggatatgttttaccatatggtcaaatgagtttatgaggtgctacagt

SEQ ID NO: 19 - DNA, 99 bp, Leveillula taurica
atgaacaattggtacagtaatattcatattaatgatggctactgcattcctgggttatgttctacctttcggacagatgtcgctctggggtgcaaccgt

SEQ ID NO: 20 - DNA, 61 bp, Pseudoperonspora cubensis
ttatggtgttcaggtgttattatttttattttaatgatggcaacagcttttatgggttatg

SEQ ID NO: 21 - DNA, 61 bp, Alternaria solani
gtatgaactattggtactgttatctttatcttaatgatggctacagctttcctgggttatg

SEQ ID NO: 22 - DNA, 61 bp, Cercospora arachidola
ttatgatctattggagttataattttagttcttatgatggcaatagccttcttaggatatg

SEQ ID NO: 23 - DNA, 54 bp, Rhizoctonia solani
ctatcggagttgttatgcttgttatgatgatggggatcgcatttttaggttatg

SEQ ID NO: 24 - DNA, 61 bp, Mycosphaerella musicola
gtatgagttataggtactattatattagttctaatgatggctaccgcctttttaggatatg

SEQ ID NO: 25 - DNA, 101 bp, Didymella bryoniae
gtatgaacaattggtactgttatctttatcttaatgatggctacagctttcctgggttatgttcttccttatgggcaaatgtcattatgaggtgcaactgt

SEQ ID NO: 26 - DNA, 101 bp, Didymella bryoniae
gtgtgaacaattggtactgttatctttatcttaatgatggctacagctttcctgggttatgtgctgccctacgggcagatgtcattatgaggtgctacagt

SEQ ID NO: 27 - DNA, 101 bp, Didymella lycopersici
gtatgaacaattggtactgttatctttatcttaatgatggctacagctttcctgggttatgttcttccttatgggcaaatgtcattatgaggtgctacagt

SEQ ID NO: 28 - DNA, 61 bp, Plasmopora viticola
ttatggtgttcaggggtaattatttttattttaatgatggcgactgcacttatgggttatg

SEQ ID NO: 29 - DNA, 61 bp, Rhyncosporium secalis
gtatgaacaataggtacatttatattcatattaatgatcgttacagcactcttgggttatg

SEQ ID NO: 30 - DNA, 61 bp, Pyrenophora teres
gtatgaactattggtactgttatctttatcttaatgatggctacagccctcctgggttacg

SEQ ID NO: 31 - DNA, 101 bp, Pyrenophora teres
cgctatacagataaatttaggttgtagttagccggaacttagacagccctcctgggttaccaacatagcccaaaatggtttaatataagtaataaaaaaag

SEQ ID NO: 32 - DNA, 61 bp, Mycosphaerella graminicola
acatgaacaatcggtactataatactagttctgatgatggcaaccgcactcttagggtatg

SEQ ID NO: 33 - DNA, 61 bp, Mycosphaerella fijiensis var difformis
gtatgagttataggtactattatattagttctaatgatggcaactgcccttttagggtatg

SEQ ID NO: 34 - DNA, 54 bp, Sphaerotheca fuliginea
cattaggtgtagttatattcatattaatgatcgttactgcactcctgggttatg

SEQ ID NO: 35 - DNA, 54 bp, Uncinula necator
caattggtacagtaatattcattttaatgatggctacagcactcttgggttatg

SEQ ID NO: 36 - DNA, 101 bp, Colletotrichum graminicola
gtttgagttataggtgctataatacttgtagctatgatgggtataggtctcttagggtatgttttaccttacggacaaatgtcattatgaggtgctacagt

SEQ ID NO: 37 - DNA, 101 bp, Colletotrichum graminicola
gtttgagttataggttgtataatacttgtagctatgatgggtataggtctcttaggatatgttttaccttacggacaaatgtcattatgaggtgctacagt

SEQ ID NO: 38 - DNA, 101 bp, Colletotrichum graminicola
gtttgagttataggtactataatacttgtagctatgatgggtataggtctcctgggttatgttttaccttacggacaaatgtcactatgaggtgcaactgt

SEQ ID NO: 39 - DNA, 101 bp, Colletotricum graminicola
gtttgagttataggtactataatacttgtagctatgatgggtataggtctcctgggttacttcaacatagcccaaaatgatatgcaattattaggatttca

SEQ ID NO: 40 - DNA, 61 bp, Pythium aphanidermatum
ttatggtgttcaggtgttgttatttttattttaatgatggcaacagctctcatgggttatg

SEQ ID NO: 41 - DNA, 61 bp, Colletotrichum gloeosporiodes chilli
gtttgagttataggtgctataatacttgtagctatgatgggtataggtctcctgggttatg

SEQ ID NO: 42 - DNA, 101 bp, Colletotrichum gloeosporiodes mango
gtttgagtaataggtgctataattcttgtagctatgatgggtataggtctcttgggttatgttttaccttacgggcaaatgtcattatgaggtgcaacagt

SEQ ID NO: 43 - DNA, 61 bp, Oidium lycopersicum
acatgaactataggtacagttatattcatattaatgatggctacagcactcctgggttatg

SEQ ID NO: 44 - DNA, 101 bp, Leveillula taurica
acatgaacaataggtgtggtaatatttatattaatgatggctacagctctcttgggttatgttttaccgtacggtcaaatgagtttatgaggtgcaacagt

SEQ ID NO: 45 - DNA, 101 bp, Leveillula taurica
acatgaacaataggtgttgtaatatttatattaatgatggctacagctctcctaggttacgttttaccatacggacaaatgtcattatgaggtgcaacagt

SEQ ID NO: 46 - DNA, 101 bp, Leveillula taurica
acatgaactattggtgttgttatctttatattaatgatggctactgcccttttaggatatgttttaccatatggtcaaatgagtttatgaggtgctacagt

SEQ ID NO: 47 - DNA, 99 bp, Leveillula taurica
atgaacaattggtacagtaatattcatattaatgatggctactgcactcctgggttatgttctacctttcggacagatgtcgctctggggtgcaaccgt

SEQ ID NO: 48 - DNA, 61 bp, Pseudoperonospora cubensis
ttatggtgttcaggtgttattatttttattttaatgatggcaacagctcttatgggttatg

SEQ ID NO: 49 - DNA, 61 bp, Alternaria solani
gtatgaactattggtactgttatctttatcttaatgatggctacagctctcctgggttatg

SEQ ID NO: 50 - DNA, 61 bp, Cercospora arachidola
ttatgatctattggagttataattttagttcttatgatggcaatagccctcttaggatatg

SEQ ID NO: 51 - DNA, 54 bp, Rhizoctonia solani
ctatcggagttgttatgcttgttatgatgatggggatcgcacttttaggttatg

SEQ ID NO: 52 - DNA, 61 bp, Mycosphaerella musicola
gtatgagttataggtactattatattagttctaatgatggctaccgcccttttaggatatg

SEQ ID NO: 53 - DNA, 101 bp, Didymella bryoniae
gtatgaacaattggtactgttatctttatcttaatgatggctacagctctcctgggttatgttcttccttatgggcaaatgtcattatgaggtgcaactgt

SEQ ID NO: 54 - DNA, 101 bp, Didymella bryoniae
gtgtgaacaattggtactgttatctttatcttaatgatggctacagctctcctgggttatgtgctgccctacgggcagatgtcattatgaggtgctacagt

SEQ ID NO: 55 - DNA, 101 bp, Didymella lycopersici
gtatgaacaattggtactgttatctttatcttaatgatggctacagctctcctgggttatgttcttccttatgggcaaatgtcattatgaggtgctacagt

SEQ ID NO: 56 - DNA, 61 bp, Plasmopora viticola
ttatggtgttcaggggtaattatttttattttaatgatggcgactgcattaatgggttatg

SEQ ID NO: 57 - DNA, 61 bp, Rhynchosporium secalis
gtatgaacaataggtacatttatattcatattaatgatcgttacagcattattgggttatg

SEQ ID NO: 58 - DNA, 61 bp, Pyrenophora teres
gtatgaactattggtactgttatctttatcttaatgatggctacagccttactgggttacg

SEQ ID NO: 59 - DNA, 101 bp, Pyrenophora teres
cgctatacagataaatttaggttgtagttagccggaacttagacagccttactgggttaccaacatagcccaaaatggtttaatataagtaataaaaaaag

SEQ ID NO: 60 - DNA, 61 bp, Mycosphaerella graminicola
acatgaacaatcggtactataatactagttctgatgatggcaaccgcattattagggtatg

SEQ ID NO: 61 - DNA, 61 bp, Mycosphaerella fijiensis var difformis
gtatgagttataggtactattatattagttctaatgatggcaactgccttattagggtatg

SEQ ID NO: 62 - DNA, 54 bp, Sphaerotheca fulginea
cattaggtgtagttatattcatattaatgatcgttactgcattactgggttatg

SEQ ID NO: 63 - DNA, 54 bp, Uncinula necator
caattggtacagtaatattcattttaatgatggctacagcattattgggttatg

SEQ ID NO: 64 - DNA, 101 bp, Colletotrichum graminicola
gtttgagttataggtgctataatacttgtagctatgatgggtataggtttattagggtatgttttaccttacggacaaatgtcattatgaggtgctacagt

SEQ ID NO: 65 - DNA, 101 bp, Colletotrichum graminicola
gtttgagttataggttgtataatacttgtagctatgatgggtataggtttattaggatatgttttaccttacggacaaatgtcattatgaggtgctacagt

SEQ ID NO: 66 - DNA, 101 bp, Colletotrichum graminicola
gtttgagttataggtactataatacttgtagctatgatgggtataggtttactgggttatgttttaccttacggacaaatgtcactatgaggtgcaactgt

SEQ ID NO: 67 - DNA, 101 bp, Colletotrichum graminicola
gtttgagttataggtactataatacttgtagctatgatgggtataggtttactgggttacttcaacatagcccaaaatgatatgcaattattaggatttca

SEQ ID NO: 68 - DNA, 61 bp, Pythium aphanidermatum
ttatggtgttcaggtgttgttatttttattttaatgatggcaacagctttaatgggttatg

SEQ ID NO: 69 - DNA, 61 bp, Colletotrichum gloeosporioides chilli
gtttgagttataggtgctataatacttgtagctatgatgggtataggtttactgggttatg

SEQ ID NO: 70 - DNA, 101 bp, Colletotrichum gloeosporioides mango
gtttgagtaataggtgctataattcttgtagctatgatgggtataggtttattgggttatgttttaccttacgggcaaatgtcattatgaggtgcaacagt

SEQ ID NO: 71 - DNA, 61 bp, Oidium lycopersicum
acatgaactataggtacagttatattcatattaatgatggctacagcattactgggttatg

SEQ ID NO: 72 - DNA, 101 bp, Leveillula taurica
acatgaacaataggtgtggtaatatttatattaatgatggctacagctttattgggttatgttttaccgtacggtcaaatgagtttatgaggtgcaacagt

SEQ ID NO: 73 - DNA, 101 bp, Leveillula taurica
acatgaacaataggtgttgtaatatttatattaatgatggctacagctttactaggttacgttttaccatacggacaaatgtcattatgaggtgcaacagt

SEQ ID NO: 74 - DNA, 101 bp, Leveillula taurica
acatgaactattggtgttgttatctttatattaatgatggctactgccttattaggatatgttttaccatatggtcaaatgagtttatgaggtgctacagt

SEQ ID NO: 75 - DNA, 99 bp, Leveillula taurica
atgaacaattggtacagtaatattcatattaatgatggctactgcattactgggttatgttctacctttcggacagatgtcgctctggggtgcaaccgt

SEQ ID NO: 76 - DNA, 61 bp, Pseodoperonospora cubensis
ttatggtgttcaggtgttattatttttattttaatgatggcaacagctttaatgggttatg

SEQ ID NO: 77 - DNA, 61 bp, Alternaria solani
gtatgaactattggtactgttatctttatcttaatgatggctacagctttactgggttatg

SEQ ID NO: 78 - DNA, 61 bp, Cercospora arachidola
ttatgatctattggagttataattttagttcttatgatggcaatagccttattaggatatg

SEQ ID NO: 79 - DNA, 54 bp, Rhizoctonia solani
ctatcggagttgttatgcttgttatgatgatggggatcgcattattaggttatg

SEQ ID NO: 80 - DNA, 61 bp, Mycosphaerella musicola
gtatgagttataggtactattatattagttctaatgatggctaccgccttattaggatatg

SEQ ID NO: 81 - DNA, 101 bp, Didymella bryoniae
gtatgaacaattggtactgttatctttatcttaatgatggctacagctttactgggttatgttcttccttatgggcaaatgtcattatgaggtgcaactgt

SEQ ID NO: 82 - DNA, 101 bp, Didymella bryoniae
gtgtgaacaattggtactgttatctttatcttaatgatggctacagctttactgggttatgtgctgccctacgggcagatgtcattatgaggtgctacagt

SEQ ID NO: 83 - DNA, 101 bp, Didymella lycopersici
gtatgaacaattggtactgttatctttatcttaatgatggctacagctttactgggttatgttcttccttatgggcaaatgtcattatgaggtgctacagt

SEQ ID NO: 84 - DNA, 61 bp, Plasmopara viticola
ttatggtgttcaggggtaattatttttattttaatgatggcgactgcattgatgggttatg

SEQ ID NO: 85 - DNA, 61 bp, Rhynchosporium secalis
gtatgaacaataggtacatttatattcatattaatgatcgttacagcattgttgggttatg

SEQ ID NO: 86 - DNA, 61 bp, Pyrenophora teres
gtatgaactattggtactgttatctttatcttaatgatggctacagccttgctgggttacg

SEQ ID NO: 87 - DNA, 101 bp, Pyrenophora teres
cgctatacagataaatttaggttgtagttagccggaacttagacagccttgctgggttaccaacatagcccaaaatggtttaatataagtaataaaaaaag

SEQ ID NO: 88 - DNA, 61 bp, Mycosphaerella graminicola
acatgaacaatcggtactataatactagttctgatgatggcaaccgcattgttagggtatg

SEQ ID NO: 89 - DNA, 61 bp, Mycosphaerella fijiensis var difformis
gtatgagttataggtactattatattagttctaatgatggcaactgccttgttagggtatg

SEQ ID NO: 90 - DNA, 54 bp, Sphaerotheca fuliginea
cattaggtgtagttatattcatattaatgatcgttactgcattgctgggttatg

SEQ ID NO: 91 - DNA, 54 bp, Uncinula necator
caattggtacagtaatattcattttaatgatggctacagcattgttgggttatg

SEQ ID NO: 92 - DNA, 101 bp, Colletotrichum graminicola
gtttgagttataggtgctataatacttgtagctatgatgggtataggtttgttagggtatgttttaccttacggacaaatgtcattatgaggtgctacagt

SEQ ID NO: 93 - DNA, 101 bp, Colletotrichum graminicola
gtttgagttataggttgtataatacttgtagctatgatgggtataggtttgttaggatatgttttaccttacggacaaatgtcattatgaggtgctacagt

SEQ ID NO: 94 - DNA, 101 bp, Colletotrichum graminicola
gtttgagttataggtactataatacttgtagctatgatgggtataggtttgctgggttatgttttaccttacggacaaatgtcactatgaggtgcaactgt

SEQ ID NO: 95 - DNA, 101 bp, Colletotrichum graminicola
gtttgagttataggtactataatacttgtagctatgatgggtataggtttgctgggttacttcaacatagcccaaaatgatatgcaattattaggatttca

SEQ ID NO: 96 - DNA, 61 bp, Pythium aphanidermatum
ttatggtgttcaggtgttgttatttttattttaatgatggcaacagctttgatgggttatg

SEQ ID NO: 97 - DNA, 61 bp, Colletotrichum gloeosporioides chilli
gtttgagttataggtgctataatacttgtagctatgatgggtataggtttgctgggttatg

SEQ ID NO: 98 - DNA, 101 bp, Colletotrichum gloeosporioides mango
gtttgagtaataggtgctataattcttgtagctatgatgggtataggtttgttgggttatgttttaccttacgggcaaatgtcattatgaggtgcaacagt

SEQ ID NO: 99 - DNA, 61 bp, Oidium lycopersicum
acatgaactataggtacagttatattcatattaatgatggctacagcattgctgggttatg

SEQ ID NO: 100 - DNA, 101 bp, Leveillula taurica
acatgaacaataggtgtggtaatatttatattaatgatggctacagctttgttgggttatgttttaccgtacggtcaaatgagtttatgaggtgcaacagt

SEQ ID NO: 101 - DNA, 101 bp, Leveillula taurica
acatgaacaataggtgttgtaatatttatattaatgatggctacagctttgctaggttacgttttaccatacggacaaatgtcattatgaggtgcaacagt

SEQ ID NO: 102 - DNA, 101 bp, Leveillula taurica
acatgaactattggtgttgttatctttatattaatgatggctactgccttgttaggatatgttttaccatatggtcaaatgagtttatgaggtgctacagt

SEQ ID NO: 103 - DNA, 99 bp, Leveillula taurica
atgaacaattggtacagtaatattcatattaatgatggctactgcattgctgggttatgttctacctttcggacagatgtcgctctggggtgcaaccgt

SEQ ID NO: 104 - DNA, 61 bp, Pseudoperonospora cubensis
ttatggtgttcaggtgttattatttttattttaatgatggcaacagctttgatgggttatg

SEQ ID NO: 105 - DNA, 61 bp, Alternaria solani
gtatgaactattggtactgttatctttatcttaatgatggctacagctttgctgggttatg

SEQ ID NO: 106 - DNA, 61 bp, Cercospora arachidola
ttatgatctattggagttataattttagttcttatgatggcaatagccttgttaggatatg

SEQ ID NO: 107 - DNA, 54 bp, Rhizoctonia solani
ctatcggagttgttatgcttgttatgatgatggggatcgcattgttaggttatg

SEQ ID NO: 108 - DNA, 61 bp, Mycosphaeralla musicola
gtatgagttataggtactattatattagttctaatgatggctaccgccttgttaggatatg

SEQ ID NO: 109 - DNA, 101 bp, Didymella bryoniae
gtatgaacaattggtactgttatctttatcttaatgatggctacagctttgctgggttatgttcttccttatgggcaaatgtcattatgaggtgcaactgt

SEQ ID NO: 110 - DNA, 101 bp, Didymella bryoniae
gtgtgaacaattggtactgttatctttatcttaatgatggctacagctttgctgggttatgtgctgccctacgggcagatgtcattatgaggtgctacagt

SEQ ID NO: 111 - DNA, 101 bp, Didymella lycopersici
gtatgaacaattggtactgttatctttatcttaatgatggctacagctttgctgggttatgttcttccttatgggcaaatgtcattatgaggtgctacagt

SEQ ID NO: 112 - DNA, 61 bp, Plasmopara viticola
ttatggtgttcaggggtaattatttttattttaatgatggcgactgcactaatgggttatg

SEQ ID NO: 113 - DNA, 61 bp, Rhynchosporium secalis
gtatgaacaataggtacatttatattcatattaatgatcgttacagcactattgggttatg

SEQ ID NO: 114 - DNA, 61 bp, Pyrenophora teres
gtatgaactattggtactgttatctttatcttaatgatggctacagccctactgggttacg

SEQ ID NO: 115 - DNA, 101 bp, Pyrenophora teres
cgctatacagataaatttaggttgtagttagccggaacttagacagccctactgggttaccaacatagcccaaaatggtttaatataagtaataaaaaaag

SEQ ID NO: 116 - DNA, 61 bp, Mycosphaerella graminicola
acatgaacaatcggtactataatactagttctgatgatggcaaccgcactattagggtatg

SEQ ID NO: 117 - DNA, 61 bp, Mycosphaerella fijiensis var difformis
gtatgagttataggtactattatattagttctaatgatggcaactgccctattagggtatg

SEQ ID NO: 118 - DNA, 54 bp, Sphaerotheca fuliginea
cattaggtgtagttatattcatattaatgatcgttactgcactactgggttatg

SEQ ID NO: 119 - DNA, 54 bp, Uncinula necator
caattggtacagtaatattcattttaatgatggctacagcactattgggttatg

SEQ ID NO: 120 - DNA, 101 bp, Colletotrichum graminicola
gtttgagttataggtgctataatacttgtagctatgatgggtataggtctattagggtatgttttaccttacggacaaatgtcattatgaggtgctacagt

SEQ ID NO: 121 - DNA, 101 bp, Colletotrichum graminicola
gtttgagttataggttgtataatacttgtagctatgatgggtataggtctattaggatatgttttaccttacggacaaatgtcattatgaggtgctacagt

SEQ ID NO: 122 - DNA, 101 bp, Colletotrichum graminicola
gtttgagttataggtactataatacttgtagctatgatgggtataggtctactgggttatgttttaccttacggacaaatgtcactatgaggtgcaactgt

SEQ ID NO: 123 - DNA, 101 bp, Colletotrichum graminicola
gtttgagttataggtactataatacttgtagctatgatgggtataggtctactgggttacttcaacatagcccaaaatgatatgcaattattaggatttca

SEQ ID NO: 124 - DNA, 61 bp, Pythium aphanidermatum
ttatggtgttcaggtgttgttatttttattttaatgatggcaacagctctaatgggttatg

SEQ ID NO: 125 - DNA, 61 bp, Colletotrichum gloeosporioides chilli
gtttgagttataggtgctataatacttgtagctatgatgggtataggtctactgggttatg

SEQ ID NO: 126 - DNA, 101 bp, Colletotrichum gloeosporioides mango
gtttgagtaataggtgctataattcttgtagctatgatgggtataggtctattgggttatgttttaccttacgggcaaatgtcattatgaggtgcaacagt

SEQ ID NO: 127 - DNA, 61 bp, Oidium lycopersicum
acatgaactataggtacagttatattcatattaatgatggctacagcactactgggttatg

SEQ ID NO: 128 - DNA, 101 bp, Leveillula taurica
acatgaacaataggtgtggtaatatttatattaatgatggctacagctctattgggttatgttttaccgtacggtcaaatgagtttatgaggtgcaacagt

SEQ ID NO: 129 - DNA, 101 bp, Leveillula taurica
acatgaacaataggtgttgtaatatttatattaatgatggctacagctctactaggttacgttttaccatacggacaaatgtcattatgaggtgcaacagt

SEQ ID NO: 130 - DNA, 101 bp, Leveillula taurica
acatgaactattggtgttgttatctttatattaatgatggctactgccctattaggatatgttttaccatatggtcaaatgagtttatgaggtgctacagt

SEQ ID NO: 131 - DNA, 99 bp, Leveillula taurica
atgaacaattggtacagtaatattcatattaatgatggctactgcactactgggttatgttctacctttcggacagatgtcgctctggggtgcaaccgt

SEQ ID NO: 132 - DNA, 61 bp, Pseudoperonspora cubensis
ttatggtgttcaggtgttattatttttattttaatgatggcaacagccttaatgggttatg

SEQ ID NO: 133 - DNA, 61 bp, Alternaria solani
gtatgaactattggtactgttatctttatcttaatgatggctacagctctactgggttatg

SEQ ID NO: 134 - DNA, 61 bp, Cercospora arachidola
ttatgatctattggagttataattttagttcttatgatggcaatagccctattaggatatg

SEQ ID NO: 135 - DNA, 54 bp, Rhizoctonia solani
ctatcggagttgttatgcttgttatgatgatggggatcgcactattaggttatg

SEQ ID NO: 136 - DNA, 61 bp, Mycosphaerella musicola
gtatgagttataggtactattatattagttctaatgatggctaccgccctattaggatatg

SEQ ID NO: 137 - DNA, 101 bp, Didymella bryoniae
gtatgaacaattggtactgttatctttatcttaatgatggctacagctctactgggttatgttcttccttatgggcaaatgtcattatgaggtgcaactgt

SEQ ID NO: 138 - DNA, 101 bp, Didiymella bryoniae
gtgtgaacaattggtactgttatctttatcttaatgatggctacagctctactgggttatgtgctgccctacgggcagatgtcattatgaggtgctacagt

SEQ ID NO: 139 - DNA, 101 bp, Didymella lycopersici
gtatgaacaattggtactgttatctttatcttaatgatggctacagctctactgggttatgttcttccttatgggcaaatgtcattatgaggtgctacagt

SEQ ID NO: 140 - DNA, 61 bp, Plasmopara viticola
ttatggtgttcaggggtaattatttttattttaatgatggcgactgcactgatgggttatg

SEQ ID NO: 141 - DNA, 61 bp, Rhynchosporium secalis
gtatgaacaataggtacatttatattcatattaatgatcgttacagcactgttgggttatg

SEQ ID NO: 142 - DNA, 61 bp, Pyrenophora teres
gtatgaactattggtactgttatctttatcttaatgatggctacagccctgctgggttacg

SEQ ID NO: 143 - DNA, 101 bp, Pyrenopora teres
cgctatacagataaatttaggttgtagttagccggaacttagacagccctgctgggttaccaacatagcccaaaatggtttaatataagtaataaaaaaag

SEQ ID NO: 144 - DNA, 61 bp, Mycosphaerella graminicola
acatgaacaatcggtactataatactagttctgatgatggcaaccgcactgttagggtatg

SEQ ID NO: 145 - DNA, 61 bp, Mycosphaerella fijiensis var difformis
gtatgagttataggtactattatattagttctaatgatggcaactgccctgttagggtatg

SEQ ID NO: 146 - DNA, 54 bp, Sphaerotheca fuliginea
cattaggtgtagttatattcatattaatgatcgttactgcactgctgggttatg

SEQ ID NO: 147 - DNA, 54 bp, Uncinula necator
caattggtacagtaatattcattttaatgatggctacagcactgttgggttatg

SEQ ID NO: 148 - DNA, 101 bp, Colletotrichum graminicola
gtttgagttataggtgctataatacttgtagctatgatgggtataggtctgttagggtatgttttaccttacggacaaatgtcattatgaggtgctacagt

SEQ ID NO: 149 - DNA, 101 bp, Colletotrichum graminicola
gtttgagttataggttgtataatacttgtagctatgatgggtataggtctgttaggatatgttttaccttacggacaaatgtcattatgaggtgctacagt

SEQ ID NO: 150 - DNA, 101 bp, Colletotrichum graminicola
gtttgagttataggtactataatacttgtagctatgatgggtataggtctgctgggttatgttttaccttacggacaaatgtcactatgaggtgcaactgt

SEQ ID NO: 151 - DNA, 101 bp, Colletotrichum graminicola
gtttgagttataggtactataatacttgtagctatgatgggtataggtctgctgggttacttcaacatagcccaaaatgatatgcaattattaggatttca

SEQ ID NO: 152 - DNA, 61 bp, Pythium aphanidermatum
ttatggtgttcaggtgttgttatttttattttaatgatggcaacagctctgatgggttatg

SEQ ID NO: 153 - DNA, 61 bp, Colletotrichum gloeosporioides chilli
gtttgagttataggtgctataatacttgtagctatgatgggtataggtctgctgggttatg

SEQ ID NO: 154 - DNA, 101 bp, Colletotrichum gloesporioides mango
gtttgagtaataggtgctataattcttgtagctatgatgggtataggtctgttgggttatgttttaccttacgggcaaatgtcattatgaggtgcaacagt

SEQ ID NO: 155 - DNA, 61 bp, Oidium lycopersicum
acatgaactataggtacagttatattcatattaatgatggctacagcactgctgggttatg

SEQ ID NO: 156 - DNA, 101 bp, Leveillula taurica
acatgaacaataggtgtggtaatatttatattaatgatggctacagctctgttgggttatgttttaccgtacggtcaaatgagtttatgaggtgcaacagt

SEQ ID NO: 157 - DNA, 101 bp, Leveillula taurica
acatgaacaataggtgttgtaatatttatattaatgatggctacagctctgctaggttacgttttaccatacggacaaatgtcattatgaggtgcaacagt

SEQ ID NO: 158 - DNA, 101 bp, Leveillula taurica
acatgaactattggtgttgttatctttatattaatgatggctactgccctgttaggatatgttttaccatatggtcaaatgagtttatgaggtgctacagt

SEQ ID NO: 159 - DNA, 99 bp, Leveillula taurica
atgaacaattggtacagtaatattcatattaatgatggctactgcactgctgggttatgttctacctttcggacagatgtcgctctggggtgcaaccgt

SEQ ID NO: 160 - DNA, 61 bp, Pseudoperonospora cubensis
ttatggtgttcaggtgttattatttttattttaatgatggcaacagctctgatgggttatg

SEQ ID NO: 161 - DNA, 61 bp, Alternaria solani
gtatgaactattggtactgttatctttatcttaatgatggctacagctctgctgggttatg

SEQ ID NO: 162 - DNA, 61 bp, Cercospora arachidola
ttatgatctattggagttataattttagttcttatgatggcaatagccctgttaggatatg

SEQ ID NO: 163 - DNA, 54 bp, Rhizoctonia solani
ctatcggagttgttatgcttgttatgatgatggggatcgcactgttaggttatg

SEQ ID NO: 164 - DNA, 61 bp, Mycosphaerella musicola
gtatgagttataggtactattatattagttctaatgatggctaccgccctgttaggatatg

SEQ ID NO: 165 - DNA, 101 bp, Didymella bryoniae
gtatgaacaattggtactgttatctttatcttaatgatggctacagctctgctgggttatgttcttccttatgggcaaatgtcattatgaggtgcaactgt

SEQ ID NO: 166 - DNA, 101 bp, Didymella bryoniae
gtgtgaacaattggtactgttatctttatcttaatgatggctacagctctgctgggttatgtgctgccctacgggcagatgtcattatgaggtgctacagt

SEQ ID NO: 167 - DNA, 101 bp, Didymella lycopersici
gtatgaacaattggtactgttatctttatcttaatgatggctacagctctgctgggttatgttcttccttatgggcaaatgtcattatgaggtgctacagt

SEQ ID NO: 168 - DNA, 25 bp, Artificial Sequence
  Table 9, primer #1
cccaaggcaaaacataacccatcag

SEQ ID NO: 169 - DNA, 26 bp, Artificial Sequence
  Table 9, primer #2
attcatattaatgatcgttactgccc

SEQ ID NO: 170 - DNA, 26 bp, Artificial Sequence
  Table 9, primer #3
attcatattaatgatcgttacagcgc

SEQ ID NO: 171 - DNA, 26 bp, Artificial Sequence
  Table 9, primer #4
ctttatcttaatgatggctacagcgc

SEQ ID NO: 172 - DNA, 26 bp, Artificial Sequence
  Table 9, primer #5
gtagttagccggaacttagacagcgc

SEQ ID NO: 173 - DNA, 26 bp, Artificial Sequence
  Table 9, primer #6
actagttctgatgatggcaaccgcgc

SEQ ID NO: 174 - DNA, 26 bp, Artificial Sequence
  Table 9, primer #7 Mycosphaerella fijiensis var difformis
attagttctaatgatggcaactgcgc

SEQ ID NO: 175 - DNA, 26 bp, Artificial Sequence
  Table 9, primer #8 Sphaerotheca fuliginea
attcatattaatgatcgttactgcgc

SEQ ID NO: 176 - DNA, 26 bp, Artificial Sequence
  Table 9, primer #9
attcattttaatgatggctacagcgc

SEQ ID NO: 177 - DNA, 26 bp, Artificial Sequence
  Table 9, primer #10
acttgtagctatgatgggtataggcc

SEQ ID NO: 178 - DNA, 26 bp, Artificial Sequence
  Table 9, primer #11
acttgtagctatgatgggtataggcc

SEQ ID NO: 179 - DNA, 26 bp, Artificial Sequence
  Table 9, primer #12
acttgtagctatgatgggtataggac

SEQ ID NO: 180 - DNA, 26 bp, Artificial Sequence
  Table 9, primer #13
ttttattttaatgatggcaacagcgc

SEQ ID NO: 181 - DNA, 26 bp, Artificial Sequence
  Table 9, primer #14
acttgtagctatgatgggtataggac

SEQ ID NO: 182 - DNA, 26 bp, Artificial Sequence
  Table 9, primer #15
tcttgtagctatgatgggtataggac

SEQ ID NO: 183 - DNA, 26 bp, Artificial Sequence
  Table 9, primer #16
attcatattaatgatggctacagcgc

SEQ ID NO: 184 - DNA, 26 bp, Artificial Sequence
  Table 9, primer #17
atttatattaatgatggctacagcgc

SEQ ID NO: 185 - DNA, 26 bp, Artificial Sequence
  Table 9, primer #18
atttatattaatgatggctacagcgc

SEQ ID NO: 186 - DNA, 26 bp, Artificial Sequence
  Table 9, primer #19
ctttatattaatgatggctactgcgc

SEQ ID NO: 187 - DNA, 26 bp, Artificial Sequence
  Table 9, primer #20
attcatattaatgatggctactgcgc

SEQ ID NO: 188 - DNA, 26 bp, Artificial Sequence
  Table 9, primer #21
ttttattttaatgatggcaacagcgc

SEQ ID NO: 189 - DNA, 26 bp, Artificial Sequence
  Table 9, primer #22
ctttatcttaatgatggctacagcgc

SEQ ID NO: 190 - DNA, 26 bp, Artificial Sequence
  Table 9, primer #23
tttagttcttatgatggcaatagcgc

SEQ ID NO: 191 - DNA, 26 bp, Artificial Sequence
  Table 9, primer #24
gcttgttatgatgatggggatcgcgc

SEQ ID NO: 192 - DNA, 26 bp, Artificial Sequence
  Table 9, primer #25
ctttatattaatgatagttacagcgc

SEQ ID NO: 193 - DNA, 26 bp, Artificial Sequence
  Table 9, primer #26
ttttattttaatgatggctactgcgc

SEQ ID NO: 194 - DNA, 26 bp, Artificial Sequence
  Table 9, primer #27
attagttctaatgatggctaccgcgc

SEQ ID NO: 195 - DNA, 26 bp, Artificial Sequence
  Table 9, primer #28
ctttatcttaatgatggctacagcgc

SEQ ID NO: 196 - DNA, 26 bp, Artificial Sequence
  Table 9, primer #29
ctttatcttaatgatggctacagcgc

SEQ ID NO: 197 - DNA, 26 bp, Artificial Sequence
  Table 9, primer #30
ctttatcttaatgatggctacagcgc

SEQ ID NO: 198 - DNA, 25 bp, Artificial Sequence
  Table 10, primer #1
tccccaaggcaaaacataacccagt

SEQ ID NO: 199 - DNA, 26 bp, Artificial Sequence
  Table 10, primer #2
tcatattaatgatcgttactgcatga

SEQ ID NO: 200 - DNA, 26 bp, Artificial Sequence
  Table 10, primer #3
tcatattaatgatcgttacagcatga

SEQ ID NO: 201 - DNA, 26 bp, Artificial Sequence
  Table 10, primer #4
ttatcttaatgatggctacagcctga

SEQ ID NO: 202 - DNA, 26 bp, Artificial Sequence
  Table 10, primer #5
agttagccggaacttagacagcctga

SEQ ID NO: 203 - DNA, 26 bp, Artificial Sequence
  Table 10, primer #6
tagttctgatgatggcaaccgcatga

SEQ ID NO: 204 - DNA, 26 bp, Artificial Sequence
  Table 10, primer #7
tagttctaatgatggcaactgcctga

SEQ ID NO: 205 - DNA, 26 bp, Artificial Sequence
  Table 10, primer #8
tcatattaatgatcgttactgcatga

SEQ ID NO: 206 - DNA, 26 bp, Artificial Sequence
  Table 10, primer #9
tcattttaatgatggctacagcatga

SEQ ID NO: 207 - DNA, 26 bp, Artificial Sequence
  Table 10, primer #10
ttgtagctatgatgggtataggttga

SEQ ID NO: 208 - DNA, 26 bp, Artificial Sequence
  Table 10, primer #11
ttgtagctatgatgggtataggttga

SEQ ID NO: 209 - DNA, 26 bp, Artificial Sequence
  Table 10, primer #12
ttgtagctatgatgggtataggttga

SEQ ID NO: 210 - DNA, 25 bp, Artificial Sequence
  Table 10 primer #13
accccaaggtaatacataacacgtt

SEQ ID NO: 211 - DNA, 26 bp, Artificial Sequence
  Table 10, primer #14
ttgtagctatgatgggtataggttga

SEQ ID NO: 212 - DNA, 26 bp, Artificial Sequence
  Table 10 primer #15
tcatattaatgatggctacagcatga

SEQ ID NO: 213 - DNA, 26 bp, Artificial Sequence
  Table 10 primer #16
ttatattaatgatggctacagcttga

SEQ ID NO: 214 - DNA, 26 bp, Artificial Sequence
  Table 10 primer #17
ttatattaatgatggctacagcttga

SEQ ID NO: 215 - DNA, 26 bp, Artificial Sequence
  Table 10 primer #18
ttatattaatgatggctactgcctga

SEQ ID NO: 216 - DNA, 26 bp, Artificial Sequence
  Table 10 primer #19
tcatattaatgatggctactgcatga

SEQ ID NO: 217 - DNA, 26 bp, Artificial Sequence
  Table 10 primer #20
ttattttaatgatggcaacagcttga

SEQ ID NO: 218 - DNA, 26 bp, Artificial Sequence
  Table 10 primer #21
ttatcttaatgatggctacagcttga

SEQ ID NO: 219 - DNA, 26 bp, Artificial Sequence
  Table 10 primer #22
tagttcttatgatggcaatagcctga

SEQ ID NO: 220 - DNA, 26 bp, Artificial Sequence
  Table 10 primer #23
ttgttatgatgatggggatcgcatga

SEQ ID NO: 221 - DNA, 26 bp, Artificial Sequence
  Table 10 primer #24
ttatattaatgatagttacagcctga

SEQ ID NO: 222 - DNA, 26 bp, Artificial Sequence
  Table 10 primer #25
ttattttaatgatggctactgcttga

SEQ ID NO: 223 - DNA, 26 bp, Artificial Sequence
  Table 10 primer #26
tagttctaatgatggctaccgcctga

SEQ ID NO: 224 - DNA, 26 bp, Artificial Sequence
  Table 10 primer #27
ttatcttaatgatggctacagcttga

SEQ ID NO: 225 - DNA, 26 bp, Artificial Sequence
  Table 10 primer #28
ttatcttaatgatggctacagcttga

SEQ ID NO: 226 - DNA, 26 bp, Artificial Sequence
  Table 10 primer #29
ttatcttaatgatggctacagcttga

SEQ ID NO: 227 - DNA, 25 bp, Artificial Sequence
  Table 11 primer #1
tccccaaggcaaaacataacccagc

SEQ ID NO: 228 - DNA, 26 bp, Artificial Sequence
  Table 11 primer #2
tcatattaatgatcgttactgcatcg

SEQ ID NO: 229 - DNA, 26 bp, Artificial Sequence
  Table 11 primer #3
tcatattaatgatcgttacagcatcg

SEQ ID NO: 230 - DNA, 26 bp, Artificial Sequence
  Table 11 primer #4
ttatcttaatgatggctacagcctcg

SEQ ID NO: 231 - DNA, 26 bp, Artificial Sequence
  Table 11 primer #5
agttagccggaacttagacagcctcg

SEQ ID NO: 232 - DNA, 26 bp, Artificial Sequence
  Table 11 primer #6
tagttctgatgatggcaaccgcatcg

SEQ ID NO: 233 - DNA, 26 bp, Artificial Sequence
  Table 11 primer #7
tagttctaatgatggcaactgcctcg

SEQ ID NO: 234 - DNA, 26 bp, Artificial Sequence
  Table 11 primer #8
tcatattaatgatcgttactgcatcg

SEQ ID NO: 235 - DNA, 26 bp, Artificial Sequence
  Table 11 primer #9
tcattttaatgatggctacagcatcg

SEQ ID NO: 236 - DNA, 26 bp, Artificial Sequence
  Table 11 primer #10
ttgtagctatgatgggtataggttcg

SEQ ID NO: 237 - DNA, 26 bp, Artificial Sequence
  Table 11 primer #11
ttgtagctatgatgggtataggttcg

SEQ ID NO: 238 - DNA, 26 bp, Artificial Sequence
  Table 11 primer #12
ttgtagctatgatgggtataggttcg

SEQ ID NO: 239 - DNA, 26 bp, Artificial Sequence
  Table 11 primer #13
ttattttaatgatggcaacagcttcg

SEQ ID NO: 240 - DNA, 26 bp, Artificial Sequence
  Table 11 primer #14
ttgtagctatgatgggtataggttcg

SEQ ID NO: 241 - DNA, 26 bp, Artificial Sequence
  Table 11 primer #15
tcatattaatgatggctacagcatcg

SEQ ID NO: 242 - DNA, 26 bp, Artificial Sequence
  Table 11 primer #16
ttatattaatgatggctacagcttcg

SEQ ID NO: 243 - DNA, 26 bp, Artificial Sequence
  Table 11 primer #17
ttatattaatgatggctacagcttcg

SEQ ID NO: 244 - DNA, 26 bp, Artificial Sequence
  Table 11 primer #18
ttatattaatgatggctactgcctcg

SEQ ID NO: 245 - DNA, 26 bp, Artificial Sequence
  Table 11 primer #19
tcatattaatgatggctactgcatcg

SEQ ID NO: 246 - DNA, 26 bp, Artificial Sequence
  Table 11 primer #20
ttattttaatgatggcaacagcttcg

SEQ ID NO: 247 - DNA, 26 bp, Artificial Sequence
  Table 11 primer #21
ttatcttaatgatggctacagcttcg

SEQ ID NO: 248 - DNA, 26 bp, Artificial Sequence
  Table 11 primer #22
tagttcttatgatggcaatagcctcg

SEQ ID NO: 249 - DNA, 26 bp, Artificial Sequence
  Table 11 primer #23
ttgttatgatgatggggatcgcatcg

SEQ ID NO: 250 - DNA, 26 bp, Artificial Sequence
  Table 11 primer #24
ttatattaatgatagttacagcctcg

SEQ ID NO: 251 - DNA, 26 bp, Artificial Sequence
  Table 11 primer #25
ttattttaatgatggctactgcttcg

SEQ ID NO: 252 - DNA, 26 bp, Artificial Sequence
  Table 11 primer #26
tagttctaatgatggctaccgcctcg

SEQ ID NO: 253 - DNA, 26 bp, Artificial Sequence
  Table 11 primer #27
ttatcttaatgatggctacagcttcg

SEQ ID NO: 254 - DNA, 26 bp, Artificial Sequence
  Table 11 primer #28
ttatcttaatgatggctacagcttcg

SEQ ID NO: 255 - DNA, 26 bp, Artificial Sequence
  Table 11 primer #29
ttatcttaatgatggctacagcttcg

SEQ ID NO: 256 - DNA, 26 bp, Artificial Sequence
  Table 12 primer #1
ttattttaatgatggcgactgcacga

SEQ ID NO: 257 - DNA, 26 bp, Artificial Sequence
  Table 12 primer #2
tcatattaatgatcgttactgcacga

SEQ ID NO: 258 - DNA, 26 bp, Artificial Sequence
  Table 12 primer #3
tcatattaatgatcgttacagcacga

SEQ ID NO: 259 - DNA, 26 bp, Artificial Sequence
  Table 12 primer #4
ttatcttaatgatggctacagcccga

SEQ ID NO: 260 - DNA, 26 bp, Artificial Sequence
  Table 12 primer #5
agttagccggaacttagacagcccga

SEQ ID NO: 261 - DNA, 26 bp, Artificial Sequence
  Table 12 primer #6
tagttctgatgatggcaaccgcacga

SEQ ID NO: 262 - DNA, 26 bp, Artificial Sequence
  Table 12 primer #7
tagttctaatgatggcaactgcccga

SEQ ID NO: 263 - DNA, 26 bp, Artificial Sequence
  Table 12 primer #8
tcatattaatgatcgttactgcacga

SEQ ID NO: 264 - DNA, 26 bp, Artificial Sequence
  Table 12 primer #9
tcattttaatgatggctacagcacga

SEQ ID NO: 265 - DNA, 26 bp, Artificial Sequence
  Table 12 primer #10
ttgtagctatgatgggtataggtcga

SEQ ID NO: 266 - DNA, 26 bp, Artificial Sequence
  Table 12 primer #11
ttgtagctatgatgggtataggtcga

SEQ ID NO: 267 - DNA, 26 bp, Artificial Sequence
  Table 12 primer #12
ttgtagctatgatgggtataggtcga

SEQ ID NO: 268 - DNA, 26 bp, Artificial Sequence
  Table 12 primer #13
ttattttaatgatggcaacagctcga

SEQ ID NO: 269 - DNA, 26 bp, Artificial Sequence
  Table 12 primer #14
ttgtagctatgatgggtataggtcga

SEQ ID NO: 270 - DNA, 26 bp, Artificial Sequence
  Table 12 primer #15
tcatattaatgatggctacagcacga

SEQ ID NO: 271 - DNA, 26 bp, Artificial Sequence
  Table 12 primer #16
ttatattaatgatggctacagctcga

SEQ ID NO: 272 - DNA, 26 bp, Artificial Sequence
  Table 12 primer #17
ttatattaatgatggctacagctcga

SEQ ID NO: 273 - DNA, 26 bp, Artificial Sequence
  Table 12 primer #18
ttatattaatgatggctactgcccga

SEQ ID NO: 274 - DNA, 26 bp, Artificial Sequence
  Table 12 primer #19
tcatattaatgatggctactgcacga

SEQ ID NO: 275 - DNA, 26 bp, Artificial Sequence
  Table 12 primer #20
ttattttaatgatggcaacagctcga

SEQ ID NO: 276 - DNA, 26 bp, Artificial Sequence
  Table 12 primer #21
ttatcttaatgatggctacagctcga

SEQ ID NO: 277 - DNA, 26 bp, Artificial Sequence
  Table 12 primer #22
tagttcttatgatggcaatagcccga

SEQ ID NO: 278 - DNA, 26 bp, Artificial Sequence
  Table 12 primer #23
ttgttatgatgatggggatcgcacga

SEQ ID NO: 279 - DNA, 26 bp, Artificial Sequence
  Table 12 primer #24
ttatattaatgatagttacagcccga

SEQ ID NO: 280 - DNA, 26 bp, Artificial Sequence
  Table 12 primer #25
ttattttaatgatggctactgctcga

SEQ ID NO: 281 - DNA, 26 bp, Artificial Sequence
  Table 12 primer #26
tagttctaatgatggctaccgcccga

SEQ ID NO: 282 - DNA, 26 bp, Artificial Sequence
  Table 12 primer #27
ttatcttaatgatggctacagctcga

SEQ ID NO: 283 - DNA, 26 bp, Artificial Sequence
  Table 12 primer #28
ttatcttaatgatggctacagctcga

SEQ ID NO: 284 - DNA, 26 bp, Artificial Sequence
  Table 12 primer #29
ttatcttaatgatggctacagctcga

SEQ ID NO: 285 - DNA, 26 bp, Artificial Sequence
  Table 13 primer #1
ttattttaatgatggcgactgcaccg

SEQ ID NO: 286 - DNA, 26 bp, Artificial Sequence
  Table 13 primer #2
tcatattaatgatcgttactgcacgg

SEQ ID NO: 287 - DNA, 26 bp, Artificial Sequence
  Table 13 primer #3
tcatattaatgatcgttacagcaccg

SEQ ID NO: 288 - DNA, 26 bp, Artificial Sequence
  Table 13 primer #4
ttatcttaatgatggctacagccccg

SEQ ID NO: 289 - DNA, 26 bp, Artificial Sequence
  Table 13 primer #5
agttagccggaacttagacagccccg

SEQ ID NO: 290 - DNA, 26 bp, Artificial Sequence
  Table 13 primer #6
tagttctgatgatggcaaccgcaccg

SEQ ID NO: 291 - DNA, 26 bp, Artificial Sequence
  Table 13 primer #7
tagttctaatgatggcaactgccccg

SEQ ID NO: 292 - DNA, 26 bp, Artificial Sequence
  Table 13 primer #8
tcatattaatgatcgttactgcaccg

SEQ ID NO: 293 - DNA, 26 bp, Artificial Sequence
  Table 13 primer #9
tcattttaatgatggctacagcaccg

SEQ ID NO: 294 - DNA, 26 bp, Artificial Sequence
  Table 13 primer #10
ttgtagctatgatgggtataggtccg

SEQ ID NO: 295 - DNA, 26 bp, Artificial Sequence
  Table 13 primer #11
ttgtagctatgatgggtataggtccg

SEQ ID NO: 296 - DNA, 26 bp, Artificial Sequence
  Table 13 primer #12
ttgtagctatgatgggtataggtccg

SEQ ID NO: 297 - DNA, 26 bp, Artificial Sequence
  Table 13 primer #13
ttattttaatgatggcaacagctccg

SEQ ID NO: 298 - DNA, 26 bp, Artificial Sequence
  Table 13 primer #14
ttgtagctatgatgggtataggtccg

SEQ ID NO: 299 - DNA, 26 bp, Artificial Sequence
  Table 13 primer #15
tcatattaatgatggctacagcaccg

SEQ ID NO: 300 - DNA, 26 bp, Artificial Sequence
  Table 13 primer #16
ttatattaatgatggctacagctccg

SEQ ID NO: 301 - DNA, 26 bp, Artificial Sequence
  Table 13 primer #17
ttatattaatgatggctacagctccg

SEQ ID NO: 302 - DNA, 26 bp, Artificial Sequence
  Table 13 primer #18
ttatattaatgatggctactgccccg

SEQ ID NO: 303 - DNA, 26 bp, Artificial Sequence
  Table 13 primer #19
tcatattaatgatggctactgcaccg

SEQ ID NO: 304 - DNA, 26 bp, Artificial Sequence
  Table 13 primer #20
ttattttaatgatggcaacagctccg

SEQ ID NO: 305 - DNA, 26 bp, Artificial Sequence
  Table 13 primer #21
ttatcttaatgatggctacagctccg

SEQ ID NO: 306 - DNA, 26 bp, Artificial Sequence
  Table 13 primer #22
tagttcttatgatggcaatagccccg

SEQ ID NO: 307 - DNA, 26 bp, Artificial Sequence
  Table 13 primer #23
ttgttatgatgatggggatcgcaccg

SEQ ID NO: 308 - DNA, 26 bp, Artificial Sequence
  Table 13 primer #24
ttatattaatgatagttacagccccg

SEQ ID NO: 309 - DNA, 26 bp, Artificial Sequence
  Table 13 primer #25
ttattttaatgatggctactgctccg

SEQ ID NO: 310 - DNA, 26 bp, Artificial Sequence
  Table 13 primer #26
tagttctaatgatggctaccgccccg

SEQ ID NO: 311 - DNA, 26 bp, Artificial Sequence
  Table 13 primer #27
ttatcttaatgatggctacagctccg

SEQ ID NO: 312 - DNA, 26 bp, Artificial Sequence
  Table 13 primer #28
ttatcttaatgatggctacagctccg

SEQ ID NO: 313 - DNA, 26 bp, Artificial Sequence
  Table 13 primer #29
ttatcttaatgatggctacagctccg

SEQ ID NO: 314 - DNA, 25 bp, Artificial Sequence
  Table 14 primer #1
cccaaggcaaaacataacccatgaa

SEQ ID NO: 315 - DNA, 26 bp, Artificial Sequence
  Table 14 primer #2
attcatattaatgatcgttactgcgt

SEQ ID NO: 316 - DNA, 26 bp, Artificial Sequence
  Table 14 primer #3
attcatattaatgatcgttacagcgt

SEQ ID NO: 317 - DNA, 26 bp, Artificial Sequence
  Table 14 primer #4
ctttatcttaatgatggctacagcgt

SEQ ID NO: 318 - DNA, 26 bp, Artificial Sequence
  Table 14 primer #5
gtagttagccggaacttagacagcgt

SEQ ID NO: 319 - DNA, 26 bp, Artificial Sequence
  Table 14 primer #6
actagttctgatgatggcaaccgcgt

SEQ ID NO: 320 - DNA, 26 bp, Artificial Sequence
  Table 14 primer #7
attagttctaatgatggcaactgcgt

SEQ ID NO: 321 - DNA, 26 bp, Artificial Sequence
  Table 14 primer #8
attcatattaatgatcgttactgcgt

SEQ ID NO: 322 - DNA, 26 bp, Artificial Sequence
  Table 14 primer #9
attcattttaatgatggctacagcgt

SEQ ID NO: 323 - DNA, 26 bp, Artificial Sequence
  Table 14 primer #10
acttgtagctatgatgggtataggat

SEQ ID NO: 324 - DNA, 26 bp, Artificial Sequence
  Table 14 primer #11
acttgtagctatgatgggtataggat

SEQ ID NO: 325 - DNA, 26 bp, Artificial Sequence
  Table 14 primer #12
acttgtagctatgatgggtataggat

SEQ ID NO: 326 - DNA, 26 bp, Artificial Sequence
  Table 14 primer #13
ttttattttaatgatggcaacagcgt

SEQ ID NO: 327 - DNA, 26 bp, Artificial Sequence
  Table 14 primer #14
acttgtagctatgatgggtataggat

SEQ ID NO: 328 - DNA, 26 bp, Artificial Sequence
  Table 14 primer #15
tcttgtagctatgatgggtataggat

SEQ ID NO: 329 - DNA, 26 bp, Artificial Sequence
  Table 14 primer #16
attcatattaatgatggctacagcgt

SEQ ID NO: 330 - DNA, 26 bp, Artificial Sequence
  Table 14 primer #17
atttatattaatgatggctacagcat

SEQ ID NO: 331 - DNA, 26 bp, Artificial Sequence
  Table 14 primer #18
atttatattaatgatggctacagcat

SEQ ID NO: 332 - DNA, 26 bp, Artificial Sequence
  Table 14 primer #19
ctttatattaatgatggctactgcat

SEQ ID NO: 333 - DNA, 26 bp, Artificial Sequence
  Table 14 primer #20
attcatattaatgatggctactgcat

SEQ ID NO: 334 - DNA, 26 bp, Artificial Sequence
  Table 14 primer #21
ttttattttaatgatggcaacagcgt

SEQ ID NO: 335 - DNA, 26 bp, Artificial Sequence
  Table 14 primer #22
ctttatcttaatgatggctacagcgt

SEQ ID NO: 336 - DNA, 26 bp, Artificial Sequence
  Table 14 primer #23
tttagttcttatgatggcaatagcgt

SEQ ID NO: 337 - DNA, 26 bp, Artificial Sequence
  Table 14 primer #24
gcttgttatgatgatggggatcgcgt

SEQ ID NO: 338 - DNA, 26 bp, Artificial Sequence
  Table 14 primer #25
ctttatattaatgatagttacagcgt

SEQ ID NO: 339 - DNA, 26 bp, Artificial Sequence
  Table 14 primer #26
ttttattttaatgatggctactgcgt

SEQ ID NO: 340 - DNA, 26 bp, Artificial Sequence
  Table 14 primer #27
attagttctaatgatggctaccgcgt

SEQ ID NO: 341 - DNA, 26 bp, Artificial Sequence
  Table 14 primer #28
ctttatcttaatgatggctacagcgt

SEQ ID NO: 342 - DNA, 26 bp, Artificial Sequence
  Table 14 primer #29
ctttatcttaatgatggctacagcgt

SEQ ID NO: 343 - DNA, 26 bp, Artificial Sequence
  Table 14 primer #30
ctttatcttaatgatggctacagcgt

SEQ ID NO: 344 - DNA, 25 bp, Artificial Sequence
  Table 15 primer #1
tccccaaggcaaaacataacccaga

SEQ ID NO: 345 - DNA, 26 bp, Artificial Sequence
  Table 15 primer #2
tcatattaatgatcgttactgcatgc

SEQ ID NO: 346 - DNA, 26 bp, Artificial Sequence
  Table 15 primer #3
tcatattaatgatcgttacagcatcc

SEQ ID NO: 347 - DNA, 26 bp, Artificial Sequence
  Table 15 primer #4
ttatcttaatgatggctacagcctgc

SEQ ID NO: 348 - DNA, 26 bp, Artificial Sequence
  Table 15 primer #5
agttagccggaacttagacagcctgc

SEQ ID NO: 349 - DNA, 26 bp, Artificial Sequence
  Table 15 primer #6
tagttctgatgatggcaaccgcatcc

SEQ ID NO: 350 - DNA, 26 bp, Artificial Sequence
  Table 15 primer #7
tagttctaatgatggcaactgcctgt

SEQ ID NO: 351 - DNA, 26 bp, Artificial Sequence
  Table 15 primer #8
tcatattaatgatcgttactgcatgc

SEQ ID NO: 352 - DNA, 26 bp, Artificial Sequence
  Table 15 primer #9
tcattttaatgatggctacagcatcc

SEQ ID NO: 353 - DNA, 26 bp, Artificial Sequence
  Table 15 primer #10
ttgtagctatgatgggtataggttgc

SEQ ID NO: 354 - DNA, 26 bp, Artificial Sequence
  Table 15 primer #11
ttgtagctatgatgggtataggttgc

SEQ ID NO: 355 - DNA, 26 bp, Artificial Sequence
  Table 15 primer #12
ttgtagctatgatgggtataggttgc

SEQ ID NO: 356 - DNA, 25 bp, Artificial Sequence
  Table 15 primer #13
accccaaggtaatacataacccttg

SEQ ID NO: 357 - DNA, 26 bp, Artificial Sequence
  Table 15 primer #14
ttgtagctatgatgggtataggttgc

SEQ ID NO: 358 - DNA, 26 bp, Artificial Sequence
  Table 15 primer #15
tcatattaatgatggctacagcatac

SEQ ID NO: 359 - DNA, 26 bp, Artificial Sequence
  Table 15 primer #16
ttatattaatgatggctacagcttac

SEQ ID NO: 360 - DNA, 26 bp, Artificial Sequence
  Table 15 primer #17
ttatattaatgatggctacagcttac

SEQ ID NO: 361 - DNA, 26 bp, Artificial Sequence
  Table 15 primer #18
ttatattaatgatggctactgcctgt

SEQ ID NO: 362 - DNA, 26 bp, Artificial Sequence
  Table 15 primer #19
tcatattaatgatggctactgcatac

SEQ ID NO: 363 - DNA, 26 bp, Artificial Sequence
  Table 15 primer #20
ttattttaatgatggcaacagcttgt

SEQ ID NO: 364 - DNA, 26 bp, Artificial Sequence
  Table 15 primer #21
ttatcttaatgatggctacagcttac

SEQ ID NO: 365 - DNA, 26 bp, Artificial Sequence
  Table 15 primer #22
tagttcttatgatggcaatagcctac

SEQ ID NO: 366 - DNA, 26 bp, Artificial Sequence
  Table 15 primer #23
ttgttatgatgatggggatcgcatct

SEQ ID NO: 367 - DNA, 26 bp, Artificial Sequence
  Table 15 primer #24
ttatattaatgatagttacagcctac

SEQ ID NO: 368 - DNA, 26 bp, Artificial Sequence
  Table 15 primer #25
ttattttaatgatggctactgcttgt

SEQ ID NO: 369 - DNA, 26 bp, Artificial Sequence
  Table 15 primer #26
tagttctaatgatggctaccgcctgt

SEQ ID NO: 370 - DNA, 26 bp, Artificial Sequence
  Table 15 primer #27
ttatcttaatgatggctacagcttac

SEQ ID NO: 371 - DNA, 26 bp, Artificial Sequence
  Table 15 primer #28
ttatcttaatgatggctacagcttac

SEQ ID NO: 372 - DNA, 26 bp, Artificial Sequence
  Table 15 primer #29
ttatcttaatgatggctacagcttac

SEQ ID NO: 373 - DNA, 26 bp, Artificial Sequence
  Table 16 primer #1
catatttttaggggtttgtattacgg

SEQ ID NO: 374 - DNA, 29 bp, Artificial Sequence
  Table 16 primer #2
aacacctaaaggattaccagatcctgcac

SEQ ID NO: 375 - DNA, 29 bp, Artificial Sequence
  Table 16 primer #3
tacacctaaaggattacctgaccctgcac

SEQ ID NO: 376 - DNA, 40 bp, Artificial Sequence
  Table 16 primer #4
ttacagagaaaccacctcaaatgaactcaactatgtccac

SEQ ID NO: 377 - DNA, 40 bp, Artificial Sequence
  Table 16 primer #5
tttttattatacttttgttaaacagtcttttattgtttaa

SEQ ID NO: 378 - DNA, 29 bp, Artificial Sequence
  Table 16 primer #6
aaatccacctcatacgaattcaactatgt

SEQ ID NO: 379 - DNA, 29 bp, Artificial Sequence
  Table 16 primer #7
aaacctcctcaaataaactcaactatatc

SEQ ID NO: 380 - DNA, 40 bp, Artificial Sequence
  Table 16 primer #8
taactgagaaaccccctcagagaaactccacaatatcttg

SEQ ID NO: 381 - DNA, 40 bp, Artificial Sequence
  Table 16 primer #9
ttacagaaaaaccacctcaaagaaactccacgatatcttg

SEQ ID NO: 382 - DNA, 40 bp, Artificial Sequence
  Table 16 primer #10
taactgagaaacctcctcaaacgaattcaacaatatcttg

SEQ ID NO: 383 - DNA, 40 bp, Artificial Sequence
  Table 16 primer #11
taacagagaaacctcctcaaacgaattcaacaatatcttg

SEQ ID NO: 384 - DNA, 40 bp, Artificial Sequence
  Table 16 primer #12
taacagagaaacctcctcaaacgaactcaacaatatcttg

SEQ ID NO: 385 - DNA, 40 bp, Artificial Sequence
  Table 16 primer #13
tatttttaattgtagtcttgcctttcctcggagaggacaa

SEQ ID NO: 386 - DNA, 28 bp, Artificial Sequence
  Table 16 primer #14 -forward
tatattatggttcatatattactccaag

SEQ ID NO: 387 - DNA, 40 bp, Artificial Sequence
  Table 16 primer #15
taacagagaaacctcctcaaacgaattcaactatatcttg

SEQ ID NO: 388 - DNA, 40 bp, Artificial Sequence
  Table 16 primer #16
taacagagaaacctcctcaaacgaactcaacgatatcttg

SEQ ID NO: 389 - DNA, 40 bp, Artificial Sequence
  Table 16 primer #17
ttacagaaaaacctcctcaaagaaactccacgatatcttg

SEQ ID NO: 390 - DNA, 40 bp, Artificial Sequence
  Table 16 primer #18
ttacagagaaaccacctcaaataaattcaactatatcttg

SEQ ID NO: 391 - DNA, 40 bp, Artificial Sequence
  v19
ttacagagaaacctcctcaaataaattcaactatatcttg

SEQ ID NO: 392 - DNA, 40 bp, Artificial Sequence
  Table 16 primer #20
taacactgaaacctcctcaaataaattcaactacatcttg

SEQ ID NO: 393 - DNA, 40 bp, Artificial Sequence
  Table 16 primer #21
ttacagaaaaacctcctcaaataaactcgacgatatcttg

SEQ ID NO: 394 - DNA, 40 bp, Artificial Sequence
  Table 16 primer #22
ctacagcaaaaccgccccacaaccaatcaacaatatcttt

SEQ ID NO: 395 - DNA, 40 bp, Artificial Sequence
  Table 16 primer #23
taacactgaaacctcctcaaatgaactcaacaatatcttg

SEQ ID NO: 396 - DNA, 40 bp, Artificial Sequence
  Table 16 primer #24
aaacagagaaacctcctcatataaattcaactaaatcttg

SEQ ID NO: 397 - DNA, 40 bp, Artificial Sequence
  Table 16 primer #25
acacggaaaagccaccccagattaactctacaaaatcttg

SEQ ID NO: 398 - DNA, 40 bp, Artificial Sequence
  Table 16 primer #26
tcactgaaaagcctccccacagaaattcgactatatcttg

SEQ ID NO: 399 - DNA, 40 bp, Artificial Sequence
  Table 16 primer #27
ttggtccactaatagcctttcaactacagcttggtataag

SEQ ID NO: 400 - DNA, 40 bp, Artificial Sequence
  Table 16 primer #28
caacagcaaaacctccccataaccaatcaacaatatcttt

SEQ ID NO: 401 - DNA, 40 bp, Artificial Sequence
  Table 16 primer #29
taacagaaaacccacctcaaataaattcaactatatcttg

SEQ ID NO: 402 - DNA, 40 bp, Artificial Sequence
  Table 16 primer #30
taacgctgaaacctcctcatatgaactcaacaatatcttg

SEQ ID NO: 403 - DNA, 40 bp, Artificial Sequence
  Table 16 primer #31
taactgagaaaccacctcaaatgaactcaacgatatcttg

SEQ ID NO: 404 - DNA, 40 bp, Artificial Sequence
  Table 16 primer #32
taacagaaaaacctcctcatatgaactcaacaatatcttg

SEQ ID NO: 405 - DNA, 25 bp, Artificial Sequence
  Table 17 control primer #1
gtccccaaggcaaaacataacccat

SEQ ID NO: 406 - DNA, 24 bp, Artificial Sequence
  Table 17 control primer #2
attcatattaatgatcgttactgc

SEQ ID NO: 407 - DNA, 26 bp, Artificial Sequence
  Table 17 control primer #3
atattcatattaatgatcgttacagc

SEQ ID NO: 408 - DNA, 26 bp, Artificial Sequence
  Table 17 control primer #4
atctttatcttaatgatggctacagc

SEQ ID NO: 409 - DNA, 26 bp, Artificial Sequence
  Table 17 control primer #5
ttgtagttagccggaacttagacagc

SEQ ID NO: 410 - DNA, 26 bp, Artificial Sequence
  Table 17 control primer #6
atactagttctgatgatggcaaccgc

SEQ ID NO: 411 - DNA, 26 bp, Artificial Sequence
  Table 17 control primer #7
atattagttctaatgatggcaactgc

SEQ ID NO: 412 - DNA, 26 bp, Artificial Sequence
  Table 17 control primer #8
atattcatattaatgatcgttactgc

SEQ ID NO: 413 - DNA, 26 bp, Artificial Sequence
  Table 17 control primer #9
atattcattttaatgatggctacagc

SEQ ID NO: 414 - DNA, 26 bp, Artificial Sequence
  Table 17 control primer #10
atacttgtagctatgatgggtatagg

SEQ ID NO: 415 - DNA, 26 bp, Artificial Sequence
  Table 17 control primer #11
atacttgtagctatgatgggtatagg

SEQ ID NO: 416 - DNA, 26 bp, Artificial Sequence
  Table 17 control primer #12
atacttgtagctatgatgggtatagg

SEQ ID NO: 417 - DNA, 25 bp, Artificial Sequence
  Table 17 control primer #13
ttgaccccaaggtaatacataaccc

SEQ ID NO: 418 - DNA, 26 bp, Artificial Sequence
  Table 17 control primer #14
atacttgtagctatgatgggtatagg

SEQ ID NO: 419 - DNA, 26 bp, Artificial Sequence
  Table 17 control primer #15
attcttgtagctatgatgggtatagg

SEQ ID NO: 420 - DNA, 26 bp, Artificial Sequence
  Table 17 control primer #16
atattcatattaatgatggctacagc

SEQ ID NO: 421 - DNA, 26 bp, Artificial Sequence
  Table 17 control primer #17
atatttatattaatgatggctacagc

SEQ ID NO: 422 - DNA, 26 bp, Artificial Sequence
  Table 17 control primer #18
atatttatattaatgatggctacagc

SEQ ID NO: 423 - DNA, 26 bp, Artificial Sequence
  Table 17 control primer #19
atctttatattaatgatggctactgc

SEQ ID NO: 424 - DNA, 26 bp, Artificial Sequence
  Table 17 control primer #20
atattcatattaatgatggctactgc

SEQ ID NO: 425 - DNA, 26 bp, Artificial Sequence
  Table 17 control primer #21
atttttattttaatgatggcaacagc

SEQ ID NO: 426 - DNA, 26 bp, Artificial Sequence
  Table 17 control primer #22
atctttatcttaatgatggctacagc

SEQ ID NO: 427 - DNA, 26 bp, Artificial Sequence
  Table 17 control primer #23
attttagttcttatgatggcaatagc

SEQ ID NO: 428 - DNA, 26 bp, Artificial Sequence
  Table 17 control primer #24
atgcttgttatgatgatggggatcgc

SEQ ID NO: 429 - DNA, 26 bp, Artificial Sequence
  Table 17 control primer #25
atctttatattaatgatagttacagc

SEQ ID NO: 430 - DNA, 26 bp, Artificial Sequence
  Table 17 control primer #26
atttttattttaatgatggctactgc

SEQ ID NO: 431 - DNA, 26 bp, Artificial Sequence
  Table 17 control primer #27
atattagttctaatgatggctaccgc

SEQ ID NO: 432 - DNA, 26 bp, Artificial Sequence
  Table 17 control primer #28
atctttatcttaatgatggctacagc

SEQ ID NO: 433 - DNA, 26 bp, Artificial Sequence
  Table 17 control primer #29
atctttatcttaatgatggctacagc

SEQ ID NO: 434 - DNA, 26 bp, Artificial Sequence
  Table 17 control primer #30
atctttatcttaatgatggctacagc

SEQ ID NO: 435 - DNA, 21 bp, Artificial Sequence
  Primer 17F
aaataacggttggttaattcg

SEQ ID NO: 436 - DNA, 21 bp, Artificial Sequence
  Primer 15R
tcttaaaattgcataaaaagg

SEQ ID NO: 437 - DNA, 27 bp, Artificial Sequence
  primer Pt129-1
tttattttaatgatggcaacagcttac

SEQ ID NO: 438 - DNA, 27 bp, Artificial Sequence
  primer Pt129-2
tttattttaatgatggcaacagcttcc

SEQ ID NO: 439 - DNA, 27 bp, Artificial Sequence
  primer Pt129-3
tttattttaatgatggcaacagcttgc

SEQ ID NO: 440 - DNA, 27 bp, Artificial Sequence
  primer Pt129-4
tttattttaatgatggcaacagcttaa

SEQ ID NO: 441 - DNA, 27 bp, Artificial Sequence
  primer Pt129-5
tttattttaatgatggcaacagcttca

SEQ ID NO: 442 - DNA, 27 bp, Artificial Sequence
  primer Pt129-6
tttattttaatgatggcaacagcttga

SEQ ID NO: 443 - DNA, 27 bp, Artificial Sequence
  primer Pt129-S
tatttttattttaatgatggcaacagc

SEQ ID NO: 444 - DNA, 25 bp, Artificial Sequence
  Table 16 primer #14 - reverse
tatttaaagttggattatctacagc

SEQ ID NO: 445 - DNA, 59 bp, Artificial Sequence
  P. aphanidermatum Scorpion primer
cccgcccgatattgttgattggttatggggcgggtatttaaagttggattatctacagc

SEQ ID NO: 446 - DNA, 25 bp, Artificial Sequence
  primer Pt129-C1
accccaaggtaatacataacccaag

SEQ ID NO: 447 - DNA, 25 bp, Artificial Sequence
  primer Pt129-C2
accccaaggtaatacataacccacg

SEQ ID NO: 448 - DNA, 25 bp, Artificial Sequence
  primer Pt129-C3
accccaaggtaatacataacccagg

SEQ ID NO: 449 - DNA, 25 bp, Artificial Sequence
  primer Pt129-C4
accccaaggtaatacataacccttg

SEQ ID NO: 450 - DNA, 25 bp, Artificial Sequence
  primer Pt129-C5
accccaaggtaatacataacccctg

SEQ ID NO: 451 - DNA, 25 bp, Artificial Sequence
  primer Pt129-C6
accccaaggtaatacataacccgtg

SEQ ID NO: 452 - DNA, 25 bp, Artificial Sequence
  primer Pt129-A1
accccaaggtaatacataacccaat

SEQ ID NO: 453 - DNA, 25 bp, Artificial Sequence
  primer Pt129-A2
accccaaggtaatacataacccact

SEQ ID NO: 454 - DNA, 25 bp, Artificial Sequence
  primer Pt129-A3
accccaaggtaatacataacccagt

SEQ ID NO: 455 - DNA, 25 bp, Artificial Sequence
  primer Pt129-A4
accccaaggtaatacataacccttt

SEQ ID NO: 456 - DNA, 25 bp, Artificial Sequence
  primer Pt129-A5
accccaaggtaatacataacccctt

SEQ ID NO: 457 - DNA, 25 bp, Artificial Sequence
  primer Pt129-A6
accccaaggtaatacataacccgtt

SEQ ID NO: 458 - DNA, 25 bp, Artificial Sequence
  primer Pt129-A7
accccaaggtaatacataactcact

SEQ ID NO: 459 - DNA, 25 bp, Artificial Sequence
  primer Pt129-A8
accccaaggtaatacataacacact

SEQ ID NO: 460 - DNA, 25 bp, Artificial Sequence
  primer Pt129-A9
accccaaggtaatacataacgcact

SEQ ID NO: 461 - DNA, 25 bp, Artificial Sequence
  primer Pt129-A10
accccaaggtaatacataactcctt

SEQ ID NO: 462 - DNA, 25 bp, Artificial Sequence
  primer Pt129-A11
accccaaggtaatacataacacctt

SEQ ID NO: 463 - DNA, 25 bp, Artificial Sequence
  primer Pt129-A12
accccaaggtaatacataacgcctt

SEQ ID NO: 464 - DNA, 25 bp, Artificial Sequence
  primer Pt129-A13
accccaaggtaatacataactcgtt

SEQ ID NO: 465 - DNA, 25 bp, Artificial Sequence
  primer Pt129-A14
accccaaggtaatacataacacgtt

SEQ ID NO: 466 - DNA, 25 bp, Artificial Sequence
  primer Pt129-A15
accccaaggtaatacataacgcgtt

SEQ ID NO: 467 - DNA, 25 bp, Artificial Sequence
  primer Pt129-S4
ttgaccccaaggtaatacataaccc

SEQ ID NO: 468 - DNA, 25 bp, Artificial Sequence
  primer Pt129-S6
ttgaccccaaggtaatacataactc

SEQ ID NO: 469 - DNA, 64 bp, Artificial Sequence
  P. aphanidermatum Scorpion primer from Example 7
cggccgccaacacctgaacaccataaaccgcggccgtatattatggttcatatattactccaag

SEQ ID NO: 470 - DNA, 27 bp, Artificial Sequence
  primer Pt129-7
tttttattttaatgatggcaacagcat

SEQ ID NO: 471 - DNA, 27 bp, Artificial Sequence
  primer Pt129-8
tttttattttaatgatggcaacagcgt

SEQ ID NO: 472 - DNA, 27 bp, Artificial Sequence
  primer Pt129-9
tttttattttaatgatggcaacagcct

SEQ ID NO: 473 - DNA, 27 bp, Artificial Sequence
  primer Pt129-10
tttttattttaatgatggcaacagcac

SEQ ID NO: 474 - DNA, 27 bp, Artificial Sequence
  primer Pt129-11
tttttattttaatgatggcaacagcgc

SEQ ID NO: 475 - DNA, 27 bp, Artificial Sequence
  primer Pt129-12
tttttattttaatgatggcaacagccc

SEQ ID NO: 476 - DNA, 27 bp, Artificial Sequence
  primer Pt129-S2
tatttttattttaatgatggcaacagc

SEQ ID NO: 477 - DNA, 25 bp, Artificial Sequence
  primer Pt129-13
accccaaggtaatacataacccaaa

SEQ ID NO: 478 - DNA, 25 bp, Artificial Sequence
  primer Pt129-14
accccaaggtaatacataacccaca

SEQ ID NO: 479 - DNA, 25 bp, Artificial Sequence
  primer Pt129-15
accccaaggtaatacataacccaga

SEQ ID NO: 480 - DNA, 25 bp, Artificial Sequence
  primer Pt129-A14
accccaaggtaatacataacacgtt

SEQ ID NO: 481 - DNA, 25 bp, Artificial Sequence
  primer Pt129-C4
accccaaggtaatacataacccttg

SEQ ID NO: 482 - DNA, 25 bp, Artificial Sequence
  primer Pt129-16
accccaaggtaatacataacccaac

SEQ ID NO: 483 - DNA, 25 bp, Artificial Sequence
  primer Pt129-17
accccaaggtaatacataacccacc

SEQ ID NO: 484 - DNA, 25 bp, Artificial Sequence
  primer Pt129-18
accccaaggtaatacataacccagc

SEQ ID NO: 485 - DNA, 25 bp, Artificial Sequence
  primer Pt129-S4
ttgaccccaaggtaatacataaccc

SEQ ID NO: 486 - DNA, 21 bp, Artificial Sequence
  Primer 17
aaataacggttggttaattcg

SEQ ID NO: 487 - DNA, 21 bp, Artificial Sequence
  Primer 15
tcttaaaattgcataaaaagg

SEQ ID NO: 488 - DNA, 25 bp, Artificial Sequence
  Primer PV129-T1
cccaaggcaaaacataacccatata

SEQ ID NO: 489 - DNA, 25 bp, Artificial Sequence
  Primer PV129-T2
cccaaggcaaaacataacccataca

SEQ ID NO: 490 - DNA, 25 bp, Artificial Sequence
  Primer PV129-T3
cccaaggcaaaacataacccataga

SEQ ID NO: 491 - DNA, 25 bp, Artificial Sequence
  Primer PV129-T4
cccaaggcaaaacataacccattaa

SEQ ID NO: 492 - DNA, 25 bp, Artificial Sequence
  Primer PV129-T5
cccaaggcaaaacataacccatcaa

SEQ ID NO: 493 - DNA, 25 bp, Artificial Sequence
  Primer PV129-T6
cccaaggcaaaacataacccatgaa

SEQ ID NO: 494 - DNA, 25 bp, Artificial Sequence
  Primer PV129-C1
cccaaggcaaaacataacccatatg

SEQ ID NO: 495 - DNA, 25 bp, Artificial Sequence
  Primer PV129-C2
cccaaggcaaaacataacccatacg

SEQ ID NO: 496 - DNA, 25 bp, Artificial Sequence
  Primer PV129-C3
cccaaggcaaaacataacccatagg

SEQ ID NO: 497 - DNA, 25 bp, Artificial Sequence
  Primer PV129-C4
cccaaggcaaaacataacccattag

SEQ ID NO: 498 - DNA, 25 bp, Artificial Sequence
  Primer PV129-C5
cccaaggcaaaacataacccatcag

SEQ ID NO: 499 - DNA, 25 bp, Artificial Sequence
  Primer PV129-C6
cccaaggcaaaacataacccatgag

SEQ ID NO: 500 - DNA, 25 bp, Artificial Sequence
  Primer PV129-S
gtccccaaggcaaaacataacccat

SEQ ID NO: 501 - DNA, 63 bp, Artificial Sequence
  P. viticola Scorpion primer Example 13
ccgcgcgccataaagcttctctaggtgtaacgcgcggcatatttttaggggtttgtattacgg

SEQ ID NO: 502 - DNA, 63 bp, Artificial Sequence
  P. viticola Scorpion primer Example 15
ccggcccccataaagcttctctaggtgtaagggccggcatatttttaggggtttgtattacgg

SEQ ID NO: 503 - DNA, 25 bp, Artificial Sequence
  Primer PV129-1
tccccaaggcaaaacataacccaaa

SEQ ID NO: 504 - DNA, 25 bp, Artificial Sequence
  Primer PV129-2
tccccaaggcaaaacataacccaca

SEQ ID NO: 505 - DNA, 25 bp, Artificial Sequence
  Primer PV129-3
tccccaaggcaaaacataacccaga

SEQ ID NO: 506 - DNA, 25 bp, Artificial Sequence
  Primer PV129-4
tccccaaggcaaaacataacccaat

SEQ ID NO: 507 - DNA, 25 bp, Artificial Sequence
  Primer PV129-5
tccccaaggcaaaacataacccact

SEQ ID NO: 508 - DNA, 25 bp, Artificial Sequence
  Primer PV129-6
tccccaaggcaaaacataacccagt

SEQ ID NO: 509 - DNA, 25 bp, Artificial Sequence
  Primer PV129-7
tccccaaggcaaaacataacccaag

SEQ ID NO: 510 - DNA, 25 bp, Artificial Sequence
  Primer PV129-8
tccccaaggcaaaacataacccacg

SEQ ID NO: 511 - DNA, 25 bp, Artificial Sequence
  Primer PV129-9
tccccaaggcaaaacataacccagg

SEQ ID NO: 512 - DNA, 25 bp, Artificial Sequence
  Primer PV129-10
tccccaaggcaaaacataacccaac

SEQ ID NO: 513 - DNA, 25 bp, Artificial Sequence
  Primer PV129-11
tccccaaggcaaaacataacccacc

SEQ ID NO: 514 - DNA, 25 bp, Artificial Sequence
  Primer PV129-12
tccccaaggcaaaacataacccagc

SEQ ID NO: 515 - DNA, 25 bp, Artificial Sequence
  Primer PV129-S2
ttgtccccaaggcaaaacataaccc

SEQ ID NO: 516 - DNA, 66 bp, Artificial Sequence
  P. viticola Scorpion primer Example 17
cccgccgtaattgtaggggctgtactaatacggcggggatacctaatggattatttgaacctacct

SEQ ID NO: 517 - DNA, 27 bp, Artificial Sequence
  Primer PV129-13
tttttattttaatgatggcgactgctt

SEQ ID NO: 518 - DNA, 27 bp, Artificial Sequence
  Primer PV129-14
tttttattttaatgatggcgactgcct

SEQ ID NO: 519 - DNA, 27 bp, Artificial Sequence
  Primer PV129-15
tttttattttaatgatggcgactgcgt

SEQ ID NO: 520 - DNA, 27 bp, Artificial Sequence
  Primer PV129-16
tttttattttaatgatggcgactgctc

SEQ ID NO: 521 - DNA, 27 bp, Artificial Sequence
  Primer PV129-17
tttttattttaatgatggcgactgccc

SEQ ID NO: 522 - DNA, 27 bp, Artificial Sequence
  Primer PV129-18
tttttattttaatgatggcgactgcgc

SEQ ID NO: 523 - DNA, 27 bp, Artificial Sequence
  Primer PV129-S3
tatttttattttaatgatggcgactgc

SEQ ID NO: 524 - DNA, 30 bp, Artificial Sequence
  Forward primer Example 18
cggatcttatattacacctagagaagcttt

SEQ ID NO: 525 - DNA, 20 bp, Artificial Sequence
  Reverse primer Example 18
ttgtccccaaggcaaaacat

SEQ ID NO: 526 - DNA, 17 bp, Artificial Sequence
  Mutant probe (L129 allele specific) example 18
aacccataagtgcagtc

SEQ ID NO: 527 - DNA, 17 bp, Artificial Sequence
  Wild type probe (F129 allele specific) example 18
acccataaatgcagtcg

SEQ ID NO: 528 - DNA, 24 bp, Artificial Sequence
  Forward primer example 19
ctgttatctttatcttaatgatgg

SEQ ID NO: 529 - DNA, 24 bp, Artificial Sequence
  Reverse primer example 19
ggaatagatcttaatatagcatag

SEQ ID NO: 530 - DNA, 634 bp, Pythium aphanidermatum
aaataacggttggttaattcgatatacacatgcaaatggtgcatcttttttttttatagttgtttatatccatatttttagaggtttatattatggttcatatattactccaagagaaggtttatggtgttcaggtgttgttatttttattttaatgatggcaacagctttaatgggttatgtattaccttggggtcaaatgagtttttggggtgctactgttattactaatttattttcagcagtacctttagtaggtaaagatattgttgattggttatgggggggatttgctgtagataatccaactttaaatagattttttagtttacattttacaatgccttttgtaataacaggtgctgtattaattcatttagttttattacatgaagttggttctaataatcctttaggtataactttaaaaacagaaaatgtacctttttatccttatttttatactaaagatttatttggaataatggttttattcttagtgttttttacttttattttttattatcctaacacattaggacacccagataattatattgaagcaaatcctatgaaaacaccattacatattgtacctgaatggtattttttacctttttatgcaattttaaga

SEQ ID NO: 531 - DNA, 634 bp, Pythium aphanidermatum; identical to SEQ ID NO: 530

SEQ ID NO: 532 - DNA, 634 bp, Pythium aphanidermatum
aaataacggttggttaattcgatatacacatgcaaatggtgcatcttttttttttatagttgtttatatccatatttttagaggtttatattatggttcatatattactccaagagaaggtttatggtgttcaggtgttgttatttttattttaatgatggcaacagctttcatgggttatgtattaccttggggtcaaatgagtttttggggtgctactgttattactaatttattttcagcagtacctttagtaggtaaagatattgttgattggttatgggggggatttgctgtagataatccaactttaaatagattttttagtttacattttacaatgccttttgtaataacaggtgctgtattaattcatttagttttattacatgaagttggttctaataatcctttaggtataactttaaaaacagaaaatgtacctttttatccttatttttatactaaagatttatttggaataatggttttattcttagtgttttttacttttattttttattatcctaacacattaggacacccagataattatattgaagcaaatcctatgaaaacaccattacatattgtacctgaatggtattttttacctttttatgcaattttaaga

SEQ ID NO: 533 - DNA, 592 bp, Plasmopara viticola
atatatacatgcgaatggtgcatcttttttttttattgttgtatatatacatatttttaggggtttgtattacggatcttatattacacctagagaagctttatggtgttcaggggtaattatttttattttaatgatggcgactgcatttatgggttatgttttgccttggggacaaatgagtttttggggtgcaacagttattacaaatttattctcggctatcccattaattggaaaagaagttgttgactggttatggggtggattcgccgttgataatccaacattaaatcgtttttttagtttacatttcacctttccatttgtaattgtaggggctgtactaatacatttaattttattacatgaggtaggttcaaataatccattaggtatcactttaaaaactgagaatataccattttatccttatttctatacaaaagatttatttggtttaatcgtattatttttaattttttttatatttattttttactaccctaatactttaggtcatccggataattatattgaagcaaatcctatgaaaactcctttacatattgttcctgagtggtatttttta

SEQ ID NO: 534 - DNA, 592 bp, Plasmopara viticola
atatatacatgcgaatggtgcatcttttttttttattgttgtatatatacatatttttaggggtttgtattacggatcttatattacacctagagaagctttatggtgttcaggggtaattatttttattttaatgatggcgactgcatttatgggttatgttttgccttggggacaaatgagtttttgggctgcaacagttattacaaatttattctcggctatcccattaattggaaaagaagttgttgactggttatggggtggattcgccgttgataatccaacattaaatcgtttttttagtttacatttcacctttccatttgtaattgtaggggctgtactaatacatttaattttattacatgaggtaggttcaaataatccattaggtatcactttaaaaactgagaatataccattttatccttatttctatacaaaagatttatttggtttaatcgtattatttttaattttttttatatttattttttactaccctaatactttaggtcatccggataattatattgaagcaaatcctatgaaaactcctttacatattgttcctgagtggtatttttta

SEQ ID NO: 535 - DNA, 634 bp, Plasmopara viticola
aaataacggttggttaattcgatatatacatgcgaatggtgcatcttttttttttattgttgtatatatacatatttttaggggtttgtattacggatcttatattacacctagagaagctttatggtgttcaggggtaattatttttattttaatgatggcgactgcacttatgggttatgttttgccttggggacaaatgagtttttggggtgcaacagttattacaaatttattctcggctatcccattaattggaaaagaagttgttgactggttatggggtggattcgccgttgataatccaacattaaatcgtttttttagtttacatttcacctttccatttgtaattgtaggggctgtactaatacatttaattttattacatgaggtaggttcaaataatccattaggtatcactttaaaaactgagaatataccattttatccttatttctatacaaaagatttatttggtttaatcgtattatttttaattttttttatatttattttttactaccctaatactttaggtcatccggataattatattgaagcaaatcctatgaaaactcctttacatattgttcctgagtggtattttttacctttttatgcaattttaaga

SEQ ID NO: 536 - DNA, 634 bp, Plasmopara viticola
aaataacggttggttaattcgatatatacatgcgaatggtgcatcttttttttttattgttgtatatatacatatttttaggggtttgtattacggatcttatattacacctagagaagctttatggtgttcaggggtaattatttttattttaatgatggcgactgcacttatgggttatgttttgccttggggacaaatgagtttttggggtgcaacagttattacaaatttattctcggctatcccattaattggaaaagaagttgttgactggttatggggtggattcgccgttgataatccaacattgaatcgtttttttagtttacatttcacctttccatttgtaattgtaggggctgtactaatacatttaattttattacatgaggtaggttcaaataatccattaggtatcactttaaaaactgagaatataccatcttatccttatttctatacaaaagatttatttggtttaatcgtattatttttaattttttttatatttattttttactaccctaatactttaggtcatccggataattatattgaagcaaatcctatgaaaactcctttacatattgttcctgagtggtattttttacctttttatgcaattttaaga

SEQ ID NO: 537 - DNA, 634 bp, Plasmopara viticola; identical to SEQ ID NO: 535

SEQ ID NO: 538 - DNA, 634 bp, Plamsopara viticola
aaataacggttggttaattcgatatatacatgcgaatggtgcatcttttttttttattgttgtatatatacatatttttaggggtttgtattacggatcttatattacacctagagaagctttatggtgttcaggggtaattatttttattttaatgatggcgactgcacttatgggttatgttttgccttggggacaaatgagtttttggggtgcaacagttattacaaatttattctcggctatcccattaattggaaaagaagttgttgactggttatggggtggattcgccgttgataatccaacattaaatcgtttttttagtttacatttcacctttccatttgtaattgtaggggctgtactaatacatttaattttatkacatgaggtaggttcaaataatccattaggtatcactttaaaamctgagaatataccattttatccttatttctatacaaaagatttatttggtttaatcgtattatttttaattttttttatatttattttttactmccctaatactttaggtcatccggataattatattgaagcaratcctatgaaaactcctttacatattgttcctgagtggtattttttacctttttatgcaattttaaga

SEQ ID NO: 539 - DNA, 634 bp, Plasmopara viticola
aaataacggttggttaattcgatatatacatgcgaatggtgcatcttttttttttattgttgtatatatacatatttttaggggtttgtattacggatcttatattacacctagagaagctttatggtgttcaggggtaattatttttattttaatgatggcgactgcatttatgggttatgttttgccttggggacaaatgagtttttggggtgcaacagttattacaaatttattctcggctatcccattaattggaaaagaagttgttgactggttatggggtggattcgccgttgataatccaacattaaatcgtttttttagtttacatttcacctttccatttgtaattgtaggggctgtactaatacatttaattttattacatgaggtaggttcaaataatccattaggtatcactttaaaaactgagaatataccattttatccttatttctatacaaaagatttatttggtttaatcgtattatttttaattttttttatatttattttttactaccctaatactttaggtcatccggataattatattgaagcaaatcctatgaaaactcctttacatattgttcctgagtggtattttttacctttttatgcaattttaaga

SEQ ID NO: 540 - DNA, 574 bp, Plasmopara viticola
aaataacggttggttaattcgatatatacatgcgaatggtgcatcttttttttttattgttgtatatatacatatttttaggggtttgtattacggatcttatattacacctagagaagctttatggtgttcaggggtaattatttttattttaatgatggcgactgcatttatgggttatgttttgccttggggacaaatgagttyttggggtgcaacagttattacaaakttattstcggctatcccattaattggaaaagaagktgwtgactggttatggggtggattcgccgttgataatccaacattaaatcgyttttttagttyacatttcacctttccatttgtaattgkaggggctgtactaatacatttaattttattacatgaggtaggttcaaataatccattaggtatcactttaaaaactgagaatataccattttatccttatttctatacaaaagatttatttggtttaatsgtattatttttaattttttttatatttattttttactaccctaatactttaggtcatccggataattatattgaagcagatcctatg

SEQ ID NO: 541 - protein, 197 aa, Plasmopara viticola
YIHANGASFFFIVVYIHIFRGLYYGSYITPREALWCSGVIIFILMMATAFMGYVLPWGQMSFWAATVITNLFSAIPLIGKEVVDWLWGGFAVDNPTLNRFFSLHFTFPFVIVGAVLIHLILLHEVGSNNPLGITLKTENIPFYPYFYTKDLFGLIVLFLIFFIFIFYYPNTLGHPDNYIEANPMKTPLHIVPEWYFL

SEQ ID NO: 542 - protein, 197 aa, Plasmopara viticola
YIHANGASFFFIVVYIHIFRGLYYGSYITPREALWCSGVIIFILMMATAFMGYVLPWGQMSFWGATVITNLFSAIPLIGKEVVDWLWGGFAVDNPTLNRFFSLHFTFPFVIVGAVLIHLILLHEVGSNNPLGITLKTENIPFYPYFYTKDLFGLIVLFLIFFIFIFYYPNTLGHPDNYIEANPMKTPLHIVPEWYFL

SEQ ID NO: 543 - protein, 211 aa, Plasmopara viticola
NNGWLIRYIHANGASFFFIVVYIHIFRGLYYGSYITPREALWCSGVIIFILMMATALMGYVLPWGQMSFWGATVITNLFSAIPLIGKEVVDWLWGGFAVDNPTLNRFFSLHFTFPFVIVGAVLIHLILLHEVGSNNPLGITLKTENIPFYPYFYTKDLFGLIVLFLIFFIFIFYYPNTLGHPDNYIEANPMKTPLHIVPEWYFLPFYAILR

SEQ ID NO: 544 - protein, 211 aa, Plasmopara viticola
NNGWLIRYIHANGASFFFIVVYIHIFRGLYYGSYITPREALWCSGVIIFILMMATALMGYVLPWGQMSFWGATVITNLFSAIPLIGKEVVDWLWGGFAVDNPTLNRFFSLHFTFPFVIVGAVLIHLILLHEVGSNNPLGITLKTENIPSYPYFYTKDLFGLIVLFLIFFIFIFYYPNTLGHPDNYIEANPMKTPLHIVPEWYFLPFYAILR

SEQ ID NO: 545 - protein, 211 aa, Plasmopara viticola; identical to SEQ ID NO: 543

SEQ ID NO: 546 - protein, 211 aa, Plasmopara viticola
NNGWLIRYIHANGASFFFIVVYIHIFRGLYYGSYITPREALWCSGVIIFILMMATAFMGYVLPWGQMSFWGATVITNLFSAIPLIGKEVVDWLWGGFAVDNPTLNRFFSLHFTFPFVIVGAVLIHLILLHEVGSNNPLGITLKTENIPFYPYFYTKDLFGLIVLFLIFFIFIFYYPNTLGHPDNYIEANPMKTPLHIVPEWYFLPFYAILR

SEQ ID NO: 547 - DNA, 547 bp, Alternaria solani
cagagcacctagaactctagtatgaactattggtactgttatctttatcttaatgatggctacagctttcctgggttatgttcttccttatgggcaaatgtctttatgaggtgctacagttattactaaccttatgagtgctatcccttgagtaggacaagatattgttgagttcatttgaggaggtwtcagtgttaacaatgcaacattaaatagattcttctcattacatttcgttttacctttcgtattagctgctttagcactaatgcacttaatcgttttacacgatacagctggatcaggaaatcctttaggtgtatcaggaaactacgaaagaatatcttttgctccttatttcatatttaaagatcttattacaatatttgcatttatatttgtattatctttatttgtgttctttatgcctaatgtattaggagatagtgaaaactatgttgtggcaaaccctatgcaaactcctgcagctatcgtgccagaatgataccttctgcctttctatgctatattaagatctatacctaac

SEQ ID NO: 548 - DNA, 508 bp, Alternaria solani
ctgttatctttatcttaatgatggctacagctctcctgggttatgttcttccttatgggcaaatgtctttatgaggtgctacagttattactaaccttatgagtgctatcccttgagtaggacaagatattgttgagttcatttgaggaggtttcagtgttaacaatgcaacattaaatagattcttctcattacatttcgttttacctttcgtattagctgctttagcactaatgcacttaatcgttttacacgatacagctggatcaggaaatcctttaggtgtatcaggaaactacgaaagaatatcttttgctccttatttcatatttaaagatcttattacaatatttgcatttatatttgtattatctttatttgtgttctttatgcctaatgtattaggagatagtgaaaactatgttgtggcaaaccctatgcaaactcctgcagctatcgtgccagaatgataccttctgcctttctatgctatattaagatctattcc

SEQ ID NO: 549 - DNA, 508 bp, Alternaria solani
ctgttatctttatcttaatgatggctacagctttactgggttatgttcttccttatgggcaaatgtctttatgaggtgctacagttattactaaccttatgagtgctatcccttgagtaggacaagatattgttgagttcatttgaggaggtttcagtgttaacaatgcaacattaaatagattcttctcattacatttcgttttacctttcgtattagctgctttagcactaatgcacttaatcgttttacacgatacagctggatcaggaaatcctttaggtgtatcaggaaactacgaaagaatatcttttgctccttatttcatatttaaagatcttattacaatatttgcatttatatttgtattatctttatttgtgttctttatgcctaatgtattaggagatagtgaaaactatgttgtggcaaaccctatgcaaactcctgcagctatcgtgccagaatgataccttctgcctttctatgctatattaagatctattcc

SEQ ID NO: 550 - protein, 182 aa, Alternaria solani
  MISC_FEATURE (63)..(63): X denotes undetermined amino acid
RAPRTLVWTIGTVIFILMMATAFLGYVLPYGQMSLWGATVITNLMSAIPWVGQDIVEFIWGGXSVNNATLNRFFSLHFVLPFVLAALALMHLIVLHDTAGSGNPLGVSGNYERISFAPYFIFKDLITIFAFIFVLSLFVFFMPNVLGDSENYVVANPMQTPAAIVPEWYLLPFYAILRSIPN

SEQ ID NO: 551 - protein, 168 aa, Alternaria solani
VIFILMMATALLGYVLPYGQMSLWGATVITNLMSAIPWVGQDIVEFIWGGFSVNNATLNRFFSLHFVLPFVLAALALMHLIVLHDTAGSGNPLGVSGNYERISFAPYFIFKDLITIFAFIFVLSLFVFFMPNVLGDSENYVVANPMQTPAAIVPEWYLLPFYAILRSI

SEQ ID NO: 552 - protein, 168 aa, Alternaria solani; identical to SEQ ID NO: 551

SEQ ID NO: 553 - protein, 211 aa, Pythium aphanidermatum
NNGWLIRYTHANGASFFFIVVYIHIFRGLYYGSYITPREGLWCSGVVIFILMMATALMGYVLPWGQMSFWGATVITNLFSAVPLVGKDIVDWLWGGFAVDNPTLNRFFSLHFTMPFVITGAVLIHLVLLHEVGSNNPLGITLKTENVPFYPYFYTKDLFGIMVLFLVFFTFIFYYPNTLGHPDNYIEANPMKTPLHIVPEWYFLPFYAILR

SEQ ID NO: 554 - protein, 211 aa, Pythium aphanidermatum; identical to SEQ ID NO: 553

(19) United States
(12) Patent Application Publication
(10) Pub. No.: US 2005/0014144 A1
(43) Pub. Date: Jan. 20, 2005

(54) Methods

(76) Inventors: Judith Mary Burbidge, Berkshire (GB); Sally Michele Cleere, Berkshire (GB); Carole Patricia Stanger, Berkshire (GB); John David Windass, Berkshire (GB)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(21) Appl. No.: 10/483,979

(22) PCT Filed: Mar. 25, 2002
(86) PCT No.: PCT/GB02/01411

(30) Foreign Application Priority Data
Apr. 2, 2001 (GB) 0108227.0
Sep. 20, 2001 (GB) 0122697.6

(51) Int. Cl. C12Q 1/68

(52) U.S. Cl. 435/6

(57) ABSTRACT

This invention relates to disgnostic methods for the detection of one or more cytochrome b mutations in fungi at the position corresponding to Saccharomyces cerevisiae cytochrome b residue 129 that leads to resistance to strobilurin analogues or compounds in the same cross resistance group using any (or a) single nucleotide polymorphism detection technique, preferably using either an allele specific amplilication technique such as the amplification refractory mutation system (ARMS) or preferably using an allele selective hybridisation probe technique such as Molecular Beacons or TaqMan. The invention also relates to mutation specific oligonucleotides for use in the method and to diagnostic kits containing these (oligonucleotides.

### FIELD OF THE INVENTION

This invention relates to diagnostic methods for the detection of one or more cytochrome b mutations in fungi at the position corresponding to Saccharomyces cerevisiae cytochrome b residue 129 that leads to resistance to strobilurin analogues or compounds in the same cross resistance group using any (or a) single nucleotide polymorphism detection technique, preferably using either an allele specific amplification technique such as the amplification refractory mutation system (ARMS) or preferably using an allele selective hybridisation probe technique such as Molecular Beacons or TaqMan. The invention also relates to mutation specific oligonucleotides for use in the methods of the inventionand to diagnostic kits containing these oligonucleotides.

### BACKGROUND OF THE INVENTION

The widespread use of fungicides in agriculture is a relatively recent phenomenon, and most of the major developments have taken place during the last 40 years. Previously, farmers often ignored or did not recognise the effect that fungal pathogens had on the yield and quality of their crops. Nowadays, however these losses are unacceptable, and farmers rely on the use of fungicidal chemicals to control fungal diseases. As a consequence, commercial fungicides have become an important component of the total agrochemical business, with world-wide sales in 1996 of about $5.9 billion, equivalent to 18.9% of the total agrochemical market (Wood Mackenzie, 1997a ‘Agchem products—The key agrochemical product groups’, in Agrochemical Service, Update of the Products Section, May 1997, 1-74). A large number of fungicides are already available to the farmer; a recent edition of The Pesticide Manual (Tomlin, 1994 10^{th} Edition, British Crop Protection Council, Farnham, UK, and the Royal Society of Chemistry, Cambridge, UK) contains 158 different fungicidal active ingredients in current use. Nevertheless, further industrial research aimed at the discovery and development of new compounds is extremely intensive and product management procedures are extremely important in securing the best and longest lasting performance from fungicides with a particular mode of action and/or belonging to a particular compound series. In particular it is vital to develop effective resistance management strategies when fungicides with new modes of action are introduced (Fungicide Resistance Management: Into The Next Millenium (Russell) 1999, in Pesticide Outlook, October 1999 (213-215).

The strobilurin analogues constitute a major new series of agricultural fungicides, which are considered the most exciting development on the agricultural fungicide scene since the discovery of the 1,2,4-triazoles in the 1970s.

The fungicidal activity of the strobilurin analogues is a result of their ability to inhibit mitochondrial respiration in fungi. More specifically, it has been established that these compounds have a novel single site mode of action, exerting their effect on fungi by blocking the ubiquinol:cytochrome c oxidoreductase complex (cytochrome bc1) thus reducing the generation of energy rich ATP in the fungal cell (Becker et al 1981 FEBS Letts. 132: 329-33). This family of inhibitors prevents electron transfer at the ubiquinone redox site Qₒ on the multimeric cytochrome b protein (Esposti et al 1993 Biochim. et Biophys Acta 1143(3): 243-271). Unlike many mitochondrial proteins, the cytochrome b protein is mitochondrially encoded.

Reports in the literature show that specific amino acid changes at the cytochrome b target site can affect the activity of strobilurin analogues. In depth mutagenesis studies in Saccharomyces cerevisiae (hereinafter referred to as S. cerevisiae) (JP Rago et al 1989 J. Biol. Chem. 264:14543-14548), mouse (Howell et al 1988 J. Mol. Biol. 203:607-618), Chlamydomonas reinhardtii (Bennoun et al 1991 Genetics 127:335-343) and Rhodobacter spp (Daldal et al 1989 EMBO J. 8(13):3951-3961) have been carried out. Relevant information was also gathered from studying the natural basis for resistance to strobilurin analogues in the sea urchin Paracentrotus lividus (Esposti et al 1990 FEBS 263:245-247) and the Basidiomycete fungi Mycena galopoda and Strobilurus tenacellus (Kraiczy et al 1996 Eur. J. Biochem. 235:54-63), both of which produce natural variants of the strobilurin analogues. There are two distinct regions of the cytochrome b gene where amino acid changes have a dramatic effect on strobilurin analogue activity. These areas cover amino acid residues 125-148 and 250-295 (based on S. cerevisiae residue numbering system). More precisely amino acid changes at residues 126, 129, 132, 133, 137, 142, 143, 147, 148, 256, 275 and 295 have been shown to give rise to resistance to strobilurin analogues (Brasseur et al 1996 Biochim. Biophys. Acta 1275:61-69 and Esposti et al (1993) Biochimica et Biophysica Acta, 1143:243-271).

Published International Patent Application Number WO 00/66773 describes the identification of a mutation in a fungal cytochrome b gene resulting in a glycine to alanine replacement at the position corresponding to S. cerevisiae cytochrome b residue 143 (G₁₄₃A) in the encoded protein. The present invention identifies for the first time the key importance of a further mutation(s) in cytochrome b gene of field isolates of important plant pathogenic fungi showing resistance to a strobilurin analogue or a compound in the same cross resistance group.

### SUMMARY OF THE INVENTION

According to a first aspect of the invention we now provide a method for the detection of the presence or absence of one or more mutations in a fungal cytochrome b gene resulting in an amino acid replacement at the position corresponding to S. cerevisiae cytochrome b residue 129 in the encoded protein wherein the presence of said mutation(s) gives rise to fungal resistance to a strobilurin analogue or any other compound in the same cross resistance group said method comprising identifying the presence or absence of said mutation(s) in fungal nucleic acid using any (or a) single nucleotide polymorphism detection technique.

According to a preferred embodiment of the first aspect of the invention we now provide a method for the detection of the presence or absence of one or more mutations in a fungal cytochrome b gene resulting in a phenyalanine to leucine replacement at the position corresponding to S. cerevisiae cytochrome b residue 129 in the encoded protein wherein the presence of said mutation(s) gives rise to fungal resistance to a strobilurin analogue or any other compound in the same cross resistance group said method comprising identifying the presence or absence of said mutation(s) in fungal nucleic acid using any (or a) single nucleotide polymorphism detection technique.

In the present invention we have now devised diagnostic methods for the detection of one or more point mutations in a fungal cytochrome b gene based on single nucleotide polymorphism detection methods including allele specific amplification. It will be apparent to the person skilled in the art that there are a large number of analytical procedures which may be used to detect the presence or absence of variant nucleotides at one or more polymorphic positions according to the invention. In general, the detection of allelic variation requires a mutation discrimination technique, optionally an amplification reaction and optionally a signal generation system. Many current methods for the detection of allelic variation are reviewed by Nollau et al, Clin. Chem. 43:1114-1120, 1997 and in standard textbooks, for example ‘Laboratory Protocols for Mutation Detection’, Ed, by U. Landegren, Oxford University Press, 1996 and ‘PCR’ 2^{nd} Edition by Newton and Graham, BIOS Scientific Publishers limited, 1997. Allele specific amplification reactions include primer based methods such as PCR based methods and more specifically, allele specific polymerase chain reaction (PCR) extension (ASPCR). One such ASPCR based method is ARMS (Amplification Refractory Mutagenesis System). The technique of ASPCR is described in U.S. Pat. No. 5,639,611 and the ARMS technique is described fully in European Patent No. EP 332435.

All such PCR based methods are suitable for use in methods of the current invention, and the use of ARMS based methods are particularly preferred. The methods of the invention also include the use of indiscriminate PCR followed by specific probing of the amplicon generated.

All of these methods are suitable for the detection of the specific alleles which can confer resistance to any of the strobilurin analogues or any other compound in the same cross resistance group and Robust tests have been developed for the detection of point mutations conferring such resistance iii a range of fungal plant pathogens Compounds may be considered to be in the same cross resistance group when the resistance mechanism to one compound also confers resistance to another, even-when the modes of action are not the same.

Other single nucleotide polymorphism (SNP) detection techniques which may be used in any aspect of the invention described herein to detect one or more mutation include, for example, restriction fragment length polymorphism (RFLP), single strand conformation polymorphism, multiple clonal analysis, allele-specific oligonucleotide hybridisation, single nucleotide primer extension (Juvonen et al, (1994) Hum Genet 93 16-20; Huoponen et al, (1994) Hum Mutat 3 29-36; Mashima et al (1995), Invest Opthelmol. Vision. Sci 36,1714-20; Howell et al (1994) Am J Hum Genet. 55 203-206; Koyabashi et al; (1994) Am. J. Hum. Genet. 55 206-209; Johns and Neufeld (1993) Am J Hum Genet 53 916-920; Chomyn et al, (1992) Proc. Natl. Acad. Sci USA 89 4221-4225) and Invader™ technology (available from Third Wave Technologies Inc. 502 South Rosa Road, Madison, Wis. 53719 USA).

The use of PCR based detection systems is preferred for use in all aspects and embodiments of the invention described herein. The use of allele selective hybridisation probe techniques, often in combination with PCR based target DNA fragment amplification, is also preferred for all aspects and embodiments of the invention described herein.

In a preferred embodiment of the first aspect of the invention we now provide a diagnostic method for the detection of the presence of absence of one or more mutation(s) in a fungal cytochrome b gene resulting in a F129L replacement in the encoded protein wherein the presence of said mutation(s) gives rise to fungal resistance to a strobilurin analogue or any other compound in the same cross resistance group said method comprising detecting the presence of an amplicon generated during a PCR reaction wherein said PCR reaction comprises contacting a test sample comprising fungal nucleic acid with a diagnostic primer in the presence of appropriate nucleotide triphosphates and an agent for polymerisation wherein the detection of said amplicon is directly related to presence or absence of said mutation(s) in said nucleic acid.

The detection of the amplicon generated during the PCR reaction may be directly dependent on the extension of a primer specific for the presence of the mutation i.e. where primer extension is dependent on the presence of the mutation and hence an amplicon is generated only when the primer binds and/or is extended when the mutation is present (as is the case with ARMS technology), similarly it may be directly dependent on the extension of a primer specific for the absence of the mutation e.g. wild type sequence or may be directly linked to the PCR extension product containing the mutant DNA sequence i.e. where the detection is of an amplicon comprising the mutant DNA sequence. The first alternative is particularly preferred. In the above method of the invention where allele selective amplification is used the said diagnostic method comprises detecting the presence of an amplicon generated during a PCR reaction wherein said PCR reaction comprises contacting a test sample comprising fungal nucleic acid with a diagnostic primer in the presence of appropriate nucleotide triphosphates and an agent for polymerisation wherein the generation of said amplicon is directly related to presence or absence of said mutation(s) in said nucleic acid.

The amplicon can be from any PCR cycle and this includes a first allele specific primer extension product.

In an alternative preferred example the method of the invention uses an allele selective hybridisation probe technique such as Molecular Beacons or TaqMan (as described herein, see Example 18).

In a particularly preferred embodiment of the first aspect of the invention we now provide a diagnostic method for the detection of the presence or absence of one or more mutation(s) in a fungal cytochrome b gene resulting in a F129L replacement in the encoded protein wherein the presence of said mutation(s) gives rise to fungal resistance to a strobilurin analogue or any other compound in the same cross resistance group said method comprising contacting a test sample, comprising a fungal nucleic acid, with a diagnostic primer appropriate for the mutation(s) resulting in a F129L replacement in the encoded protein, in the presence of appropriate nucleotide triphosphates and an agent for polymerisation, such that the diagnostic primer is extended when the mutation(s) is (are) present in the sample that results in an F129L replacement in the encoded protein or when wild type sequence is present; and detecting the presence orabsence of the said mutation(s) by reference to the presence or absence of the diagnostic primer extension product.

In a further preferred embodiment the invention provides a method for detecting the presence or absence one or more mutation(s) in a fungal cytochrome b gene resulting in a F129L replacement in the encoded protein wherein the presence of said mutation(s) gives rise to fungal resistance to a strobilurin analogue or any other compound in the same cross resistance group which method comprises contacting a test sample comprising a fungal nucleic acid with a diagnostic primer for the specific mutation(s) in the presence of appropriate nucleotide triphosphates and an agent for polymerisation, such that the diagnostic primer is extended when the said mutation(s) is (are) present in the sample; and detecting the presence or absence of the mutation(s) by reference to the presence or absence of a diagnostic primer extension product.

According to a particularly preferred embodiment of the first aspect of the invention we provide a method for detecting the presence or absence of one or more mutation(s) in a fungal cytochrome b gene resulting in a F129L replacement in the encoded protein wherein the presence of said mutation(s) gives rise to fungal resistance to a strobilurin analogue or any other compound in the same cross resistance group which method comprises contacting a test sample comprising a fungal nucleic acid with a diagnostic primer for the specific mutation(s) in the presence of appropriate nucleotide triphosphates and an agent for polymerisation, such that the diagnostic primer is extended only when the mutation(s) is (are) present in the sample; and detecting the presence or absence of the mutation(s) by reference to the presence or absence of a diagnostic primer extension product.

As used herein the term diagnostic primer is used to indicate a primer which is used specifically to identify the presence or absence of a mutation or wild type sequence and the term common primer is used to denote a primer binding to the opposite strand of DNA to that to which the diagnostic primer and 3′ to the region recognised by that diagnostic primer and which, by acting in concert with said diagnostic primer allows amplification of the intervening tract of DNA during the PCR. Where the diagnostic primer is an ARMS primer it can have a 3′ mismatch when compared to the mutant or wild type sequence.

In this and in further aspects and embodiments of the invention it is preferred that the extension of the primer extension product is detected using a detection system which is an integral part of either the diagnostic primer or the common primer on the opposite strand. Alternatively where a Taqman® or Taqman®MGB probe is used in conjunction with a diagnostic primer and a common primer, the Taqman® or Taqman®MGB probe will comprise the detection means. This is described more fully herein.

Compounds may be considered to be in the same cross resistance group when the resistance mechanism to one compound also confers resistance to another, even when the modes of action are not the same. The strobilurin analogues and compounds in the same cross resistance group include for example, azoxystrobin, picoxystrobin, kresoxim-methyl, trifloxystrobin, pyraclostrobin, famoxadone and fenamidone. (Details of pyraclostrobin were presented at the BCPC Conference in Brighton in November 2000—see Abstract 5A-2). It should also be noted that strobilurin analogues or compounds in the same cross resistance group are now frequently referred to as Qo site inhibitors (QoIs) because of their action on the complex III Qo site.

We have found that the position in fungal nucleic acids encoding cytochrome b that corresponds to the 129^{th} codon/amino acid in the cytochrome b of S. cerevisiae sequence is a key determinant of fungal resistance to strobilurin analogues or any other compound in the same cross resistance group in field isolates of strobilurin analogue resistant plant pathogenic fungi. The methods of the invention described herein are particularly suitable for the detection of a mutation at the position corresponding to that endcoding Saccharomyces cerevisiae cytochrome b residue 129 where the encoded phenylalanine residue is replaced by another amino acid which prevents the activity of strobilurin analogues or any other compound in the same cross resistance group and results in a resistant phenotype in the fungus carrying the mutant cytochrome b gene thereby giving rise to fungal resistance to strobilurin analogues or any other compound in the same cross resistance group.

The method is preferably used for the detection of a mutation resulting in the replacement of said phenylalanine residue at the position corresponding to S. cerevisiae cytochrome b residue 129 with an amino acid selected from the group isoleucine, leucine, serine, cysteine, valine, tyrosine. It is most preferable that the mutation(s) to be detected results in the phenylalanine residue being replaced by leucine.

The mutation in the fungal cytochrome b gene resulting in a F129L replacement in the encoded protein is usually a thymine to cytosine base change at the first position (base) of the codon or a thymine or cytosine to adenine or guanine base change at the third position (base) of the codon and the detection of these single nucleotide polymorphisms is preferred for all aspects and embodiments of the invention described herein. It is further possible that a rare combination of a change from thymine to cytosine at the first position and a thymine or cytosine to adenine or guanine base change at the third position (base) of the codon may also occur and this is covered in all aspects and embodiments of the invention.

It should be noted that in this patent application frequent reference is made to both mutations and allelic variants (alleles) of the cytochrome b gene which confer resistance to strobilurin analogues or compounds in the same cross resistance group. Such references are essentially synonymous although the term mutation tends to imply a new or recent genetic change whereas allelic variant implies that an alternate, and in this case resistance conferring, form of the gene may have been present for some time in the population under analysis. These alternatives are indistinguishable during analysis of natural populations.

It should also be noted that the nature in the difference in properties of the wild type and mutant/allelic variant form of the cytochrome b protein, encoded by the gene conferring resistance to strobilurin analogues or compounds in the same cross resistance group, requires an amino acid substitution within the so-called Qo site of the respective respiration complex m species which are, in part, comprised of the cytochrome b protein. Such amino acid substitutions are caused by changes in the codon for the altered amino acid. Typically, and in the specific examples considered herein, the amino acid substitution of interest is caused by a change in only one of the three nucleotide residues in that codon. Such changes may therefore be described as single nucleotide polymorphisms (SNPs).

Occasionally because of the degeneracy of the genetic code amino acid substitutions that can be caused by a single nucleotide polymorphism may also be caused by two, closely linked (within 3 nucleotides) substitutions. Such situations are referred to herein as.“simple nucleotide polymorphisms”. By their nature it would be anticipated that such polymorphisms would be much rarer than SNPs since the sequence change required to bring it about requires at least two separate base changes, within the same codon, rather than just one.

As used herein the term F129L is used to denote the substitution of a phenylalanine residue by a leucine residue in a fungal cytochrome b sequence at the equivalent of the position of the 129^{th} codon/amino acid of the S. cerevisiae cytochrome b sequence. This nomenclature is used for all other residue changes quoted herein i.e. all positions are quoted relative to the S. cerevisiae cytochrome b protein sequence. The S. cerevisiae cytochrome b gene and protein sequences are available on the EMBL and SWISSPROT databases (See EMBL ACCESSION NO. X84042 and SWISSPROT ACCESSION NO. P00163). The skilled man will appreciate that the precise length and register of equivalent proteins from different species may vary as a result of amino or carboxy terminal and/or one or more internal deletions or insertions. However, since the amino acid tract containing the residue corresponding to F₁₂₉ in S. cerevisiae is well conserved (Widger et al. Proc.Nat.Acad.Sci., U.S.A. 81 (1984) 674-678) it is straightforward and easily within the capability of the skilled man to identify the precisely corresponding residue in a newly obtained fungal cytochrome b sequence either by visual inspection or use of one of several sequence alignment programmes including Megalign or Macaw. Though designated F₁₂₉ in this application (because of positional and functional equivalence) the precise position of this phenylalanine in the new cytochrome b may not be the 129w residue from its amino terminal end. The S. cerevisiae cytochrome b consensus sequence is provided in SWISSPROT ACCESSION NO. P00163. In all aspects and embodiments of the invention described herein the positions in the cytochrome b sequence are preferably as defined relative to the S. cerevisiae cytochrome b sequence provided in EMBL ACCESSION NO. X84042. Alternatively, in all aspects and embodiments of the invention described herein the positions in the cytochrome b sequence are preferably as defined relative to the S. cerevisiae cytochrome b consensus sequence as provided in SWISSPROT ACCESSION NO. P00163.

According to one aspect of the invention there is provided a method for the diagnosis of one or more nucleotide polymorphisms in a fungal cytochrome b gene which method comprises determining the sequence of the fungal nucleic acid at a position corresponding to one or more of the bases in the triplet coding for the amino acid at the position that corresponds to S. cerevisiae cytochrome b residue 129 in the cytochrome b protein and determining the resistance status of the fungus to a strobilurin analogue or a compound in the same cross resistance group by reference to one or more polymorphisms in the cytochrome b gene.

In all aspects and embodiments of the invention described herein it is preferred that only one base in the triplet coding for the amino acid at the position corresponding to S. cerevisiae cytochrome b residue 129 in the cytochrome b protein shows a mutation i.e. there is a single nucleotide polymorphism occurring at one position only and it is further preferred that it is at the first or third base of the triplet.

According to a preferred embodiment of this aspect of the invention there is provided a method for the diagnosis of a single nucleotide polymorphism in a fungal cytochrome b gene which method comprises determining the sequence of fungal nucleic acid at a position corresponding to the first or third base in the triplet coding for the amino acid at the position corresponding to S. cerevisiae cytochrome b residue 129 in the cytochrome b protein and determining the resistance status of the said fungi to a strobilurin analogue or a compound in the same cross resistance group by reference to a polymorphism in the cytochrome b gene.

In an embodiment of the above aspect of the invention the method for diagnosis described herein is one in which the single nucleotide polymorphism at positions in the DNA corresponding to the first or third base in the triplet coding for the amino acid at the position corresponding to S. cerevisiae cytochrome b residue 129 in the cytochrome b protein is presence of T or C at the first base in the codon and T, C, A or G at the third base in the codon.

TABLE 1
Codon sequences encoding phenylalanine and leucine.

| First | Second | Third |
|---|---|---|
| position | position | position |
| | T | |
| T | Phe | T |
| | Phe | C |
| | Leu | A |
| | Leu | G |
| C | Leu | T |
| | Leu | C |
| | Leu | A |
| | Leu | G |

In wild type cytochrome b, if the phenylalanine residue at position 129 is encoded by either a TTT or TTC codon, then a single base mutation at the first position of the codon to a C (cytosine) would result in substitution of the phenylalanine residue in the Qₒ site of the strobilurin resistant mutant. Likewise if in wild type cytochrome b the phenylalanine at position 129 is encoded by either a TTT or TTC codon, then a single base position at the third position of the codon to an A (adenine) or a G (guanine) would result in substitution of the phenylalanine residue in the Qₒ site of the strobilurin resistant mutant. A double substitution at the first position (from a T to a C) together with a substitution at the third position (from either a T or a C to either an A or a G) could also cause such a phenylalanine to leucine amino acid substitution (see Table 1).

In order to define whether a given plant pathogen or population of a plant pathogen is resistant to Qₒ site inhibitor fungicides or contains significant levels of a resistant allele as a result of the presence of a leucine residue at position 129, it is simply necessary to establish and/or measure whether that pathogen or population has a C residue at the first position of its cognate codon and/or an A or G residue at the third position. One method of achieving such an assessment is to use technology based on diagnostic primers such as ARMS primers. (The concept of ARMS primers is described fully in Newton et al, Nucleic Acid Research 17 (7) 2503-2516 1989).

As a consequence of the above features of phenylalanine and leucine codons (see Table 1), when ARMS technology is used to detect and/or measure the status of residue 129, it is possible to design appropriate PCR primers that are capable of defining the identity and/or amounts of particular residues at either the first or third positions of the cognate codon in the pathogen cytochrome b gene. Such design requires the knowledge of only wild type sequence. There is no need to have access to a resistant isolate in a new fungus of interest where resistance results from a F129L mutation. Some examples of relevant plant pathogenic fungi are listed in Table 2. This list is not meant to be in any way to be exclusive. The skilled plant pathologist will be able to readily identify those fungi to which the methods of this invention are relevant.

TABLE 2
Example of species where F129L can be assayed.

| | Examples of species in which |
|---|---|
| | F129L can be assayed: |
| 1 | Plasmopara viticola |
| 2 | Erysiphe graminis f. sp. trilici/hordei |
| 3 | Rhynchosporium secalis |
| 4 | Pyrenophora teres |
| 5 | Mycosphaerella graminicola |
| 6 | Mycosphaerella fijiensis var. difformis |
| 7 | Sphaerotheca fuliginea |
| 8 | Uncinula necator |
| 9 | Colletotrichum graminicola |
| 10 | Pythium aphanidermatum |
| 11 | Colletotrichum gloeosporioides |
| 12 | Oidium lycopersicum |
| 13 | Leveillula taurica |
| 14 | Pseudoperonospora cubensis |
| 15 | Alternaria solani |
| 16 | Cercospora arachidola |
| 17 | Rhizoctonia solani |
| 18 | Venturia inaequalis |
| 19 | Phytophthora infestans |
| 20 | Mycosphaerella musicola |
| 21 | Colletotrichum acutatum |
| 22 | Wilsonomyces carpophillum |
| 23 | Didymella bryoniae |
| 24 | Didymella lycopersici |
| 25 | Peronospora tabacina |
| 26 | Puccinia recondita |
| 27 | Puccinia horiana |

The methods of the invention described herein are particularly useful in connection with plant pathogenic fungi and especially with any of the following fungal species: Plasmopara viticola, Erysiphe graminis f.sp. tritic/hordei, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Collectotrichum gloeosporioides, Oidium lycopersicurm, Phytophthora infestans, Leveillula taurica, Pseudoperonospora cubensis, Alternaria solani, Rhizoctonia solani, Mycosphaerella musicola, Cercospora arachidola, Colletotrichum acutatum, Wilsonomyces carpophillum, Didymella bryoniae, Didymella lycopersici, Peronospora tabacina, Puccinia recondita and Puccinia horiana, and most especially with any of the following fungal species: Plasmopara viticola, Erysiphe graminis f.sp. tritici/hordei, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Didymella bryoniae, Didymella lycopersici, Mycosphaerella musicola and Cercospora arachidola.

In a further aspect the invention provides a method for detecting fungal resistance to a strobilurin analogue or any other compound in the same cross resistance group said method comprising identifying the presence or absence of one or more mutation(s) in a fungal nucleic acid that encodes a fungal cytochrome b protein wherein the presence of said mutation(s) gives rise to resistance to a strobilurin analogue or any other compound in the same cross resistance group said method comprising identifying the presence or absence of a single nucleotide polymorphism occurring at positions corresponding to one or more of the bases in the triplet coding for the amino acid at the position corresponding to S. cerevisiae cytochrome b residue 129 in the fungal cytochrome b protein.

In a further preferred embodiment of this aspect the invention provides a method for detecting fungal resistance to a strobilurin analogue or any other compound in the same cross resistance group said method comprising identifying the presence or absence of a mutation in a fungal nucleic acid that encodes a fungal cytochrome b protein wherein the presence of said mutation gives rise to resistance to a strobilurin analogue or any other compound in the same cross resistance group said method comprising identifying the presence or absence of a single nucleotide polymorphism occurring at a position corresponding to the first and/or third base in the triplet coding for the amino acid at the position corresponding to S. cerevisiae cytochrome b residue 129 in the fungal cytochrome b protein.

In a preferred embodiment of this aspect of the invention the presence or absence of a single nucleotide polymorphism at a position corresponding to the first and/or third base in the triplet coding for the amino acid at the position corresponding to S. cerevisiae cytochrome b residue 129 in the cytochrome b gene in fungal nucleic acid are identified using any (or a) single nucleotide polymorphism detection technique.

The invention further provides a fungal DNA sequence encoding all or part of a wild type cytochrome b protein wherein said DNA sequence encodes a phenylalanine residue at the position corresponding to S. cerevisiae cytochrome b residue 129 in the wild type protein wherein said sequence is obtainable or obtained from a fungus selected from the group consisting of: Plasmopara viticola, Rhynchosporium secalis, Pyrenophora teres, Mycosphaerella graminicola, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Leveillula taurica, Pseudoperonospora cubensis, Alternaria solani, Rhizoctania solani, Mycosphaerella musicola, Cercospora arachidola, Colletotrichum acutatum, Wilsonomyces carpophillum, Didymella bryoniae, Didymella lycopersici, Peronospora tabacina, Puccinia recondita and Puccinia horiana, preferably from the group consisting of: Plasmopara viticola, Rhynchosporium secalis, Pyrenophora teres, Mycosphaerella graminicola, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Leveillula taurica, Pseudoperonospora cubensis, Alternaria solani, Rhizoctania solani, Didymella bryoniae, Didymella lycopersici, Mycosphaerella musicola and Cercospora arachidola.

A fungal DNA sequence according to the above aspects of the invention preferably comprises around 30 nucleotides on either or both sides of the position in the DNA that corresponds to one or more of the bases in the triplet (preferably the third base) coding for the amino acid at the position corresponding to S. cerevisiae cytochrome b residue 129 in the protein since this extent of nucleic acid provides the skilled man with all information necessary to design species- and mutation-specific reagents and/or methods for use in/with all single nucleotide polymorphism detection techniques as described herein. As used herein the term around 30 means that the sequence may comprise up to 30 nucleotides, for example 5, up to 10, 15, 20, or 25 nucleotides or may comprise more than 30 nucleotides, for example around 50 nucleotides i.e. up to 35, 40, 45 or 50 or more nucleotides.

As used herein in connection with all DNA and protein sequences the term ‘all or part of’ is used to denote a DNA sequence or protein sequence or a fragment thereof. A fragment of DNA or protein may for example be 10%, 20%, 25%, 30%, 40%, 50%, 60%, 70%, 75%, 80%, 85%, 90%, or 95% of the length of the whole sequence.

The invention extends also to novel protein sequences encoded by the DNA sequences of the present invention.

It will be evident to the man skilled in the art that both samples containing genomic (mitochondrial) DNA and cDNA may be analysed according to the invention. Where the sample contains genomic DNA, intron organisation needs to be taken into account when using the sequence information. Examnples of wild type fungal DNA sequences comprising part of the wild type cytochrome b gene sequence according to the above aspect of the invention are provided in Table 3 below and said sequences form a further aspect of the invention.

TABLE 3

Tracts of wild type
cytochrome b genomic and/or cDNA sequence
flanking the codon corresponding to codon 129
in the S. cerevisiae cytochrome b sequence (first
and last residues shown in bold and underlined)
for a series of important plant pathogens.

| | |
|---|---|
| Species | Sequence |

| | |
|---|---|
| Plasmopara viticola | 5′TTATGGTGTTCAGGGGTAAT |
| (cDNA & genomic) | TATTTTTATTTTAATGATGGCG |
| | ACTGCATTTATGGGTTATG 3′ |
| Rhynchosporium secalis | 5′GTATGAACAATAGGTACATT |
| (cDNA & genomic, | TATATTCATATTAATGATCGTT |
| | ACAGCATTCTTGGGTTATG 3′ |
| Pyrenophora teres | 5′GTATGAACTATTGGTACTGT |
| (cDNA) | TATCTTTATCTTAATGATGGCT |
| | ACAGCCTTCCTGGGTTACG 3′ |
| Pyrenophora teres | 5′CGCTATACAGATAAATTTAG |
| (genomic) | GTTGTAGTTAGCCGGAACTTAG |
| | ACAGCCTTCCTGGGTTACCAAC |
| | ATAGCCCAAAATGGTTTAATAT |
| | AAGTAATAAAAAAAG 3′ |
| Mycosphaerella graminicola | 5′ACATGAACAATCGGTACTAT |
| (cDNA & genomic) | AATACTAGTTCTGATGATGGCA |
| | ACCGCATTCTTAGGGTATG 3′ |
| Sphaerotheca fuliginea | 5′CATTAGGTGTAGTTATATTC |
| (cDNA) | ATATTAATGATCGTTACTGCAT |
| | TCCTGGGTTATG 3′ |
| Uncinula necator | 5′CAATTGGTACAGTAATATTC |
| (cDNA) | ATTTTAATGATGGCTACAGCAT |
| | TCTTGGGTTATG 3′ |
| Colletotrichum graminicola— | 5′GTTTGAGTTATAGGTGCTAT |
| Cgr1 (genomic & cDNA) | AATACTTGTAGCTATGATGGGT |
| | ATAGGTTTCTTAGGGTATGTTT |
| | TACCTTACGGACAAATGTCATT |
| | ATGAGGTGCTACAGT 3′ |
| Colletotrichum graminicola— | 5′GTTTGAGTTATAGGTTGTAT |
| Cgr2 (genomic & cDNA) | AATACTTGTAGCTATGATGGGT |
| | ATAGGTTTCTTAGGATATGTTT |
| | TACCTTACGGACAAATGTCATT |
| | ATGAGGTGCTACAGT 3′ |
| Colletotrichum graminicola— | 5′GTTTGAGTTATAGGTACTAT |
| Cgr3 (cDNA) | AATACTTGTAGCTATGATGGGT |
| | ATAGGTTTCCTGGGTTATGTTT |
| | TACCTTACGGACAAATGTCACT |
| | ATGAGGTGCAACTGT 3′ |
| Colletotrichum graminicola— | 5′GTTTGAGTTATAGGTACTAT |
| Cgr3 (genomic) | AATACTTGTAGCTATGATGGGT |
| | ATAGGTTTCCTGGGTTACTTCA |
| | ACATAGCCCAAAATGATATGCA |
| | ATTATTAGGATTTCA 3′ |
| Pythium aphanidermatum | 5′TTATGGTGTTCAGGTGTTGT |
| (genomic & cDNA) | TATTTTTATTTTAATGATGGCA |
| | ACAGCTTTCATGGGTTATG 3′ |
| Colletotrichum | 5′GTTTGAGTTATAGGTGCTAT |
| gloeosporioides—chilli | AATACTTGTAGCTATGATGGGT |
| (genomic & cDNA) | ATAGGTTTCCTGGGTTATG 3′ |
| Colletotrichum | 5′GTTTGAGTAATAGGTGCTAT |
| gloeosporioides—mango | AATTCTTGTAGCTATGATGGGT |
| (genomic & cDNA) | ATAGGTTTCTTGGGTTATGTTT |
| | TACCTTACGGGCAAATGTCATT |
| | ATGAGGTGCAACAGT 3′ |
| Oidium lycopersicum | 5′ACATGAACTATAGGTACAGT |
| (cDNA) | TATATTCATATTAATGATGGCT |
| | ACAGCATTCCTGGGTTATG 3′ |
| Leveillula taurica—Lt1 | 5′ACATGAACAATAGGTGTGGT |
| (cDNA) | AATATTTATATTAATGATGGCT |
| | ACAGCTTTCTTGGGTTATGTTT |
| | TACCGTACGGTCAAATGAGTTT |
| | ATGAGGTGCAACAGT 3′ |
| Leveillula taurica—Lt4 | 5′ACATGAACAATAGGTGTTGT |
| (cDNA) | AATATTTATATTAATGATGCTA |
| | CAGCTTTCCTAGGTTACGTTTT |
| | ACCATACGGGACAAATGTCATT |
| | ATGAGGTGCAACAGT 3′ |
| Leveillula taurica—Lt2 | 5′ACATGAACTATTGGTGTTGT |
| (cDNA) | TATCTTTATATTAATGATGGCT |
| | ACTGCCTTTTTAGGATATGTTT |
| | TACCATATGGTCAAATGAGTTT |
| | ATGAGGTGCTACAGT 3′ |
| Leveillula taurica—Lt3 | 5′ATGAACAATTGGTACAGTAA |
| (cDNA) | TATTCATATTAATGATGGCTAC |
| | TGCATTCCTGGGTTATGTTCTA |
| | CCTTTCGGACAGATGTCGCTCT |
| | GGGGTGCAACCGT 3′ |
| Pseudoperonospora cubensis | 5′TTATGGTGTTCAGGTGTTAT |
| (cDNA & genomic) | TATTTTTATTTTAATGATGGCA |
| | ACAGCTTTTATGGGTTATG 3′ |
| Alternaria solani | 5′GTATGAACTATTGGTACTGT |
| (cDNA & genomic) | TATCTTTATCTTAATGATGGCT |
| | ACAGCTTTCCTGGGTTATG 3′ |
| Cercospora arachidola | 5′TTATGATCTATTGGAGTTAT |
| (cDNA & genomic) | AATTTTAGTTCTTATGATGGCA |
| | ATAGCCTTCTTAGGATATG 3′ |
| Rhizoctonia solani | 5′CTATCGGAGTTGTTATGCTT |
| (cDNA) | CTTGTTATGATGATGGGGATCG |
| | CATTTTTAGGTTATG 3′ |
| Mycosphaerella musicola | 5′GTATGAGTTATAGGTACTAT |
| (genomic & cDNA) | TATATTAGTTCTAATGATGGCT |
| | ACCGCCTTTTTAGGATATG 3′ |
| Didymella bryoniae—Db1 | 5′GTATGAACAATTGGTACTGT |
| (cDNA) | TATCTTTATCTTAATGATGGCT |
| | ACAGCTTTCCTGGGTTATGTTC |
| | TTCCTTATGGGCAAATGTCATT |
| | ATGAGGTGCAACTGT 3′ |
| Didymella bryoniae—Db2 | 5′GTGTGAACAATTGGTACTGT |
| (cDNA) | TATCTTTATCTTAATGATGGCT |
| | ACAGCTTTCCTGGGTTATGTGC |
| | TGCCCTACGGGCAGATGTCATT |
| | ATGAGGTGCTACAGT 3′ |
| Didymella lycopersici | 5′GTATGAACAATTGGTACTGT |
| (cDNA) | TATCTTTATCTTAATGATGGCT |
| | ACAGCTTTCCTGGGTTATGTTC |
| | TTCCTTATGCGCAAATGTCATT |
| | ATGAGGTGCTACAGT 3′ |

In the above table the first and third bases in the triplet coding for the amino acid at the position corresponding to S. cerevisiae cytochrome b residue 129 which, when appropriately substituted, result in the replacement of the normal phenylalanine residue with an alternative amino acid wherein said replacement confers resistance to strobilurin analogues or a compound within the same cross resistance group are in bold and underlined.

The invention also extends to a fungal DNA sequence showing homology or sequence identity to said DNA sequences in Table 3 and covers for example, variations in DNA sequences found in different samples or isolates of the same species. These variations may, for example, be due to the use of alternative codon usage, varying intron/exon mitochondrial organisation and amino acid replacement.

In a further aspect the invention provides a fungal DNA sequence, which encodes all or part of a fungal cytochrome b protein wherein, when said fungal DNA sequence is lined up against the corresponding wild type DNA sequence that encodes a cytochrome b protein, it is seen that the fungal DNA sequence contains a single nucleotide polymorphism mutation at a position in the DNA that corresponds to one or more of the bases in the triplet coding for the amino acid at the position corresponding to S. cerevisiae cytochrome b residue 129 in the protein which results in the replacement of the normal phenylalanine residue with an alternative amino acid.

In a further preferred embodiment of this aspect the invention provides a fungal DNA sequence encoding all or part of a cytochrome b protein which, when said sequence is lined up against the corresponding wild type DNA sequence encoding a cytochrome b protein, is seen to contain a single nucleotide polymorphism mutation at a position in the DNA corresponding to the first or third base in the triplet coding for the amino acid at the position corresponding to S. cerevisiae cytochrome b residue 129 in the protein which results in the replacement of the normal phenylalanine residue with an alternative amino acid.

The fungal DNA sequence according to the above aspect of the invention preferably comprises around 30 nucleotides on either or both sides of the position in the DNA corresponding to one or more of the bases in the triplet, preferably corresponding to the third base in the triplet coding for the amino acid at the position corresponding to S. cerevisiae cytochrome b residue 129 in the protein since this extent of nucleic acid provides the skilled man with all information necessary to design species and mutation specific reagents and/or methods for use in all single nucleotide polymorphism techniques. As used herein the term around 30 means that the sequence may comprise up to 30 nucleotides, for example 5, up to 10, 15, 20, or 25 nucleotides or may comprise more than 30 nucleotides.

The invention further provides a fungal DNA sequence encoding all or part of a mutant cytochrome b protein wherein the presence of one or more mutation(s) in said DNA confers resistance to a strobilurin analogue or a compound within the same cross resistance group, said mutation(s) occurring at a position in the DNA corresponding to one or more of the bases in the triplet coding for the amino acid at the position corresponding to S. cerevisiae cytochrome b residue 129 in the protein.

In a preferred embodiment of this aspect the invention further provides a fungal DNA sequence encoding all or part of a mutant cytochrome b protein wherein the presence of a mutation in said DNA confers resistance to a strobilurin analogue or a compound within the same cross resistance group, said mutation occurring at a position in the DNA corresponding to the first or third base in the triplet coding for the amino acid at the position corresponding to S. cerevisiae cytochrome b residue 129 in the protein.

In the above aspects of the invention the mutation occurring at positions in the DNA corresponding to the first and third base in the triplet coding for the amino acid at the position corresponding to S. cerevisiae cytochrome b residue 129 in the protein are preferably a thymine to a cytosine and a thymine or cytosine to adenine or guanine respectively.

The fungal DNA sequence encoding all or part of a mutant cytochrome b protein wherein the presence of one or more mutation(s) in said DNA confers resistance to a strobilurin analogue or a compound within the same cross resistance group, according to the above aspects of the invention is preferably obtainable or obtained from a fungus selected from the group consisting of Plasmopara viticola, Erysiphe graminis f.sp. tritici/hordei, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Mycosphaerella musicola, Cercospora arachidola, Colletotrichum acutatum, Wilsonomyces carpophillum, Didymella bryoniae, Didymella lycopersici, Peronospora tabacina, Puccinia recondita and Puccinia horiana, preferably from the group consisting of Plasmopara viticola, Erysiphe graminisf.sp. tritici/hordei, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Didymella bryoniae, Didymella lycopersici, Mycosphaerella musicola and Cercospora arachidola.

The invention extends also to DNA sequences comprising all or part of the sequences provided in Table 4 wherein the residue at a position in the DNA corresponding to the first base in the triplet coding for the amino acid at the position corresponding to S. cerevisiae cytochrome b residue 129 in the protein is a cytosine residue. Such sequences, including those comprising the sequences described in Table 4 form a further aspect of the invention.

TABLE 4

Tracts of plant pathogen cytochrome
b gene sequence where the residue
(shown in bold) corresponding to the
first base in the codon at the position
corresponding to S. cerevisiae cytochrome
b residue 129 in the protein is a cytosine
residue and, as a result, encodes leucine.

| | |
|---|---|
| Species | Sequence |

| | |
|---|---|
| Plasmopara viticola | 5′TTATGGTGTTCAGGGGTAAT |
| (cDNA & genomic) | TATTTTTATTTTAATGATGGCG |
| | ACTGCACTTATGGGTTATG 3′ |
| Rhynchosporium secalis | 5′GTATGAACAATAGGTACATT |
| (cDNA & genomic) | TATATTCATATTAATGATCGTT |
| | ACAGCACTCTTGGGTTATG 3′ |
| Pyrenophora teres | 5′GTATGAACTATTGGTACTGT |
| (cDNA) | TATCTTTATCTTAATGATGGCT |
| | ACAGCCCTCCTGGGTTACG 3′ |
| Pyrenophora teres | 5′CGCTATACAGATAAATTTAG |
| (genomic) | GTTGTAGTTAGCCGGAACTTAG |
| | ACAGCCCTCCTGGGTTACCAAC |
| | ATAGCCCAAAATGGTTTAATAT |
| | AAGTAATAAAAAAAG 3′ |
| Mycosphaerella graminicola | 5′ACATGAACAATCGGTACTAT |
| (cDNA & genomic) | AATACTAGTTCTGATGATGGCA |
| | ACCGCACTCTTAGGGTATG 3′ |
| Mycosphaerella fijiensis | 5′GTATGAGTTATAGGTACTAT |
| var. difformis | TATATTAGTTCTAATGATGGCA |
| (cDNA & genomic) | ACTGCCCTTTTAGGGTATG 3′ |
| Sphaerotheca fuliginea | 5′CATTAGGTGTAGTTATATTC |
| (cDNA) | ATATTAATGATCGTTACTGCAC |
| | TCCTGGGTTATG 3′ |
| Uncinula necator | 5′CAATTGGTACAGTAATATTC |
| (cDNA) | ATTTTAATGATGGCTACAGCAC |
| | TCTTGGGTTATG 3′ |
| Colletotrichum graminicola— | 5′GTTTGAGTTATAGGTGCTAT |
| Cgr1 (genomic & cDNA) | AATACTTGTAGCTATGATGGGT |
| | ATAGGTCTCTTAGGGTATGTTT |
| | ACCTTACGGACAAATGTCATTA |
| | TGAGGTGCTACAGT 3′ |
| Colletotrichum graminicola— | 5′GTTTGAGTTATAGGTTGTAT |
| Cgr2 (genomic & cDNA) | AATACTTGTAGCTATGATGGGT |
| | ATAGGTCTCTTAGGATATGTTT |
| | TACCTTACGGACAAATGTCATT |
| | ATGAGGTGCTACAGT 3′ |
| Colletotrichum graminicola— | 5′GTTTGAGTTATAGGTACTAT |
| Cgr3 (cDNA) | AATACTTGTAGCTATGATGGGT |
| | ATAGGTCTCCTGGGTTATGTTT |
| | TACCTTACGGACAAATGTCACT |
| | ATGAGGTGCAACTGT 3′ |
| Colletotrichum graminicola— | 5′GTTTGAGTTATAGGTACTAT |
| Cgr3 (genomic) | AATACTTGTAGCTATGATGGGT |
| | ATAGGTCTCCTGGGTTACTTCA |
| | ACATAGCCCAAAATGATATGCA |
| | ATTATTAGGATTTCA 3′ |
| Pythium aphanidermatum | 5′TTATGGTGTTCAGGTGTTGT |
| (genomic & cDNA) | TATTTTTATTTTAATGATGGCA |
| | ACAGCTCTCATGGGTTATG 3′ |
| Colletotrichum | 5′GTTTGAGTTATAGGTGCTAT |
| gloeosporioides—chilli | AATACTTGTAGCTATGATGGGT |
| (genomic & cDNA) | ATAGGTCTCCTGGGTTATG 3′ |
| Colletotrichum | 5′GTTTGAGTAATAGGTGCTAT |
| gloeosporioides—mango | AATTCTTGTAGCTATGATGGGT |
| (genomic & cDNA) | ATAGGTCTCTTGGGTTATGTTT |
| | TACCTTACGGGCAAATGTCATT |
| | ATGAGGTGCAACAGT 3′ |
| Oidium lycopersicum | 5′ACATGAACTATAGGTACAGT |
| (cDNA) | TATATTCATATTAATGATGGCT |
| | ACAGCACTCCTGGGTTATG 3′ |
| Leveillula taurica—Lt1 | 5′ACATGAACAATAGGTGTGGT |
| (cDNA) | AATATTTATATTAATGATGGCT |
| | ACAGCTCTCTTGGGTTATGTTT |
| | TACCGTACGGTCAAATGAGTTT |
| | ATGAGGTGCAACAGT 3′ |
| Leveillula taurica—Lt4 | 5′ACATGAACAATAGGTGTTGT |
| (cDNA) | AATATTTATATTAATGATGGCT |
| | ACAGCTCTCCTAGGTTACGTTT |
| | TACCATACGGACAAATGTCATT |
| | ATGAGGTGCAACAGT 3′ |
| Leveillula taurica—Lt2 | 5′ACATGAACTATTGGTGTTGT |
| (cDNA) | TATCTTTATATTAATGATGGCT |
| | ACTGCCCTTTTAGGATATGTTT |
| | ACCATATGGTCAAATGAGTTTA |
| | TGAGGTGCTACAGT 3′ |
| Leveillula taurica—Lt3 | 5′ATGAACAATTGGTACAGTAA |
| (cDNA) | TATTCATATTAATGATGGCTAC |
| | TGCACTCCTGGGTTATGTTCTA |
| | CCTTTGGACAGATGTCGCTCTG |
| | CGGTGCAACCGT 3′ |
| Pseudoperonospora cubensis | 5′TTATGGTGTTCAGGTGTTAT |
| (cDNA & genomic) | TATTTTTATTTTAATGATGGCA |
| | ACAGCTCTTATGGGTTATG 3′ |
| Alternaria solani | 5′GTATGAACTATTGGTACTGT |
| (cDNA & genomic) | TATCTTTATCTTAATGATGGCT |
| | ACAGCTCTCCTGGGTTATG 3′ |
| Cercospora arachidola | 5′TTATGATCTATTGGAGTTAT |
| (cDNA & genomic) | AATTTTATTCTTATGATGGCAA |
| | TAGCCCTCTTAGGATATG 3′ |
| Rhizoctonia solani | 5′CTATCGGAGTTGTTATGCTT |
| (cDNA) | GTTATGATGATGGGGATCGCAC |
| | TTTTAGGTTATG 3′ |
| Mycosphaerella musicola | 5′GTATGAGTTATAGGTACTAT |
| (genomic & cDNA) | TATATTAGTTCTAATGATGGCT |
| | ACCGCCCTTTTAGGATATG 3′ |
| Didymella bryoniae—Db1 | 5′GTATGAACAATGGTACTGTT |
| (cDNA) | ATCTTTATCTTAATGATGGCTA |
| | CAGCTCTCCTGGGTATGTTCTT |
| | CCTTATGGGCAAATGTCATTAT |
| | GAGGTGCAACTGT 3′ |
| Didymella bryoniae—Db2 | 5′GTGTGAACAATTGGTACTGT |
| (cDNA) | TATCTTTATCTTAATGATGGCT |
| | ACAGCTCTCCTGGGTTATGTGC |
| | TGCCCTACGGGCAGATGTCATT |
| | ATGAGGTGCTACAGT3′ |
| Didymella lycopersici | 5′GTATGAACAATTGGTACTGT |
| (cDNA) | TATCTTTATCTTAATGATGGCT |
| | ACAGCTCTCCTGGGTTATGTTC |
| | TTCCTTATGGGCAAATGTCATT |
| | ATGAGGTGCTACAGT 3′ |

The invention extends also to DNA sequences comprising all or part of the sequences provided in Table 5 wherein the residue at a position in the DNA corresponding to the third base in the triplet coding for the amino acid at the position corresponding to S. cerevisiae cytochrome b residue 129 in the protein is an adenine residue. Such sequences form a further aspect of the invention:

TABLE 5

Tracts of plant pathogen cytochrome
b gene sequence where the residue (shown in
bold) corresponding to the third base in the codon
at the position corresponding to S. cerevisiae
cytochrome b residue 129 in the protein is an
adenine residue and, as a result, encodes leucine.

| | |
|---|---|
| Species | Sequence |

| | |
|---|---|
| Plasmopara viticola | 5′TTATGGTGTTCAGGGGTAAT |
| (cDNA & genomic) | TATTTTTATTTTAATGATGGCG |
| | ACTGCATTAATGGGTTATG 3′ |
| Rhynchosporium secalis | 5′GTATGAACAATAGGTACATT |
| (cDNA & genomic) | TATATTCATATTAATGATCGTT |
| | ACAGCATTATTGGGTTATG 3′ |
| Pyrenophora teres | 5′GTATGAACTATTGGTACTGT |
| (cDNA) | TATCTTTATCTTAATGATGGCT |
| | ACAGCCTTACTGGGTTACG 3′ |
| Pyrenophora teres | 5′CGCTATACAGATAAATTTAG |
| (genomic) | GTTGTAGTTAGCCGGAACTTAG |
| | ACAGCCTTACTGGGTTACCAAC |
| | ATAGCCCAAAATGGTTTAATAT |
| | AAGTAATAAAAAAAG 3′ |
| Mycosphaerella graminicola | 5′ACATGAACAATCGGTACTAT |
| (cDNA & genomic) | AATACTAGTTCTGATGATGGCA |
| | ACCGCATTATTAGGGTATG 3′ |
| Mycosphaerella fijiensis | 5′GTATGAGTTATAGGTACTAT |
| var. difformis | TATATTAGTTCTAATGATGGCA |
| (cDNA & genomic) | ACTGCCTTATTAGGGTATG 3′ |
| Sphaerotheca fuliginea | 5′CATTAGGTGTAGTTATATTC |
| (cDNA) | ATATTAATGATCGTTACTGCAT |
| | TACTGGGTTATG 3′ |
| Uncinula necator | 5′CAATTGGTACAGTAATATTC |
| (cDNA) | ATTTTAATGATGGCTACAGCAT |
| | TATTGGGTTATG 3′ |
| Colletotrichum graminicola— | 5′GTTTGAGTTATAGGTGCTAT |
| Cgr1 (genomic & cDNA) | AATACTTGTAGCTATGATGGGT |
| | ATAGGTTTATTAGGGTATGTTT |
| | TACCTTACGGACAAATGTCATT |
| | ATGAGGTGCTACAGT 3′ |
| Colletotrichum graminicola— | 5′GTTTGAGTTATAGGTTGTAT |
| Cgr2 (genomic & cDNA) | AATACTTGTAGCTATGATGGGT |
| | ATAGGTTTATTAGGATATGTTT |
| | TACCTTACGGACAAATGTCATT |
| | ATGAGGTGCTACAGT 3′ |
| Colletotrichum graminicola— | 5′GTTTGAGTTATAGGTACTAT |
| Cgr3 (cDNA) | AATACTTGTAGCTATGATGGGT |
| | ATAGGTTTACTGGGTTATGTTT |
| | TACCTTACGGACAAATGTCACT |
| | ATGAGGTGCAACTGT 3′ |
| Colletotrichum graminicola— | 5′GTTTGAGTTATAGGTACTAT |
| Cgr3 (genomic) | AATACTTGTAGCTATGATGGGT |
| | ATAGGTTTACTGGGTTACTTCA |
| | ACATAGCCCAAAATGATATGCA |
| | ATTATTAGGATTTCA 3′ |
| Pythium aphanidermatum | 5′TTATGGTGTTCAGGTGTTGT |
| (genomic & cDNA) | TATTTTTATTTTAATGATGGCA |
| | ACAGCTTTAATGGGTTATG 3′ |
| Colletotrichum | 5′GTTTGAGTTATAGGTGCTAT |
| gloeosporioides—chilli | AATACTTGTAGCTATGATGGGT |
| (genomic & cDNA) | ATAGGTTTACTGGGTTATG 3′ |
| Colletotrichum | 5′GTTTGAGTAATAGGTGCTAT |
| gloeosporioides—mango | AATTCTTGTAGCTATGATGGGT |
| (genomic & cDNA) | ATAGGTTTATTGGGTTATGTTT |
| | TACCTTACGGGCAAATGTCATT |
| | ATGAGGTGCAACAGT 3′ |
| Oidium lycopersicum | 5′ACATGAACTATAGGTACAGT |
| (cDNA) | TATATTCATATTAATGATGGCT |
| | ACAGCATTACTGGGTTATG 3′ |
| Leveillula taurica—Lt1 | 5′ACATGAACAATAGGTGTGGT |
| (cDNA) | AATATTTATATTAATGATGGCT |
| | ACAGCTTTATTGGGTTATGTTT |
| | TACCGTACGGTCAAATGAGTTT |
| | ATGAGGTGCAACAGT 3′ |
| Leveillula taurica—Lt4 | 5′ACATGAACAATAGGTGTTGT |
| (cDNA) | AATATTTATATTAATGATGGCT |
| | ACAGCTTTACTAGGTTACGTTT |
| | TACCATACGGACAAATGTCATT |
| | ATGAGGTGCAACAGT 3′ |
| Leveillula taurica—Lt2 | 5′ACATGAACTATTGGTGTTGT |
| (cDNA) | TATCTTTATATTAATGATGGCT |
| | ACTGCCTTATTAGGATATGTTT |
| | TACCATATGGTCAAATGAGTTT |
| | ATGAGGTGCTACAGT 3′ |
| Leveillula taurica—Lt3 | 5′ATGAACAATTGGTACAGTAA |
| (cDNA) | TATTCATATTAATGATGGCTAC |
| | TGCATTACTGGGTTATGTTCTA |
| | CCTTTCGGACAGATGTCGCTCT |
| | GGGGTGCAACCGT 3′ |
| Pseudoperonospora cubensis | 5′TTATGGTGTTCAGGTGTTAT |
| (cDNA & genomic) | TATTTTTATTTTAATGATGGCA |
| | ACAGCTTTAATGGGTTATG 3′ |
| Alternaria solani | 5′GTATGAACTATTGGTACTGT |
| (cDNA & genomic) | TATCTTTATCTTAATGATGGCT |
| | ACAGCTTTACTGGGTTATG 3′ |
| Cercospora arachidola | 5′TTATGATCTATTGGAGTTAT |
| (cDNA & genomic) | AATTTTAGTTCTTATGATGGCA |
| | ATAGCCTTATTAGGATATG 3′ |
| Rhizoctonia solani | 5′CTATCGGAGTTGTTATGCTT |
| (cDNA) | GTTATGATGATGGGGATCGCAT |
| | TATTAGGTTATG 3′ |
| Mycosphaerella musicola | 5′GTATGAGTTATAGGTACTAT |
| (genomic & cDNA) | TATATTAGTTCTAATGATGGCT |
| | ACCGCCTTATTAGGATATG 3′ |
| Didymella bryoniae—Db1 | 5′GTATGAACAATTGGTACTGT |
| (cDNA) | TATCTTTATCTTAATGATGGCT |
| | ACAGCTTTACTGGGTTATGTTC |
| | TTCCTTATGCGCAAATGTCATT |
| | ATGAGGTGCAACTGT 3′ |
| Didymella bryoniae—Db2 | 5′GTGTGAACAATTGGTACTGT |
| (cDNA) | TATCTTTATCTTAATGATGGCT |
| | ACAGCTTTACTGGGTTATGTGC |
| | TGCCCTACGGGCAGATGTCATT |
| | ATGAGGTGCTACAGT 3′ |
| Didymella lycopersici | 5′GTATGAACAATTGGTACTGT |
| (cDNA) | TATCTTTATCTTAATGATGGCT |
| | ACAGCTTTACTGGGTTATGTTC |
| | TTCCTTATGGGCAAATGTCATT |
| | ATGAGGTGCTACAGT 3′ |

The invention extends also to DNA sequences comprising all or part of the sequences provided in Table 6 wherein the residue at a position in the DNA corresponding to the third base in the triplet coding for the amino acid at the position corresponding to S. cerevisiae cytochrome b residue 129 in the protein is a guanine residue. Such sequences form a further aspect of the invention.

TABLE 6

Tracts of plant pathogen cytochrome
b gene sequence where the residue (shown in
bold) corresponding to the third base in the codon
at the position corresponding to S. cerevisiae
cytochrome b residue 129 in the protein is a
guanine residue and, as a result, encodes leucine.

| | |
|---|---|
| Species | Sequence |

| | |
|---|---|
| Plasmopara viticola | 5′TTATGGTGTTCAGGGGTAAT |
| (cDNA & genomic) | TATTTTTATTTTAATGATGGCg |
| | ACTGCATTGATGGGTTATG 3′ |
| Rhynchosporium secalis | 5′GTATGAACAATAGGTACATT |
| (cDNA & genomic) | TATATTCATATTAATGATCGTT |
| | ACAGCATTGTTGGGTTATG 3′ |
| Pyrenophora teres | 5′GTATGAACTATTGGTACTGT |
| (cDNA) | TATCTTTATCTTTATCTTAATG |
| | ATGGCTACAGCCTTGCTGGGTT |
| | ACG 3′ |
| Pyrenophora teres | 5′CGCTATACAGATAAATTTAG |
| (genomic) | GTTGTAGTTAGCCGGAACTTAG |
| | ACAGCCTTGCTGGGTTACCAAC |
| | ATAGCCCAAAATGGTTTAATAT |
| | AAGTAATAAAAAAAG 3′ |
| Mycosphaerella graminicola | 5′ACATGAACAATCGGTACTAT |
| (cDNA & genomic) | AATACTAGTTCTGATGATGGCA |
| | ACCGCATTGTTAGGGTATG 3′ |
| Mycosphaerella fijiensis | 5′GTATGAGTTATAGGTACTAT |
| var. difformis | TATATTAGTTCTAATGATGGCA |
| (cDNA & genomic) | ACTGCCTTGTTAGGGTATG 3′ |
| Sphaerotheca fuliginea | 5′CATTAGGTGTAGTTATATTC |
| (cDNA) | ATATTAATGATCGTTACTGCAT |
| | TGCTGGGTTATG 3′ |
| Uncinula necator | 5′CAATTGTACAGTAATATTCA |
| (cDNA) | TTTTAATGATGGCTACAGCATT |
| | GTTGGGTTATG 3′ |
| Colletotrichum graminicola— | 5′GTTTGAGTTATAGGTGCTAT |
| Cgr1 (genomic & cDNA) | AATACTTGTAGCTATGATGGGT |
| | ATAGGTTTGTTAGGGTATGTTT |
| | TACCTTACGGACAAATGTCATT |
| | ATGAGGTGCTACAGT 3′ |
| Colletotrichum graminicola— | 5′GTTTGAGTTATAGGTTGTAT |
| Cgr2 (genomic & cDNA) | AATACTTGTAGCTATGATGGGT |
| | ATAGGTTTGTTAGGATATGTTT |
| | TACCTTACGGACAAATGTCATT |
| | ATGAGGTGCTACAGT 3′ |
| Colletotrichum graminicola— | 5′GTTTGAGTTATAGGTACTAT |
| Cgr3 (cDNA) | AATACTTGTAGCTATGATGGGT |
| | ATAGGTTTGCTGGGTTATGTTT |
| | TACCTTACGGACAAATGTCACT |
| | ATGAGGTGCAACTGT 3′ |
| Colletotrichum graminicola— | 5′GTTTGAGTTATAGGTACTAT |
| Cgr3 (genomic) | AATACTTGTAGCTATGATGGGT |
| | ATAGGTTTGCTGGGTTACTTCA |
| | ACATAGCCCAAAATGATATGCA |
| | ATTATTAGGATTTC A 3′ |
| Pythium aphanidermatum | 5′TTATGGTGTTCAGGTGTTGT |
| (genomic & cDNA) | TATTTTTATTTTAATGATGGCA |
| | ACAGCTTTGATGGGTTATG 3′ |
| Colletotrichum | 5′GTTTGAGTTATAGGTGCTAT |
| gloeosporioides—chilli | AATACTTGTAGCTATGATGGGT |
| (genomic & cDNA) | ATAGGTTTGCTGGGTTATG 3′ |
| Colletotrichum | 5′GTTTGAGTAATAGGTGCTAT |
| gloeosporioides—mango | AATTCTTGTAGCTATGATGGGT |
| (genomic & cDNA) | ATAGGTTTGTTGGGTTATGTTT |
| | TACCTTACGGGCAAATGTCATT |
| | ATGAGGTGCAACAGT 3′ |
| Oidium lycopersicum | 5′ACATGAACTATAGGTACAGT |
| (cDNA) | TATATTCATATTAATGATGGCT |
| | ACAGCATTGCTGGGTTATG 3′ |
| Leveillula taurica—Lt1 | 5′ACATGAACAATAGGTGTGGT |
| (cDNA) | AATATTTATATTAATGATGGCT |
| | ACAGCTTTGTTGGGTTATGTTT |
| | TACCGTACGGTCAAATGAGTTT |
| | ATGAGGTGCAACAGT 3′ |
| Leveillula taurica—Lt4 | 5′ACATGAACAATAGGTGTTGT |
| (cDNA) | AATATTTATATTAATGATGGCT |
| | ACAGCTTTGCTAGGTTACGTTT |
| | TACCATACGGACAAATGTCATT |
| | ATGAGGTGCAACAGT 3′ |
| Leveillula taurica—Lt2 | 5′ACATGAACTATTGGTGTTGT |
| (cDNA) | TATCTTTATATTAATGATGGCT |
| | ACTGCCTTGTTAGGATATGTTT |
| | TACCATATGGTCAAATGAGTTT |
| | ATGAGGTGCTACAGT 3′ |
| Leveillula taurica—Lt3 | 5′ATGAACAATTGGTACAGTAA |
| (cDNA) | TATTCATATTAATGATGGCTAC |
| | TGCATTGCTGGGTTATGTTCTA |
| | CCTTTCGGACAGATGTCGCTCT |
| | GGGGTGCAACCGT 3′ |
| Pseudoperonospora cubensis | 5′TTATGGTGTTCAGGTGTTAT |
| (cDNA & genomic) | TATTTTTATTTTAATGATGGCA |
| | ACAGCTTTGATCGGGTTATG3′ |
| Alternaria solani | 5′GTATGAACTATTGGTACTGT |
| (cDNA & genomic) | TATCTTTATCTTAATGATGGCT |
| | ACAGCTTTGCTGGGTTATG 3′ |
| Cercospora arachidola | 5′TTATGATCTATTGGAGTTAT |
| (cDNA & genomic) | AATTTTAGTTCTTATGATGGCA |
| | ATACCCTTGTTAGGATATG 3′ |
| Rhizoctonia solani | 5′CTATCGGAGTTGTTATGCTT |
| (cDNA) | GTTATGATGATGGGGATCGCAT |
| | TGTTAGGTTATG 3′ |
| Mycosphaerella musicola | 5′GTATGAGTTATAGGTACTAT |
| (genomic & cDNA) | TATATTAGTTCTAATGATGGCT |
| | ACCGCCTTGTTAGGATATG 3′ |
| Didymella bryoniae—Db1 | 5′GTATGAACAATTGGTACTGT |
| (cDNA) | GTATCTTTATCTTAATGATGCT |
| | ACAGCTTTGCTGGGTTATGTTC |
| | TTCCTTATGGGCAAATGTCATT |
| | ATGAGGTGCAACTGT 3′ |
| Didymella bryoniae—Db2 | 5′GTGTGAACAATTGGTACTGT |
| (cDNA) | TATCTTTATCTTAATGATGGCT |
| | ACAGCTTTGCTGGGTTATGTGC |
| | TGCCCTACGGGCAGATGTCATT |
| | ATGAGGTGCTACAGT 3′ |
| Didymella lycopersici | 5′GTATGAACAATTGGTACTGT |
| | TATCTTTATCTTAATGATGGCT |
| | ACAGCTTTGCTGGGTTATGTTC |
| | TTCCTTATGGGCAAATGTCATT |
| | ATGAGGTGCTACAGT 3′ |

The invention extends also to DNA sequences comprising all or part of the sequences provided in Table 7 wherein the residue at a position in the DNA corresponding to the first base in the triplet coding for the amino acid at the position corresponding to S. cerevisiae cytochrome b residue 129 is a cytosine and the residue at the third base in the corresponding codon is an adenine. Such sequences form a further aspect of the invention.

TABLE 7

Tracts of plant pathogen cytochrome
b gene sequence where the residue (shown
underlined) corresponding to the first
base in the codon at the position corresponding to
S. cerevisiae cytochrome b residue 129 in the
protein is a cytosine and the residue shown in
bold at the third base of the corresponding codon
is an adenine and, as a result, encodes leucine.

| | |
|---|---|
| Species | Sequence |

| | |
|---|---|
| Plasmopara viticola | 5′TTATGGTGTTCAGGGGTAAT |
| (cDNA & genomic) | TATTTTTATTTTAATGATGGCG |
| | ACTGCACTAATGGGTTATG 3′ |
| Rhynchosporium secalis | 5′GTATGAACAATAGGTACATT |
| (cDNA & genomic) | TATATTCATATTAATGATCGTT |
| | ACAGCACTATTGGGTTATG 3′ |
| Pyrenophora teres | 5′GTATGAACTATTGGTACTGT |
| (cDNA) | TATCTTTATCTTAATGATGGCT |
| | ACAGCCCTACTGGGTTACG 3′ |
| Pyrenophora teres | 5′CGCTATACAGATAAATTTAG |
| (genomic) | GTTGTAGTTAGCCGGAACTTAG |
| | ACAGCCCTACTGGCTTACCAAC |
| | ATAGCCCAAAATGGTTTAATAT |
| | AAGTAATAAAAAAAG 3′ |
| Mycosphaerella graminicola | 5′ACATGAACAATCGGTACTAT |
| (cDNA & genomic) | AATACTAGTTCTGATGATGGCA |
| | ACCGCACTATTAGGGTATG 3′ |
| Mycosphaerella fijiensis | 5′GTATGAGTTATAGGTACTAT |
| var. difformis | TATATTAGTTCTAATGATGGCA |
| (cDNA & genomic) | ACTGCCCTATTAGGGTATG 3′ |
| Sphaerotheca fuliginea | 5′CATTAGGTGTAGTTATATTC |
| (cDNA) | ATATTAATGATCGTTACTGCAC |
| | TACTGGGTTATG 3′ |
| Uncinula necator | 5′CAATTGGTACAGTAATATTC |
| (cDNA) | ATTTTAATGATGGCTACAGCAC |
| | TATTGGGTTATG 3′ |
| Colletotrichum graminicola— | 5′GTTTGAGTTATAGGTGCTAT |
| Cgr1 (genomic & cDNA) | AATACTTGTAGCTATGATGGGT |
| | ATAGGTCTATTAGGGTATGTTT |
| | TACCTTACGGACAAATGTCATT |
| | ATGAGGTGCTACAGT 3′ |
| Colletotrichum graminicola— | 5′GTTTGAGTTATAGGTTGTAT |
| Cgr2 (genomic & cDNA) | AATACTTGTAGCTATGATGGGT |
| | ATAGGTCTATTAGGATATGTTT |
| | TACCTTACGGACAAATGTCATT |
| | ATGAGGTGCAACAGT 3′ |
| Colletotrichum graminicola— | 5′GTTTGAGTTATAGGTACTAT |
| Cgr3 (cDNA) | AATACTTGTAGCTATGATGGGT |
| | ATAGGTCTACTGGGTTATGTTT |
| | TACCTTACGGACAAATGTCACT |
| | ATGAGGTGCAACTGT 3′ |
| Colletotrichum graminicola— | 5′GTTTGAGTTATAGGTACTAT |
| Cgr3 (genomic) | AATACTTGTAGCTATGATGGGT |
| | ATAGGTCTACTGGGTTACTTCA |
| | ACATAGCCCAAAATGATATGCA |
| | ATTATTAGGATTTCA 3′ |
| Pythium aphanidermatum | 5′TTATGGTGTTCAGGTGTTGT |
| (genomic & cDNA) | TATTTTTATTTTAATGATGGCA |
| | ACAGCTCTAATGGGTTATG 3′ |
| Colletotrichum | 5′GTTTGAGTTATAGGTGCTAT |
| gloeosporioides—chilli | AATACTTGTAGCTATGATGGGT |
| (genomic & cDNA) | ATAGGTCTACTGGGTTATG 3′ |
| Colletotrichum | 5′GTTTGAGTAATAGGTGCTAT |
| gloeosporioides—mango | AATTCTTGTAGCTATGATGGGT |
| (genomic & cDNA) | ATAGGTCTATTGGGTTATGTTT |
| | TACCTTACGGGCAAATGTCATT |
| | ATGAGCTGCAACAGT 3′ |
| Oidium lycopersicum | 5′ACATGAACTATAGGTACAGT |
| (cDNA) | TATATTCATATTAATGATGGCT |
| | ACAGCACTACTGGGTTATG 3′ |
| Leveillula taurica—Lt1 | 5′ACATGAACAATAGGTGTGGT |
| (cDNA) | AATATTTATATTAATGATGGCT |
| | ACAGCTCTATTGGGTTATGTTT |
| | TACCGTACGGTCAAATGAGTTT |
| | ATGAGGTGCAACAGT 3′ |
| Leveillula taurica—Lt4 | 5′ACATGAACAATAGGTGTTGT |
| (cDNA) | AATATTTATATTAATGATGGCT |
| | ACAGCTCTACTAGGTTACGTTT |
| | TACCATACGGACAAATGTCATT |
| | ATGAGGTGCAACAGT 3′ |
| Leveillula taurica—Lt2 | 5′ACATGAACTATTGGTGTTGG |
| (cDNA) | TTATCTTTATATTAATGATGGC |
| | TACTGCCCTATTAGGATATGTT |
| | TTACCATATGGTCAAATGAGTT |
| | TATGAGGTGCTACAGT 3′ |
| Leveillula taurica—Lt3 | 5′ATGAACAATTGGTACAGTAA |
| (cDNA) | TATTCATATTAATGATGGCTAC |
| | TGCACTACTGGGTTATGTTCTA |
| | CCTTTCGGACAGATGTCGCTCT |
| | GGGGTGCAACCGT 3′ |
| Pseudoperonospora cubensis | 5′TTATGGTGTTCAGGTGTTAT |
| (cDNA & genomic) | TATTTTTATTTTAATGATGGCA |
| | ACAGCCTTAATGGGTTATG 3′ |
| Alternaria solani | 5′GTATGAACTATTGGTACTGT |
| (cDNA & genomic) | TATCTTTATCTTAATGATGGCT |
| | ACAGCTCTACTGGGTTATG 3′ |
| Cercospora arachidola | 5′TTATGATCTATTGGAGTTAT |
| (cDNA & genomic) | AATTTTAGTTCTTATGATGGCA |
| | ATAGCCCTATTAGGATATG 3′ |
| Rhizoctonia solani | 5′CTATCGGAGTTGTTATGCTT |
| (cDNA) | GTTATGATGATGGGGATCGCAC |
| | TATTAGGTTATG 3′ |
| Mycosphaerella musicola | 5′GTATGAGTTATAGGTACTAT |
| (genomic & cDNA) | TATATTAGTTCTAATGATGGCT |
| | ACCGCCCTATTAGGATATG 3′ |
| Didymella bryoniae—Db1 | 5′GTATGAACAATTGGTACTGT |
| (cDNA) | TATCTTTATCTTAATGATGGCT |
| | ACAGCTCTACTGGGTTATGTTC |
| | TTCCTTATGGGCAAATGTCATT |
| | ATGAGGTGCAACTGT 3′ |
| Didymella bryoniae—Db2 | 5′GTGTGAACAATTGGTACTGT |
| (cDNA) | TATCTTTATCTTTATGATGGCT |
| | ACAGCTCTACTGGGTTATGTGC |
| | TGCCCTACGGGCAGATGTCATT |
| | ATGAGGTGCTACAGT 3′ |
| Didymella lycopersici | 5′GTATGAACAATTGGTACTGT |
| (cDNA) | TATCTTTATCTTAATGATGGCT |
| | ACAGCTCTACTGGGTTATGTTC |
| | TTCCTTATGGGCAAATGTCATT |
| | ATGAGGTGCTACAGT 3′ |

The invention extends also to DNA sequences comprising all or part of the sequence provided in Table 8 wherein the residue at a position in the DNA corresponding to the first base in the triplet coding for the amino acid at the position corresponding to S. cerevisiae cytochrome b residue 129 is a cytosine and the residue at the third base in the corresponding codon is a guanine. Such sequences form a further aspect of the invention.

TABLE 8

Tracts of plant pathogen
cytochrome b gene sequence where the residue
(shown underlined) corresponding to the first
base in the codon at the position corresponding
to S. cerevisiae cytochrome b residue 129 in the
protein is a cytosine and the residue shown in
bold at the third base of the corresponding codon
is an guanine and, as a result, encodes leucine.

| | |
|---|---|
| Species | Sequence |

| | |
|---|---|
| Plasmopara viticola | 5′TTATGGTGTTCAGGGGTAAT |
| (cDNA & genomic) | TATTTTTATTTTAATGATGGCG |
| | ACTGCACTGATGGGTTATG 3′ |
| Rhynchosporium secalis | 5′GTATGAACAATAGGTACATT |
| (cDNA & genomic) | TATATTCATATTAATGATCGTT |
| | ACAGCACTGTTGGGTTATG 3′ |
| Pyrenophora teres | 5′GTATGAACTATTGGTACTGT |
| (cDNA) | TATCTTTATCTTAATGATGGCT |
| | ACAGCCCTGCTGGGTTACG 3′ |
| Pyrenophora teres | 5′CGCTATACAGATAAATTTAG |
| (genomic) | GTTGTAGTTAGCCGGAACTTAG |
| | ACAGCCCTGCTGGGTTACCAAC |
| | ATAGCCCAAAATGGTTTAATAT |
| | AAGTAATAAAAAAAG 3′ |
| Mycosphaerella graminicola | 5′ACATGAACAATCGGTACTAT |
| (cDNA & genomic) | AATACTAGTTCTGATGATGGCA |
| | ACCGCACTGTTAGGGTATG 3′ |
| Mycosphaerella fijiensis | 5′GTATGAGTTATAGGTACTAT |
| var. difformis | TATATTAGTTCTAATGATGGCA |
| (cDNA & genomic) | ACTGCCCTGTTAGGGTATG 3′ |
| Sphaerotheca fuliginea | 5′CATTAGGTGTAGTTATATTC |
| (cDNA) | ATATTAATGATCGTTACTGCAC |
| | TGCTGGGTTATG 3′ |
| Uncinula necator | 5′CAATTGGTACAGTAATATTC |
| (cDNA) | ATTTTAATGATGGCTACAGCAC |
| | TGTTGGGTTATG 3′ |
| Colletotrichum graminicola— | 5′GTTTGAGTTATAGGTGCTAT |
| Cgr1 (genomic & cDNA) | AATACTTGTAGCTATGATGGGT |
| | ATAGGTCTGTTAGGGTATGTTT |
| | TACCTTACGGACAAATGTCATT |
| | ATGAGGTGCTACAGT 3′ |
| Colletotrichum graminicola— | 5′GTTTGAGTTATAGGTTGTAT |
| Cgr2 (genomic & cDNA) | AATACTTGTAGCTATGATGGGT |
| | ATAGGTCTGTTAGGATATGTTT |
| | TACCTTACGGACAAATGTCATT |
| | ATGAGGTGCTACAGT 3′ |
| Colletotrichum graminicola— | 5′GTTTGAGTTATAGGTACTAT |
| Cgr3 (cDNA) | AATACTTGTAGCTATGATGGGT |
| | ATAGGTCTGCTGGGTTATGTTT |
| | TACCTTACGGACAAATGTCACT |
| | ATGAGGTGCAACTGT 3′ |
| Colletotrichum graminicola— | 5′GTTTGAGTTATAGGTACTAT |
| Cgr3 (genomic) | AATACTTGTAGCTATGATGGGT |
| | ATAGGTCTGCTGGGTTACTTCA |
| | ACATAGCCCAAAATGATATGCA |
| | ATTATTAGGATTTCA 3′ |
| Pythium aphanidermatum | 5′TTATGGTGTTCAGGTGTTGT |
| (genomic & cDNA) | TATTTTTATTTTAATGATGGCA |
| | ACAGCTCTGATGGGTTATG 3′ |
| Colletotrichum | 5′GTTTGAGTTATAGGTGCTAT |
| gloeosporioides—chilli | AATACTTGTAGCTATGATGGGT |
| (genomic & cDNA) | ATAGGTCTGCTGGGTTATG 3′ |
| Colletotrichum | 5′GTTTGAGTAATAGGTGCTAT |
| gloeosporioides—mango | AATTCTTGTAGCTATGATGGGT |
| (genomic & cDNA) | ATAGGTCTGTTGGGTTATGTTT |
| | TACCTTACGGGCAAATGTCATT |
| | ATGAGGTGCAACAGT 3′ |
| Oidium lycopersicum | 5′ACATGAACTATAGGTACAGT |
| (cDNA) | TATATTCATATTAATGATGGCT |
| | ACAGCACTGCTGGGTTATG 3′ |
| Leveillula taurica—Lt1 | 5′ACATGAACAATAGGTGTGGT |
| (cDNA) | AATATTTATATTAATGATGGCT |
| | ACAGCTCTGTTGGGTTATGTTT |
| | TACCGTACGGTCAAATGAGTTT |
| | ATGAGGTGCAACAGT 3′ |
| Leveillula taurica—Lt4 | 5′ACATGAACAATAGGTGTTGT |
| (cDNA) | AATATTTATATTAATGATGGCT |
| | ACAGCTCTGCTAGGTTACGTTT |
| | TACCATACGGACAAATGTCATT |
| | ATGAGGTGCAACAGT 3′ |
| Leveillula taurica—Lt2 | 5′ACATGAACTATTGGTGTTGT |
| (cDNA) | TATCTTTATATTAATGATGGCT |
| | ACTGCCCTGTTAGGATATGTTT |
| | TACCATATGGTCAAATGAGTTT |
| | ATGAGGTGCTACAGT 3′ |
| Leveillula taurica—Lt3 | 5′ATGAACAATTGGTACAGTAA |
| (cDNA) | TATTCATATTAATGATGGCTAC |
| | TGCACTGCTGGGTTATGTTCTA |
| | CCTTTCGGACAGATGTCGCTCT |
| | GGGGTGCAACCGT 3′ |
| Pseudoperonospora cubensis | 5′TTATGGTGTTCAGGTGTTAT |
| (cDNA & genomic) | TATTTTTATTTTAATGATGGCA |
| | ACAGCTCTGATGGGTTATG 3′ |
| Alternaria solani | 5′GTATGAACTATTGGTACTGT |
| (cDNA & genomic) | TATCTTTATCTTAATGATGGCT |
| | ACAGCTCTGCTGGGTTATG 3′ |
| Cercospora arachidola | 5′TTATGATCTATTGGAGTTAT |
| (cDNA & genomic) | AATTTTAGTTCTTATGATGGCA |
| | ATAGCCCTGTTAGGATATG 3′ |
| Rhizoctonia solani | 5′CTATCGGAGTTGTTATGCTT |
| (cDNA) | GTTATGATGATGGGGATCGCAC |
| | TGTTAGGTTATG 3′ |
| Mycosphaerella musicola | 5′GTATGAGTTATAGGTACTAT |
| (genomic & cDNA) | TATATTAGTTCTAATGATGGCT |
| | ACCGCCCTGTTAGGATATG 3′ |
| Didymella bryoniae—Db1 | 5′GTATGAACAATTGGTACTGT |
| (cDNA) | TATCTTTATCTTAATGATGGCT |
| | ACAGCTCTGCTGGGTTATGTTC |
| | TTCCTTATGGGCAAATGTCATT |
| | ATGAGGTGCAACTGT 3′ |
| Didymella bryoniae—Db2 | 5′GTGTGAACAATTGGTACTGT |
| (cDNA) | TATCTTTATCTTAATGATGGCT |
| | ACAGCTCTGCTGGGTTATGTGC |
| | TGCCCTACGGGCAGATGTCATT |
| | ATGAGGTGCTACAGT 3′ |
| Didymella lycopersici | 5′GTATGAACAATTGGTACTGT |
| (cDNA) | TATCTTTATCTTAATGATGGCT |
| | ACAGCTCTGCTGGGTTATGTTC |
| | TTCCTTATGGGCAAATGTCATT |
| | ATGAGGTGCTACAGT 3′ |

The invention also extends to a fungal DNA sequence showing homology or sequence identity to said DNA sequences containing said polymorphisms and covers for example, variations in DNA sequences found in different samples of the same species. These variations may, for example, be due to the use of alternative codon usage, varying intron/exon organisation and amino acid replacement.

The DNA sequences encoding all or part of a wild type or mutant cytochrome b protein as described herein are preferably in isolated form. For example through being partially purified from any substance with which it occurs naturally. The DNA sequence is isolatable (obtainable) or isolated (obtained) from the fungi disclosed herein.

Further sequence information downstream of the 3′ end of the wild type sequences provided herein may be found in Published International Patent Application Number WO 00/66773 the teachings of which are incorporated herein by reference. The sequence information provided and teachings provided herein may be used in conjunction with that in Published International Patent Application Number WO 00/66773 to design a method of identifying the presence and absence of a mutation(s) at positions corresponding to S. cerevisiae cytochrome b residues 129 and/or 143 and the invention extends to any such method.

The invention further provides a computer readable medium having stored thereon any of the sequences described and claimed herein and including all or part of a DNA sequence encoding a mutant cytochrome b protein as herein described preferably a cytochrome b protein sequence wherein the amino acid residue at the position equivalent to residue 129 of the amino acid residue at the position equivalent to residue 129 of S. cerevisiae is a leucine and the presence of one or more mutations gives rise to fungal resistance to a strobilurin analogue or any compound in the same cross resistance group said mutation(s) occurring at a position in the DNA corresponding to one or more of the bases in the triplet coding for the amino acid at the position corresponding to S. cerevisiae cytochrome b residue 129 in the protein; all or part of a DNA encoding, or amino acid sequence of, a mutant cytochrome b protein said mutation(s) occurring at a position in the DNA corresponding to one or more of the bases in the triplet coding for the amino acid at the position corresponding to S. cerevisiae cytochrome b residue 129 in the protein wherein said protein confers fungal resistance to a strobilurin analogue or a compound in the same cross resistance group from a fungus selected from the group Plasmopara viticola, Erysiphe graminis f.sp. tritici/hordei, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Mycosphaerella musicola, Cercospora arachidola, Colletotrichum acutatum, Wilsonomyces carpophillum, Didymella bryoniae, Didymella lycopersici, Peronospora tabacina, Puccinia recondita and Puccinia horiana, preferably from the group consisting of Plasmopara viticola, Erysiphe graminis f.sp. tritici/hordei, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Didymella bryoniae, Didymella lycopersici, Mycosphaerella musicola and Cercospora arachidola; all or part of a DNA encoding, or amino acid sequence of a wild type cytochrome b sequence from a fungus selected from the group Plasmopara viticola, Rhynchosporium secalis, Pyrenophora teres, Mycosphaerella graminicola, Sphaerotheca fuliginea, Uncinula necator, Colletotrichum graminicola, Pythium aphanidermatum, Colletotrichum gloeosporioides, Oidium lycopersicum, Leveillula taurica, Pseudoperonospora cubensis, Alternaria solani, Rhizoctonia solani, Mycosphaerella musicola, Cercospora arachidola, Colletotrichum acutatum, Wilsonomyces carpophillum, Didymella bryoniae, Didymella lycopersici, Peronospora tabacina, Puccinia recondita and Puccinia horiana, preferably from the group consisting of gives rise to fungal resistance to a strobilurin analogue or any other compound in the same cross resistance group Plasmopara viticola, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Didymella bryoniae, Didymella lycopersici, Mycosphaerella musicola and Cercospora arachidola; or any allele specific oligonucleotide; allele specific oligonucleotide probe, allele specific primer, common or diagnostic primer disclosed herein.

The computer readable medium may be used, for example, in homology searching, mapping, haplotyping, genotyping or any other bioinformatic analysis. Any computer readable medium may be used, for example, compact disk, tape, floppy disk, hard drive or computer chips.

The polynucleotide sequences of the invention, or parts thereof, particularly those relating to and identifying the single nucleotide polymorphisms identified herein, especially the T to C (first base) and/or T to A or G and C to A or G (third base) changes in fungal cytochrome b causing the F129L change in the encoded protein, represent a valuable information source. The use of this information source is most easily facilitated by storing the sequence information in a computer readable medium and then using the information in standard bioinformatics programs. The polynucleotide sequences of the invention are particularly useful as components in databases for sequence identity and other search analyses. As used herein, storage of the sequence information in a computer readable medium and use in sequence databases in relation to polynucleotide or polynucleotide sequence of the invention covers any detectable chemical or physical characteristic of a polynucleotide of the invention that may be reduced to, converted into or stored in a tangible medium, such as a computer disk, preferably in a computer readable form. For example, chromatographic scan data or peak data, photographic scan or peak data, mass spectrographic data, sequence gel (or other) data.

A computer based method is also provided for performing sequence identification, said method comprising the steps of providing a polynucleotide sequence comprising a polymorphism of the invention in a computer readable medium and comparing said polymorphism containing polynucleotide sequence to at least one other polynucleotide or polypeptide sequence to identify identity (homology) i.e. screen for the presence of the polymorphism.

The invention further provides a fungal cytochrome b protein which confers fungal resistance to a strobilurin analogue or a compound within the same cross resistance group wherein in said protein a normal phenylalanine residue is altered due to the presence of one or more mutation(s) in the DNA coding for said protein said mutation(s) occurring at a position in the DNA corresponding to the first and/or third bases in the triplet coding for the amino acid at the position corresponding to S. cerevisiae cytochrome b residue 129 in the protein.

In a preferred embodiment of this aspect the invention further provides a fungal cytochrome b protein which confers fungal resistance to a strobilurin analogue or a compound within the same cross resistance group wherein in said protein a normal phenylalanine residue is altered due to the presence of a mutation in the DNA coding for said protein said mutation occurring at a position in the DNA corresponding to the first or third base in the triplet coding for the amino acid at the position corresponding to S. cerevisiae cytochrome b residue 129 in the protein.

The phenylalanine residue in the protein according to the above aspect of the invention is preferably replaced by an alternative amino acid and said replacement results in the fungus showing resistance to a strobilurin analogue or any other compound in the same cross resistance group.

The mutation according to the above aspect of the invention preferably results in the replacement of said phenylalanine residue with an amino acid selected from the group isoleucine, leucine, cysteine, serine, valine, tyrosine and most preferably leucine.

In a further aspect the invention provides an antibody capable of recognising said mutant cytochrome b protein.

In a further aspect the invention provides a method for the detection of the presence or absence of one or more mutation(s) in a fungal cytochrome b gene resulting in replacement in the encoded protein of a phenyalanine residue at the position corresponding to S. cerevisiae cytochrome b residue 129 said method comprising identifying the presence or absence of said mutation(s) in a sample of fungal nucleic acid using any or a single nucleotide polymorphism detection method wherein said single nucleotide polymorphism detection method is based on the sequence information from around 30 to 90 nucleotides upstream and/or downstream of the position corresponding to one or more of the bases in the triplet coding for the amino acid at the position corresponding to S. cerevisiae cytochrome b residue 129. in either the wild type or mutant protein.

In a further preferred embodiment of this aspect the invention provides a method for the detection of the presence or absence of one or more mutation(s) in a fungal cytochrome b gene resulting in a F129L replacement in the encoded protein said method comprising identifying the presence or absence of said mutation(s) in a sample of fungal nucleic acid using any (or a) single nucleotide polmorphism detection method wherein said single nucleotide polymorphism detection method is based on the sequence information from around 30 to 90 nucleotides upstream and/or downstream of the position corresponding to the first or third base in the triplet coding for the amino acid at the position corresponding to S. cerevisiae cytochrome b residue 129 in either the wild type or mutant protein.

In a further preferred embodiment of this aspect the invention provides a method for the detection of a first base thymine to a cytosine mutation and/or a third base thymine to adenine or guanine mutation or a cytosine to a adenine or guanine mutation in a fungal cytochrome b gene resulting in a F129L replacement in the encoded protein said method comprising identifying the presence or absence of said mutation(s) in a sample of fungal nucleic acid using any (or a) single nucleotide polymorphism detection method wherein said single nucleotide polymorphism detection method is based on the sequence information from around 30 to 90 nucleotides upstream and/or downstream of the position corresponding to the first or third base in the triplet coding for the amino acid at the position corresponding to S. cerevisiae cytochrome b residue 129 in either the wild type or mutant protein.

In a further particularly preferred embodiment of this aspect the invention provides a method for the detection of a first base thymine to cytosine mutation or a third base thymine to adenine or guanine mutation or a cytosine to a adenine or guanine mutation in a fungal cytochrome b gene resulting in a F129L replacement in the encoded protein said method comprising identifying the presence or absence of said mutation(s) in a sample of fungal nucleic acid using any (or a) single nucleotide polymorphism detection method wherein said single nucleotide polymorphism detection method is based on the sequence information from around 30 to 90 nucleotides upstream and/or downstream of the position corresponding to the first or third base in the triplet coding for the amino acid at the position corresponding to S. cerevisiae cytochrome b residue 129 in either the wild type or mutant protein.

In a further particularly preferred embodiment of this aspect the invention provides a:method for the detection of a third base cytosine to adenine mutation in a fungal cytochrome b gene resulting in a F129L replacement in the encoded protein said method comprising identifying the presence or absence of said mutation(s) in a sample of fungal nucleic acid using any (or a) single nucleotide polymorphism detection method wherein said single nucleotide polymorphism detection method is based on the sequence information from around 30 to 90 nucleotides upstream and/or downstream of the position corresponding to the first or third base in the triplet coding for the amino acid at the position corresponding to S. cerevisiae cytochrome b residue 129 in either the wild type or mutant protein.

In the above aspects of the invention the single nucleotide polymorphism detection method is preferably based on the sequence information from around 30 to 90 nucleotides upstream and/or downstream of the position corresponding to the third base in the triplet coding for the amino acid at the position corresponding to S. cerevisiae cytochrome b residue 129 in either the wild type or mutant protein.

As used herein the term “upstream” is used to denote sequences “5′to” and the term “downstream” to denote sequences “3′ to”.

The sequence information according to the above aspect of the invention is preferably derived from a fungus selected from the group comprising: Plasmopara viticola, Erysiphe graminis f.sp. tritici/hordei, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Mycosphaerella musicola, Cercospora arachidola, Colletotrichum acutatum, Wilsonomyces carpophillum, Didymella bryoniae, Didymella lycopersici, Peronospora tabacina, Puccinia recondita and Puccinia horiana, preferably from the group consisting of Plasmopara viticola, Erysiphe graminis f.sp. tritici/hordei, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Didymella bryoniae, Didymella lycopersici; Mycosphaerella musicola and Cercospora arachidola and more preferably from the group Plasmopara viticola, Erysiphe graminis f.sp. tritici/hordei, Rhynchosporium secalis, Pyrenophora teres, Mycosphaerella graminicola, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Leveillula taurica, Pseudoperonospora cubensis, Alternaria solani, Rhizoctania solani, Didymella bryoniae, Didymella lycopersici, Mycosphaerella musicola and Cercospora arachidola.

As used herein the term around 30 means that the sequence may comprise up to 30 nucleotides, for example 5, up to 10, 15, 20, or 25 nucleotides or may comprise more than 30 nucleotides. In the above aspects of the invention it is preferred that the sequence information used is around 30, preferably 30 nucleotides upstream and/or downstream of the position corresponding to the first or third base in the triplet coding for the amino acid at the position corresponding to S. cerevisiae cytochrome b residue 129 in either the wild type or mutant protein.

The nucleic acid according to the invention is preferably DNA. The test sample of nucleic acid is conveniently a total DNA preparation from fungal material, a cDNA preparation from fungal material or the fungal material itself or plant or seed extracts containing fungal nucleic acid. In this specification, we describe the detection of the F129L mutation by using total genomic DNA or cDNA preparations. However, it will be appreciated that the test sample may equally be a nucleic acid, the sequence of which corresponds to the sequence in the test sample. That is to say that all or a part of the region in the sample nucleic acid may firstly be isolated or amplified using any convenient technique such as PCR before use in a method of the invention.

The present invention provides a means of analysing mutations in the DNA of agricultural field samples which by their very origin are normally considerably less well defined compared with analogous situations involving human samples with which the diagnostic methods described herein are more commonly used. Agricultural field samples are considerably more difficult to work with and it is more technically demanding to detect a mutation event occurring at a low frequency amongst a very large amount of wild type DNA and/or extraneous DNA from other organisms that is/are present in a field isolate when compared with a human sample that frequently contains DNA from only one individual.

Any convenient enzyme for polymerisation may be used provided that it has no intrinsic ability to discriminate between normal and mutant template sequences to any significant extent. Examples of convenient enzymes include thermostable enzymes which have no significant 3′-5′ exonuclease activity, for example Taq DNA polymerase, particularly ‘Ampli Taq Gold’™ DNA polymerase (Applied Biosystems), Stoffel fragment, or other appropriately N-terminal deleted modifications of Taq (Thermus aquaticus) or Tth (Thermus thermophilus) DNA polymerases.

In a further aspect the current invention provides an allele specific oligonucleotide capable of binding to a fungal nucleic acid sequence encoding a wild type cytochrome b protein wherein said oligonucleotide comprises a sequence which recognises a nucleic acid sequence encoding a phenylalanine residue at the position corresponding to S. cerevisiae cytochrome b residue 129.

In a preferred embodiment the said fungal nucleic acid sequence is selected from a fungus from the group consisting of Plasmopara viticola, Erysiphe graminis f.sp. tritici/hordei, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Mycosphaerella musicola, Cercospora arachidola, Colletotrichum acutatum, Wilsonomyces carpophillum, Didymella bryoniae, Didymella lycopersici, Peronospora tabacina, Puccinia recondita and Puccinia horiana.

In a preferred embodiment of this aspect of the invention the said fungal nucleic acid sequence is selected from the group consisting of Plasmopara viticola, Erysiphe graminis f.sp. tritici/hordei, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Didymella bryoniae, Didymella lycopersici, Mycosphaerella musicola, and Cercospora arachidola and in a particularly preferred embodiment the fungal nucleic acid is selected from the group consisting of Plasmopara viticola, Erysiphe graminis f.sp. tritici/hordei, Rhynchosporium secalis, Pyrenophora teres, Mycosphaerella graminicola, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Leveillula taurica, Pseudoperonospora cubensis, Alternaria solani, Rhizoctonia solani, Didymella bryoniae, Didymella lycopersici, Mycosphaerella musicola and Cercospora arachidola.

The fungal nucleic acid according to the above aspects of the invention is preferably DNA.

In a further aspect of the invention we provide an allele specific oligonucleotide capable of binding to a fungal nucleic acid sequence encoding a mutant cytochrome b protein wherein said oligonucleotide comprises a sequence which recognises a nucleic acid sequence encoding an amino acid selected from the group isoleucine, leucine, serine, cysteine, valine, tyrosine, and most preferably leucine at the position corresponding to S. cerevisiae cytochrome b residue 129.

In a preferred embodiment of this aspect of the invention we provide an allele specific oligonucleotide capable of binding to a fungal nucleic acid sequence encoding a mutant cytochrome b protein selected from the group consisting of Plasmopara viticola, Erysiphe graminis f.sp. tritici/hordei, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Mycosphaerella musicola, Cercospora arachidola, Colletotrichum acutatum, Wilsonomyces carpophillum, Didymella bryoniae, Didymella lycopersici, Peronospora tabacina, Puccinia recondita and Puccinia horiana wherein said oligonucleotide comprises a sequence which recognises a nucleic acid sequence encoding an amino acid selected from the group isoleucine, leucine; serine, cysteine, valine, tyrosine, and most preferably leucine at the position corresponding to S. cerevisiae cytochrome b residue 129.

In a further preferred embodiment of this aspect of the invention we provide an allele specific oligonucleotide capable of binding to a fungal nucleic acid sequence encoding a mutant cytochrome b protein selected from the group consisting of Plasmopara viticola, Erysiphe graminis f.sp. tritici/hordei, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Didymella bryoniae, Didymella lycopersici, Mycosphaerella musicola and Cercospora arachidola.

The fungal nucleic acid according to the above aspects of the invention is preferably DNA.

In a further aspect the invention provides an allele specific oligonucleotide probe capable of detecting a wild type cytochrome b gene sequence at a position in the DNA corresponding to one or more of the bases in the triplet coding for the amino acid at the position corresponding to S. cerevisiae cytochrome b residue 129 in the protein.

In a further aspect the invention provides an allele specific oligonucleotide probe capable of detecting a fungal cytochrome b gene polymorphism at a position in the DNA corresponding to one or more of the bases in the triplet coding for the amino acid at the position corresponding to S. cerevisiae cytochrome b residue 129 in the protein.

In a further preferred embodiment of this aspect the invention provides an allele specific oligonucleotide probe capable of detecting a fungal cytochrome b gene polymorphism at positions in the DNA corresponding to the first and/or third base in the triplet coding for the amino acid at the position corresponding to S. cerevisiae cytochrome b residue 129.

In a further preferred embodiment of this aspect the invention provides an allele specific oligonucleotide probe capable of detecting a fungal cytochrome b gene polymorphism at positions in the DNA corresponding to the first or third base in the triplet coding for the amino acid at the position corresponding to S. cerevisiae cytochrome b residue 129.

In a further preferred embodiment of this aspect the invention provides an allele specific oligonucleotide probe capable of detecting,a fungal cytochrome b gene polymorphism at positions in the DNA corresponding to the third base in the triplet coding for the amino acid at the position corresponding to S. cerevisiae cytochrome b residue 129.

In further preferred embodiments of the above aspect of the invention said polymorphism is a thymine to cytosine base change at the first base a thymine or cytosine to adenine or guanine change at the third base of the codon, the mutation is in a fungus selected from the group consisting of Plasmopara viticola, Erysiphe graminis f.sp. tritici/hordei, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Mycosphaerella musicola, Cercospora arachidola, Colletotrichum acutatum, Wilsonomyces carpophillum, Didymella bryoniae, Didymella lycopersici, Peronospora tabacina, Puccinia recondita and Puccinia horiana.

In further preferred embodiments of the above aspect of the invention said polymorphism is a thymine to cytosine base change at the first base a thymine or cytosine to adenine or guanine change at the third base of the codon, the mutation is in a fungus selected from the group consisting of Plasmopara viticola, Erysiphe graminis f.sp. tritici/hordei, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Didymella bryoniae, Didymella lycopersici, Mycosphaerella musicola and Cercospora arachidola.

The allele-specific oligonucleotide probe is preferably 12 to 50 nucleotides long, more preferable about 12-35 nucleotides long and most preferably about 12-30 nucleotides long.

The design of such probes will be apparent to the molecular biologist of ordinary skill and may be based on DNA or RNA sequence information. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection (e.g. fluorescent labels including for example FAM and VIC).

The invention further provides nucleotide primers which can detect the nucleotide polymorphisms according to the invention.

According to another aspect of the invention there is provided an allele specific primer capable of detecting a cytochrome b gene polymorphism at a position in the DNA corresponding to one or more of the bases in the triplet coding for the amino acid at the position corresponding to S. cerevisiae cytochrome b residue 129 in the protein.

According to a preferred embodiment of this aspect of the invention there is provided an allele specific primer capable of detecting a cytochrome b gene polymorphism at a position in the DNA corresponding to the first and/or third base in the triplet coding for the amino acid at the position corresponding to S. cerevisiae cytochrome b residue 129.

According to a further preferred embodiment of this aspect of the invention there is provided an allele specific primer capable of detecting a cytochrome b gene polymorphism at a position in the DNA corresponding to the first or third base in the triplet coding for the amino acid at the position corresponding to S. cerevisiae cytochrome b residue 129.

According to a further preferred embodiment of this aspect of the invention there is provided an allele specific primer capable of detecting a cytochrome b gene polymorphism at a position in the DNA corresponding to the third base in the triplet coding for the amino acid at the position corresponding to S. cerevisiae cytochrome b residue 129 in the protein.

In the above aspects the said mutation in the DNA sequence is preferably a thymine to cytosine base change at the first base of the triplet and a thymine or cytosine to adenine or guanine change at the third base of the triplet, most preferably a cytosine to adenine change at the third position.

In a further aspect the invention provides an allele specific primer capable of detecting a fungal DNA sequence encoding a wild type cytochrome b protein selected from the group consisting of Plasmopara viticola, Erysiphe graminis f.sp. tritici/hordei, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Mycosphaerella musical, Cercospora arachidola, Colletotrichum acutatum, Wilsonomyces carpophillum, Didymella bryoniae, Didymella lycopersici, Peronospora tabacina, Puccinia recondita and Puccinia horiana wherein said primer is capable of detecting a DNA sequence encoding a phenylalanine residue at the position corresponding to S. cerevisiae cytochrome b residue 129.

In a preferred embodiment of this aspect the invention provides an allele specific primer capable of detecting a fungal DNA sequence encoding a wild type cytochrome b protein selected from the group consisting of Plasmopara viticola, Erysiphe graminis f.sp. tritici/hordei, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Didymella bryoniae, Didymella lycopersici, Mycosphaerella musicol, and Cercospora arachidola horiana wherein said primer is capable of detecting a DNA sequence encoding a phenylalanine residue at the position corresponding to S. cerevisiae cytochrome b residue 129.

In a preferred embodiment of this aspect of the invention the said fungal DNA sequence is selected from the group consisting of Plasmopara viticola, Erysiphe graminis f.sp. tritici/hordei, Rhynchosporium secalis, Pyrenophora teres, Mycosphaerella graminicola, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Leveillula taurica, Pseudoperonospora cubensis, Alternaria solani, Rhizoctonia solani, Didymella bryoniae, Didymella lycopersici, Mycosphaerella musicola and Cercospora arachidola.

In a further aspect of the invention we provide an allele specific primer capable of detecting a fungal DNA sequence encoding part of a mutant cytochrome b protein wherein said allele specific primer is capable of detecting a DNA sequence encoding an amino acid selected from the group isoleucine, leucine, serine, cysteine, valine, tyrosine, and most preferably leucine at the position corresponding to S. cerevisiae cytochrome b residue 129.

In a further embodiment of this aspect of the invention we provide an allele specific primer capable of detecting a fungal DNA sequence encoding part of a mutant cytochrome b protein wherein said allele specific primer is capable of detecting a DNA sequence encoding an amino acid selected from the group isoleucine, leucine, serine, cysteine, valine, tyrosine, and most preferably leucine at the position corresponding to S. cerevisiae cytochrome b residue 129.

In a preferred embodiment of this aspect of the invention we provide an allele specific primer capable of detecting a fungal DNA sequence encoding part of a mutant cytochrome b protein selected from the group consisting of Plasmopara viticola, Erysiphe graminis f.sp. tritici/hordei, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Mycosphaerella musicola, Cercospora arachidola, Colletotrichum acutatum, Wilsonomyces carpophillum, Didymella bryoniae, Didymella lycopersici, Peronospora tabacina, Puccinia recondita and Puccinia horiana wherein said primer is capable of detecting a DNA sequence encoding an amino acid selected from the group isoleucine, leucine, serine, cysteine, valine, tyrosine, and most preferably leucine at the position corresponding to S. cerevisiae cytochrome b residue 129.

In a further preferred embodiment of this aspect of the invention we provide an allele specific primer capable of detecting a fungal DNA sequence encoding part of a mutant cytochrome b protein selected from the group consisting of Plasmopara viticola, Erysiphe graminis f.sp. tritici/hordei, Rhynchosporium secalis, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Didymella bryoniae, Didymella lycopersici, Mycosphaerella musicola and Cercospora arachidola wherein said primer is capable of detecting a DNA sequence encoding an amino acid selected from the group isoleucine, leucine, serine, cysteine, valine, tyrosine, and most preferably leucine at the position corresponding to S. cerevisiae cytochrome b residue 129.

An allele specific primer is used generally with a common primer in an amplification reaction such as a PCR reaction which provides the discrimination between alleles through selective amplification of one allele at a particular sequence position e.g as used in the ARMS assay.

We are now able to devise primers for the F129L mutation in the above-listed fungal species which have been shown to detect the specific mutations reliably and robustly. The primers detect either the thymine to cytosine base change at a position in the DNA corresponding to the first base in the triplet coding for the amino acid at the position corresponding to S. cerevisiae cytochrome b residue 129 in the protein and/or the thymine or cytosine to adenine or guanine base changes at a position in the DNA corresponding to the third base in the triplet coding for the amino acid at the position corresponding to S. cerevisiae cytochrome b residue 129 in the protein. The allele specific primers are herein referred to as diagnostic primers.

In a further aspect the invention therefore provides a diagnostic primer capable of binding to a template comprising a mutant type fungal cytochrome b nucleotide sequence at a position corresponding to the first and/or third base in the triplet coding for the amino acid at the position corresponding to S. cerevisiae cytochrome b residue 129 in the cytochrome b protein wherein the final 3′ nucleotide of the primer corresponds to a nucleotide present in said mutant form of a fungal cytochrome b gene and the presence of said nucleotide gives rise to fungal resistance to a strobilurin analogue or any other compound in the same cross resistance group.

In a further embodiment of this aspect the invention therefore provides a diagnostic primer capable of binding to a template comprising a mutant type fungal cytochrome b nucleotide sequence at a position corresponding to first or the third base in the triplet coding for the amino acid at the position corresponding to S. cerevisiae cytochrome b residue 129 in the cytochrome b protein wherein the final 3′ nucleotide of the primer corresponds to a nucleotide present in said mutant form of a fungal cytochrome b gene and the presence of said nucleotide gives rise to fungal resistance to a strobilurin analogue or any other compound in the same cross resistance group.

In a further embodiment of this aspect the invention therefore provides a diagnostic primer capable of binding to a template comprising a mutant type fungal cytochrome b nucleotide sequence at a position corresponding to the first and/or third base in the triplet coding for the amino acid at the position corresponding to S. cerevisiae cytochrome b residue 129 in the cytochrome b protein wherein the final 3′ nucleotide of the primer corresponds to a nucleotide present in said mutant form of a fungal cytochrome b gene and the presence of said nucleotide gives rise to fungal resistance to a strobilurin analogue or any other compound in the same cross resistance group.

In a further preferred embodiment of this aspect the invention therefore provides a diagnostic primer capable of binding to a template comprising a mutant type fungal cytochrome b nucleotide sequence at a position corresponding to the first or third base in the triplet coding for the amino acid at the position corresponding to S. cerevisiae cytochrome b residue 129 in the cytochrome b protein wherein the final 3′ nucleotide of the primer corresponds to a nucleotide present in said mutant form of a fungal cytochrome b gene and the presence of said nucleotide gives rise to fungal resistance to a strobilurin analogue or any other compound in the same cross resistance group.

In a further aspect the invention therefore provides a diagnostic primer capable of binding to a template comprising a mutant type fungal cytochrome b nucleotide sequence at a position corresponding to the third base in the triplet coding for the amino acid at the position corresponding to S. cerevisiae cytochrome b residue 129 in the cytochrome b protein wherein the final 3′ nucleotide of the primer corresponds to a nucleotide present in said mutant form of a fungal cytochrome b gene and the presence of said nucleotide gives rise to fungal resistance to a strobilurin analogue or any other compound in the same cross resistance group.

In a further aspect the invention therefore provides a diagnostic primer capable of binding to a template comprising a mutant type fungal cytochrome b nucleotide sequence at a position corresponding to the first base in the triplet coding for the amino acid at the position corresponding to S. cerevisiae cytochrome b residue 129 in the cytochrome b protein wherein the final 3′ nucleotide of the primer corresponds to a nucleotide present in said mutant form of a fungal cytochrome b gene and the presence of said nucleotide gives rise to fungal resistance to a strobilurin analogue or any other compound in the same cross resistance group.

The diagnostic primer of the invention is preferably at least 20 nucleotides in length, and most preferably about 26 nucleotides in length. However, diagnostic primers of the invention may also be between 15 and 20 nucleotides in length. It will be appreciated by th eskilled man that diagnostic primers of the invention may be such thtat they hybridise to either the sense or the antisense strand of nucleic acid encoding the fungal cytochrome b protein.

In a preferred embodiment of the above aspect of the invention the penultimate nucleotide (−2) of the primer is not the same as that present in the corresponding position in the wild type cytochrome b sequence.

In a further preferred embodiment it is the −3 nucleotide of the primer which is not the same as that present in the corresponding position in the wild type cytochrome b sequence.

Other destabilising components may be incorporated along with the −2 or −3 nucleotide.

In a further particularly preferred embodiment of the above aspect of the invention we provide diagnostic primers capable of binding to a template comprising a mutant type fungal cytochrome b nucleotide sequence corresponding to the first and/or third base in the triplet coding for the amino acid at the position corresponding to S. cerevisiae cytochrome b residue 129. in the cytochrome b protein wherein the final 3′ nucleotide of the primer corresponds to a nucleotide present in said mutant form of a fungal cytochrome b gene and wherein up to 10, such as up to 8, 6, 4, 2, 1, of the remaining nucleotides may be varied with respect to the wild type sequence without significantly affecting the properties of the diagnostic primer.

In a further particularly preferred embodiment of the above aspect of the invention we provide diagnostic primers capable of binding to a template comprising a mutant type fungal cytochrome b nucleotide sequence corresponding to the first or third base in the triplet coding for the amino acid at the position corresponding to S. cerevisiae cytochrome b residue 129 in the cytochrome b protein wherein the final 3′ nucleotide of the primer corresponds to a nucleotide present in said mutant form of a fungal cytochrome b gene and wherein up to 10, such as up to 8, 6, 4, 2, 1, of the remaining nucleotides may be varied with respect to the wild type sequence without significantly affecting the properties of the diagnostic primer.

In a further particularly preferred embodiment of the above aspect of the invention we provide diagnostic primers capable of binding to a template comprising a mutant type fungal cytochrome b nucleotide sequence corresponding to the third base in the triplet coding for the amino acid at the position corresponding to S. cerevisiae cytochrome b residue 129 in the cytochrome b protein wherein the final 3′ nucleotide of the primer corresponds to a nucleotide present in said mutant form of a fungal cytochrome b gene and wherein up to 10, such as up to 8, 6, 4, 2, 1, of the remaining nucleotides may be varied with respect to the wild type sequence without significantly affecting the properties of the diagnostic primer.

In a further particularly preferred embodiment of the above aspect of the invention we provide diagnostic primers capable of binding to a template comprising a mutant type fungal cytochrome b nucleotide sequence corresponding to the first base in the triplet coding for the amino acid at the position corresponding to S. cerevisiae cytochrome b residue 129 in the cytochrome b protein wherein the final 3′ nucleotide of the primer corresponds to a nucleotide present in said mutant form of a fungal cytochrome b gene and wherein up to 10, such as up to 8, 6, 4, 2, 1, of the remaining nucleotides may be varied with respect to the wild type sequence without significantly affecting the properties of the diagnostic primer.

In a further particularly preferred embodiment of the above aspect of the invention we provide diagnostic primers comprising the sequences given below and derivatives thereof wherein the final nucleotide at the 3′ end is identical to the sequences given below and wherein up to 10, such as up to 8, 6, 4, 2, 1, of the remaining nucleotides may be varied without significantly affecting the properties of the diagnostic primer.

Diagnostic (e.g. ARMS) primers will have a high Tm as will be appreciated by the man skilled in the art, and it is preferred that the ARMS primers in all aspects of the invention are about 26 nucleotides in length. Conveniently, the sequence of the diagnostic primer may be exactly as provided below (see Tbales 9 to 13). In all the primers listed below int Table 9 to 13, the penultimate nucleotide has been altered from wild type cyt b sequence to destabilise the template/primer hybrid thereby making it more selective for the desired template and these primers are particularly preferred according to the invention. It will be apparent to the man skilled in the art of primer design that bases alternative to or in addition to those discussed above may also be varied without adversely affecting the ability of the primer to bind to the template.

TABLE 9

ARMS primer design for the detection of
the F129L mutation where the first base in the
triplet coding for the leucine residue is a cytosine.

| | | primer sequence |
|---|---|---|
| | | for the detection |
| | | of F129L (thymine to |
| | | cytosine change at first |
| Primer # | species: | base of triplet 5′ to 3′) |
| 1 | Plasmopara viticola (reverse) | CCCAAGGCAAAACATAACCCATCAG |
| 2 | Erysiphe graminis f. sp. tritici/hordei | ATTCATATTAATGATCGTTACTGCCC |
| 3 | Rhynchosporium secalis | ATTCATATTAATGATCGTTACAGCGC |
| 4 | Pyrenophora teres (cDNA) | CTTTATCTTAATGATGGCTACAGCGC |
| 5 | Pyrenophora teres (genomic) | GTAGTTAGCCGGAACTTAGACAGCGC |
| 6 | Mycosphaerella graminicola | ACTAGTTCTGATGATGGCAACCGCGC |
| 7 | Mycosphaerella fijiensis var. difformis | ATTAGTTCTAATGATGGCAACTGCGC |
| 8 | Sphaerotheca fuliginea | ATTCATATTAATGATCGTTACTGCGC |
| 9 | Uncinula necator | ATTCATTTTAATGATGGCTACAGCGC |
| 10 | Colletotrichum graminicola—Cgr1 | ACTTGTAGCTATGATGGGTATAGGCC |
| 11 | Colletotrichum graminicola—Cgr2 | ACTTGTAGCTATGATGGGTATAGGCC |
| 12 | Colletotrichum graminicola—Cgr3 | ACTTGTAGCTATGATGGGTATAGGAC |
| 13 | Pythium aphanidermatum (forward) | TTTTATTTTAATGATGGCAACAGCGC |
| 14 | Colletotrichum gloeosporioides—chilli | ACTTGTAGCTATGATGGGTATAGGAC |
| 15 | Colletotrichum gloeosporioides—mango | TCTTGTAGCTATGATGGGTATAGGAC |
| 16 | Oidium lycopersicum | ATTCATATTAATGATGGCTACAGCGC |
| 17 | Leveillula taurica—Lt1 | ATTTATATTAATGATGGCTACAGCGC |
| 18 | Leveillula taurica—Lt4 | ATTTATATTAATGATGGCTACAGCGC |
| 19 | Leveillula taurica—Lt2 | CTTTATATTAATGATGGCTACTGCGC |
| 20 | Leveillula taurica—Lt3 | ATTCATATTAATGATGGCTACTGCGC |
| 21 | Pseudoperonospora cubensis | TTTTATTTTAATGATGGCAACAGCGC |
| 22 | Alternaria solani | CTTTATCTTAATGATGGCTACAGCGC |
| 23 | Cercospora arachidola | TTTAGTTCTTATGATGGCAATAGCGC |
| 24 | Rhizoctonia solani | GCTTGTTATGATGATGGGGATCGCGC |
| 25 | Venturia inaequalis | CTTTATATTAATGATAGTTACAGCGC |
| 26 | Phytophthora infestans | TTTTATTTTAATGATGGCTACTGCGC |
| 27 | Mycosphaerella musicola | ATTAGTTCTAATGATGGCTACCGCGC |
| 28 | Didymella bryoniae—Db1 | CTTTATCTTAATGATGGCTACAGCGC |
| 29 | Didymella bryoniae—Db2 | CTTTATCTTAATGATGGCTACAGCGC |
| 30 | Didymella lycopersici | CTTTATCTTAATGATGGCTACAGCGC |

TABLE 10

ARMS primer design for the detection of the
F129L mutation where the third base in the triplet
coding for the leucine residue is an adenine residue.

| | | primer sequence |
|---|---|---|
| | | for the detection |
| | | of F129L (thymine to |
| | | cytosine change at third |
| Primer # | species: | base of triplet 5′ to 3′) |
| 1 | Plasmopara viticola (reverse) | TCCCCAAGCCAAAACATAACCCAGT |
| 2 | Erysiphe graminis f. sp. tritici/hordei | TCATATTAATGATCGTTACTGCATGA |
| 3 | Rhynchosporium secalis | TCATATTAATGATCGTTACAGCATGA |
| 4 | Pyrenophora teres (cDNA) | TTATCTTAATGATGGCTACAGCCTGA |
| 5 | Pyrenophora teres (genomic) | AGTTAGCCGGAACTTAGACAGCCTGA |
| 6 | Mycosphaerella graminicola | TAGTTCTGATGATGGCAACCGCATGA |
| 7 | Mycosphaerella fijiensis var. difformis | TAGTTCTAATGATGGCAACTGCCTGA |
| 8 | Sphaerotheca fuliginea | TCATATTAATGATCGTTACTGCATGA |
| 9 | Uncinula necator | TCATTTTAATGATGGCTACAGCATGA |
| 10 | Colletotrichum graminicola—Cgr1 | TTGTAGCTATGATGGGTATAGGTTGA |
| 11 | Colletotrichum graminicola—Cgr2 | TTGTAGCTATGATGGGTATAGGTTGA |
| 12 | Colletotrichum graminicola—Cgr3 | TTGTAGCTATGATGGGTATAGGTTGA |
| 13 | Pythium aphanidermatum (reverse) | ACCCCAAGGTAATACATAACACGTT |
| 14 | Colletotrichum gloeosporioides—chilli | TTGTAGCTATGATGGGTATAGGTTGA |
| | and mango | |
| 15 | Oidium lycopersicum | TCATATTAATGATGGCTACAGCATGA |
| 16 | Leveillula taurica Lt1 | TTATATTAATGATGGCTACAGCTTGA |
| 17 | Leveillula taurica Lt4 | TTATATTAATGATGGCTACAGCTTGA |
| 18 | Leveillula taurica Lt2 | TTATATTAATGATGGCTACTGCCTGA |
| 19 | Leveillula taurica Lt3 | TCATATTAATGATGGCTACTGCATGA |
| 20 | Pseudoperonospora cubensis | TTATTTTAATGATGGCAACAGCTTGA |
| 21 | Alternaria solani | TTATCTTAATGATGGCTACAGCTTGA |
| 22 | Cercospora arachidola | TAGTTCTTATGATGGCAATAGCCTGA |
| 23 | Rhizoctonia solani | TTGTTATGATGATGGGGATCGCATGA |
| 24 | Venturia inaequalis | TTATATTAATGATAGTTACAGCCTGA |
| 25 | Phytophthora infestans | TTATTTTAATGATGGCTACTGCTTGA |
| 26 | Mycosphaerella musicola | TAGTTCTAATGATGGCTACCGCCTGA |
| 27 | Didymella bryoniae—Db1 | TTATCTTAATGATGGCTACAGCTTGA |
| 28 | Didymella bryoniae—Db2 | TTATCTTAATGATGGCTACAGCTTGA |
| 29 | Didymella lycopersici | TTATCTTAATGATGGCTACAGCTTGA |

TABLE 11

ARMS primer design for the detection of
the F129L mutation where the third base in
the triplet coding for the leucine residue is guanine.

| | | |
|---|---|---|
| | | primer sequence for |
| | | the detection of F129L |
| | | (thymine to cytosine |
| | | to guanine change at third |
| Primer # | species: | base of triplet 5′ to 3′) |

| | | |
|---|---|---|
| 1 | Plasmopara viticola (reverse) | TCCCCAAGGCAAAACATAACCCAGC |
| 2 | Erysiphe graminis f. sp. tritici/hordei | TCATATTAATGATCGTTACTGCATCG |
| 3 | Rhynchosporium secalis | TCATATTAATGATCGTTACAGCATCG |
| 4 | Pyrenophora teres (cDNA) | TTATCTTAATGATGGCTACAGCCTCG |
| 5 | Pyrenophora teres (genomic) | AGTTAGCCGGAACTTAGACAGCCTCG |
| 6 | Mycosphaerella graminicola | TAGTTCTGATGATGGCAACCGCATCG |
| 7 | Mycosphaerella fijiensis var. difformis | TAGTTCTAATGATGGCAACTGCCTCG |
| 8 | Sphaerotheca fuliginea | TCATATTAATGATCGTTACTGCATCG |
| 9 | Uncinula necator | TCATTTTAATGATGGCTACAGCATCG |
| 10 | Colletotrichum graminicola—Cgr1 | TTGTAGCTATGATGGGTATAGGTTCG |
| 11 | Colletotrichum graminicola—Cgr2 | TTGTAGCTATGATGGGTATAGGTTCG |
| 12 | Colletotrichum graminicola—Cgr3 | TTGTAGCTATGATGGGTATAGGTTCG |
| 13 | Pythium aphanidermatum (forward) | TTATTTTAATGATGGCAACAGCTTCG |
| 14 | Colletotrichum gloeosporioides—chilli | TTGTAGCTATGATGGGTATAGGTTCG |
| | and mango | |
| 15 | Oidium lycopersicum | TCATATTAATGATGGCTACAGCATCG |
| 16 | Leveillula taurica Lt1 | TTATATTAATGATGGCTACAGCTTCG |
| 17 | Leveillula taurica Lt4 | TTATATTAATGATGGCTACAGCTTCG |
| 18 | Leveillula taurica Lt2 | TTATATTAATGATGGCTACTGCCTCG |
| 19 | Leveillula taurica Lt3 | TCATATTAATGATGGCTACTGCATCG |
| 20 | Pseudoperonospora cubensis | TTATTTTAATGATGGCAACAGCTTCG |
| 21 | Alternaria solani | TTATCTTAATGATGGCTACAGCTTCG |
| 22 | Cercospora arachidola | TAGTTCTTATGATGGCAATAGCCTCG |
| 23 | Rhizoctonia solani | TTGTTATGATGATGGGGATCGCATCG |
| 24 | Venturia inaequalis | TTATATTAATGATAGTTACAGCCTCG |
| 25 | Phytophthora infestans | TTATTTTAATGATGGCTACTGCTTCG |
| 26 | Mycosphaerella musicola | TAGTTCTAATGATGGCTACCGCCTCG |
| 27 | Didymella bryoniae—Db1 | TTATCTTAATGATGGCTACAGCTTCG |
| 28 | Didymella bryoniae—Db2 | TTATCTTAATGATGGCTACAGCTTCG |
| 29 | Didymella lycopersici | TTATCTTAATGATGGCTACAGCTTCG |

Table 12

ARMS primer design for the detection of the F129L
mutation where the first base on the triplet coding
for residue 129 is a cytosine and the third base
is an adenine residue such that leucine is encoded.

| | | |
|---|---|---|
| | | primer sequence for |
| | | the detection of F129L |
| | | (thymine to cytosine |
| | | change at the first base |
| | | and a thymine or cytosine |
| | | to adenine change at third |
| Primer # | species: | base of triplet 5′ to 3′) |

| | | |
|---|---|---|
| 1 | Plasmopara viticola (reverse) | TTATTTTAATGATGGCGACTGCACGA |
| 2 | Erysiphe graminis f. sp. tritici/hordei | TCATATTAATGATCGTTACTGCACGA |
| 3 | Rhynchosporium secalis | TCATATTAATGATCGTTACAGCACGA |
| 4 | Pyrenophora teres (cDNA) | TTATCTTAATGATGGCTACAGCCCGA |
| 5 | Pyrenophora teres (genomic) | AGTTAGCCGGAACTTAGACAGCCCGA |
| 6 | Mycosphaerella graminicola | TAGTTCTGATGATGGCAACCGCACGA |
| 7 | Mycosphaerella fijiensis var. difformis | TAGTTCTAATGATGGCAACTGCCCGA |
| 8 | Sphaerotheca fuliginea | TCATATTAATGATCGTTACTGCACGA |
| 9 | Uncinula necator | TCATTTTAATGATGGCTACAGCACGA |
| 10 | Colletotrichum graminicola—Cgr1 | TTGTAGCTATGATGGGTATAGGTCGA |
| 11 | Colletotrichum graminicola—Cgr2 | TTGTAGCTATGATGGGTATAGGTCGA |
| 12 | Colletotrichum graminicola—Cgr3 | TTGTAGCTATGATGGGTATAGGTCGA |
| 13 | Pythium aphanidermatum (forward) | TTATTTTAATGATGGCAACAGCTCGA |
| 14 | Colletotrichum gloeosporioides—chilli | TTGTAGCTATGATGTATATAGGTCGA |
| | and mango | |
| 15 | Oidium lycopersicum | TCATATTAATGATGGCTACAGCACGA |
| 16 | Leveillula taurica Lt1 | TTATATTAATGATGGCTACAGCTCGA |
| 17 | Leveillula taurica Lt4 | TTATATTAATGATGGCTACAGCTCGA |
| 18 | Leveillula taurica Lt2 | TTATATTAATGATGGCTACTGCCCCA |
| 19 | Leveillula taurica Lt3 | TCATATTAATGATGGCTACTGCACGA |
| 20 | Pseudoperonospora cubensis | TTATTTTAATGATGCCAACAGCTCGA |
| 21 | Alternaria solani | TTATCTTAATGATGGCTACAGCTCGA |
| 22 | Cercospora arachidola | TAGTTCTTATGATGGCAATAGCCCGA |
| 23 | Rhizoctonia solani | TTGTTATGATGATGGGGATCGCACGA |
| 24 | Venturia inaequalis | TTATATTAATGATAGTTACAGCCCGA |
| 25 | Phytophthora infestans | TTATTTTAATGATGGCTACTGCTCGA |
| 26 | Mycosphaerella musicola | TAGTTCTAATGATGGCTACCGCCCGA |
| 27 | Didymella bryoniae—Db1 | TTATCTTAATGATGGCTACAGCTCGA |
| 28 | Didymella bryoniae—Db2 | TTATCTTAATGATGGCTACAGCTCGA |
| 29 | Didymella lycopersici | TTATCTTAATGATGGCTACAGCTCGA |

TABLE 13

ARMS primer design for the detection of the F129L mutation where the first base
on the triplet coding for residue 129 is a cytosine and the third base in the
triplet coding is guanine such that leucine is encoded.

| | | |
|---|---|---|
| | | primer sequence for the detection of F129L |
| Primer | | (thymine or cytosine to guanine change at |
| # | species: | third base of triplet 5′ to 3′) |

| | | |
|---|---|---|
| 1 | Plasmopara viticola | TTATTTTAATGATGGCGACTGCACCG |
| 2 | Erysiphe graminis f.sp. tritici/hordei | TCATATTAATGATCGTTACTGCACGG |
| 3 | Rhynchosporium secalis | TCATATTAATGATCGTTACAGCACCG |
| 4 | Pyrenophora teres (cDNA) | TTATCTTAATGATGGCTACAGCCCCG |
| 5 | Pyrenophora teres (genomic) | AGTTAGCCGGAACTTAGACAGCCCCG |
| 6 | Mycosphaerella graminicola | TAGTTCTGATGATGGCAACCGCACCG |
| 7 | Mycosphaerella fijiensis var. difformis | TAGTTCTAATGATGGCAACTGCCCCG |
| 8 | Sphaerotheca fuliginea | TCATATTAATGATCGTTACTGCACCG |
| 9 | Uncinula necator | TCATTTTAATGATGGCTACAGCACCG |
| 10 | Colletotrichum graminicola Cgr1 | TTGTAGCTATGATGGGTATAGGTCCG |
| 110 | Colletotrichum graminicola Cgr2 | TTGTAGCTATGATGGGTATAGGTCCG |
| 12 | Colletotrichum graminicola Cgr3 | TTGTAGCTATGATGGGTATAGGTCCG |
| 13 | Pythium aphanidermatum (forward) | TTATTTTAATGATGGCAACAGCTCCG |
| 14 | Colletotrichum gloeosporioides - chilli | TTGTAGCTATGATGGGTATAGGTCCG |
| | and mango | |
| 15 | Oidium lycopersicum | TCATATTAATGATGGCTACAGCACCG |
| 16 | Leveillula taurica Lt1 | TTATATTAATGATGGCTACAGCTCCG |
| 17 | Leveillula taurica Lt4 | TTATATTAATGATGGCTACAGCTCCG |
| 18 | Leveillula taurica Lt2 | TTATATTAATGATGGCTACTGCCCCG |
| 19 | Leveillula taurica Lt3 | TCATATTAATGATGGCTACTGCACCG |
| 20 | Pseudoperonospora cubensis | TTATTTTAATGATGGCAACAGCTCCG |
| 21 | Alternaria solani | TTATCTTAATGATGGCTACAGCTCCG |
| 22 | Cercospora arachidola | TAGTTCTTATGATGGCAATAGCCCCG |
| 23 | Rhizoctonia solani | TTGTTATGATGATGGGGATCGCACCG |
| 24 | Venturia inaequalis | TTATATTAATGATAGTTACAGCCCCG |
| 25 | Phytophthora infestans | TTATTTTAATGATGGCTACTGCTCCG |
| 26 | Mycosphaerella musicola | TAGTTCTAATGATGGCTACCGCCCCG |
| 27 | Didymella bryoniae - Db1 | TTATCTTAATGATGGCTACAGCTCCG |
| 28 | Didymella bryoniae - Db2 | TTATCTTAATGATGGCTACAGCTCCG |
| 29 | Didymella lycopersici | TTATCTTAATGATGGCTACAGCTCCG |

For the purposes of exemplification the primers included in Tables 9 to 13 include:
ARMS primers for P.teres, C. graminicola—Cgr3 and V. ineaqualis which can be used effectively either on genomic DNA preparations or biological samples including fungal isolates, fungal cultures, fungal spores or infected plant material.
ARMS primers for S. fulginea, O.lycopersicon, L. taurica Lt1, Lt2, Lt3 and Lt4, U. necator, Phytopthora infestans, R.solani, D. bryoniae Db1 and Db2, and D. lycopersici which may only be effective on cDNA
ARMS primers for P.viticola, R. secalis, M. graminicola, M. fijiensis var.difformis, C. graminicola Cgr1 and Cgr2, P. aphanidermatum, C. gloesporoides—chilli and mango, P. cubensis, C, arachidola, Mycosphaerella musicola and A. solani which may be used effectively with either genomic DNA preparations, cDNA preparations, cDNA preparations or biological samples including fungal isolates, fungal cultures, fungal spores or infected plant material.

cDNA material is recommended for those species in which the intron/exon organisation is not currently characterised around the nucleotide polymorphisms of interest.

The ARMS primers described in Tables 9-13 above provide specific examples of diagnostic primers according to the invention.

To adapt the primers shown in the above Tables for use in a standard ASPCR reaction the last base at the 3′ end should correspond to the point mutation without a destabilising base introduced.

Such primers may be manufactured using any convenient method of synthesis. Examples of such methods may be found in standard textbooks, for example “Protocols For Oligonucleotides And Analogues: Synthesis And Properties;” Methods In Molecular Biology Series; Volume 20; Ed. Sudhir Agrawal, Humana ISBN: 0-89603-247-7; 1993; 1^{st} Edition.

It will be appreciated that diagnostic primers can be designed to indicate the absence of one or more mutation(s) resulting in a F129L replacement in the encoded protein (i.e. to detect wild type sequence encoding phenylalanine or confirm the presence of sequence encoding leucine at the position corresponding to codon 129 in S. cerevisisiae cytochrome b. The use of ARMS primers for the detection of the absence of the mutation(s) resulting in a F129L replacement in the encoded protein is preferred. Primers designed for that purpose are described herein.

The detection of the wild type sequence is useful as a control in relation to the detection of the mutation and also is necessary where quantitation of wild type and mutant alleles present in a sample is desired.

In a further aspect the invention therefore provides a diagnostic primer capable of binding to a template comprising wild type fungal cytochrome b nucleotide sequence corresponding to the first and/or third base in the triplet coding for the amino acid at the position corresponding to S. cerevisiae cytochrome b residue 129 in the cytochrome b protein wherein the final 3′ nucleotide of the primer corresponds to a nucleotide present in a wild type fungal cytochrome b gene said wild type fungus showing sensitivity to a strobilurin analogue or any other compound in the same cross resistance group.

In a further embodiment of this aspect the invention therefore provides diagnostic primers capable of binding to a template comprising wild type fungal cytochrome b nucleotide sequence corresponding to the first or the third base in the triplet coding for the amino acid at the position corresponding to S. cerevisiae cytochrome b residue 129 in the cytochrome b protein wherein the final 3′ nucleotide of the primer corresponds to a nucleotide present in a wild type fungal cytochrome b gene said wild type fungus showing sensitivity to a strobilurin analogue or any other compound in the same cross resistance group

In a further aspect the invention therefore provides a diagnostic primer capable of binding to a template comprising wild type fungal cytochrome b nucleotide sequence corresponding to the third base in the triplet coding for the amino acid at the position corresponding to S. cerevisiae cytochrome b residue 129 in the cytochrome b protein wherein the final 3′ nucleotide of the primer corresponds to a nucleotide present in a wild type fungal cytochrome b gene said wild type fungus showing sensitivity to a strobilurin analogue or any other compound in the same cross resistance group.

In a preferred embodiment of this aspect of the invention the penultimate nucleotide (−2) of the primer is not the same as that present in the corresponding position in the wild type cytochrome b sequence.

In a further preferred embodiment the -3 nucleotide of the primer is not the same as that present in the corresponding position in the wild type cytochrome b sequence.

Other destabilising components may be incorporated along with the −2 or −3 nucleotide.

The diagnostic primer of the invention is preferably at least 20 nucleotides in length, most preferably 26 nucleotides in length but may be between 15 and 20 nucleotides in length.

In a further particularly preferred embodiment of the above aspect of the invention we provide diagnostic primers capable of binding to a template comprising wild type fungal cytochrome b nucleotide sequence corresponding to the first and/or third base in the triplet coding for the amino acid at the position corresponding to S. cerevisiae cytochromeb residue 129 in the cytochrome b protein wherein the final 3′ nucleotide of the primer corresponds to a nucleotide present in a wild type fungal cytochrome b and wherein up to 10, such as up to 8, 6, 4, 2, 1, of the remaining nucleotides may be varied with respect to the wild type sequence without significantly affecting the properties of the diagnostic primer.

In a further particularly preferred embodiment of the above aspect of the invention we provide diagnostic primers capable of binding to a template comprising wild type fungal cytochrome b nucleotide sequence corresponding to the first or third base in the triplet coding for the amino acid at the position corresponding to S. cerevisiae cytochrome b residue 129 in the cytochrome bprotein wherein the final 3′ nucleotide of the primer corresponds to a nucleotide present in a wild type fungal cytochrome b and wherein up to 10, such as up to 8, 6, 4, 2, 1, of the remaining nucleotides may be varied with respect to the wild type sequence without significantly affecting the properties of the diagnostic primer.

In a further particularly preferred embodiment of this aspect of the invention we provide diagnostic primers comprising the sequences given below and derivatives thereof wherein the final nucleotide at the 3′ end is identical to the sequences given below and wherein up to 10, such as up to 8, 6, 4, 2, 1, of the remaining nucleotides may be varied without significantly affecting the properties of the diagnostic primer. Conveniently, the sequence of the diagnostic primer may be exactly as provided below (Tables 14 and 15). In the majority of the primers listed below the penultimate nucleotide has been altered from wild type cytochrome b sequence to destabilise the template/primer hybrid thereby making it more selective for the desired template. It will be apparent to the man skilled in the art of primer design that bases alternative to or in addition to those discussed above may also be varied without adversely affecting the ability of the primer to bind to the template.

TABLE 14

ARMS primer designs for the detection of wild type sequence at
position 1 of codon 129 of plant pathogen cytochrome b genes.

| | | |
|---|---|---|
| | | primer sequence for the detection of WT |
| | | sequence (focused on the thymine at the |
| Primer | Species | first base of the triplet 5′ to 3′ |

| | | |
|---|---|---|
| 1 | Plasmopara viticola | CCCAAGGCAAAACATAACCCATGAA |
| | | (reverse complement) |
| 2 | Erysiphe graminis f.sp. tritici/hordei | ATTCATATTAATGATCGTTACTGCGT |
| 3 | Rhynchosporium secalis | ATTCATATTAATGATCGTTACAGCGT |
| 4 | Pyrenophora teres (cDNA) | CTTTATCTTAATGATGGCTACAGCGT |
| 5 | Pyrenophora teres (genomic) | GTAGTTAGCCGGAACTTAGACAGCGT |
| 6 | Mycosphaerella graminicola | ACTAGTTCTGATGATGGCAACCGCGT |
| 7 | Mycosphaerella fijiensis var. difformis | ATTAGTTCTAATGATGGCAACTGCGT |
| 8 | Sphaerotheca fuliginea | ATTCATATTAATGATCGTTACTGCGT |
| 9 | Uncinula necator | ATTCATTTTAATGATGGCTACAGCGT |
| 10 | Colletotrichum graminicola - Cgr1 | ACTTGTAGCTATGATGGGTATAGGAT |
| 11 | Colletotrichum graminicola - Cgr2 | ACTTGTAGCTATGATGGGTATAGGAT |
| 12 | Colletotrichum graminicola - Cgr3 | ACTTGTAGCTATGATGGGTATAGGAT |
| 13 | Pythium aphanidermatum (forward) | TTTTATTTTAATGATGGCAACAGCGT |
| 14 | Colletotrichum gloeosporioides - chilli | ACTTGTAGCTATGATGGGTATAGGAT |
| 15 | Colletotrichum gloeosporioides - mango | TCTTGTAGCTATGATGGGTATAGGAT |
| 16 | Oidium lycopersicum | ATTCATATTAATGATGGCTACAGCGT |
| 17 | Leveillula taurica Lt1 | ATTTATATTAATGATGGCTACAGCAT |
| 18 | Leveillula taurica Lt4 | ATTTATATTAATGATGGCTACAGCAT |
| 19 | Leveillula taurica Lt2 | CTTTATATTAATGATGGCTACTGCAT |
| 20 | Leveillula taurica Lt3 | ATTCATATTAATGATGGCTACTGCAT |
| 21 | Pseudoperonospora cubensis | TTTTATTTTAATGATGGCAACAGCGT |
| 22 | Alternaria solani | CTTTATCTTAATGATGGCTACAGCGT |
| 23 | Cercospora arachidola | TTTAGTTCTTATGATGGCAATAGCGT |
| 24 | Rhizoctonia solani | GCTTGTTATGATGATGGGGATCGCGT |
| 25 | Venturia inaequalis | CTTTATATTAATGATAGTTACAGCGT |
| 26 | Phytophthora infestans | TTTTATTTTAATGATGGCTACTGCGT |
| 27 | Mycosphaerella musicola | ATTAGTTCTAATGATGGCTACCGCGT |
| 28 | Didymella bryoniae - Db1 | CTTTATCTTAATGATGGCTACAGCGT |
| 29 | Didymella bryoniae - Db2 | CTTTATCTTAATGATGGCTACAGCGT |
| 30 | Didymella lycopersici | CTTTATCTTAATGATGGCTACAGCGT |

TABLE 15

ARMS primer designs for the detection of wild type sequence at
position 3 of codon 129 of plant pathogen cytochrome b genes.

| | | |
|---|---|---|
| | | primer sequence for the detection of WT |
| | | sequence. (focused on the thymine or |
| | | cytosine at the third base of the |
| Primer | Species | triplet 5′ to 3′) |

| | | |
|---|---|---|
| 1 | Plasmopara viticola (reverse) | TCCCCAAGGCAAAACATAACCCAGA |
| 2 | Erysiphe graminis f.sp. tritici/hordei | TCATATTAATGATCGTTACTGCATGC |
| 3 | Rhynchosporium secalis | TCATATTAATGATCGTTACAGCATCC |
| 4 | Pyrenophora teres (cDNA) | TTATCTTAATGATGGCTACAGCCTGC |
| 5 | Pyrenophora teres (genomic) | AGTTAGCCGGAACTTAGACAGCCTGC |
| 6 | Mycosphaerella graminicola | TAGTTCTGATGATGGCAACCGCATCC |
| 7 | Mycosphaerella fijiensis var. difformis | TAGTTCTAATGATGGCAACTGCCTGT |
| 8 | Sphaerotheca fuliginea | TCATATTAATGATCGTTACTGCATGC |
| 9 | Uncinula necator | TCATTTTAATGATGGCTACAGCATCC |
| 10 | Colletotrichum graminicola - Cgr1 | TTGTAGCTATGATGGGTATAGGTTGC |
| 11 | Colletotrichum graminicola - Cgr2 | TTGTAGCTATGATGGGTATAGGTTGC |
| 12 | Colletotrichum graminicola - Cgr3 | TTGTAGCTATGATGGGTATAGGTTGC |
| 13 | Pythium aphanidermatum (reverse) | ACCCCAAGGTAATACATAACCCTTG |
| 14 | Colletotrichum gloeosporioides - | TTGTAGCTATGATGGGTATAGGTTGC |
| | chilli and mango | |
| 15 | Oidium lycopersicum | TCATATTAATGATGGCTACAGCATAC |
| 16 | Leveillula taurica Lt1 | TTATATTAATGATGGCTACAGCTTAC |
| 17 | Leveillula taurica Lt4 | TTATATTAATGATGGCTACAGCTTAC |
| 18 | Leveillula taurica Lt2 | TTATATTAATGATGGCTACTGCCTGT |
| 19 | Leveillula taurica Lt3 | TCATATTAATGATGGCTACTGCATAC |
| 20 | Pseudoperonospora cubensis | TTATTTTAATGATGGCAACAGCTTGT |
| 21 | Alternaria solani | TTATCTTAATGATGGCTACAGCTTAC |
| 22 | Cercospora arachidola | TAGTTCTTATGATGGCAATAGCCTAC |
| 23 | Rhizoctonia solani | TTGTTATGATGATGGGGATCGCATCT |
| 24 | Venturia inaequalis | TTATATTAATGATAGTTACAGCCTAC |
| 25 | Phytophthora infestans | TTATTTTAATGATGGCTACTGCTTGT |
| 26 | Mycosphaerella musicola | TAGTTCTAATGATGGCTACCGCCTGT |
| 27 | Didymella bryoniae - Db1 | TTATCTTAATGATGGCTACAGCTTAC |
| 28 | Didymella bryoniae - Db2 | TTATCTTAATGATGGCTACAGCTTAC |
| 29 | Didymella lycopersici | TTATCTTAATGATGGCTACAGCTTAC |

To adapt the primers shown in the above Tables for use in a standard ASPCR reaction the last base at the 3′ end should correspond to the wild type sequence without introduction of a destabilising base.

The examples described above relate to ARMS primers based on the forward strand of DNA. The use of ARMS primers based on the forward strand of DNA is particularly preferred. However ARMS primers based on the reverse (antisense) strand may also be used.

ARMS primers may also be based on the reverse strand of DNA if so desired. Such reverse strand primers are designed following the same principles above for forward strand primers namely, that the primers may be at least 20 nucleotides in length most preferably 26 nucleotides in length, but may be between 15 and 20 nucleotides in length and the final nucleotide at the 3′ end of the primer matches the relevant template i.e. mutant or wild type and preferably the penultimate residue is optimally changed such that it does not match the relevant template. Additionally up to 10, such as up to 8, 6, 4, 2, 1, of the remaining nucleotides in the primer may be varied without significantly affecting the properties of the diagnostic primer.

In many situations, it will be convenient to use a diagnostic primer of the invention with a further amplification primer referred to herein as the common primer, in one or more cycles of PCR amplification. A convenient example of this aspect is set out in European patent number EP-B1-0332435. The further amplification primer is either a forward or a reverse common primer. Examples of such common primers, which may be used with particular plant pathogens., are given in Table 16 below.

TABLE 16
Examples of common forward and reverse primers for use with ARMS primers.

| | |
|---|---|
| Species | primer sequence (5′ to 3′) |

| | | |
|---|---|---|
| 1 | Plasmopara viticola (forward) | CATATTTTTAGGGGTTTGTATTACGG |
| 2 | Erysiphe graminis f.sp. tritici/hordei | AACACCTAAAGGATTACCAGATCCTGCAC |
| 3 | Rhynchosporium secalis | TACACCTAAAGGATTACCTGACCCTGCAC |
| 4 | Pyrenophora teres (cDNA) | TTACAGAGAAACCACCTCAAATGAACTCAACTATG |
| | | TCCAC |
| 5 | Pyrenophora teres (genomic) | TTTTTATTATACTTTTGTTAAACAGTCTTTTATTGT |
| | | TTAA |
| 6 | Mycosphaerella graminicola | AAATCCACCTCATACGAATTCAACTATGT |
| 7 | Mycosphaerella fijiensis var. difformis | AAACCTCCTCAAATAAACTCAACTATATC |
| 8 | Sphaerotheca fuliginea (cDNA) | TAACTGAGAAACCCCCTCAGAGAAACTCCACAATA |
| | | TCTTG |
| 9 | Uncinula necator (cDNA) | TTACAGAAAAACCACCTCAAAGAAACTCCACGATA |
| | | TCTTG |
| 10 | Colletotrichum graminicola - Cgr1 | TAACTGAGAAACCTCCTCAAACGAATTCAACAATA |
| | | TCTTG |
| 11 | Colletotrichum graminicola - Cgr2 | TAACAGAGAAACCTCCTCAAACGAATTCAACAATA |
| | | TCTTG |
| 12 | Colletotrichum graminicola - Cgr3 | TAACAGAGAAACCTCCTCAAACGAACTCAACAATA |
| | (cDNA) | TCTTG |
| 13 | Colletotrichum graminicola - Cgr3 | TATTTTTAATTGTAGTCTTGCCTTTCCTCGGAGAGG |
| | (genomic) | ACAA |
| 14 | Pythium aphanidermatum (forward) | TATATTATGGTTCATATATTACTCCAAG |
| | (reverse) | TATTTAAAGTTGGATTATCTACAGC |
| 15 | Colletotrichum gloeosporioides - chilli | TAACAGAGAAACCTCCTCAAACGAATTCAACTATA |
| | | TCTTG |
| 16 | Colletotrichum gloeosporioides - mango | TAACAGAGAAACCTCCTCAAACGAACTCAACGATA |
| | | TCTTG |
| 17 | Oidium lycopersicum | TTACAGAAAAACCTCCTCAAAGAAACTCCACGATA |
| | | TCTTG |
| 18 | Leveillula taurica Lt1 | TTACAGAGAAACCACCTCAAATAAATTCAACTATA |
| | | TCTTG |
| 19 | Leveillula taurica Lt4 | TTACAGAGAAACCTCCTCAAATAAATTCAACTATA |
| | | TCTTG |
| 20 | Leveillula taurica Lt2 | TAACACTGAAACCTCCTCAAATAAATTCAACTACA |
| | | TCTTG |
| 21 | Leveillula taurica Lt3 | TTACAGAAAAACCTCCTCAAATAAACTCGACGATA |
| | | TCTTG |
| 22 | Psuedoperonospora cubensis | CTACAGCAAAACCGCCCCACAACCAATCAACAATA |
| | | TCTTT |
| 23 | Alternaria solani | TAACACTGAAACCTCCTCAAATGAACTCAACAATA |
| | | TCTTG |
| 24 | Cercospora arachidola | AAACAGAGAAACCTCCTCATATAAATTCAACTAAA |
| | | TCTTG |
| 25 | Rhizoctonia solani | ACACGGAAAAGCCACCCCAGATTAACTCTACAAAA |
| | | TCTTG |
| 26 | Venturia inaequalis (cDNA) | TCACTGAAAAGCCTCCCCACAGAAATTCGACTATA |
| | | TCTTG |
| 27 | Venturia inaequalis (genomic) | TTGGTCCACTAATAGCCTTTCAACTACAGCTTGGT |
| | | ATAAG |
| 28 | Phytophthora infestans | CAACAGCAAAACCTCCCCATAACCAATCAACAATA |
| | | TCTTT |
| 29 | Mycosphaerella musicola | TAACAGAAAACCCACCTCAAATAAATTCAACTATA |
| | | TCTTG |
| 30 | Didymella bryoniae - Db1 | TAACGCTGAAACCTCCTCATATGAACTCAACAATA |
| | | TCTTG |
| 31 | Didymella bryoniae - Db2 | TAACTGAGAAACCACCTCAAATGAACTCAACGATA |
| | | TCTTG |
| 32 | Didymella lycopersici | TAACAGAAAAACCTCCTCATATGAACTCAACAATA |
| | | TCTTG |

The common primers in Table 16 for Rhynchosporium secalis, Mycosphaerella fijiensis var. difformis, Sphaerotheca fulginea (cDNA), Uncinula necator (cDNA), Colletotrichum graminicola Cgr1, Colletotrichum gloeosporioides—chilli, Oidium lycipersicum, Leveillula taurica Lt4, Pseudoperonospora cubensis, Alternaria solani, Cercospora arachidola, Rhizoctonia solani, Phytopthora infestans and Mycospharella musicola have previously been described in in Published International Patent Application Number WO 00/66773.

In the case of the longer sequences provided in Tables 3-8 and in Published International Patent Application Number WO 00/66773 the skilled man will be able to use this information to design appropriate primers.

It will be evident to the man skilled in the art that the common primer can be any convenient pathogen specific sequence which recognises the complementary strand of the cytochrome b gene (or other gene of interest) lying 3′ of the mutation selective primer.

The PCR amplicon size is preferentially 50 to 400 bp long but can be from 30 to 2500 bp long, or potentially from 30 to 10,000 bp long.

A convenient control primer may be used which is designed upstream from the F129L position. It will be evident to the man skilled in the art that the control primer may be any primer which is not specific for the amplification of the mutation or wild type sequences. When using these primers along with the corresponding reverse (‘common’) primer described above, amplification will occur regardless whether the F129L point mutation is present or not.

TABLE 17
Examples of control primers suitable for use in the invention

| | | |
|---|---|---|
| Primer | Species | Control primer sequence (5′ to 3′) |

| | | |
|---|---|---|
| 1 | Plasmopara viticola (reverse) | GTCCCCAAGGCAAAACATAACCCAT |
| 2 | Erysiphe graminis f.sp. tritici/hordei | ATTCATATTAATGATCGTTACTGC |
| 3 | Rhynchosporium secalis | ATATTCATATTAATGATCGTTACAGC |
| 4 | Pyrenophora teres (cDNA) | ATCTTTATCTTAATGATGGCTACAGC |
| 5 | Pyrenophora teres (genomic) | TTGTAGTTAGCCGGAACTTAGACAGC |
| 6 | Mycosphaerella graminicola | ATACTAGTTCTGATGATGGCAACCGC |
| 7 | Mycosphaerella fijiensis var. difformis | ATATTAGTTCTAATGATGGCAACTGC |
| 8 | Sphaerotheca fuliginea | ATATTCATATTAATGATCGTTACTGC |
| 9 | Uncinula necator | ATATTCATTTTAATGATGGCTACAGC |
| 10 | Colletotrichum graminicola - Cgr1 | ATACTTGTAGCTATGATGGGTATAGG |
| 11 | Colletotrichum graminicola - Cgr2 | ATACTTGTAGCTATGATGGGTATAGG |
| 12 | Colletotrichum graminicola - Cgr3 | ATACTTGTAGCTATGATGGGTATAGG |
| 13 | Pythium aphanidermatum (reverse) | TTGACCCCAAGGTAATACATAACCC |
| 14 | Colletotrichum gloeosporioides - chilli | ATACTTGTAGCTATGATGGGTATAGG |
| 15 | Colletotrichum gloeosporioides - | ATTCTTGTAGCTATGATGGGTATAGG |
| | mango | |
| 16 | Oidium lycopersicum | ATATTCATATTAATGATGGCTACAGC |
| 17 | Leveillula taurica Lt1 | ATATTTATATTAATGATGGCTACAGC |
| 18 | Leveillula taurica Lt4 | ATATTTATATTAATGATGGCTACAGC |
| 19 | Leveillula taurica Lt2 | ATCTTTATATTAATGATGGCTACTGC |
| 20 | Leveillula taurica Lt3 | ATATTCATATTAATGATGGCTACTGC |
| 21 | Pseudoperonospora cubensis | ATTTTTATTTTAATGATGGCAACAGC |
| 22 | Alternaria solani | ATCTTTATCTTAATGATGGCTACAGC |
| 23 | Cercospora arachidola | ATTTTAGTTCTTATGATGGCAATAGC |
| 24 | Rhizoctonia solani | ATGCTTGTTATGATGATGGGGATCGC |
| 25 | Venturia inaequalis | ATCTTTATATTAATGATAGTTACAGC |
| 26 | Phytophthora infestans | ATTTTTATTTTAATGATGGCTACTGC |
| 27 | Mycosphaerella musicola | ATATTAGTTCTAATGATGGCTACCGC |
| 28 | Didymella bryoniae - Db1 | ATCTTTATCTTAATGATGGCTACAGC |
| 29 | Didymella bryoniae - Db2 | ATCTTTATCTTAATGATGGCTACAGC |
| 30 | Didymella lycopersici | ATCTTTATCTTAATGATGGCTACAGC |

It is intended that the invention extends to all novel oligonucleotides (which may be used as primers) that are disclosed in the above tables.

A variety of methods may be used to detect the presence or absence of diagnostic primer extension products and/or amplification products. These will be apparent to the person skilled in the art of nucleic acid detection procedures. Preferred methods avoid the need for radiolabelled reagents. Particularly preferred detection methods are those based on fluorescence detection of the presence and/or absence of diagnostic primer extension products. Particular detection methods include gel electrophoresis analysis, “Scorpions”™ product detection as described in PCT application number PCT/GB98/03521 filed in the name of Zeneca Limited on 25 Nov. 1998 the teachings of which are incorporated herein by reference. Further preferred detection methods include ARMS linear extension (ALEX) and PCR with ALEX as described in published PCT application number WO 99/04037. Conveniently, real-time detection is employed. The use of “Scorpions”™ product detection as described in PCT application number PCT/GB98/03521 and published UK patent application No. GB2338301 is particularly preferred for use in all aspects of the invention described herein. The combination of the ARMS and the Scorpion technology as described herein is particularly preferred for use in all aspects of the invention described herein and the preferred detection method is a fluorescence based detection method. Many of these detection methods are appropriate for quantitative work using all of the above primers. These different PCRs can be carried out in different tubes or multiplexed in one tube. Using such methods, estimates can be made on the frequency of point mutation molecules present in a background of wild type molecules.

It will be obvious to the man skilled in the art that ARMS primer based technology provides the capacity to selectively prime for the copying of the adjoining sequence following hybridisation of an allele selective hybridisation probe in which the 3′ residue matches precisely one or other of the SNP alternatives. It is possible that an ARMS primer capable, for example, of giving highly selective amplification where for example there is a C residue at the first position of codon 129 would bind well to the cyt b gene of the alternative, wild type, T residue containing gene since, apart from the 3′ and penultimate residues there would be a perfect match. The key property of ARMS is that there is no copying of the adjoining region because of the mismatch at the key 3′ residue.’

The skilled person will also appreciate that other single nucleotide polynucleotide (SNP) or simple nucleotide polymorphism detection techniques may also be employed to detect the F129L mutations given the plant pathogenic fungal cytochrome b sequence data included in this patent application. Such methods include allele selective hybridisation techniques such as: “TaqMan”™ product detection, for example as described in patent numbers U.S. Pat. No. 5,487,972 & U.S. Pat. No. 5,210,015; “TaqMan®MGB” and “turbo TaqMan®” probes described, for example in the Applied Biosystems User Bulletin: Primer Express Version 1.5 and TaqMan MGB Probes for Allelic Discrimination” (May 2000) available via: Applied Biosystems (850 Lincoln Centre Drive, Foster City, Calif. 94404, USA) and as described herein (see also Examples). Further SNP or simple nucleotide polymorphism detection techniques, which may also be used to detect the F129L mutation(s), include “Molecular Beacons”® product detection, as outlined in patent number WO-95/13399 and surface enhanced Raman resonance spectroscopy (SERRS), as outlined in patent application WO 97/05280 both of which are incorporated herein by reference. Other SNP and/or simple polynucleotide detection techniques which may be used to define the alleles present at codon 129 include, but are not limited, to: “Pyrosequencing™” (Pyrosequencing AB, Uppsala, Sweden), Locked Nucleic Acid (LNA) technology (Exiqon A/S, Bygstubben 9, 2950 Vedbæk, Denmark), Dynamic Allele Specific Hybridisation (DASH) (Hybaid US, 8 East Forge Parkway, Franklin. Mass. 02038, USA) and Denaturing High-Performance Liquid Chromatography (dHPLC) (Giordano M. et al. Genomics 56 (1999) 247-253, Oefner P. J. J.Chromatogr., B: Biomed. Sci. Appl. 739 (2000) 345-355), which are again incorporated herein by reference.

The skilled user will also appreciate that some of the SNP and/or simple nucleotide recognition sequence techniques may readily be adapted to detect alleles encoding leucine at codon 129 which differ from the wild type phenylalanine codon by 2 changes (e.g. TTT [symbol] CTA) for example by design of a TaqMan MGB probe capable of recognising the variant sequence including both substitutions or because the technique directly determines the sequence at several, closely linked, positions as is the case with Pyrosequencing technology. In other cases, particularly those dependent upon allele specific amplification methods (e.g. ARMS etc.) it may be desirable to develop several detection methods, including perhaps primers acting on sense and antisense sequences to differentiate single and double nucleotide polymorphisms. It may also be possible to combine different SNP detection techniques (e.g. ARMS with Pyrosequencing) to allow for the most sensitive and specific detection and discrimination between single and double nucleotide polymorphisms. The invention extends to the combination of different SNP detection techniques for use in the appropriate aspects and embodiments of the invention described herein. For example, Taqman® (or Taqman®MGB) probes may be used in combination with an ARMS primer and a common primer. Where this is the situation, it is preferred that the ARM-S primer provides the specificity for the detection of a SNP mutation and that the Taqman(® (or Taqman®MGB) probe provides the detection mease (e.g. the fluorophore to be detected).

As exemplified herein we have used ARMS primers based on the forward strand of DNA in combination with Scorpion detection based on the reverse strand of DNA as the detection method. We have also exemplified herein ARMS primers based on the reverse strand of DNA in combination with Scorpion detection based on the forward strand of DNA as the detection method. It will be readily apparent to the man skilled in the art that alternative combinations of ARMS primers and Scorpion detection elements could also be used. For example the primer based on the forward strand of the DNA could be a combination of an ARMS primer with a Scorpion detection system and this could be used with a common primer based on the reverse strand of DNA or the primer based on the reverse strand of DNA could be a combination of an ARMS primer with a Scorpion detection system and this could be used with a common primer based on the forward strand of DNA.

In the examples described herein, the Scorpion detection element is on the common primer. The ARMS primer specific to the mutation and the wild type sequence are used in combination with the common fluorescence labelled primer. These two reactions are carried out in different PCR tubes and the fluorescence is emitted when the probe binds to the amplicon generated. The Scorpion element may alternatively be incorporated on the ARMS primers. In this case, the two ARMS primers can be labelled with different fluorophore's and used along with the common primer (this time unlabelled). These three primers may be included in the same reaction as the resulting mutant and wild type amplicons will lead to different fluorescence being emitted. Such assays are commonly referred to as multiplex assays.

As described in published UK patent application No. GB2338301 the Scorpion technology may be used in a number of different ways such as the intercalation embodiment where the tail of the Scorpion primer carries an intercalating dye which is capable of being incorporated between the bases of a double stranded nucleic acid molecule, upon which it becomes highly fluorescent; the FRET embodiment where the dyes involved in the primer form an energy transfer pair; the No-Quencher embodiment where a fluorophore is attached to the tail of the Scorpion primer; the Bimolecular embodiment where the fluorophore and quencher may be introduced on two separate but complementary molecules; the Capture Probe embodiment where amplicons may be specifically captured and probed using the same non-amplifiable tail and the Stem embodiment where the primer tail comprises self complementary stems. These embodiments are described fully in published UK patent application No. GB2338301, the teaching of which is incorporated herein by reference.

As referred to above, TaqMan® probes or TaqMan® MGB probes may be used as allele specific hybridisation probes for the detection of the presence and/ or absence of a mutation. Under these circumstances such probes are used in combination with common forward and reverse primers that are specific for DNA sequences upstream of and downstream from the mutation respectively. Specifically a first TaqMan® probe (or a first TaqMan®MGB) probe that is labelled with a first fluorescent reporter dye (e.g. VIC™) is designed to hybridise to the wild type sequence. A second TaqMan® (or TaqMan® MGB) probe that is labelled with a second, different fluorescent reporter dye (e.g. FAM) is designed to hybridise to the mutation. Both the first and the second probes are also labelled with a quencher molecule. Each probe anneals specifically to its complementary sequence between the forward and reverse primer sites, and when a probes is annealed the fluorescence of the fluorescent reporter dye is quenched as a result of the close proximity of the quencher molecule. During PCR the Taq DNA polymerase, which has 5′ to 3′ exonuclease activity, cleaves the reporter dye only from probes that hybridise to their specific target sequence. This results in the physical separation of the reporter dye from the quencher molecule thus resulting in an increase of fluorescence of the reporter dye. As the probes are labelled with different fluorescent reporter dyes they can either be used in separate reactions to detect either wild type or mutant sequence as appropriate, or alternatively they may be used simulataneously in the same reaction tube. When used in the same reaction, such assays may be referred to as multiplex assays. TaqMan MGB probes are described in Applied Biosystems User Bulletin: Primer Express Version 1.5 and TaqMan® MGB Probes for Allelic Discrimination” (May 2000) available from Applied Biosystems (850 Lincoln Centre Drive, Foster City, Calif. 94404, USA). TaqMan® based assays are particularly useful for providing a relatively rapid “yes/no” answer as to the presence or not of a mutation in a test sample.

The methods of the invention described herein reliably detect one or more single nucleotide polymorphism mutation(s) in a fungal cytochrome b gene resulting in a phenyalanine to leucine replacement at the position corresponding to S. cerevisiae cytochrome b residue 129 (F129L) in the encoded protein wherein the presence of said mutation(s) gives rise to fungal resistance to a strobilurin analogue or any other compound in the same cross resistance group at a detection level in the range of 1 mutated allele per 1,000,000 wild type alleles to 1 mutated allele per 10,000 wild type alleles, and preferably in the range of 1 mutated allele per 100,000 wild type alleles to 1 mutated allele per 10,000 wild type alleles. The method of the invention can also detect mutations occurring at higher frequency, for example, 1 mutated allele per 100 wild type alleles, 1 mutated allele per 10 wild type alleles or where only mutated alleles are present. Similarly the methods of the invention may be used to detect the frequency of the wild type allele in a background of mutated alleles.

The combination of allele specific primer extension made more sensitive with use of the ARMS technology and quantitative detection methods that are used in the present invention make this an extremely valuable technique for the detection of fungal single and/or simple polynucleotide polymorphism mutations occurring at low frequency.

The detection of those alleles responsible for giving rise to fungal resistance to a strobilurin analogue or any other compound in the same cross resistance group present in given isolates enables the results of phenotypic bioassays to be related to the DNA profile of the target gene. The discovery of single point mutations as the resistance mechanism explains the qualitative nature of the resistance, and the confirmation of single spore isolate sequences validates the accuracy of the screens in determining frequencies of resistant and sensitive isolates in the samples tested.

The development of a method combining allele specific primer extension, the specificity of ARMS and real time fluorescent detection, as exemplified herein with the Scorpion system, enables a greater number of samples to be analysed for the presence of the resistance mutation than would be feasible in a bioassay programme. Larger sample numbers enable the identification of the resistance mutation at frequencies of a lower percentage than might be easily detected through bioassay. This enables resistance to be identified in the population before it might be apparent from field data. The high throughput nature of the method enables wider areas and more geographically distinct sites to be sampled and tested than might be possible using a bioassay. Allele specific primer extension such as ARMS linked with real time fluorescent detection allows the detection of the presence of the resistance gene in a population before the effects of the gene can be assessed phenotypically by bioassay in heteroplasmic and/or heterokaryotic cells, thus reducing the error of classifying samples as sensitive when they carry a low frequency of the resistance genotype. Results are obtained much faster through simultaneous read-out real time technology compared to waiting for disease development in planta, enabling fast responses to field situations and advice on resistance management to be given more quickly.

One or more of the diagnostic primers of the invention may be conveniently packaged with instructions for use in the methods of the invention and appropriate packaging and sold as a kit. The kits will conveniently include one or more of the following: diagnostic, wild type, control and common oligonucleotide primers: appropriate nucleotide triphosphates, for example dATP, dCTP, dGTP, dTTP, a suitable polymerase as previously described, and a buffer solution.

One or more of the allele selective hybridisation probes of the invention may also be conveniently packaged with instructions for use in the methods of the invention and appropriate packaging and sold as a kit. The kits will conveniently include one or more of the following: oligonucleotide primers which allow the selective amplification of a segment of DNA comprising the region of the target pathogen cytochrome b gene including codon 129 from both wild type and isolates resistant to strobilurin analogue or any other compound in the same cross resistance group diagnostic wild type (F₁₂₉) and resistant (A₁₂₉) selective hybridisation probes, appropriate nucleotide triphosphates, for example dATP, dCTP, dGTP, dTTP, a suitable polymerase as previously described, and a buffer solution.

In a further aspect the invention provides a method of detecting plant pathogenic fungal resistance to a fungicide, said method comprising detecting one or more mutations in a fungal cytochrome b gene resulting in a phenyalanine to leucine replacement at the position corresponding to S. cerevisiae cytochrome b residue 129 (F129L) in the encoded protein wherein the presence of said mutation(s) gives rise to fungal resistance to a strobilurin analogue or any other compound in the same cross resistance group said method comprising identifying the presence or absence of said mutation(s) in fungal nucleic acid using any (or a) single nucleotide polymorphism detection technique.

In a further embodiment of this aspect the invention provides a method of detecting plant pathogenic fungal resistance to a fungicide, said method comprising detecting the hybridisation of an allele specific hybridisation probe wherein the detection of the hybridisation of said probe is directly related to presence or absence of a mutation(s) in a fungal cytochrome b gene resulting in a phenyalanine to leucine replacement at the position corresponding to S. cerevisiae cytochrome b residue 129 (F129L) in the encoded protein wherein the presence of said mutation(s) gives rise to fungal resistance to a fungicide whose target protein is encoded by a mitochondrial gene.

In a further embodiment of this aspect the invention provides a method of detecting plant pathogenic fungal resistance to a fungicide, said method comprising detecting the presence of an amplicon generated during a PCR reaction wherein said PCR reaction comprises contacting a test sample comprising fungal nucleic acid with a diagnostic primer in the presence of appropriate nucleotide triphosphates and an agent for polymerisation wherein the detection of said amplicon is directly related to presence or absence of a mutation(s) in a fungal cytochrome b gene resulting in a phenyalanine to leucine replacement at the position corresponding to S. cerevisiae cytochrome b residue 129 (F129L) in the encoded protein wherein the presence of said mutation(s) gives rise to fungal resistance to a fungicide whose target protein is encoded by a mitochondrial gene.

In a preferred embodiment of this aspect the invention provides a method of detecting plant pathogenic fungal resistance to a fungicide whose target protein is encoded by a cytochrome b gene comprising contacting a test sample comprising fungal nucleic acid with a diagnostic primer for one or more specific mutation(s) in a fungal cytochrome b gene resulting in a phenyalanine to leucine replacement at the position corresponding to S. cerevisiae cytochrome b residue 129 (F129L) in the encoded protein wherein the presence of said mutation(s) gives rise to fungal resistance to said fungicide, in the presence of appropriate nucleotide triphosphates and an agent for polymerisation, such that the diagnostic primer is extended when the said mutation(s) is present in the sample; and detecting the presence or absence of the said mutation by reference to the presence or absence of a diagnostic primer extension product.

In a further preferred embodiment of this aspect the invention provides a method of detecting plant pathogenic fungal resistance to a fungicide: whose target protein is encoded by a mitochondrial gene comprising contacting a test sample comprising fungal nucleic acid with a diagnostic primer for one or more specific mutation(s) in a fungal cytochrome b gene resulting in a phenyalanine to leucine replacement at the position corresponding to S. cerevisiae cytochrome b residue 129 (F129L) in the encoded protein wherein the presence of said mutation(s) gives rise to fungal resistance to said fungicide, in the presence of appropriate nucleotide triphosphates and an agent for polymerisation, such that the diagnostic primer is extended only when the said mutation is present in the sample; and detecting the presence or absence of the said mutation by reference to the presence or absence of a diagnostic primer extension product.

The methods of the invention described in the above aspect and embodiments are especially suitable for use with plant pathogenic fungal strains where the presence of one or more mutation(s) in a cytochrome b gene gives rise to fungicide resistance and most especially to resistance to a strobilurin analogue or a compound in the same cross resistance group where the mutation in the fungal DNA gives rise to a replacement of a phenylalanine residue at the position corresponding to S. cerevisiae cytochrome b residue 129, more especially to a F129L replacement in the encoded protein, and especially where the mutation is a T to C base change at the first position in the codon at the position corresponding to S. cerevisiae cytochrome b residue 129 or if the mutation is a T or C to A or G base change at the third position in the codon at the position corresponding to S. cerevisiae cytochrome b residue 129.

In a further aspect the invention provides a method of detecting and quantifying the frequency of one or more mutations in a fungal cytochrome b gene resulting in a phenyalanine to leucine replacement at the position corresponding to S. cerevisiae cytochrome b residue 129 (F129L) in the encoded protein wherein the presence of said mutation(s) gives rise to fungal resistance to a strobilurin analogue, said method comprising detecting the presence or absence of a mutation(s) in a fungal gene wherein the presence of said mutation(s) gives rise to fungal resistance to said fungicide, said method comprising identifying and quantifying the presence or absence of said mutation(s) in fungal nucleic acid using any (or a) single nucleotide polymorphism detection technique.

In a further preferred embodiment of this aspect the invention provides a method of detecting and quantifying the frequency of one or more mutations in a fungal cytochrome b gene resulting in a phenyalanine to leucine replacement at the position corresponding to S. cerevisiae cytochrome b residue 129 (F129L) in the encoded protein wherein the presence of said mutation(s) gives rise to fungal resistance to a fungicide whose target protein is encoded by a mitochondrial gene, said method comprising detecting the presence or absence of a mutation(s) in a fungal gene wherein the presence of said mutation(s) gives rise to fungal resistance to a strobilurin analogue or any other compound in the same cross resistance group, said method comprising identifying and quantifying the presence or absence of said mutation(s) in fungal nucleic acid using any (or a) single nucleotide polymorphism detection technique.

In a further embodiment of this aspect the invention provides a method of detecting and quantifying the frequency of one or more mutations in a fungal cytochrome b gene resulting in a phenyalanine to leucine replacement at the position corresponding to S. cerevisiae cytochrome b residue 129 (F129L) in the encoded protein wherein the presence of said mutation(s) gives rise to plant pathogenic fungal resistance to a fungicide whose target protein is encoded by a mitochondrial gene, said method comprising detecting the hybridisation of an allele selective probe by contacting a test sample comprising fungal nucleic acid with appropriate diagnostic wild type (F₁₂₉) and resistant (A₁₂₉) selective hybridisation probes wherein the detection of hybridisation of said allele specific hybridisation probes is directly related to both the presence and absence of said mutation in said nucleic acid wherein the presence of said mutation(s) gives rise to resistance to a fungicide whose target protein is encoded by a mitochondrial gene, and detecting and quantifying the relative presence and absence of the said mutation(s) by reference to the presence or absence of an amplicon generated during the PCR reaction.

In a further embodiment of this aspect the invention provides a method of detecting and quantifying the frequency of one or more mutations in a fungal cytochrome b gene resulting in a phenyalanine to leucine replacement at the position corresponding to S. cerevisiae cytochrome b residue 129 (F129L) in the encoded protein wherein the presence of said mutation(s) gives rise to plant pathogenic fungal resistance to a fungicide whose target protein is encoded by a mitochondrial gene, said-method comprising detecting the presence of an amplicon generated during a PCR reaction wherein said PCR reaction comprises contacting a test sample comprising fungal nucleic acid with appropriate primers in the presence of appropriate nucleotide triphosphates and an agent for polymerisation wherein the detection of said amplicon is directly related to both the presence and absence of a mutation in said nucleic acid wherein the presence of said mutation(s) gives rise to resistance to a fungicide whose target protein is encoded by a mitochondrial gene, and detecting and quantifying the relative presence and absence of the said mutation(s) by reference to the presence or absence of an amplicon generated during the PCR reaction.

In a further preferred embodiment of this aspect the invention provides a method of detecting and quantifying the frequency of one or more mutations in a fungal cytochrome b gene resulting in a phenyalanine to leucine replacement at the position corresponding to S. cerevisiae cytochrome b residue 129 (F129L) in the encoded protein wherein the presence of said mutation(s) gives rise to plant pathogenic fungal resistance to a fungicide whose target protein is encoded by a mitochondrial gene, comprising contacting a test sample comprising fungal nucleic acid with diagnostic primers to detect both the presence and absence of a specific mutation in said nucleic acid, the presence of which gives rise to resistance to said fungicide, in the presence of appropriate nucleotide triphosphates and an agent for polymerisation, such that the diagnostic primers relating to the absence and the presence of the specific mutation(s) are extended only when the appropriate fungal template is present in the sample; and detecting and quantifying the relative presence and absence of the said mutation(s) by reference to the presence or absence of diagnostic primer extension products.

The methods of the invention described in the above aspect and embodiments are especially suitable for use with plant pathogenic fungal strains where the presence of a mutation in a cytochrome b gene gives rise to fungicide resistance and most especially to resistance to a strobilurin analogue or a compound in the same cross resistance group and most especially where the mutation in the fungal DNA gives rise to a replacement of a phenylalanine residue at the position corresponding to S. cerevisiae cytochrome b residue 129, due to a mutation to a T to C base change at the first position in the codon at the position corresponding to S. cerevisiae cytochrome b residue 129 and/or to a mutation to a T or C to A or G base change at the third position in the codon at the position corresponding to S. cerevisiae cytochrome b residue 129, preferably due to a mutation to a T to C base change at the first position in the codon at the position corresponding to S. cerevisiae cytochrome b residue 129 or preferably to a mutation to a T or C to A or G base change at the third position in the codon at the position corresponding to S. cerevisiae cytochrome b residue 129, most preferably to a C to A base change at the third position in the codon at the position corresponding to S. cerevisiae cytochrome b residue 129.

In a yet further aspect the invention provides a method of selecting an active fungicide and optimal application levels thereof for application to a crop comprising analysing a sample of a fungus capable of infecting said crop and detecting and/or quantifying the presence and/or absence of one or more mutation(s) in a cytochrome b gene from said fungus resulting in a phenyalanine to leucine replacement at the position corresponding to S. cerevisiae cytochrome b residue 129 (F129L) in the encoded protein, wherein the presence of said mutation(s) may give rise to resistance to a fungicide whose target protein is encoded by a mitochondrial gene and then selecting an active fungicide and optimal application levels thereof. This may be achieved for example by initially testing for the frequency of occurrence of the F129L mutation in a plant pathogenic fungus of interest, using the methods of the invention. Once an initial assessment of the naturally occurring frequency of occurrence of the F129L mutation has been made, different fungal control strategies may be tested. For example a fungicide (preferably as strobilurin fungicide) may be applied to a further test sample of the fungus at a range of different rates and/or number and/or frequency of applications (preferably taking care to maintain a constant disease selection pressure), and for each strategy the frequency of occurrence of the F129L mutation may be assessed using the method of the invention. A correlation may then be drawn between the fungal control strategy employed and the frequency of occurrence of resistance mediated by the F129L mutation. The skilled man will easily be able to assess from this correlation, which is the best fungal control strategy to maintain fungal control and low levels of resistance to the fungicidal agent employed.

In a particularly preferred embodiment of this aspect of the invention the detection method comprises any (or a) single nucleotide polymorphism detection technique and more preferably comprises contacting a test sample comprising fungal nucleic acid with a diagnostic primer for the specific mutation(s) in the presence of appropriate nucleotide triphosphates and an agent for polymerisation, such that the diagnostic primer is extended when the said mutation(s) is present in the sample; and detecting the presence or absence of the said mutation(s) by reference to the presence or absence of a diagnostic primer extension product and the quantification is achieved by contacting a test sample comprising fungal nucleic acid with diagnostic primers to detect both the presence and absence of a specific mutation(s) in said nucleic acid the presence of which gives rise to resistance to a fungicide whose target protein is encoded by a mitochondrial gene in the presence of appropriate nucleotide triphosphates and an agent for polymerisation, such that the diagnostic primers relating to the absence and the presence of the specific mutation(s) are extended when the appropriate fungal template is present in the sample; and detecting and quantifying the relative presence and absence of the said mutation(s) by reference to the presence or absence of diagnostic primer extension products.

In a further particularly preferred embodiment of this aspect of the invention the detection method comprises contacting a test sample comprising fungal nucleic acid with a diagnostic primer for the specific mutation(s) in the presence of appropriate nucleotide triphosphates and an agent for polymerisation, such that the diagnostic primer is extended only when the said mutation(s) is present in the sample; and detecting the presence or absence of the said mutation(s) by reference to the presence or absence of a diagnostic primer extension product and the quantification is achieved by contacting a test sample comprising fungal nucleic acid with diagnostic primers to detect both the presence and absence of a specific mutation(s) in said nucleic acid the presence of which gives rise to resistance to a fungicide whose target protein is encoded by a mitochondrial gene in the presence of appropriate nucleotide triphosphates and an agent for polymerisation, such that the diagnostic primers relating to the absence and the presence of the specific mutation(s) are extended only when the appropriate fungal template is present in the sample; and detecting and quantifying the relative presence and absence of the said mutation(s) by reference to the presence or absence of diagnostic primer extension products.

In a particularly preferred embodiment of this aspect of the invention the detection method comprises any (or a) single nucleotide polymorphism detection technique and more preferably comprises contacting a test sample comprising fungal nucleic acid with an allele specific hybridisation probe for the specific mutation(s), such that the hybridisation probe hybridises when the said mutation(s) is present in the sample; and detecting the presence or absence of the said mutation(s) by detection and quantitation of the amount of wild type (F₁₂₉) plant pathogen cytochrome b gene in the test sample quantification being achieved by contacting a test sample comprising fungal nucleic acid with allele specific hybridisation probes to detect both the presence and absence of a specific mutation(s) in said nucleic acid the presence of which gives rise to resistance to a fungicide whose target protein is encoded by a mitochondrial gene in the presence of appropriate nucleotide triphosphates and an agent for polymerisation, such that the hybridisation probes relating to the absence and the presence of the specific mutation(s) hybridise when the appropriate fungal template is present in the sample; and detecting and quantifying the relative presence and absence of the said mutation(s) by reference to the presence or absence of hybridisation products.

The methods of the invention described herein are especially suitable for use with plant pathogenic fungal strains where the presence of a mutation in a cytochrome b gene gives rise to fungicide resistance and most especially to resistance to a strobilurin analogue or a compound in the same cross resistance group and where the mutation in the fungal DNA gives rise to a replacement of a phenylalanine residue at the position corresponding to S. cerevisiae cytochrome b residue 129, more especially to a F129L replacement in the encoded protein, and especially where the mutation is a T to C base change at the first position in the codon at the position corresponding to S. cerevisiae cytochrome b residue 129 or where the mutation is a T or C to A or G base change preferably a C to A base change at the third position in the codon at the position corresponding to S. cerevisiae cytochrome b residue 129.

In a still further aspect the invention provides a method of controlling fungal infection of a crop comprising applying a fungicide to the crop wherein said fungicide is selected according to any of the selection methods of the invention described above.

The method of the invention described above is especially suitable for use with plant pathogenic fungal strains where the presence of one or more mutation(s) in a fungal cytochrome b gene resulting in a F129L replacement in the encoded protein wherein the presence of said mutation(s) gives rise to fungicide resistance and most especially to resistance to a strobilurin analogue or a compound in the same cross resistance group.

In a yet further aspect the invention provides an assay for the detection of fungicidally active compounds comprising screening the compounds against strains of fungi which have been tested for the presence or absence of one or more mutation(s) in a fungal cytochrome b gene resulting in a F129L replacement in the encoded protein giving rise to resistance to a fungicide whose target protein is encoded by a mitochondrial gene according to the methods of the invention described herein and then determining fungicidal activity against said strains of fungi.

The methods of the invention described herein are especially suitable for use with plant pathogenic fungal strains where the presence of one or more mutation(s) in a cytochrome b gene gives rise to fungicide resistance and most especially to resistance to a strobilurin analogue or a compound in the same cross resistance group where the mutation in the fungal DNA gives rise to a replacement of a phenylalanine residue at the position corresponding to S. cerevisiae cytochrome b residue 129, more especially to a F129L replacement in the encoded protein, and especially where the mutation is a T to C base change at the first position in the codon at the position corresponding to S. cerevisiae cytochrome b residue 143 or where the mutation is a T or C to A or G base change, preferably a C to A base change at the third position in the codon at the position corresponding to S. cerevisiae cytochrome b residue 129.

By applying the methods of the invention described herein the appropriate rate of application of fungicides and/or the appropriate combination of fungicides to be applied to the crop may be determined.

The methods of the invention described herein are particularly suitable for monitoring fungal resistance to a strobilurin analogue or a compound in the same cross resistance group in crops such as cereals, fruit and vegetables such as canola, sunflower, tobacco, sugarbeet, cotton, soya, maize, wheat, barley, rice, sorghum, tomatoes, mangoes, peaches, apples, pears, strawberries, bananas, melons, potatoes, carrot, lettuce, cabbage, onion, vines and turf.

The methods of the invention described herein are particularly sensitive for detecting low frequencies of one or more mutation(s) in a fungal cytochrome b gene resulting in a F129L replacement in the encoded protein wherein the presence of said mutation(s) gives rise to fungal resistance to a strobilurin analogue, or making this an especially useful and commercially important way of screening plant pathogenic fungi for the onset of fungicidal resistance wherein the resistance is due to the above-identified mutation.

A key difference between ARMS, and other allele selective amplification based quantitative PCR detection systems, and technologies such as Taqman and Molecular Beacons is that the latter methods rely on the capacity of allele discriminatory hybridisation probes to provide a fluorescent signal proportionately related to the amount of target PCR product present at that time. This means that a single, non-selective and conventional primer pair can be used to amplify both wild type/parental and variant/mutant alleles of the target gene. The amount of the PCR product derived from each allele and, in each PCR cycle, an amount directly related to the amount present in the starting sample is then read from the fluorescent signal derived from the allele discriminatory hydridisation probe. This signal being “released” in the case of Taqman assays through DNA polymerase associated 5′exonuclease mediated release of the fluorophore from hybridised allele selective probe and in the case of Molecular Beacons by separation of 5′ and 3′ coupled fluorophore and quencher species on hyridisation of the Beacon to the target allele.

In the case of allele selective amplification technologies, such as ARMS, differential PCR reactions based on the specificity of the primers determines the amount of the specific PCR product present at any time in the reaction and this in turn is directly related to the amount of the allele present in the starting sample. Quantitative measurement of the amount of the PCR product present is then achieved via either non-specific technologies, such intercalating double-stranded DNA specific dyes like SYBR® Green I (Molecular Probes Inc., 4849 Pitchford Ave., Eugene, Ore., USA) or target gene specific, but allele unselective, probes such as Scorpions.

As the skilled person will appreciate these differences and their associated features provide distinctive advantages and potential disadvantages, including:
The necessity to design and validate only a single pair of SNP discriminatory Taqman or Molecular Beacon probes once conventional target gene selective PCR primers are available.
The requirement to design and validate both allele selective primer pairs (ARMS) and target gene specific hybridisation probes (Scorpions) for each allele discrimination ARMS/Scorpion based assays.
The high signal strength and reaction speed when Scorpion probes are used because of the intramolecular nature of their hybridisation to the target gene product (Whitcombe D., Theaker J., Guy S. P., Brown T. & Little S. (1999) Nature Biotechnology 17 (1999), 804- 807 Thelwell N., Millington S., Solinas A., Booth J., & Brown T. (2000) Nucleic Acids Research 28 (2000) 3752-3761).
The complications which can occur with non-sequence selective double stranded DNA detection technologies in the event of any non-specific amplification, arising perhaps as a result of contaminating DNAs in field samples and/or primer dimer formation.
   Under many conditions each of these methods is however quite suitable to, for example, detect and measure the relative amounts of F₁₂₉ and/or L₁₂₉ alleles of cytochrome b genes as described in this application. This is especially true when the genotypes of relatively homogeneous isolates of particular plant pathogens are being assessed e.g. individual complaint samples where strobilurin/QoI fungicide failure is suspected.

The expert will however appreciate that there are however important circumstances in which allele selective amplification based technologies, and in particular the highly sensitive and selective capacity provided by ARMS/Scorpions based technologies, have real advantages. A particular case of direct relevance to this application being in the analysis of nucleic acid preparations derived from populations comprising mixtures of alternative SNPs such as the F₁₂₉ and L₁₂₉ alleles described herein. More specifically this is the case when the relative frequencies of the alternative SNPs vary over a wide range.

It will be appreciated that field samples of plant pathogens will normally comprise such populations and indeed that analysing representative populations from a particular geographical region, plantation, vineyard, farm, field, orchard or plot will frequently be the most significant indicator of the performance of an agrochemical affected by the presence of SNPs, such as those encoding F₁₂₉ and L₁₂₉ alleles in cytochrome b genes, in the target pest.

It will also be appreciated by the person skilled in the art that mitochondrially encoded genes such as cytochrome b genes also constitute a context where the suitability of technology to analyse populations is of particular importance. Whilst organisms such as plant pathogens, which are almost invariably haploid or diploid in their vegetative growth phase, will possess 0, 1, 1+1 or 0+2 copies of alternative nuclearly encoded alleles of a gene of interest, the situation can be much more complicated with mitochondrially encoded genes. Individual plant pathogens can have tens, sometime hundreds, of mitochondria per nucleus and individual mitochondria can also themselves possess multiple mitochondrial genomes. Intrinsically therefore mitochondrial genes are members of populations which are much larger, more complex and diverse, even when taken from samples which are nominally individual, microbiologically purified, isolates, when compared with nuclear genes from the same sample.

The greater suitability of allele selective amplification based quantitative PCR technologies for analysing populations, especially ones where important alleles are present at relatively low levels, stems from the intrinsic nature of the PCR process: it is an exponential amplification based technology (see for example: Saiki et al. Science 230 (1985) 1350, PCR (Newton & Graham) pp 3-5 (1994) BIOS Scientific Publishers ISBN 1 872748 82 1 and The Encyclopedia of Molecular Biology (ed. Kendrew & Lawrence) pp 864-866 (1994) Blackwel] Science Ltd. ISBN 0 632 02182 9).

This means firstly that if two allelic forms of a gene, capable of amplification with a common primer pair, are present in a sample at significantly different concentrations (e.g. 100 fold, 1,000 fold or 10,000 fold) that their relative abundance will be maintained throughout the PCR. The relative strength of the hybridisation signal achieved by annealing of allele specific probes such as Taqman or Molecular Beacons will therefore always reflect this difference. Fluorescence signals of 100 fold, 1,000 fold or 10,000 lower intensity are extremely difficult to detect above background levels. Secondly however PCR reactions are normally limited by complete utilisation of the nucleotides required to synthesise the PCR product. Exponential amplification of a very abundant species can therefore severely deplete or even exhaust the supply of nucleotides before significant amplification of a lower abundance species can start. Additionally hybridisation is a kinetic process and in bimolecular reactions such as Taqman and Molecular Beacons has a rate which is a function of the concentration of the reacting species. The higher concentration of the more abundant PCR product will therefore result in annealing to its cognate probe proceeding at a higher rate. As a consequence of such factors we have found that the effective range of Molecular Beacons and Taqman assays covers only about a 10-50 fold and, more specially, a less than 10 fold difference in mixed populations.

By contrast allele selective amplification technologies are not affected by such competition effects arising from varying concentrations of alleles/SNPs. The PCR cycle at which a signal reflecting the presence of a given allele/SNP in a sample becomes apparent is a simple function of the starting concentration of that allele. Since there is no competing amplification product present there is no competition for substrate and the fluorescence signal can always approach the maximum.

As a consequence when DNA populations comprising mixtures of different alleles/SNPs are analysed in parallel reactions with appropriate allele selective amplification reagents an accurate estimate of the relative concentrations of the alleles/SNPs in the initial sample can be obtained over a very wide range. Typically we find that ranges of 0.01-99.99% (10⁴) are easily achieved with ARMS/Scorpions based assays, often ranges of 0.001-99.999% (10⁵) can be obtained and sometimes ranges of 0.0001-99.9999% (10⁶) may be achievable.

The invention is further illustrated with reference to the examples and figures in which

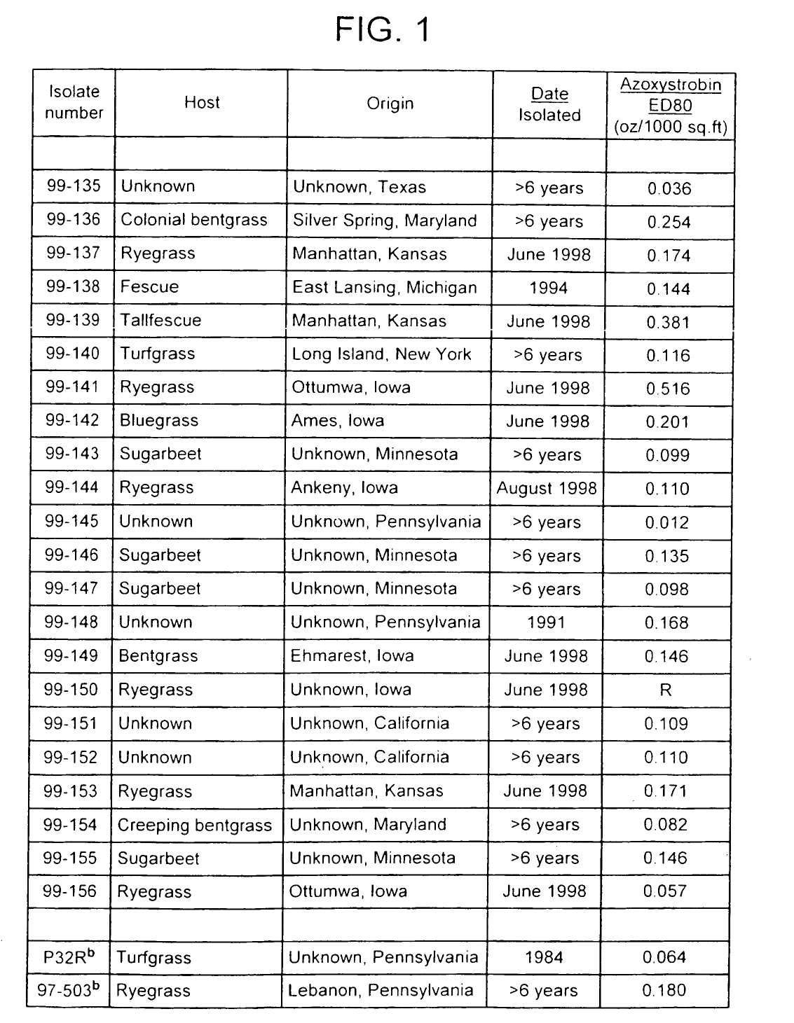

FIG. 1: table describing the origin and sensitivity to Azoxystrobin of isolates of Pythium aphanidermatum.

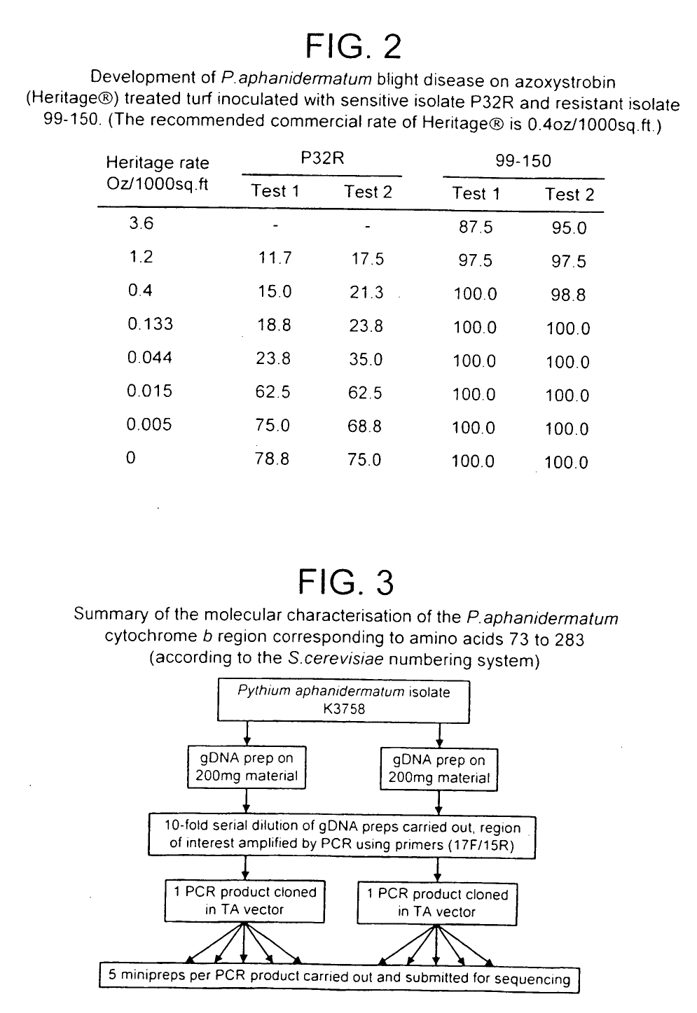

FIG. 2: table describing development of disease on azoxystrobin treated turf.

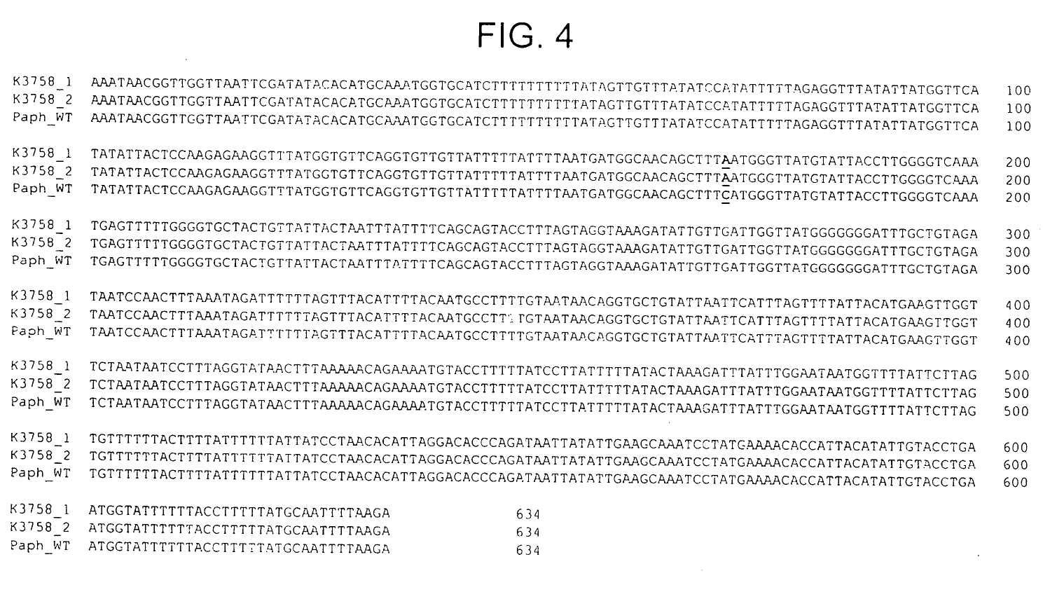

FIG. 3: Summary of the molecular characterisation of the P. aphanidermatum cyt b region corresponding to amino acids 73 to 283.

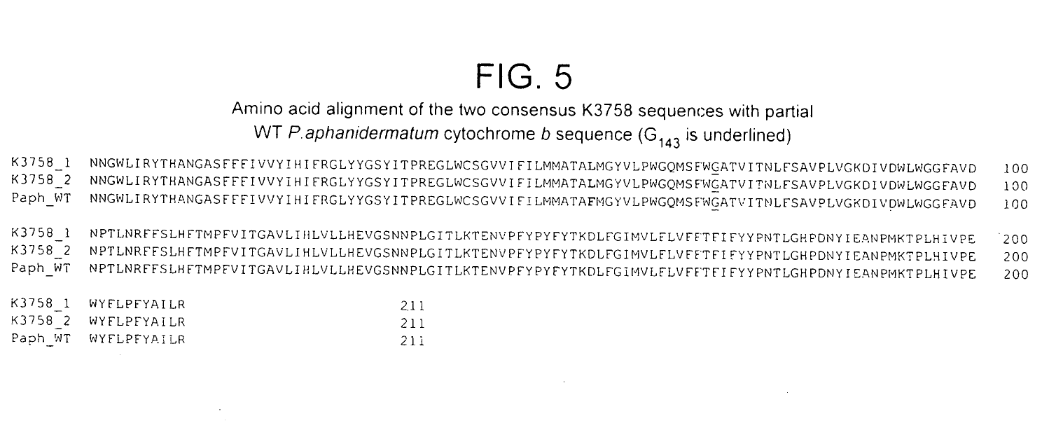

FIG. 4: Base pair alignment of two consensus K3758 sequences with a partial wild type P. aphanidermatum cyt b gene sequence.

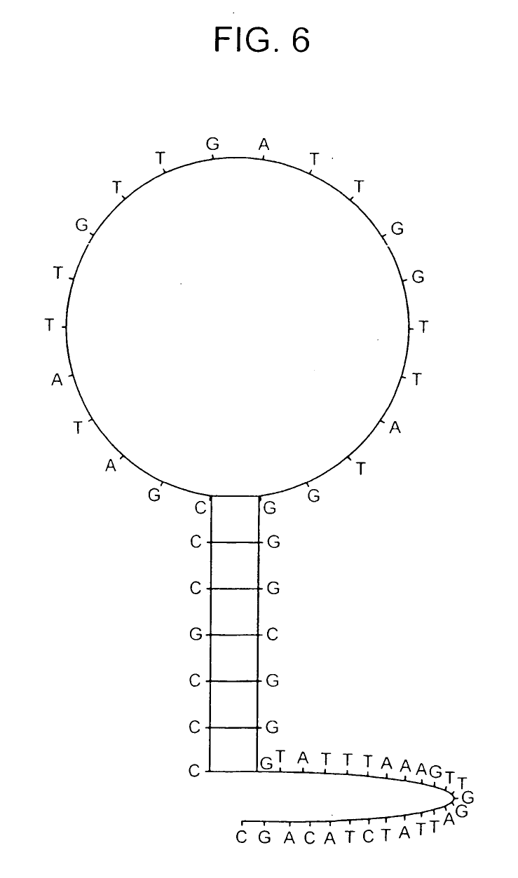

FIG. 5: amino acid alignment of two consensus K3758 sequences with a partial wild type P. aphanidermatum cyt b gene sequence

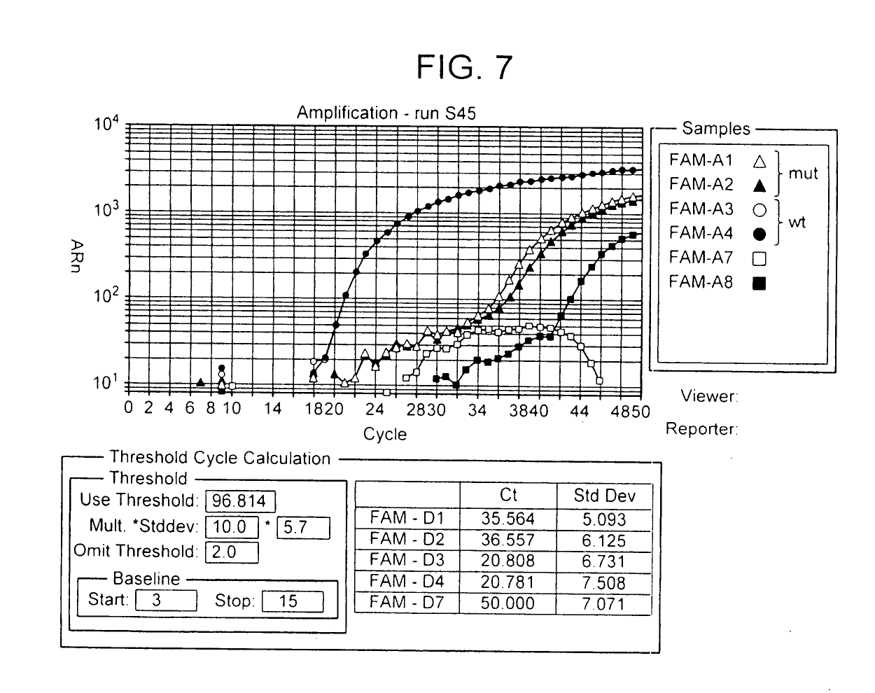

FIG. 6: Stem loop secondary structure of an antisense Scorpion primer for use in P. aphanidermatum F129L assays in combination with sense allele selective ARMS primers.

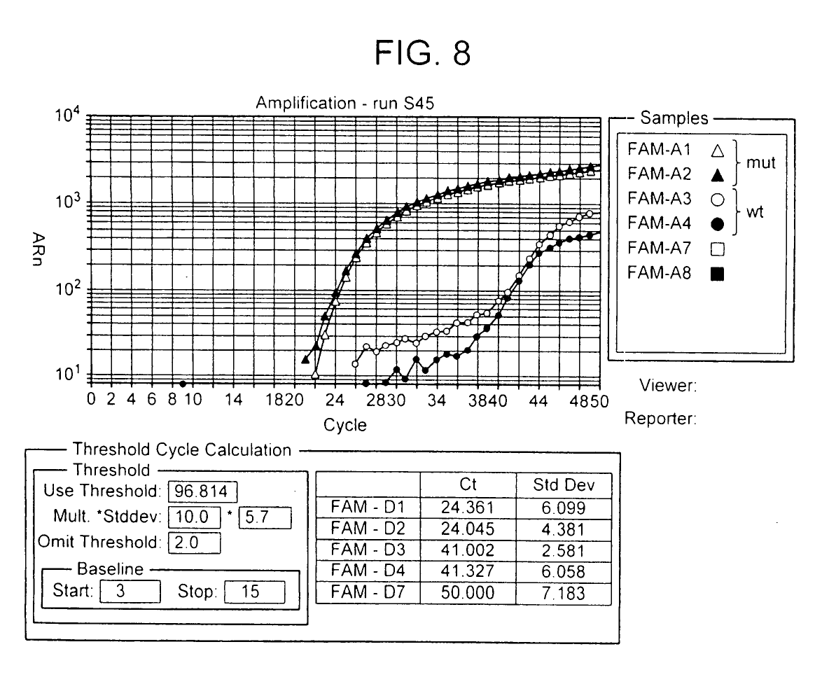

FIG. 7: A graph showing the amplification of wild type plasmid (wells A3 and A4) and amplification of mutant plasmid (wells A1 and A2) with primer Pt129-1.

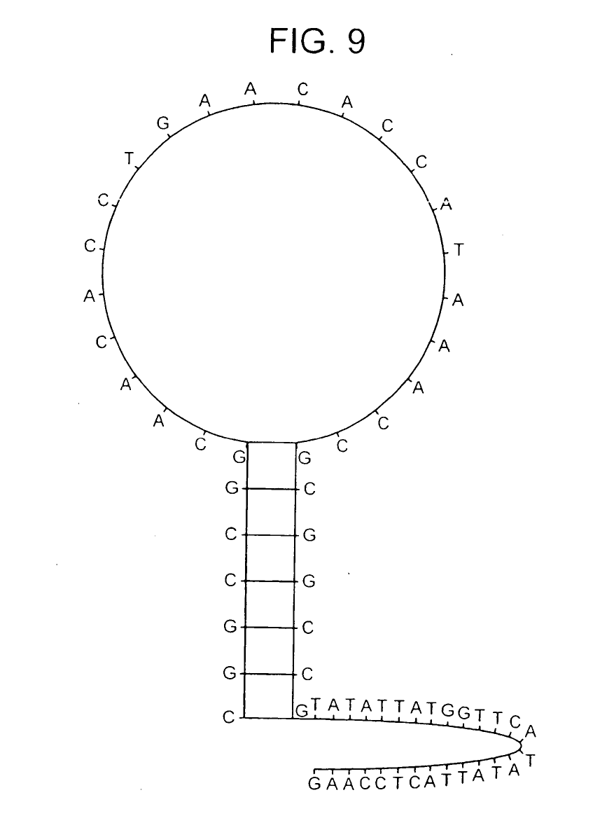

FIG. 8: A graph showing the amplification of mutant plasmid (wells A1 and A2) and amplification of wild type plasmid (wells A3 and A4) with primer Pt129-4.

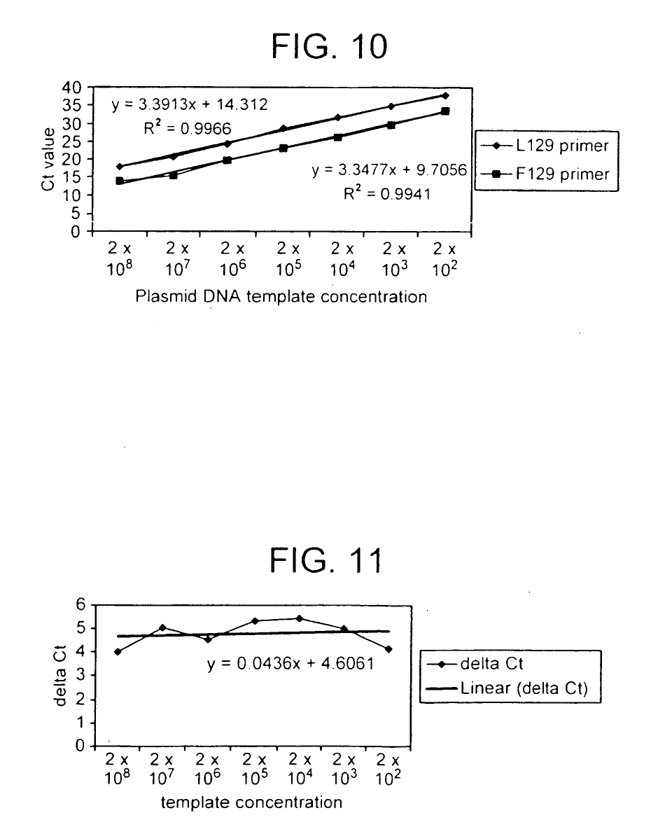

FIG. 9: Stem loop secondary structure of a sense Scorpion primer for use in P. aphanidermatum F129L assays in combination with antisense allele selective ARMS primers.

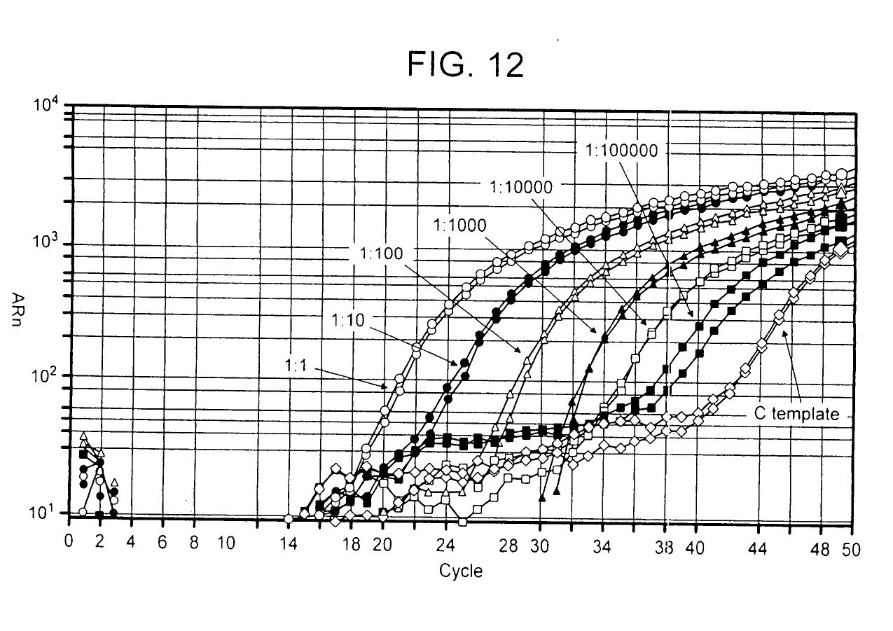

FIG. 10: A Plot of the Ct value on the appropriate template for the ARMS primers Pt129-A14 and Pt129-C4.

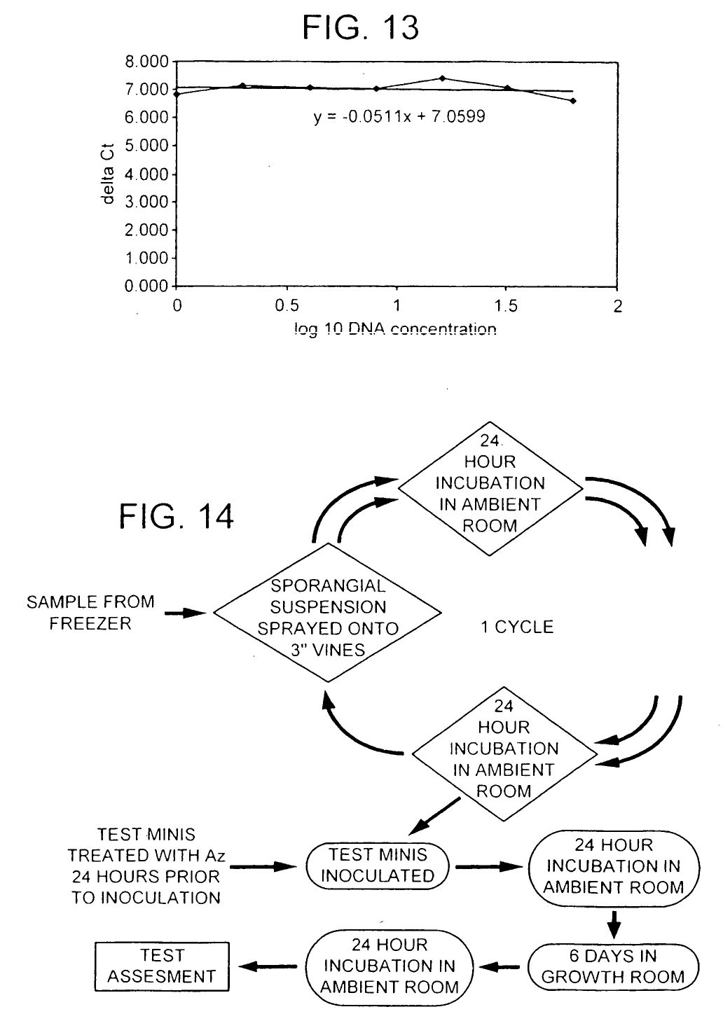

FIG. 11: A Plot of the ΔCt between ARMS primer Pt129-A14 and Pt 129-A14 vs template concentration.

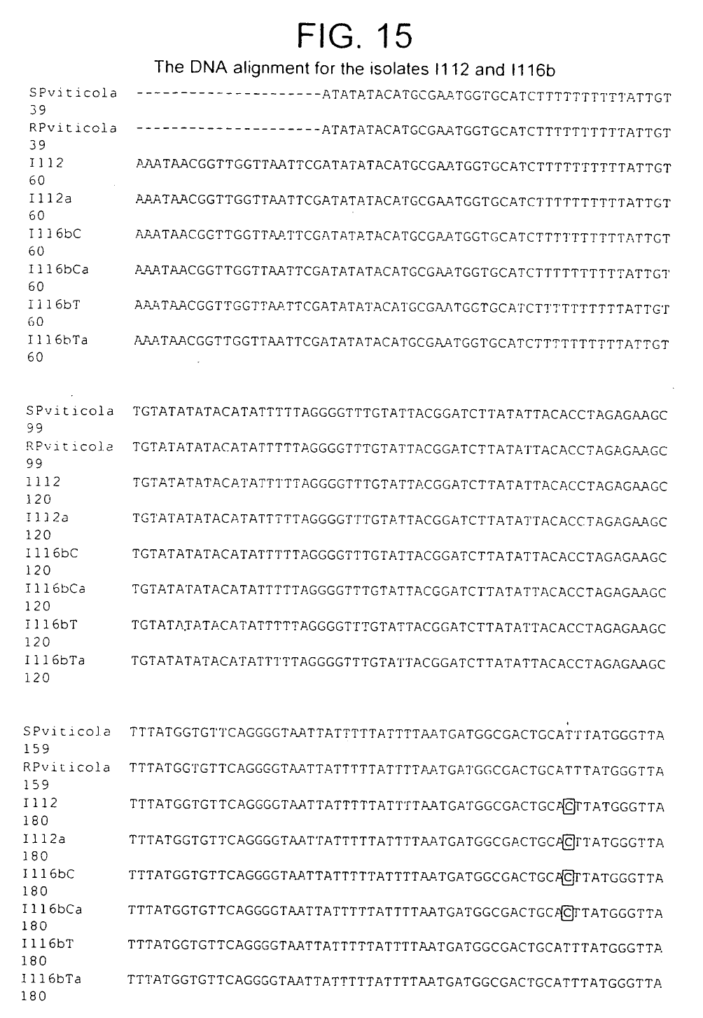

FIG. 12: A plot showing the amplification of the L129 allele with ARMS primer Pt129-A14 when 10-fold dilutions of the resistant (L129) allele are spiked into a constsant background of the wild type (F129) allele.

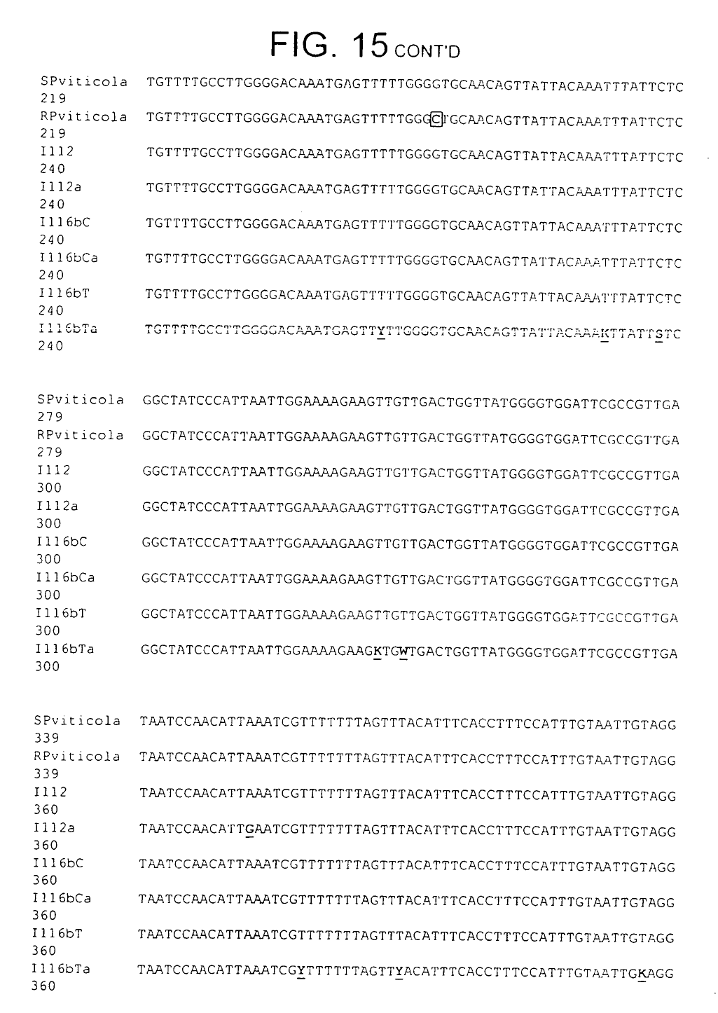

FIG. 13: A plot of log 10 DNA concentration vs ΔCt for the P aphanidermatum F129L assay.

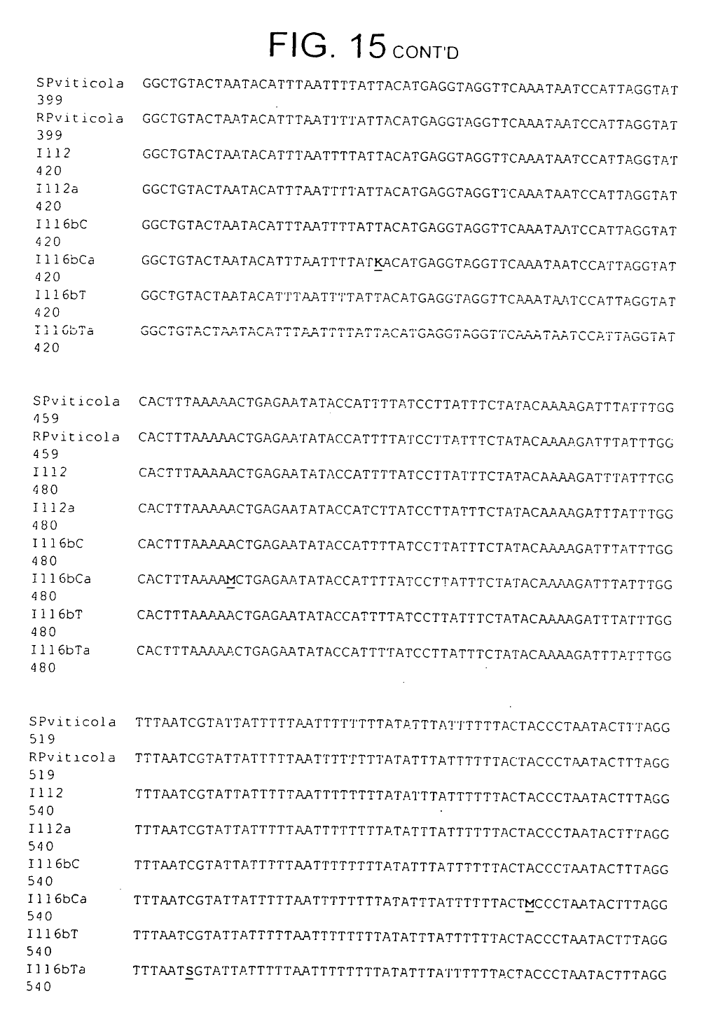

FIG. 14: A flow diagram showing preparation of P. viticola samples I112 and I116b for the in planta dose reponse assay.

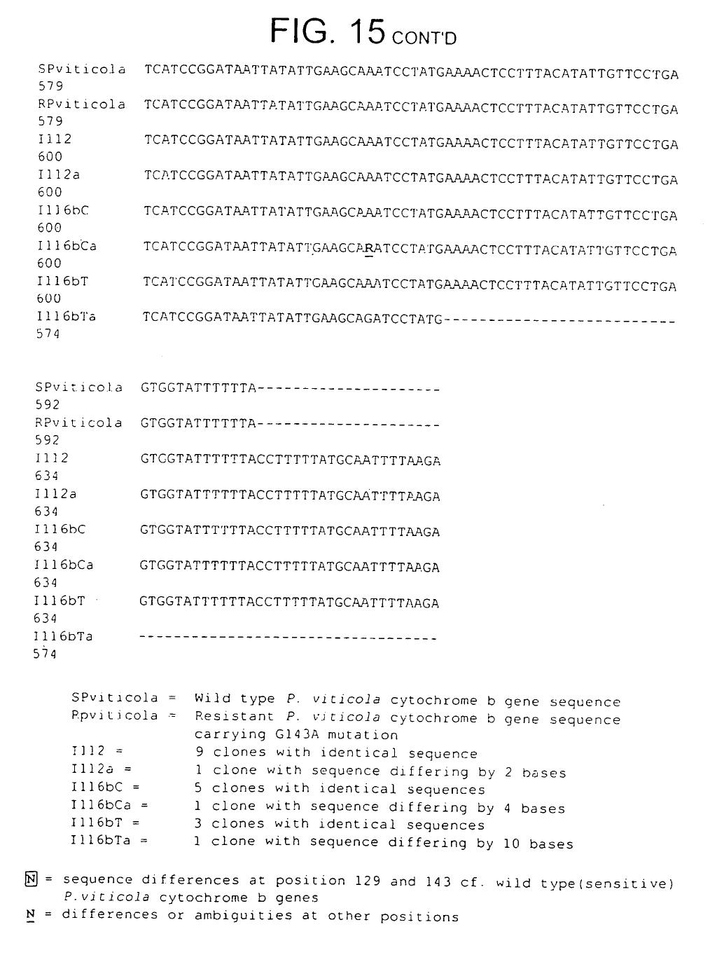

FIG. 15: show the DNA alignment for the isolates I112 and I116b.

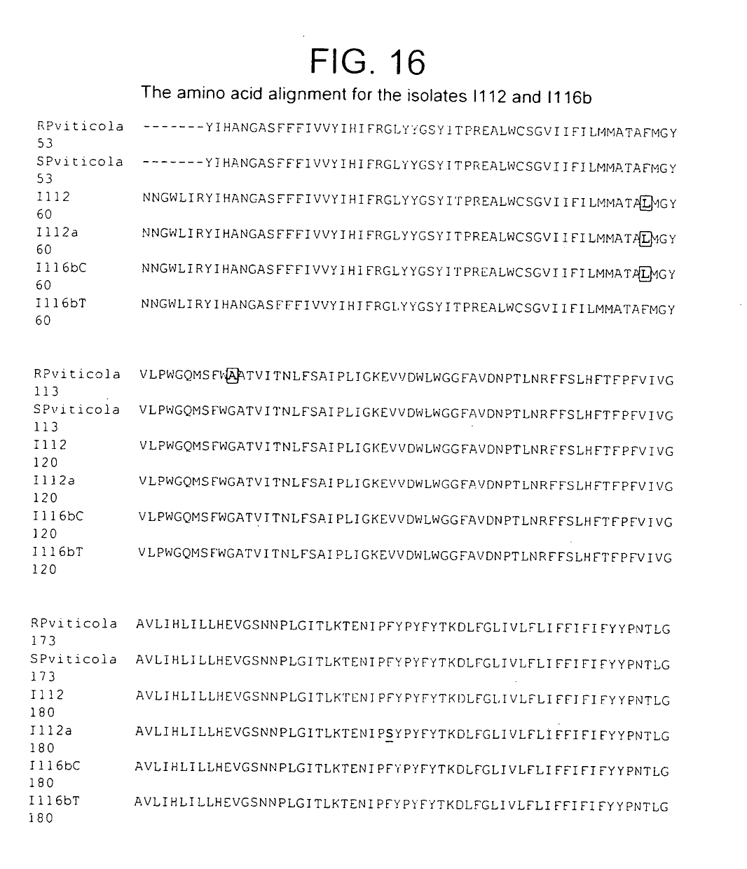

FIG. 16: shows the amino acid alignment for the isolates I112 and I116b.

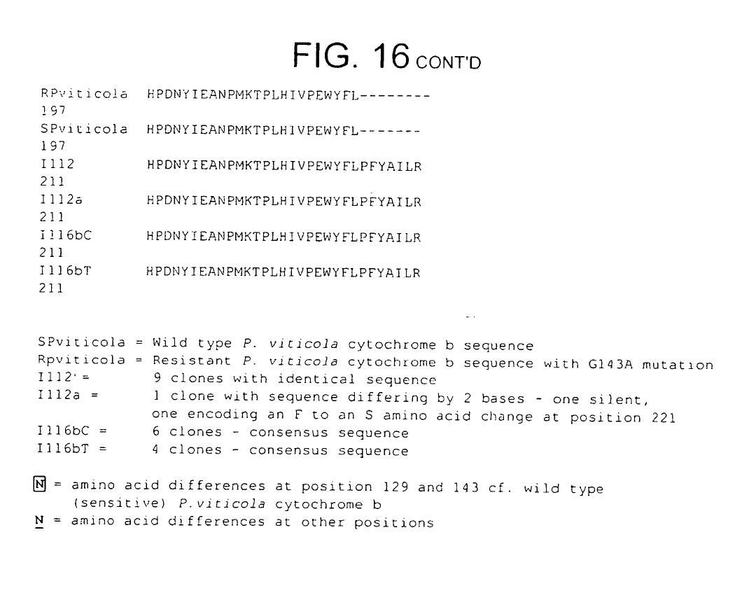

FIG. 17: shows a sense Scorpion primer for P.viticola.

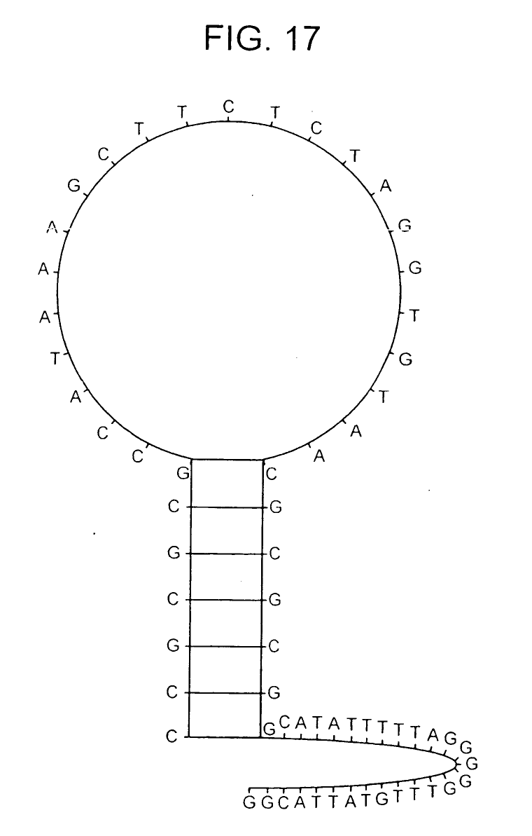

FIG. 18: A plot of log 10 DNA concentration vs ΔCt for the P. viticola F129L assay.

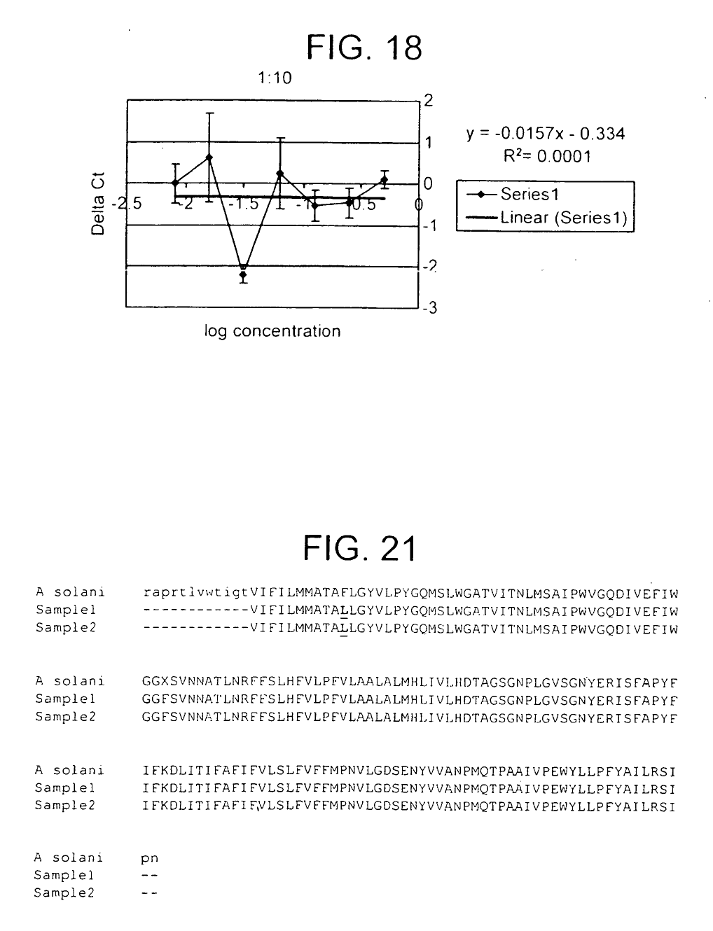

FIG. 19: shows an antisense Scorpion primer for P. viticola.

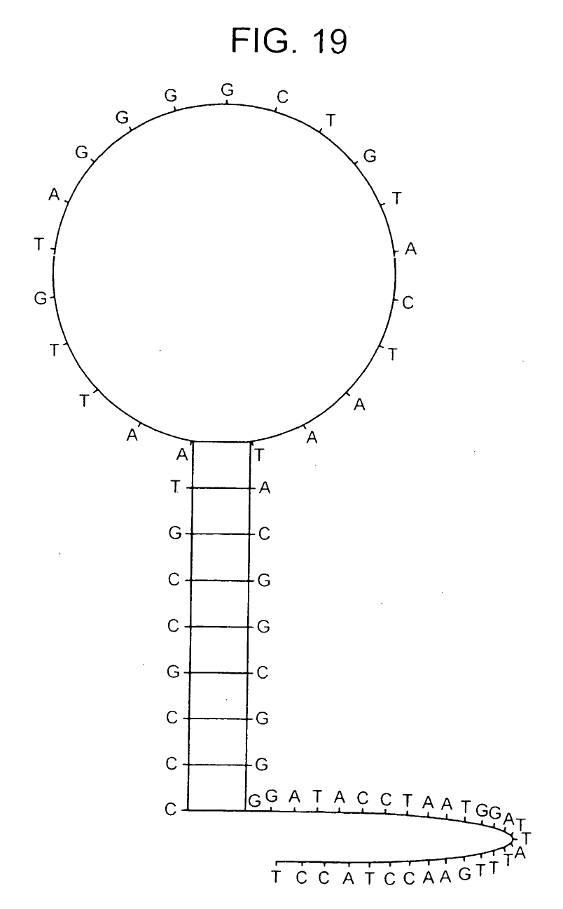

FIG. 20: shows the nucleotide alignment for the two isolates of A. solani

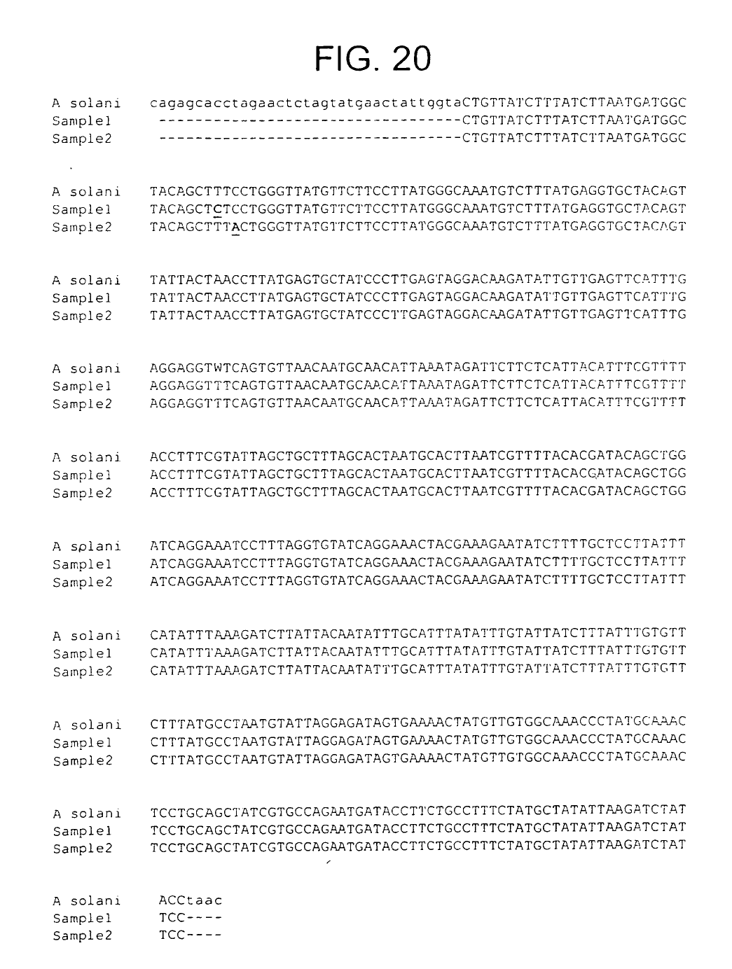

FIG. 21: shows the amino acid alignment for the two isolates of A. solani

### DETAILED DESCRIPTION OF THE INVENTION

### EXAMPLES

In Examples3, 4,5, 6, 7,8,9, 12, 13, 14, 15, 16 & 17 below the Scorpion™ system (AstraZeneca Diagnostics) was used as a product detection system. This detection system is described in full in PCT application number PCT/GB98/03521 filed in the name of Zeneca Limited on 25 Nov. 1998 the teachings of which are incorporated herein by reference. This novel detection system uses a tailed primer and an integrated signalling system. The primer has a template binding region and a tail comprising a linker and a target binding region. In use the target binding region in the tail hybridises to complementary sequence in an extension product of the primer. This target specific hybridisation event is coupled to a signalling system wherein hybridisation leads to a detectable change. The detection method of this system offers a number of significant advantages over other systems. Only a single primer/detector species is required. This provides both increased simplicity and enhanced specificity based on the ready availability of the target binding region for hybridisation with the primer extension product. The newly synthesised primer extension product is the target species so the output signal obtainable is directly related to amount of extended primer. It is not dependent on additional hybridisation events or enzymatic steps. Intra and inter-strand competition for the probe site is limited so probe design becomes simplified. As the interaction is unimolecular, the signalling reaction is very rapid, permitting increased cycling rates which is a significant feature for experimental efficiency.

The Scorpion primers designed in the example described below have the following modifications in common:
A hexethylene glycol (HEG) monomer as a blocking moiety that is sited between the template binding region of the primer and the tail region, which moiety prevents polymerase mediated chain copying of the tail region of the primer template.
A FAM fluorescent molecule is added to the 5′end of the primer. FAM is one of the fluorescence molecules that can, for example, be readily detected by the 488 nm laser of the ABI PRISM 7700 instrument (PE Biosystems)
MR (methyl red) is a non-fluorogenic quencher attached toga uracil residue

Other fluorescence molecules and quenching mechanisms can also be accommodated in Scorpion primer design and would be suitable to use in this invention.

In Example 18 an assay dependent on the TaqMan assay system is exemplified.

### Example 1

### Identification of a P.aphanidermatum Isolate Displaying High Level Resistance to Qₒ Site Inhibitor Fungicides

A series of 22 P.aphanidermatum isolates was provided by G. Peng, M. L. Gleason and F. W. Nutter from Iowa State University in 1999 to Zeneca Agricultural Products (now Syngenta Crop Protection) to allow profiling of sensitivity to azoxystrobin., the sensitivity of the isolates to the fungicide mefenoxam and the growth rate of the isolates on agar plates having previously been determined by Guangbin Peng at Iowa State University (Peng, G. et al. Phytopathology 89 (1999) S59. ) Background information on these isolates and a summary of their azoxystrobin resistance properties are shown in FIG. 1.

To enable an assessment of axoxystrobin (strobilurin) resistance of the above isolates, perennial ryegrass (Lolium perenne L.) was grown in 4×4 inch square plastic pots containing sandy soil. Agsorb absorbent clay (extra fine red) was used to anchor the seeds and provide a high level of moisture around the seed to aid germination. The pots were watered using a fog nozzle and covered with paper to reduce evaporation. A fog nozzle was used 3-5 times a day to water the turf pots. When seedlings emerged, the paper cover was removed from the pots to prevent etiolation and watering was reduced to twice daily using a fan nozzle. The pots were maintained for 12-16 days in a glass-house (17-32° C.; 14 h photoperiod) prior to treatment. During this time, the turf was cut twice with electric Black and Decker hand-held clippers.

Pythium aphanidermatum cultures of the isolates summarized in FIG. 1 were maintained on potato dextrose agar (PDA) amended with streptomycin sulfate at 0.05 g/L. Organically grown rye grains (50 g) were hydrated overnight with 40 ml deionized water placed in 250 ml plastic (polyvinyl-chloride) flasks. The flasks were autoclaved twice on successive days for 45 minutes each time. After the berries cooled, ten 1×1 cm agar plugs of P. aphanidermatum were added to each flask. Flasks containing infected rye-berries were incubated in a growth chamber (26° C.; 14 h photoperiod) for seven days to allow the fungi to colonize the rye grains. The fungal-colonized rye grains were then used to inoculate the turf; by placing four berries on the center surface of each pot.

Azoxystrobin treatments were applied in an automated spray cabinet using a flat fan nozzle (8004E) positioned 18 inches over the turf with a spray volume of 3 US gal/1000 sq.ft. To prevent carry over from treatment to treatment, applications were made starting with the lowest concentration and ending with the highest concentration. The spray nozzle was washed once with acetone and twice with deionized water (100 ml/rinse) between treatments. Turf was sprayed with Heritage® (azoxystrobin) at rates of 0.4, 0.133, 0.044, 0.015, 0.005 and 0 oz.Heritage/1000 sq.ft. Following application, the turf was allowed to dry and was transferred to a growth chamber (25-28° C.; 14 h photoperiod) overnight.

Turf was inoculated with fungal-colonized rye-berries one day after application as previously described. All treatments were then randomized and placed in a dew chamber (25-28° C.; 14 h photoperiod) for four days. The turf was moved to a growth room 24 h prior to disease assessment. The percent of Pythium-infected turf area in each pot was assessed five days after inoculation With the exception of isolate 99-150, all the isolates of P. aphanidermatum were sensitive to azoxystrobin (Table1). The ED₈₀ values of the sensitive isolates fell within the range of ED₈₀ values of the baseline distribution already established for P. aphanidermatum (FIG. 1). Isolate 99-150 was found to be resistant to azoxystrobin. This is the first report of resistance to azoxystrobin of an isolate of the plant pathogenic fungus P. aphanidermatum.

The sensitivity of the resistant isolate 99-150 and sensitive isolate P32R was then confirmed by testing in two separate experiments using rates up to nine times higher than the recommended commercial rate of Heritage® (azoxystrobin): 3.6, 1.2, 0.4, 0.133, 0.044, 0.015, 0.005 and 0 oz/1000 sq.ft. The sensitive isolate P32R responded to azoxystrobin according to the rate used and the percentage of infected turf was reduced dramatically at the highest rate. The resistant isolate 99-150 did not respond to azoxystrobin even at a rate nine times higher than the recommended one (FIG. 2)

Information about the prior treatment of the P. aphanidermatum isolates studied in this work was limited. However we believe that the isolates with no information about the collection date in FIG. 1 were never exposed to strobilurins since some were collected more than 6 six years ago, before the commercial introduction of these fungicides. It is however likely, that the isolates collected in 1998 (including the resistance isolate 99-150) were exposed to strobilurins. The number of applications is unknown.

The resistant isolate 99-150 was sent to the Syngenta Jealott's Hill International Research Station in the United Kingdom for further studies at the molecular level to investigate the mechanism of the resistance. Isolate 99-150 was assigned a collection number of K3758 on receipt at Jealott's Hill.

### Example 2

### Directed Cloning and Sequencing of the Cytochrome b Gene of P.aphanidermatum Isolate K3758 (99-150) In Comparison with that From Wild Type P.aphanidermatum

Characterisation of the cyt b gene from P.aphanidermatum isolate K3758 (99-150) was carried out using the method described below and as summarised in FIG. 3.

K3758 was prepared for analysis by culturing initially for 7 days at 25° C. under 12 hour fluorescent light on Potato Dextrose Agar (Oxoid), prepared according to the manufacturer's recommendations. An aliquot of mycelial growth was then harvested under sterile conditions and innoculated into 100 mls glycerol broth prepared as follows:

| | | |
|---|---|---|
| Glycerol | 2 | ml |
| Yeast Extract | 1 | g |
| MgSO₄ · 7H₂O | 0.05 | g |
| NaNO₃ | 0.6 | g |
| KCl | 0.05 | g |
| KH₂PO₄ | 0.15 | g |
| H₂O | to 100 | ml |

mixed well, and autoclaved at 15 psi for 15 minutes.

The culture was incubated then at 25° C. on an orbital shaker for 7 days. The resulting mycelial growth was then collected by centrifugation in a Richardson bottle and stored as a frozen pellet at −80° C. until analysis. Genomic DNA preparations were then carried out on two separate aliquots of 200 mg mycelium using Qiagen DNeasy® Plant Mini Kits, essentially according to the manufacturer's protocol, except for the initial extraction step.

In the case of the first mycelial sample, this extraction was by maceration of the mycelium in 400 μl buffer AP1 and 4 μl RNase from the Qiagen kit together with “lysing matrix combination 3” (¼″ sphere+garnet matrix) from a BIO 101 FastDNA™ kit. Extraction itself being performed for 4×40 seconds (total 160 sec) on speed setting 5 in an FP120 Fastprep™ Instrument (Anachem Ltd., Anachem House, Charles Street, Luton, Bedfordshire LU20EB, UK). The extraction tube was then centrifuged for five minutes at 13,000 rpm to pellet debris. The supernatant was then transferred to a 1.5 ml microcentrifuge tube and the DNA preparation was completed by following steps 3-13 of the Qiagen DNeasy Plant Mini Kit protocol with the final genomic DNA elutions being with 2×100 μl buffer AE.

Extraction of the second sample was achieved by adding 400 μl buffer API and 4 μl RNase to the mycelial pellet in a 2 ml microfuge tube, together with a sterile steel ball. This tube was then agitated in a Spex CertiPrep 8000 Mixer Mill (Glen Creston Ltd) for 10 minutes. As previously, the supernatant was then transferred to a 1.5 ml microcentrifuge tube and the DNA preparation completed by following steps 3-13 of the Qiagen DNeasy Plant Mini Kit protocol with the final genomic DNA elutions being with 2×100 μl buffer AE.

For PCR amplification of the cytochrome b gene, a 10-fold serial dilution of each genomic DNA preparation was then carried out using sterile double distilled H₂O (neat, 1:10 ar. 1:100). 10 μl of each dilution was then used as template for PCRs which were in each case performed in duplicate using primers 17F and 15R, which span the coding region for amino acids 73 to 283 of fungal cytochrome b genes (according to the S. cerevisiae numbering system).

| | |
|---|---|
| 17F: | 5′ AAATAACGGTTGGTTAATTCG 3′ |
| 15R: | 5′ TCTTAAAATTGCATAAAAAGG 3′) |

PCR cycling conditions included an initial incubation at 94° C. for 3 minutes, followed by 30 cycles of 94° C. for 45 seconds, 42° C. for 45 seconds and 72° C. for 1 minute 30 seconds. To conclude the PCR a final extension step at 72° C. for 10 minutes was also included. Reaction products were analysed by gel electrophoresis before cloning into pCR2.1-TOPO according to the manufacturer's protocol (Invitrogen). 10 transformants from each cloning event were then picked and used for Wizard Plus plasmid DNA preparations (Promega). To confirm that the PCR product was present, plasmid DNAs were digested with restriction enzyme EcoRI and analysed by gel electrophoresis. The inserts in five separate plasmids of the correct size from each initial K3758 sample were then sequenced using M13 forward and reverse primers (ABI377XL automated sequencer). Sequence data was analysed using appropriate analysis programmes (Seqman, Editseq and Macaw).

The deduced cytochrome b gene sequence of both samples of isolate K3758 were then aligned with P. aphanidermatum WT sequence which had been determined previously. Nucleotide and amino acid alignments are displayed in FIGS. 4 and 5, with the amino acid sequences having been predicted using the ‘Mold, Protozoan and Coelenterate Mitochondrial Code—Number 4’ as described in the Genetic Codes (NCBI taxonomy):
http://www3.ncbi.nlm.nih.pov/htbin-post/Taxonomy/wprintpc?mode=t

Striking features of this analysis were the:
High level of identity of the wild type and K3758 cytochrome b sequences, with only a single nucleotide and consequent amino acid substitution.
Identical sequences being found for wild type and K3758 isolates in the region corresponding to residue 143 where mutations for all previous Qₒ target site resistance mutants have been found by ourselves and others in plant pathogenic fungi (Windass et al., WO 00/66773; Sierotzki et al., Pest Manag. Sci. 56 (2000) 833-841; Sierotzki et al., Pest Biochem. Physiol. 68 (2000) 107-1120).
Presence of a single phenylalanine to leucine substitution at the position corresponding to yeast cytochrome b residue 129 (F129L).

Considering the results in greater detail: all 10 sequenced K3758 inserts had an A as the third base of the codon amino acid 129. Of the 5 inserts analysed for sample one, 2 contained other sequence differences (miniprep 2 contained 3 differences and mp 3 contained 2) which are suspected to be PCR errors, and of the 5 inserts in consensus sequence 2, 3 contained sequence differences (mp 1 contained 2 differences, mp 4 contained 2 and mp 5 contained 3) which are again typical of PCR errors. Only the presence of an A residue at the position corresponding to the third base of codon 129 was therefore a consistent difference between the K3758 and wild type P aphanidermatum cytochrome b sequence.

Interestingly the F129L substitution has been reported to confer resistance to myxothiazol (another Q₀ inhibitor) in S.cerevisiae, Rhodobacter capsulatus and Chlamydomonas reinhardtii (Esposti et al, Biochimica et Biophysica Acta, 1143 (1993) 243-271). There are however no previous reports of this substitution in plant pathogenic fungi.

### Example 3

### Design of ARMS Primers Capable of Discriminating F129 and L129 Alleles Found in Wild Type P.aphanidermatum and Strain K3758

Utilising the sequences of wild type P.aphanidermatum and isolate K3758 cytochrome b genes obtained according to Example 2 various specific ARMS primers were designed to detect the presence or absence of this F129L point mutation:

Three forward ARMS primers based on the wild type sequence:

| | |
|---|---|
| Pt129-1 | TTTATTTTAATGATGGCAACAGCTTAC |
| Pt129-2 | TTTATTTTAATGATGGCAACAGCTTCC |
| Pt129-3 | TTTATTTTAATGATGGCAACAGCTTGC |

Three forward ARMS primers based on the F129L mutation:

| | |
|---|---|
| Pt129-4 | TTTATTTTAATGATGGCAACAGCTTAA |
| Pt129-5 | TTTATTTTAATGATGGCAACAGCTTCA |
| Pt129-6 | TTTATTTTAATGATGGCAACAGCTTGA |

and a control primer designed upstream from the point mutation:

| | |
|---|---|
| Pt129-S | TATTTTTATTTTAATGATGGCAACAGC |

In each of the above ARMS primers, the −1 base (the 3′end base of the primer sequence) corresponds to the target point mutation site. Bases presented in bold differ from the wild type P. aphanidermatum cytochrome b sequence. In the Pt129-1 and Pt129-4 primers, the −2 position was changed from a T to an A base. In the Pt129-2 and Pt129-5 primers, the −2 position was changed from a T to a C base. In the Pt129-3 and Pt129-6 primers, the −2 position was changed from a T to a G base. These alterations to the sequence were made to destabilise the template/primer hybrid and render any primer extension more specific to the corresponding template. Examples in the literature have shown that destabilising the ARMS primer decreases the risk of the primer mispriming on the wrong template (Newton et al, Nucleic Acid Research 17 (7) 2503-2516 1989).

All primers were synthesised by Oswel DNA Service (Lab 5005, Medical and Biological Sciences Building, Southampton). Before use, the primers were diluted to 5 μM in a total volume of 500 μl double distilled nuclease free H₂O each. The primers were then further diluted to a final concentration of 500 nM in the PCRs.

### Example 4

### Design of a Scorpion Primer for use in Monitoring F129 and L129 Allele Status if P.aphanidermatum

Again utilising the sequences of wild type P.aphanidermatum and isolate K3758 cytochrome b genes obtained according to Example 2, Scorpion™ oiigonucieotides were designed to detect the selective amplification or wild type and L129 alleles by incorporating the detection system into the reverse PCR primer designed for use with the ARMS SNP detection and standard primers described in Example 3. The resulting amplicon being 172 bp long with the ARMS primers, and 176 bp long with the control primer.

Specifically the Scorpion primer was designed using Oligo 5 and MFold programs (MFold predicts optimal and suboptimal secondary structures- for RNA or DNA molecules using the energy minimization method of Zucker (Zucker, M. (1989) Science 244, 48-52; SantaLucia, J.Jr. (1998). Proc. Natl. Acad. Sci. USA 95, 1460-1465).

The sequence of the resultant P. aphanidermatum Scorpion primer was:

(SEQ ID NO 115)
5′ FAM-CCCGCCCGATATTGTTGATTGGTTATGGGGCGGG
(SEQ ID NO 116)
MR-HEG-TATTTAAAGTTGGATTATCTACAGC 3′

where: underlined regions are the hairpin forming parts (when the Scorpion primer is unreacted); FAM is the fluorescein dye; MR (methyl red) is a non-fluorigenic quencher attached to a uracil residue and HEG is the replication blocking hexethylene glycol monomer. The sequence in italics is the reverse primer sequence and the sequence in bold is the Scorpion sequence that binds to the authentic P.aphanidermatum cyt b extension product of the reverse primer.

The stem loop secondary structure of this Scorpion primer can be visualised using the MFold program (see FIG. 6) and is predicted to have an energy of −1.9 kcal/mol when not hybridised to the target cyt b gene. However in the presence of the extension product the hairpin structure is separated, as the probe sequence of the Scorpion primer binds to the extension product with a predicted energy of −4.9 kcal/mol. This separates the FAM dye from its quencher, causing emission of fluorescence detectable, for example, by an ABI Prism 7700 instrument. The annealing of the Scorpion element onto the newly synthesised strand is therefore energetically favourable compared to the Scorpion stem loop.

The Scorpion primer was synthesised by Oswel DNA Service (Lab 5005, Medical and Biological Sciences Building, Southampton). Before use, this primer were diluted to 5 μM in a total volume of 500 μl double distilled nuclease free H₂O. The primer was then further diluted to a final concentration of 500 nM in the PCRs.

### Example 5

### Validation of the use of ARMS and Scorpions Primers for Detection and Quantitation of the F129L SNP Found in Strain K3758

In all ARMS/Scorpion™ F129L SNP detection assays AmpliTaq Gold enzyme (Applied Biosystems) was included in the reaction mixes at 1 unit/25 μl reaction. The reaction mix also contained 1× buffer (10 mM Tris-HCl (pH8.3), 50 mM KCl, 3.5 mM MgCl₂, 0.01% gelatine) and 100 μM dNTPs (Amersham Pharmacia Biotech). Amplifications were performed in an ABI Prism 7700 instrument for continuous fluorescence monitoring. A preliminary cycle of 95° C. for 10 minutes was performed followed by 50 cycles of 95° C. for 15 sec and 60° C. for 45 sec. Fluorescence was monitored during the annealing/extension stage throughout all cycles.

Primers were first validated for use in such analyses by using plasmid DNA, at various concentrations, as template. This was performed in order to check the specificity and sensitivity of the primer designs. Partial wild type cytochrome b gene sequence and the corresponding tract containing the F129L mutation amplified from two P. aphanidermatum isolates were cloned into the TA pCR2.1 vector (Invitrogen) as described previously in example 2. 150 ul of the bacterial culture, from the 10 transformants from each cloning event picked for Wizard Plus plasmid DNA Preparations (Promega) (see example 2), was saved prior to carrying out these preparations and stored at 4° C. Following sequence analysis wild type and mutant plasmid DNA samples that contained no sequence differences from the consensus sequence were noted. The bacterial culture from which these wild type and mutant plasmid DNA sequences originated was picked for plasmid DNA maxipreps (Qiagen) following the manufacturers protocol. The resulting plasmid DNA was quantified and diluted to a concentration of 1 ug/ul (2×10¹¹ molecules/ul) using sterile double distilled H₂O.

The wild type and mutant plasmid DNA cassettes were diluted further to a concentration of 10 pg/ul (or 2×10⁶ molecules/ul) in double distilled H₂O and used as template to validate the specificity of the ARMS primers. Each ARMS primer was tested on wild type and mutant template as well as a no template (water only) control, under the PCR conditions described above. The Pt129-1 and Pt129-4 primers were preferred to the Pt129-2 and Pt129-3 and the Pt129-5 and Pt129-6 primers as duplicate PCRs gave more consistent results and were more specific. Wild type ARMS primer Pt129-1 gave a window of 15.27 cycles before amplification occurred on the inappropriate (mutant) template (FIG. 7). Mutant ARMS primer Pt129-4 gave a window of 16.96 cycles before amplification occurred on the inappropriate (wild type) template (FIG. 8).

Following the selection of a suitable ARMS primer for the detection of the point mutation the assay was validated further, so that its sensitivity of detection was fully understood before it was used to test biological samples for the presence of the L129 mutation. The first validation step was to establish if the window of specificity varied for the chosen ARMS primers over a 6 orders of magnitude range of wild type and mutant template DNA concentration. The wild type and mutant plasmid DNA cassettes described previously were diluted in Bovine Serum Albumin (BSA) (Fraction V Powder minimum 96%, Sigma A9647) at a concentration of 1 mg/ml through a 10 fold dilution series. The template concentrations covered the range 2×10⁸ molecules/ul to 2×10² molecules/ul. Both plasmid DNA cassettes and a no template (water only) control were tested in the ARMS/Scorpion assay using primers Pt129-1, Pt129-4 and Pt129-S, as described above.

TABLE 18
The results from the window of specificity experiment
using ARMS primer 129-4 (L129 allele selective)

| Plasmid | | | |
|---|---|---|---|
| Concentration | A Ct (mutant) | C Ct (wild type) | ΔCt |
| 2 × 10⁸ | 19.68 | 43.17 (1 rep) | 23.49 |
| 2 × 10⁷ | 21.96 | 40.74 | 18.78 |
| 2 × 10⁶ | 26.71 | no C | n/a |
| 2 × 10⁵ | 32.55 | no C | n/a |
| 2 × 10⁴ | 36.46 | no C | n/a |
| 2 × 10³ | 39.89 | no C | n/a |
| 2 × 10² | 43.45 | no C | n/a |

TABLE 19
The results from the window of specificity experiment
using ARMS primer 129-1 (F129 allele selective)

| Plasmid | | | |
|---|---|---|---|
| Concentration | A Ct (mutant) | C Ct (wild type) | ΔCt |
| 2 × 10⁸ | 41.68 | 19.73 | 21.95 |
| 2 × 10⁷ | 42.56 | 22.25 | 20.31 |
| 2 × 10⁶ | 44.24 | 25.86 | 18.38 |
| 2 × 10⁵ | 47.0 | 29.56 | 17.44 |
| 2 × 10⁴ | 46.12 (1 rep) | 33.38 | 12.32 |
| 2 × 10³ | 44.21 | 36.55 | 7.66 |
| 2 × 10² | no A | 40.39 | n/a |

These results show that the ARMS primers Pt129-1 and Pt129-4 do not amplify from their respective correct template with the same Ct (cycle threshold, the PCR cycle number where the change in fluorescence increases above background levels) value and this difference appears to be dependant on the concentration of plasmid DNA. Also for primer Pt129-1 the Ct between amplification on the correct and incorrect template does not remain constant across the range of template DNA concentrations tested in the assay. Whilst these initial ARMS primers do show some of the necessary features they would not provide the basis of an ideal assay, it was therefore decided to redesign the assay using a forward Scorpion primer upstream from the point mutation, in combination with a pair of reverse ARMS primers.

### Example 6

### Design of Further ARMS Primers Capable of Discriminating F129 and L129 Alleles Found in Wild Type P.aphanidermatum and Strain K3758

Utilising the sequences of wild type P.aphanidermatum and isolate K3758 cytochrome b genes obtained according to Example 2 a second set of specific ARMS primers was designed to detect the presence or absence of this F129L point mutation:

Six reverse ARMS primers based on the wild type sequence:

| | |
|---|---|
| Pt129-C1 | 5′ ACC CCA AGG TAA TAC ATA ACC CAA G 3′ |
| Pt129-C2 | 5′ ACC CCA AGG TAA TAC ATA ACC CAC G 3′ |
| Pt129-C3 | 5′ ACC CCA AGG TAA TAC ATA ACC CAG G 3′ |
| Pt129-C4 | 5′ ACC CCA AGG TAA TAC ATA ACC CTT G 3′ |
| Pt129-C5 | 5′ ACC CCA AGG TAA TAC ATA ACC CCT G 3′ |
| Pt129-C6 | 5′ ACC CCA AGG TAA TAC ATA ACC CGT G 3′ |

Fifteen reverse ARMS primers based on the F129L mutation:

| | |
|---|---|
| Pt129-A1 | 5′ ACC CCA AGG TAA TAC ATA ACC CAA T 3′ |
| Pt129-A2 | 5′ ACC CCA AGG TAA TAC ATA ACC CAC T 3′ |
| Pt129-A3 | 5′ ACC CCA AGG TAA TAC ATA ACC CAG T 3′ |
| Pt129-A4 | 5′ ACC CCA AGG TAA TAC ATA ACC CTT T 3′ |
| Pt129-A5 | 5′ ACC CCA AGG TAA TAC ATA ACC CCT T 3′ |
| Pt129-A6 | 5′ ACC CCA AGG TAA TAC ATA ACC CGT T 3′ |
| Pt129-A7 | 5′ ACC CCA AGG TAA TAC ATA ACT CAC T 3′ |
| Pt129-A8 | 5′ ACC CCA AGG TAA TAC ATA ACA CAC T 3′ |
| Pt129-A9 | 5′ ACC CCA AGG TAA TAC ATA ACG CAC T 3′ |
| Pt129-A10 | 5′ ACC CCA AGG TAA TAC ATA ACT CCT T 3′ |
| Pt129-A11 | 5′ ACC CCA AGG TAA TAC ATA ACA CCT T 3′ |
| Pt129-A12 | 5′ ACC CCA AGG TAA TAC ATA ACG CCT T 3′ |
| Pt129-A13 | 5′ ACC CCA AGG TAA TAC ATA ACT CGT T 3′ |
| Pt129-A14 | 5′ ACC CCA AGG TAA TAC ATA ACA CGT T 3′ |
| Pt129-A15 | 5′ ACC CCA AGG TAA TAC ATA ACG CGT T 3′ |

and two reverse control primers downstream from the point mutation were designed:

| | |
|---|---|
| Pt129-S4 | 5′ TTG ACC CCA AGG TAA TAC ATA ACC C 3′ |
| Pt129-S6 | 5′ TTG ACC CCA AGG TAA TAC ATA ACT C 3′ |

In each of the above ARMS primers, the −1 base (the 3′end base of the primer sequence) corresponds to the target point mutation site. Bases presented in bold differ from the wild type P. aphanidermatum cytochrome b sequence. In the Pt129-C1 and Pt129-A1 primers, the −2 position was changed from a T to an A base (in the reverse complement). In the Pt129-C2 and Pt129-A2 primers, the −2 position was changed from a T to a C base (in the reverse complement). In the Pt129-C3 and Pt129-A3 primers, the −2 position was changed from a T to a G base (in the reverse complement). In the Pt129-C4 and Pt129-A4 primers, the −3 position was changed from an A to a T base (in the reverse complement). In the Pt129-C5 and Pt129-A5 primers, the −3 position was changed from an A to a C base (in the reverse complement). In the Pt129-C6 and Pt129-A6 primers, the −3 position was changed from an A to a G base (in the reverse complement). In the Pt129-A7 primer the −5 position was changed from a C to a T base and the −2 position was changed from a T to a C base (in the reverse complement). In the Pt129-A8 primer the −5 position was changed from a C to an A base and the −2 position was changed from a T to a C base (in the reverse complement). In the Pt129-A9 primer the −5 position was changed from a C to a G base and the −2 position was changed from a T to a C base (in the reverse complement). In the Pt129-A10 primer the −5 position was changed from a C to a T base and the −3 position was changed from an A to a C base (in the reverse complement). In the Pt129-A11 primer the −5 position was changed from a C to an A base and the −3 position was changed from an A to a C base (in the reverse complement). In the Pt129-A12 primer the −5 position was changed from a C to a G base and the −3 position was changed from an A to a C base (in the reverse complement). In the Pt129-A13 primer the −5 position was changed from a C to a T base and the −3 position was changed from an A to a G base (in the reverse complement). In the Pt129-A14 primer the −5 position was changed from a C to an A base and the −3 position was changed from an A to a G base (in the reverse complement). In the Pt129-A15 primer the −5 position was changed from a C to a G base and the −3 position was changed from an A to a G base (in the reverse complement). These alterations to the sequence were made to destabilise the template/primer hybrid and render any primer extension more specific to the corresponding template. Examples in the literature have shown that destabilising the ARMS primer decreases the risk of the primer mispriming on the wrong template (Newton et al, Nucleic Acid Research 17 (7) 2503-2516 1989).

All primers were synthesised by Oswel DNA Service (Lab 5005, Medical and Biological Sciences Building, Southampton). Before use, the primers were diluted to 5 μM in a total volume of 500 μl double distilled nuclease free H₂O each. The primers were then further diluted to a final concentration of 500 nM in the PCRs.

### Example 7

### Design of a Further Scorpion Primer for use in Monitoring F129 and L129 Allele Status of P. aphanidermatum

Again utilising the sequences of wild type P.aphanidermatum and isolate K3758 cytochrome b genes obtained according to Example 2, Scorpion™ oligonucleotides were designed to detect the selective amplification of wild type and L129 alleles by incorporating the detection system into the forward PCR primer designed for use with the ARMS SNP detection and standard primers described in Example 3. The resulting amplicon was 126 bp long with the ARMS primers, and 129 bp long with the control primer.

Specifically the Scorpion primer was designed using Oligo 5 and MFold programs (MFold predicts optimal and suboptimal secondary structures for RNA or DNA molecules using the energy minimization method of Zucker (Zucker, M. (1989) Science 244, 48-52; SantaLucia, J.Jr. (1998). Proc. Natl. Acad. Sci. USA 95, 1460-1465).

The sequence of the resultant P. aphanidermatum Scorpion primer was: 5′ FAM-CGGCCGCCAACACCTGAACACCATAAACCGCGGCCG MR-HEG-TATATTATGGTTCATATATTACTCCAAG 3′ where: underlined regions are the hairpin forming parts (when the Scorpion primer is unreacted); FAM is the fluorescein dye; MR (methyl red) is a non-fluorigenic quencher attached to a uracil residue and HEG is the replication blocking hexethylene glycol monomer. The sequence in italics is the forward primer sequence and the sequence in bold is the Scorpion sequence that binds to the authentic P.aphanidermatum cyt b extension product of the forward primer.

The stem loop secondary structure of this Scorpion primer can be visualised using the MFold program (see FIG. 9) and is predicted to have an energy of −1.9 kcal/mol when not hybridised to the target cyt b gene. However in the presence of the extension product the hairpin structure is separated, as the probe sequence of the Scorpion primer binds to the extension product with a predicted energy of −5.6 kcal/mol. This separates the FAM dye from its quencher, causing emission of fluorescence detectable, for example, by an ABI Prism 7700 instrument. The annealing of the Scorpion element onto the newly synthesised strand is therefore energetically favourable compared to the Scorpion stem loop.

The Scorpion primer was synthesised by Oswel DNA Service (Lab 5005, Medical and Biological Sciences Building, Southampton). Before use, this primer were diluted to 5 μM in a total volume of 500 μl double distilled nuclease free H₂O. The primer was then further diluted to a final concentration of 500 nM in the PCRs.

### Example 8

### Validation of the use of ARMS and Scorpions Primers for Detection and Quantitation of the F129L SNP Found in Strain K3758

In all ARMS/Scorpion™ F129L SNP detection assays AmpliTaq Gold enzyme (Perkin-Elmer/ABI) was included in the reaction mixes at 1 unit/25 μl reaction. The reaction mix also contained 1× buffer (10 mM Tris-HCl (pH8.3), 50 mM KCl, 3.5 mM MgCl₂, 0.01% gelatine) and 100 μM dNTPs (Amersham Pharmacia Biotech). Amplifications were performed in an ABI Prism 7700 instrument for continuous fluorescence monitoring. A preliminary cycle of 95° C. for 10 minutes was performed followed by 50 cycles of 95° C. for 15 sec and 60° C. for 45 sec. Fluorescence was monitored during the annealing/extension stage throughout all cycles.

The first step involved in validating an assay is to test the ARMS primers for their potential to distinguish between the F129 (wild type) and L129 (mutant) alleles during amplification. The wild type and mutant P. aphanidermatum cyt b plasmid DNA constructs, as described in example 5, were diluted to a concentration 10 pg/ul (or 2×10⁶ molecules/ul) in double distilled H₂O and used as template to validate the selectivity of the ARMS primers. Each ARMS primer was tested on wild type and mutant template as well as in a no template (water only) control, under the PCR conditions described above. The results are summarised in Table 20 below.

TABLE 20
Validation of the selectivity of the ARMS primers designed.

| | | | | Observed | NTC |
|---|---|---|---|---|---|
| Primer | C Ct | A Ct | ΔCt | A:C ratio | amplification |
| Pt129-C1 | 25.79 | 42.6 | 16.81 | 1:114898 | none |
| Pt129-C2 | 20.42 | 43.57 | 23.15 | 1:9307743 | none |
| Pt129-C3 | 29.7 | no A | n/a | all C | none |
| Pt129-C4 | 21.28 | no A | n/a | all C | none |
| Pt129-C5 | 20.1 | 42.36 | 22.26 | 1:5022589 | none |
| Pt129-C6 | 19.63 | 44.17 | 24.54 | 1:24393610 | none |
| Pt129-A1 | 32.33 | 26.54 | 5.79 | 55:1 | none |
| Pt129-A2 | 34.01 | 20.87 | 13.14 | 9026:1 | none |
| Pt129-A3 | 40.2 | 31.29 | 8.91 | 481:1 | none |
| Pt129-A4 | 38.8 | 24.42 | 14.38 | 21321:1 | none |
| Pt129-A5 | 32.51 | 21.42 | 11.09 | 2179:1 | none |
| Pt129-A6 | 33.47 | 21.3 | 12.17 | 4608:1 | none |
| Pt129-A7 | 22.28 | 36.33 | 14.05 | 16961:1 | none |
| Pt129-A8 | 22.11 | 35.87 | 13.76 | 13873:1 | none |
| Pt129-A9 | 22.14 | 37.1 | 14.96 | 31871:1 | none |
| Pt129-A10 | 27.2 | 31.4 | 4.2 | 18.4:1 | none |
| Pt129-A11 | 24.85 | 31.25 | 6.4 | 84.4:1 | none |
| Pt129-A12 | 27.46 | 30.16 | 2.7 | 6.5:1 | none |
| Pt129-A13 | 23.19 | 41.99 | 18.8 | 456419:1 | none |
| Pt129-A14 | 23.1 | 43.5 | 20.4 | 1383604:1 | none |
| Pt129-A15 | 30.05 | no C | n/a | all A | none |
| Pt129-S4 | 19.06 | 18.98 | — | — | none |
| Pt129-S6 | 20.43 | 20.84 | — | — | none |

The results show that the L129 allele selective (mutant) primers Pt129-A14 and Pt129-A15 gave the largest window of specificity between amplification on the appropriate template and the inappropriate template (20.4 cycles, and no amplification of the inappropriate template respectively). Primer Pt129-A15 did not bind to the inappropriate template, however amplification on the correct template was very late, cycle 30. Primer Pt129-A14 is therefore the preferred L129 allele selective (mutant) ARMS primer as it has an earlier Ct value on the correct template. Primer Pt129-C4 is the preferred F129 allele selective (wild type) primer, as it does not bind to the inappropriate template. Primer Pt129-S4 is the preferred standard primer. These were therefore used to complete the assay validation. The L129 allele selective (mutant) primer Pt129-A14, however appears to give a Ct value approximately 5 cycles later than the F129 allele selective (wild type) primer Pt129-C4, even though the template is at the same concentration. This issue was assessed further by, checking whether the difference in Ct value between primers remains constant across a range of template concentrations.

Following the selection of a suitable ARMS primer pair for the detection of the point mutation the assay was further validated to fully understand the sensitivity of detection, before its use to test biological samples for the presence of the L129 mutation. The next study undertaken was therefore to establish if the window of specificity varied for the chosen ARMS primers, Pt129-C4 and PT129-A14, over a 6 order of magnitude range in template DNA concentration, and also if the difference in Ct value between the F129 and the L129 allele selective ARMS primers, when both primers are amplifying their respective correct template, remained constant across this template DNA concentration range. The wild type and mutant cyt b plasmid DNA constructs described previously were diluted in Bovine Serum Albumin (BSA) (Fraction V Powder minimum 96%, Sigma A9647) at a concentration of 1 mg/ml through a 10 fold dilution series. The template concentrations covered the range 2×10⁸ molecules/ul to 2×10² molecules/ul. The ARMS primers Pt129-C4 and Pt129-A14 and the standard primer Pt129-S4 were tested in the assay using both plasmid DNA cassettes at every concentration as template, and also in a no template (water only) control as described above.

TABLE 21
The results for primer Pt129-A14 across the
dilution range of plasmid DNA template

| Plasmid | A (L129) | C (F129) | | Observed | NTC |
|---|---|---|---|---|---|
| concentration | Ct | Ct | ΔCt | A:C ratio | amplification |
| 2 × 10⁸ | 17.9 | 35.42 | 17.52 | 187951:1 | none |
| 2 × 10⁷ | 20.56 | 38.75 | 18.19 | 299044:1 | none |
| 2 × 10⁶ | 24.2 | 44.59 | 20.39 | 1374047:1 | none |
| 2 × 10⁵ | 28.58 | no C | n/a | all A | none |
| 2 × 10⁴ | 31.56 | no C | n/a | all A | none |
| 2 × 10³ | 34.63 | no C | n/a | all A | none |
| 2 × 10² | 36.97 | no C | n/a | all A | none |

TABLE 22
The results for primer Pt129-C4 across the
dilution range of plasmid DNA template

| Plasmid | A (L129) | C (F129) | | Observed | NTC |
|---|---|---|---|---|---|
| concentration | Ct | Ct | ΔCt | A:C ratio | amplification |
| 2 × 10⁸ | no A | 13.91 | n/a | all C | none |
| 2 × 10⁷ | no A | 15.51 | n/a | all C | none |
| 2 × 10⁶ | no A | 19.67 | n/a | all C | none |
| 2 × 10⁵ | no A | 23.23 | n/a | all C | none |
| 2 × 10⁴ | no A | 26.15 | n/a | all C | none |
| 2 × 10³ | no A | 29.64 | n/a | all C | none |
| 2 × 10² | no A | 33.58 | n/a | all C | none |

TABLE 23
The results for primer Pt129-S4 across the
dilution range of plasmid DNA template

| Plasmid | A (L129) | C (F129) | NTC |
|---|---|---|---|
| concentration | Ct | Ct | amplification |
| 2 × 10⁸ | 13.86 | 14.08 | none |
| 2 × 10⁷ | 15.02 | 15.33 | none |
| 2 × 10⁶ | 18.85 | 19.77 | none |
| 2 × 10⁵ | 22.33 | 22.75 | none |
| 2 × 10⁴ | 25.44 | 25.46 | none |
| 2 × 10³ | 29.33 | 29.17 | none |
| 2 × 10² | 32.15 | 33.31 | none |

These results show that with the L129 allele selective (mutant) primer, Pt129-A14, the ΔCt between amplification on the appropriate and inappropriate template is approximately 18-20 cycles, for the most concentrated plasmid DNA templates, but as the plasmid DNA template concentration decreases further, the amplification on the inappropriate template ceases to be observed. The F129 allele selective (wild type) primer, Pt129-C4, only binds to the appropriate template, across the whole template concentration range. The specificity window was therefore constant across the whole template concentration range for the L129 and F129 allele selective primers Pt129-A14 and Pt129-C4.

TABLE 24
A comparison of the Ct values on the appropriate template,
for the L129 and the F129 allele selective primers

| Plasmid | Pt129-A14 Ct | Pt129-C4 Ct | ΔCt |
|---|---|---|---|
| concentration | (L129 template) | (F129 template) | (L129 − F129) |
| 2 × 10⁸ | 17.9 | 13.91 | 3.99 |
| 2 × 10⁷ | 20.56 | 15.51 | 5.05 |
| 2 × 10⁶ | 24.2 | 19.67 | 4.53 |
| 2 × 10⁵ | 28.58 | 23.23 | 5.35 |
| 2 × 10⁴ | 31.56 | 26.15 | 5.41 |
| 2 × 10³ | 34.63 | 29.64 | 4.99 |
| 2 × 10² | 36.97 | 33.58 | 3.39 |

The Ct (cycle threshold) values for each allele selective primer were plotted against the plasmid DNA template concentration (FIG. 10). The gradient of the plots for both primers was virtually identical, and the R² (correlation coefficient) values are also approximately the same. The plots are also parallel across the whole template concentration range, indicating that the difference in Ct value between the primers remains constant irrespective of the template DNA concentration.

The ΔCt (difference between two Ct values) between the L129 and F129 allele selective primer on the appropriate template was also plotted against plasmid DNA template concentration (FIG. 11). The gradient of the line was less than 0.1, which shows that the ΔCt between the L129 and F129 allele selective primers on their appropriate template does not vary with template DNA concentration.

The average ΔCt between the two primers binding to their appropriate template is 4.67. This value therefore needs to be subtracted from the ΔCt calculated between the L129 allele selective primer and the F129 allele selective primer, to give a true representation of the proportion of the two alleles in a sample.

The L129 allele selective primer, Pt129-A14, and the F129 allele selective primer, Pt129-C4, can therefore be used in this assay to directly compare the level of the L129 and the F129 alleles in a sample.

The second validation study involved testing the sensitivity of detection of the chosen ARMS primers Pt129-C4 and Pt129-A14. Plasmid DNA with the L129 allele, at a concentration of 2×10⁷ molecules/ ul was diluted into a background of plasmid DNA, with the F129 allele, at a constant concentration of 2×10⁷ molecules/ul, to give the following ratios: 1:1, 1:10, 1:100, 1:1,000, 1:10,000 and 1:100,000 of L129 to F129 alleles. The final plasmid concentration in the PCR is 1×10⁸ molecules/ul. These were tested, along with the wild type plasmid, mutant plasmid and water only control, with the primers Pt129-A 14, Pt129-C4 and Pt129-S4 in the assay, as described above.

TABLE 25
The results from the wild type and mutant
plasmid DNA control templates

| | Ct value - C template | Ct value - A template | |
|---|---|---|---|
| Primer | (F129 allele) | (L 129 allele) | ΔCt |
| Pt129-A14 | 43.16 | 27.33 | 15.83 |
| Pt129-C4 | 23.03 | no A | n/a |
| Pt129-S4 | 20.55 | 21.18 | n/a |

Primers Pt129-A14 and Pt129-C4 do not give the same Ct value on their correct template, as was noted above. This difference in Ct value was 27.33−23.03=4.3. This needs to be subtracted from every ΔCt calculated in the spiking experiment to give a true representation of the proportion of each allele in the samples.

TABLE 26
The results showing the sensitivity of detection
of the ARMS primers Pt129-A14 and Pt129-C4.

| | | | | Adjusted | | |
|---|---|---|---|---|---|---|
| | | | ΔCt | ΔCt | | |
| DNA | A primer | C primer | (ACt − | (ΔCt − | Observed | Observed |
| template | Ct | Ct | CCt) | 4.3) | A:C ratio | % C |
| 1:1 | 22.1 | 18.3 | 3.8 | −0.5 | | 58.6 |
| 1:10 | 25.53 | 18.05 | 7.48 | 3.18 | 1:9.08 | 9.9 |
| 1:100 | 29.83 | 18.23 | 11.14 | 6.84 | 1:114 | 0.86 |
| 1:1000 | 33.47 | 18.33 | 15.14 | 10.84 | 1:1826 | 0.05 |
| 1:10000 | 36.09 | 18.41 | 17.68 | 13.38 | 1:10638 | 0.009 |
| 1:100000 | 38.95 | 18.12 | 20.83 | 16.53 | 1:94269 | 0.001 |
| C template | 43.16 | 23.03 | 20.13 | n/a | n/a | n/a |
| NTC | none | none | — | — | — | — |

The results therefore show that the assay can detect levels of the L129 allele (A template) in a background of the F129 allele (C template) at less than 1:100000, before the A ARMS primer (L129 allele selective primer) binds to the inappropriate template (FIG. 12).

The third validation study undertaken was designed to test whether the ARMS primers Pt129-C4 and Pt129-A14 amplify with the same efficiency. For an ARMS/Scorpion assay to be reliable it is important that the chosen allele selective ARMS primers amplify with approximately the same efficiency over the range of template DNA concentrations that are likely to be tested in the assay. This is because the difference in Ct between the two primers corresponds directly to the frequency of the resistant allele in the sample. One way to test this is to compare how the ΔCt varies with template concentration. The log DNA input is plotted against the ΔCt and resulting slope should be less than 0.1

To check the efficiency of amplification of primers Pt129-A14 and Pt129-C4 a 1:10 mixture of mutant: wild type plasmid DNA (described previously) was diluted through a 2-fold dilution series across approximately a 100 fold range. The dilutions were all carried out in BSA at a concentration of 1 mg/ml. The following template dilutions, (neat, 1:2, 1:4, 1:8, 1:16, 1:32, 1:64 and 1:128) wild type only, and mutant only plasmid DNA controls, and a water only control, were tested in the ARMS/Scorpion assay with the primers Pt129-C4, Pt129-A14 Pt129-S4 primers as described above.

TABLE 27
The results for the wild type and mutant
plasmid DNA control templates.

| | Ct value - C template | Ct value - A template | |
|---|---|---|---|
| Primer | (F129 allele) | (L 129 allele) | ΔCt |
| Pt129-A14 | 37.77 | 22.6 | 15.17 |
| Pt129-C4 | 19.07 | no A | n/a |
| Pt129-S4 | 18.28 | 17.77 | — |

The difference in the Ct value for primer Pt129-A14 and Pt129-C4 on their appropriate templates is 22.6−19.07=3.53. This needs to be subtracted from every ΔCt calculated in the relative efficiency experiment to give a true representation of the proportion of each allele in the samples.

TABLE 28
The results from the relative efficiency experiment, showing
the ΔCt's for each template concentration.

| DNA | Log DNA | | | | Adjusted |
|---|---|---|---|---|---|
| concentration | concentration | C Ct | A Ct | ΔCt | ΔCt |
| 128 | 2.107 | 18.46 | 24.92 | 6.46 | 2.93 |
| 64 | 1.806 | 19.14 | 25.74 | 6.6 | 3.07 |
| 32 | 1.505 | 20.86 | 27.92 | 7.06 | 3.53 |
| 16 | 1.204 | 22.1 | 29.52 | 7.42 | 3.89 |
| 8 | 0.903 | 22.39 | 29.43 | 7.04 | 3.51 |
| 4 | 0.602 | 23.45 | 30.5 | 7.05 | 3.52 |
| 2 | 0.301 | 24.31 | 34.44 | 7.13 | 3.6 |
| 1 | 0 | 25.14 | 31.95 | 6.82 | 3.29 |

The ΔCt was plotted against the log DNA concentration and the trendline was calculated using Microsoft Excel (FIG. 13). This showed the gradient of the line was 0.05, which is less than 0.1 so primers Pt129-A14 and Pt129-C4 amplify with essentially the same relative efficiency.

The fourth stage in the validation of the assay was to investigate if host plant DNA (in this case the grass Lollium perenne) can influence the assay, e.g. by containing sequences which can adventitiously act as template in the PCR. L. perenne DNA will be present in samples where P.aphanidermatum infected leaf material is collected and tested directly.

For this study genomic DNA was extracted from a sample of L. perenne (100 mg) using the Qiagen DNeasy plant mini kit (the sample was first ground in a 1.5 ml microcentrifuge tube containing a steel ball by agitation for 10 minutes in the Centriprep mixer mill), and diluted across a 5-fold serial dilution in double distilled H₂O, to give the following concentrations: “neat” (as obtained directly from the mini kit preparation), 1 in 5, 1 in 25 and 1 in 125 (plant DNA stock solution to H₂O). Two mixtures of L129 allele: F129 allele plasmid DNA, 1:100 and 1:10,000 were also made (see above). These stock solutions were mixed with a decreasing background of plant material giving the following PCR inputs: 1:100 L129 allele: F129 allele+neat plant DNA, 1:100 L129 allele: F129 allele+1 in 5 plant DNA, 1:100 L129.allele: F129 allele+1 in 25 plant, DNA 1:100 L129 allele: F129 allele+1 in 125 plant DNA, 1:10,000 L129 allele: F129 allele+neat plant DNA, 1:10,000 L129 allele: F129 allele 30 1 in 5 plant DNA, 1:10,000 L129 allele: F129 allele+1 in 25 plant DNA, 1:10,000 L129 allele: F129 allele+1 in 125 plant DNA. These were all tested with primers Pt129-C4, Pt129-A14 and Pt129-S4 in the assay, along with the L.perenne DNA dilutions, the ratios of L129 allele: F129 allele plasmid DNA alone, the wild type and mutant plasmid DNA at a concentration of 2×10⁷ molecules/ul and a water only control, as described above.

TABLE 29
Wild type and mutant plasmid DNA controls

| | Ct value - C template | Ct value - A template | |
|---|---|---|---|
| Primer | (F129 allele) | (L 129 allele) | ΔCt |
| Pt129-A14 | 37.23 | 22.49 | 14.74 |
| Pt129-C4 | 19.95 | no A | n/a |
| Pt129-S4 | 19.36 | 18.61 | — |

The difference in the Ct value for primer Pt129-A14 and Pt129-C4 on their appropriate templates is 22.49−19.95=2.54. This needs to be subtracted from every ΔCt calculated to give a true representation of the proportion of each allele in the samples.

TABLE 30
The results from the validation experiment assessing the impact
of host plant DNA (L. perenne) on assay performance.

| | | | | | Adjusted | Observed | Observed |
|---|---|---|---|---|---|---|---|
| Template | C Ct | A Ct | S Ct | ΔCt | ΔCt | A:C ratio | % A |
| 1:100 (L129:F129) | 18.61 | 29.27 | — | 10.66 | 8.12 | 1:277 | 0.36 |
| 1:100 + neat plant | 20.57 | 30.56 | — | 9.98 | 7.44 | 1:173 | 0.57 |
| 1:100 + 1:5 plant | 19.41 | 29.09 | — | 9.69 | 7.15 | 1:142 | 0.7 |
| 1:100 + 1:25 plant | 18.83 | 29.3 | — | 10.47 | 7.93 | 1:243 | 0.41 |
| 1:100 + 1:125 plant | 18.54 | 29.99 | — | 9.45 | 6.91 | 1:120 | 0.82 |
| 1:10000 (L129:F129) | 19.18 | 34.76 | — | 15.58 | 13.05 | 1:8480 | 0.012 |
| 1:10000 + neat plant | 21.99 | 36.74 | — | 14.76 | 12.22 | 1:4764 | 0.02 |
| 1:10000 + 1:5 plant | 20.49 | 34.62 | — | 14.12 | 11.58 | 1:3069 | 0.03 |
| 1:10000 + 1:25 plant | 19.78 | 36.45 | — | 15.3 | 12.76 | 1:6936 | 0.014 |
| 1:10000 + 1:125 | 20.68 | 35.74 | — | 15.06 | 12.52 | 1:5877 | 0.017 |
| plant | | | | | | | |
| Neat plant | — | — | 35.36 | — | — | — | — |
| 1:5 plant | — | — | 36.27 | — | — | — | — |
| 1:25 plant | — | — | 38.07 | — | — | — | — |
| | | | (1 rep) | | | | |
| 1:125 plant | — | — | none | — | — | — | — |

When the mixtures of fungal plasmid DNA and L. perenne DNA were tested in the assay, there were no consistent changes in the Ct values and therefore the observed % C compared to when the fungal plasmid DNA was tested alone. L. perenne DNA therefore has no significant affect on the sensitivity of detection of the resistant allele in the assay.

The standard primer Pt129-S4 and the Scorpion primer are able to interact with the L. perenne DNA to give a detectable PCR product. However, neither the L129 nor the F129 allele specific primer in combination with the Scorpion primer is able to interact with the L. perenne DNA.

The presence of L. perenne DNA in a sample of P. aphanidermatumn will not have any affect on the amplification of the L129 or the F129 alleles, and the resulting estimation of the level of the L129 allele.

The final step in validation is to test the primers Pt129-C4, Pt129-A14 and Pt129-S4 on biological samples. There are different possible forms of biological samples that may be used as starting material for resistance monitoring assays.

These include mycelial growth from agar plates and P. aphanidermatum infected turf (L. perenne). To test P. aphanadiermatum samples from agar plates, a small amount of sterile double distilled H₂O (approximately 1-2 mls) was added to the plate and the mycelia scraped from the agar using a sterile disposable scraper. The mycelia solution was collected in a 1.5 or 2 ml sterile microcentrifuge tube and centrifuged. The supernatant was removed and the resulting mycelia sample then ground using either a FP120 Fastpre™ Instrument (Anachem Ltd., Anachem House, Charles Street, Luton, Bedfordshire LU20EB, UK) or a Spex CertiPrep 8000 Mixer Mill (Glen Creston Ltd) as described in example 2, and a genomic DNA preparation was then carried out using a Qiagen DNeasy plant mini kit, also described in Example 2. Alternatively 100 mg of L. perenne turf infected with P. aphanidermatum may be collected in a 1.5 or 2 ml microcentrifuge tube and ground in a similar manner to the mycelial material described above. The Qiagen DNeasy plant mini kit was then used to extract total infected plant DNA following the manufacturers protocol.

The resulting P.aphanidermatum and/or P.aphanidermatum/L.perenne DNA preparations was diluted 1:10 and 1:10 in sterile double distilled H₂O, and these template dilutions were tested in the ARMS/Scorpion assay as described above. Each template dilution was tested with primers Pt129-A14, Pt129-C4 and Pt129-S4; wild type and mutant plasmid DNA and a water only control were also included as positive and negative controls, respectively.

### Example 9

### Design of ARMS/Scorpions Assays Capable of Detection of F129 and L129 Alleles in any P.aphanidermatum Isolate

In order to detect other single nucleotide polymorphism capable of converting F₁₂₉ to L₁₂₉, a sense ARMS oligo pair/antisense Scorpion combination capable of distinguishing only position 1 of the 129 codon (i.e whether a thymine or a cytosine residue is present at position 1) may be used. Likewise an antisense ARMS oligo pair/sense Scorpion combination capable of distinguishing all possible residues at position 3 of the 129 codon, i.e. a thymine, cytosine, adenine or a guanine residue may detect alternative position 3 substitutions which can result in L₁₂₉ mediated resistance. In combination these position 1 and 3 assays also provide a means of assessing the level of double mutations which might result in conversion of F₁₂₉ to L₁₂₉ (codons: CTA and CTG).

The sense ARMS oligo pair/antisense Scorpion combination may utilise the Scorpion primer design previously and as detailed in example 4 where the detection system is incorporated on the reverse PCR primer, used in combination with the following SNP detection ARMS primer and control primer.

Three forward ARMS primers based on a thymine residue at position 1 of the 129 codon:

| | |
|---|---|
| Pt129-7 | TTTTTATTTTAATGATGGCAACAGCAT |
| Pt129-8 | TTTTTATTTTAATGATGGCAACAGCGT |
| Pt129-9 | TTTTTATTTTAATGATGGCAACAGCCT |

Three forward ARMS primers based on a cytosine residue at position 1 of the 129 codon:

| | |
|---|---|
| Pt129-10 | TTTTTATTTTAATGATGGCAACAGCAC |
| Pt129-11 | TTTTTATTTTAATGATGGCAACAGCGC |
| Pt129-12 | TTTTTATTTTAATGATGGCAACAGCCC |

and a control primer designed upstream from the point mutation:

| | |
|---|---|
| Pt129-S2 | TATTTTTATTTTAATGATGGCAACAGC |

In each of the above ARMS primers, the −1 base (the 3′end base of the primer sequence) corresponds to the target point mutation site. Bases presented in bold differ from the wild type P. aphanidermatum cytochrome b sequence. In the Pt129-7 and Pt129-10 primers, the −2 position is changed from a T to an A. In the Pt129-8 and Pt129-11 primers, the −2 position is changed from a T to a G. In the Pt129-9 and Pt129-12 primers, the −2 position is changed from a T to a C. These alterations to the sequence are again made to destabilise the template/primer hybrid and render any primer extension more specific to the corresponding template. When used with the antisesne Scorpion described in Example 4 the resulting amplicon will be 174 bp long with the ARMS primers and 176 bp long with the control primer.

In order to detect an adenine, thymine, cytosine or guanine residue at position three of the 129 codon using an antisense ARMS oligo pair/sense Scorpion combination, the sense Scorpion primer previously detailed in example 6, where the detection system is incorporated on the sense (forward) PCR primer, may be utilised in combination with the following SNP detection antisense (reverse) ARMS primers and control primer.

Three reverse ARMS primers based on a thymine residue at position 3 of the 129 codon:

| | |
|---|---|
| Pt129-13 | ACCCCAAGGTAATACATAACCCAAA |
| Pt129-14 | ACCCCAAGGTAATACATAACCCACA |
| Pt129-15 | ACCCCAAGGTAATACATAACCCAGA |

The following reverse ARMS primer based on an adenine residue at position 3 or the 129 codon:

| | |
|---|---|
| Pt129-A14 | ACCCCAAGGTAATACATAACACGTT |

The following reverse ARMS primer based on a cytosine residue at position 3 of the 129 codon:

| | |
|---|---|
| Pt129-C4 | ACCCCAAGGTAATACATAACCCTTG |

Three reverse ARMS primers based on a guanine residue at position 3 of the 129 codon:

| | |
|---|---|
| Pt129-16 | ACCCCAAGGTAATACATAACCCAAC |
| Pt129-17 | ACCCCAAGGTAATACATAACCCACC |
| Pt129-18 | ACCCCAAGGTAATACATAACCCAGC |

and a control primer designed downstream from the point mutation:

| | |
|---|---|
| Pt129-S4 | TTGACCCCAAGGTAATACATAACCC |

In each of the above ARMS primers, the −1 base (the 3′end base of the primer sequence) corresponds to the target point mutation site. Bases presented in bold differ from the wild type P. aphanidermatum cytochrome b sequence. In the Pt129-13 and Pt129-16, primers, the −2 position is changed from an A to a T. In the Pt129-14 and Pt129-17 primers, the −2 position is changed from an A to a G. In the Pt129-15 and Pt129-18 primers, the −2 position is changed from an A to a C. In the Pt129-C4 primer the −3 position is changed from a T to and A. In the Pt129-A14 primer the −3 position is changed from a T to a C and the −5 position is changed from a G to a T. These alterations to the sequence are made to destabilise the template/primer hybrid and render any primer extension more specific to the corresponding template. With the above sense Scorpion primer the resulting amplicon will be 110 bp long with the ARMS primers and 113 bp long with the control primer.

All primers can be synthesised by Oswel DNA Service (Lab 5005, Medical and Biological Sciences Building, Southampton). Before use, the primers are diluted to 5 μM in a total volume of 500 μl double distilled nuclease free H₂O each. The primers are then further diluted to a final concentration of 500 nM in the PCRs.

### Example 10

### Identification of two Novel Plasmopara viticola Samples Displaying Reduced Sensitivity to Qo Site Inhibitor Fungicides

During 2000 approximately 300 samples of P. viticola were collected from field sites across Europe. These were tested in both an in planta discriminatory dose bioassay to monitor for reduced sensitivity to Qo site inhibitor fungicides, and also in an ARMS/Scorpion G143A quantitative PCR assay (see Published International Patent Application WO 00/66773). Resistance to QoI's has been shown to be commonly associated with the presence of an alanine amino acid at position 143 (according to S. cerrivisae numbering system). This glycine to alanine amino acid change at position 143 in cytochrome b is caused by a single nucleotide polymorphism of a guanine to a cytosine at the second position of codon 143; the levels of the SNP are monitored in the assay. During this monitoring programme two samples, I112 and I116b, were found which showed reduced sensitivity to the QoI's on the in planta discriminatory dose bioassay, but only the wild type, glycine, amino acid at position 143. These were investigated further.

To confirm the reduced sensitivity to QoI's detected on the discriminatory dose bioassay, an in planta dose response bioassay was carried out on these samples. The in planta dose response bioassay was performed on 10 to 14 days old vine seedlings grown in 1.5″ pots. Six rates of azoxystrobin were chosen from preparatory tests, as the most suitable for showing any shifts in sensitivity to azoxystrobin in the dose response bioassay (Table 31).

TABLE 31
The rates of azoxystrobin used in the
in planta dose response bioassay.

| | Concentration | |
|---|---|---|
| Rate | of azoxystrobin | |
| 1 | 8.0 | ppm |
| 2 | 4.0 | ppm |
| 3 | 2.0 | ppm |
| 4 | 1.0 | ppm |
| 5 | 0.5 | ppm |
| 6 | 0.25 | ppm |

The azoxystrobin preparation used for all the tests was P53, a technical grade powder with a purity of >97% azoxystrobin. This was kept consistent across all the tests to eliminate any variation caused by differences in chemical purity or physical state. Five seedlings were used per azoxystrobin rate per sample, and 10 seedlings were used for the no azoxystrobin control (sprayed with de-ionised water only).

The chemical was sprayed, with a Devilbiss spray gun at 10 psi, to maximum retention onto the adaxial surface of the 2nd true leaf (test leaf) of each seedling, with care being taken to continuously swirl the solution in the spray gun to prevent local fluctuations in azoxystrobin concentration: The seedlings were then carefully placed in a growth room (Day: 24° C., 60% RH, 4-5000 lux; Night: 17° C., 95% RH; Day length: 16 hours) for 24 hours.

After this time each test leaf was inoculated with the samples I112 and I116b. Both samples had passed through 1 cycle of subculture on 3″ vines since being removed from low temperature storage (FIG. 14). The inoculum from each sample, obtained from the 3″ vines, was diluted to 5,000 sporangia per ml using a dry haemocytometer (Improved Neubayer, depth 0.1 mm, 1/400 mm², Hawksley Crystallite, BS 748).

The samples were inoculated, with a Devilbiss spray gun at 10 psi, to maximum retention onto the abaxial surface of the test leaf of each seedling, with the suspension being swirled frequently in the spray gun to keep sporangia from accumulating at the bottom. The test seedlings were incubated in an ambient room for twenty-four hours. The seedlings were then randomised and placed in a growth room (Day: 24° C., 60% RH, 4-5000 lux; Night: 17° C., 95% RH; Day length: 16 hours) for 6 days, before being returned to the ambient room for a further 24 hours to stimulate sporulation.

The seedlings were assessed directly after their removal from the ambient room for the second time. Disease assessment was based on the percentage area of the leaf covered by sporulating lesions and discolouration, and recorded in 5% increments. A score of 2% was given to leaves with a single, small lesion to indicate that complete control of the pathogen was not being exhibited. The data was then analysed using the statistical program AGSTAT. Both an OLS regression and arcsine transformation were used in the analysis, and the EC50 and EC95 values with their respective 95% confidence limits were calculated and are displayed in the Table 32 below.

TABLE 32
The EC50 and EC95 values for the P. viticola isolates
I112 and I116b.

| | | 95% confidence | | 95% confidence |
|---|---|---|---|---|
| Variate | EC50 | level | EC95 | level |
| K2075 (sensitive | 0.04 | 0.00-0.10 | 0.51 | 0.30-0.72 |
| baseline isolate) | | | | |
| 00I116B | 0.49 | 0.29-0.73 | 2.84 | 2.06-4.32 |
| 00I112 | 1.98 | 1.10-4.05 | 11.75 | 5.68-53.25 |

Both samples I116B and I112 showed reduced sensitivity to azoxystrobin, with higher EC50 and EC95 values compared to the sensitive baseline isolate. These samples were both then studied further at the molecular level to investigate the mechanism of the reduced sensitivity to azoxystrobin.

### Example 11

### Directed Cloning and Sequencing of the Cytochrome b Gene of P. viticola Samples I112 and I116b in Comparison with that from Wild Type and a Previously Confirmed Resistant Isolate

Characterisation of the cyt b gene from P. viticola samples I112 and I116b was carried out using the method described below.

The P. viticola samples I112 and I116b were prepared for analysis by the following method. Vine leaves displaying sporulating downy mildew symptoms were placed in a glass beaker. Approximately 400 mls of deionised water was then added to each sample, and the leaves were swirled to release the sporangia. The resulting sporangial suspension was filtered through two layers of muslin, then poured into sterile plastic 50 ml universal tubes and centrifuged for approximately 2 minutes at 4000 rpm, in a benchtop centrifuge (MSE, Centaur 2). After 2 minutes a sporangial pellet was visible in the bottom of each tube; the supernatants were decanted and the equivalent pellets were recombined and resuspended in 1 ml sterile deionised water. The sporangial sample was then transferred to a 1.5 ml sterile microcentrifuge tube, and centrifuged at 13000 rpm for one minute. The supernatant was removed and the samples placed at −80° C. until needed.

Genomic DNA was isolated from the above samples using Qiagen DNeasy Plant Mini Kits (catalogue number 69014) according to the manufacturers protocol with the following modifications. 400 uls of buffer API and 4 uls of RNaseA solution was added to each sample, and the pellet resuspended. The sporangial solution was then transferred to a sterile 2 ml microcentrifuge tube, a steel ball was added and the samples were agitated for 10 minutes in a Spex Certiprep 8000 Mixer Mill (Glen Creston Ltd) to grind the sample. The supernatant was then transferred to a 1.5 ml microcentrifuge tube, and the DNA preparation completed by following steps 3-13 of the Qiagen DNeasy Plant Mini Kit protocol, with the final genomic DNA elution being carried out with 2×100 ul of buffer AE.

For PCR amplification of the cyt b gene a 10 fold serial dilution of both genomic DNA preparations was then carried out using sterile double distilled H₂O giving the following dilutions 1:10, 1:100 and 1:1000 and these were all used as template in PCRs.

The “hot spot” region of the cytochrome b gene was PCR amplified from isolates I112 and I116b using primer pair 17/15.

Primer 17 = 5′ AAA TAA CGG TTG GTT AAT TCG 3′
Primer 15 = 5′ TCT TAA AAT TGC ATA AAA AGG 3′

These primers are specific to the P. viticola cyt b gene, and cover amino acids 73-283. This region includes all the amino acids positions that have been known in the literature to confer resistance to the QoI's in model organisms except for position 292.

PCRs were set up using Ready.To.Go® Taq polymerase PCR beads (Amersham Pharmacia Biotech). To each PCR bead 10 ul template, 2.5 ul forward primer 17, 2.5 ul reverse primer 15 both at a concentration of 10 pmol/ul to give a final concentration of 1 pmol/ul and 10 ul double distilled H₂O were added to give a total reaction volume of 25 ul. PCR cycling conditions were as follows: an initial incubation at 94° C. for 10 minutes, followed by 30 cycles of 94° C. for 45 seconds, 52° C. for 45 seconds and 72° C. for 1 minute 30 seconds. To conclude there was a final extension step at 72° C. for 10 minutes. PCR products were visualised on a 1% TBE agarose gel, and products of the expected size (600 bp) were present in all the lanes (except the water only control).

The three PCR products from each sample were pooled i.e. from 1:10, 1:100 and 1:1000 template DNA dilution, and these mixtures were cloned into the vector pCR2.1-TOPO using the TOPO TA Cloning kit from Invitrogen (K4500-01), following the manufacturers instructions. 16 colonies from each sample were picked for plasmid minipreps. The minipreps were carried out on a Qiagen Biorobot. Plasmid DNAs were then digested with EcoR1 and the digest products were visualised on a 1% TBE agarose gel, to establish which samples contained the correct sized inserts. 10 clones with the expected size inserts, from each sample, were then sequenced using M13 forward and reverse primers (Sequenced using AB1377XL automated sequencer). Sequence data was analysed using the appropriate bioinformatics software, Seqman, Editseq and Macaw.

The deduced cytochrome b gene sequence of each sample, I112 and I116b was then compared to the known wild type and resistant P. viticola cyt b sequence that had been determined previously. Nucleotide and amino acid alignments are displayed in FIGS. 15 and 16, with the amino acid sequences having been predicted using the ‘Mold, Protozoan and Coelenterate Mitochondrial Code—Number 4’ as described in the Genetic Codes (NCBI taxonomy):
http://www3.ncbi.nlm.nih.Rov/htbin-post/Taxonomy/wprintgc?mode=t
   Key points from this analysis were:
I112—the cytochrome b sequence from all 10 clones showed a single nucleotide polymorphism (SNP) of a T to a C at the first position of codon 129, compared to the wild type sequence. This encoded the amino acid leucine at position 129. All clones encoded the wild type amino acid at position 143 and no other point mutations were present. Wild type 129=TTT (phenylalanine, F) Mutant 129=CTT (leucine, L)
I16b—4 clones from the 10 clones sequenced were identical—these were identical to the known wild type P. viticola sequence encoding glycine at position 143 and phenylalanine at position 129. The other 6 clones from the 10 clones sequenced were also identical. These all encoded the amino acid leucine at position 129. This is again due to the SNP of a T to C at the first position of codon 129. These 6 clones all encoded the wild type amino acid glycine at position 143 and no other point mutations were present.
The amino acid change of phenylalanine to leucine at position 129 in samples I112 and I116b is likely to be associated with resistance to strobilurins, as these showed the largest dose response to azoxystrobin in the in planta bioassay.
This is the first time a single nucleotide polymorphism (SNP) conferring the F to L amino acid change has been seen in P. viticola.
The F129L amino acid change had however previously been observed in a field isolate of P. aphanidermatum and was shown to be associated with resistance to Qo site inhibitor fungicides (see above).
In these two pathogens the F129L amino acid change was caused by a different SNP
P. viticola TTT to CTT
P. aphanidermatum TTC to TTA

Considering the results in greater detail, for sample I112, 9 of the 10 clones had an identical sequence, and 1 clone had other sequence differences that differed by 2 bases. Again these are likely to be due to errors incorporated in the PCR. For sample I116b, 1 of the 4 clones that gave identical sequences had other sequence differences that differed by 4 bases, and 1 of the 6 clones that gave identical sequences had other sequence differences, that differed by 10 bases. These are due to unclear sequencing traces. These are shown in figure f.

### Example 12

### Design of ARMS Primers Capable of Distinguishing Between the F129 and L129 Alleles Found in Wild Type P.viticola and the I112 and I 116b Samples

Utilising the sequences of wild type P. viticola and sample I112 and I116b cytochrome b genes obtained according to Example 11 various specific ARMS primers were designed to detect the presence or absence of this F129L mutation: Six reverse ARMS primers based on the wild type sequence:

| | |
|---|---|
| PV129-T1 | 5′ CCCAAGGCAAAACATAACCCATATA 3′ |
| PV129-T2 | 5′ CCCAAGGCAAAACATAACCCATACA 3′ |
| PV129-T3 | 5′ CCCAAGGCAAAACATAACCCATAGA 3′ |
| PV129-T4 | 5′ CCCAAGGCAAAACATAACCCATTAA 3′ |
| PV129-T5 | 5′ CCCAAGGCAAAACATAACCCATCAA 3′ |
| PV129-T6 | 5′ CCCAAGGCAAAACATAACCCATGAA 3′ |

Six reverse ARMS primers based on the F129L mutation:

| | |
|---|---|
| PV129-C1 | 5′ CCCAAGGCAAAACATAACCCATATG 3′ |
| PV129-C2 | 5′ CCCAAGGCAAAACATAACCCATACG 3′ |
| PV129-C3 | 5′ CCCAAGGCAAAACATAACCCATAGG 3′ |
| PV129-C4 | 5′ CCCAAGGCAAAACATAACCCATTAG 3′ |
| PV129-C5 | 5′ CCCAAGGCAAAACATAACCCATCAG 3′ |
| PV129-C6 | 5′ CCCAAGGCAAAACATAACCCATGAG 3′ |

And a control reverse primer designed downstream from the point mutation:

| | |
|---|---|
| PV129-S | 5′ GTCCCCAAGGCAAAACATAACCCAT 3′ |

In each of the above ARMS primers, the −1 base (the 3′ end base of the primer sequence) corresponds to the target point mutation site. Bases presented in bold differ from the wild type P. viticola cytochrome b sequence. In the PV129-T1 and PV129-C1 primers the −2 position was changed from an A to a T base (reverse complement). In the PV129-T2 and PV129-C2 primers the −2 position was changed from an A to a C base (in the reverse complement). In the PV129-T3 and PV129-C3 primers the −2 position was changed from an A to a G base (in the reverse complement). In the PV]29-T4 and PV129-C4 primers the −3 position was changed from an A to a T base (in the reverse complement). In the PV129-T5 and PV129-C5 primers the −3 position was changed from an A to a C base (in the reverse complement). In the PV129-T6 and PV129-C6 primers the −3 position was changed from an A to a G base (in the reverse complement). As previously, these alterations to the sequence were made to destabilise the P.viticola cytochrome b gene template/primer hybrid and render any primer extension more specific to the corresponding template. Examples in the literature have shown that destabilising the ARMS primer decreases the risk of the primer mispriming on the wrong template (Newton et al, Nucleic Acid Research 17 (7) 2503-2516 1989).

All primers were synthesised by Oswel DNA Service (Lab 5005, Medical and Biological Sciences Building, Southampton). Before use, the primers were diluted to 5 uM in a total volume of 500 ul double distilled nuclease free H₂O each. The primers were then further diluted to a final concentration of 500 nm in the PCR's.

### Example 13

### Design of a Scorpion Primer for use in Monitoring the F129 and L129 Allele Status of P. viticola

Again utilising the sequences of wild type P. viticola and samples I112 and I116b cytochrome b genes obtained according to Example 11, Scorpion™ oligonucleotides were designed to detect the selective amplification of wild type and L129 alleles by incorporating the detection system into the forward PCR primer designed for use with the ARMS SNP detection and standard primers described in Example 12. The resulting amplicon was 161 bp long with the ARMS primers, and 164 bp long with the control primer.

Specifically the Scorpion primer was designed using Oligo 5 and MFold programs (MFold predicts optimal and suboptimal secondary structures for RNA or DNA molecules using the energy minimization method of Zucker (Zucker, M. (1989) Science 244, 48-52; SantaLucia, J.Jr. (1998). Proc. Natl. Acad. Scd. USA 95, 1460-1465).

The sequence of the resultant P. viticola Scorpion primer was: 5′ FAM-CCGCGCGCCATAA AGCTTCTCTAGGTGTAACGCGCGG MR-HEG-CATATTTTTAGGGGTTTGTATTACGG 3′ where: underlined regions are the hairpin forming parts (when the Scorpion primer is unreacted); FAM is the fluorescein dye; MR (methyl red) is a non-fluorigenic quencher attached to a uracil residue and HEG is the replication blocking hexethylene glycol monomer. The sequence in italics is the reverse primer sequence and the sequence in bold is the Scorpion sequence that binds to the authentic P. viticola cyt b extension product of the reverse primer.

The stem loop secondary structure of this Scorpion primer can be visualised using the MFold program (see FIG. 17) and is predicted to have an energy of −2.3 kcal/mol when not hybridised to the target cyt b gene. However in the presence of the extension product the hairpin structure is separated, as the probe sequence of the Scorpion primer hybridises to the extension product with a predicted energy of −5.1 kca/mol. This separates the FAM dye from its quencher, causing emission of fluorescence detectable, for example, by an ABI. Prism 7700 instrument. The annealing of the Scorpion element onto the newly synthesised strand is therefore energetically favourable compared to the Scorpion stem loop.

The Scorpion primer was synthesised by Oswel DNA Service (Lab 5005, Medical and Biological Sciences Building, Southampton). Before use, this primer were diluted to 5 μM in a total volume of 500 μl double distilled nuclease free H₂O. The primer was then further diluted to a final concentration of 500 nM in the PCRs.

### Example 14

### Validation of the use of ARMS and Scorpion Primers for Detection and Quantitation of the F129L SNP Found in Sample It112 and I116b

In all ARMS/Scorpion F129L SNP detection assays AmpliTaq Gold enzyme (Applied Biosystems) is included in the reaction mixes at 1 unit/25 ul reaction. The reaction mix also contains 1× buffer (10 mM Tris-HCl (pH8.3), 50 mM KCl, 3.5 mM MgCl₂, 0.01% gelatine) and 100 uM dNTP's (Amersham Pharmacia Biotech). Amplifications are performed in an ABI 7700 instrument for continuous fluorescence monitoring. The cycling conditions comprise a preliminary cycle of 95° C. for 10 minutes, followed by 50 cycles of 95° C. for 15 seconds and 60° C. for 45 seconds. Fluorescence is monitored during the annealing/extension stage throughout all cycles.

The ARMS primers are validated for use in such analyses by using plasmid DNA, at various concentrations as template. This is performed in order to check the specificity and sensitivity of the primer designs. Partial wild type cytochrome b gene sequence and the corresponding tract containing the F129L mutation amplified from two P. viticola samples has been cloned into the TA pCR2.1 vector (Invitrogen) as described previously in example 11. 150 ul of the bacterial culture, from the 10 transformants from each cloning event picked for Qiagen Biorobot plasmid DNA Preparations (Qiazen) (see example 11), was saved prior to carrying out these preparations and stored at 4° C. Following sequence analysis wild type and mutant plasmid DNA samples that contained no sequence differences from the consensus sequence were noted. The bacterial culture from which these wild type and mutant plasmid DNA sequences originated was picked for plasmid DNA maxipreps (Qiagen) following the manufacturers protocol. The resulting plasmid DNA was quantified and diluted to a concentration of 1 ug/ul (2×10¹¹ molecules/ul) using sterile double distilled H₂O.

The wild type and mutant cyt b plasmid DNA constructs were diluted further to a concentration of 10 pg/ul (or 2×10⁶ molecules/ul) in double distilled H₂O and used as template to validate the specificity of the ARMS primers. Each ARMS primer was tested on wild type and mutant plasmid DNA template as well as in a no template (water only) control, under the PCR conditions described above. The results are given in Table 33.

TABLE 33
The results from the validation experiment testing
the specificity of the ARMS primers designed to amplify
the F129 and L129 alleles in P. viticola.

| | | | | Observed | NTC ampli- |
|---|---|---|---|---|---|
| Primer | Wt Ct | Mut Ct | ΔCt | Wt:Mut ratio | fication |
| Pv129-Wt1 | 17.72 | 32.31 | 14.59 | 24662:1 | 1 rep |
| Pv129-Wt2 | 16.96 | 30.88 | 13.92 | 15500:1 | None |
| Pv129-Wt3 | 17.606 | 31.8 | 14.19 | 18690:1 | 1 rep |
| Pv129-Wt4 | 17.10 | 27.26 | 10.16 | 1144:1 | None |
| Pv129-Wt5 | 16.32 | 28.39 | 12.07 | 4300:1 | None |
| Pv129-Wt6 | 18.24 | 32.99 | 14.75 | 27554:1 | 1 rep |
| Pv129-Mut1 | 30.35 | 17.99 | 12.36 | 1:5257 | 1 rep |
| Pv129-Mut2 | 29.56 | 16.45 | 13.11 | 1:8841 | 1 rep |
| Pv129-Mut3 | 27.28 | 16.29 | 10.99 | 1:2034 | None |
| Pv129-Mut4 | 30.8 | 18.44 | 12.36 | 1:5257 | 2 reps |
| Pv129-Mut5 | 28.97 | 16.14 | 12.83 | 1:7281 | 2 reps |
| Pv129-Mut6 | 31.29 | 20.42 | 10.87 | 1:1872 | None |
| Pv129S | 17.0 | 17.22 | — | — | None |

The F129 selective ARMS primer PV129-Wt6 gave the largest window (ΔCt) between amplification on the appropriate and inappropriate templa and the L129 selective ARMS primer PV129-Mut5 gave the second largest window (ΔCt) between amplification on the appropriate and inappropriate template, but the earliest Ct on the correct template sowere chosen for further analysis.

Following the selection of the wild type ARMS primer PV129-T6 and the mutant ARMS primer PV129-C5 for the detection of the point mutalon, the assay required further validation to fully understand its sensitivity of detection, before it can be used to test biological samples for the presence of the L129 mutation. First the chosen ARMS primer pair, PV129-T6 and PV129-C5, was tested through a 10 fold dilution series of wild type (F129) and mutant (L129) plasmid DNA template to see how the specificity window varies with template concentration. The wild type and mutant plasmid DNA cassettes described previously were diluted in Bovine Serum Albumin (BSA) (Fraction V Powder minimum 96%, Sigma A9647) at a concentration of 1 mg/ml through a 10 fold dilution series across a 6 orders of magnitude range covering the concentration 2×10⁸ molecules/ul to 2×10² molecules/ul. Both plasmid DNA templates and a no template (water only) control were tested in the ARMS/Scorpion assay using the chosen ARMS primers and the control primer as described above. The results are summarised in Tables34 and 35.

TABLE 34
F129 Selective ARMS primer, PV129-Wt6, tested across
a dilution range of plasmid DNA template.

| | | | | Observed |
|---|---|---|---|---|
| Plasmid | Wt (L129) | Mut (F129) | | Wt:Mut |
| concentration | Ct | Ct | ΔCt | ratio |
| 2 × 10⁸ | 15.46 | 30.166 | 14.71 | 26801:1 |
| 2 × 10⁷ | 16.88 | 32.66 | 15.78 | 56267:1 |
| 2 × 10⁶ | 21.38 | 36.441 | 15.061 | 34183:1 |
| 2 × 10⁵ | 23.042 | — | — | — |
| 2 × 10⁴ | 26.83 | — | — | — |
| 2 × 10³ | 30.938 | — | — | — |
| 2 × 10² | 35.75 | — | — | — |

TABLE 35
L129 Selective ARMS primer, PV129-Mut5, tested across
a dilution range of plasmid DNA template.

| | | | | Observed |
|---|---|---|---|---|
| Plasmid | Wt (L129) | Mut (F129) | | Wt:Mut |
| concentration | Ct | Ct | ΔCt | ratio |
| 2 × 10⁸ | 29.96 | 14.90 | 15.06 | 1:34159 |
| 2 × 10⁷ | 31.11 | 15.94 | 15.18 | 1:37122 |
| 2 × 10⁶ | 32.76 | 17.79 | 14.97 | 1:32094 |
| 2 × 10⁵ | 38.26 | 22.55 | 15.71 | 1:53602 |
| 2 × 10⁴ | 39.47 | 24.83 | 14.64 | 1:25532 |
| 2 × 10³ | 45.36 | 28.41 | 16.95 | 1:126607 |
| 2 × 10² | | 32.927 | — | — |

The specificity range was constant across the whole template concentration range for the F129 and L129 allele selective primers PV129-Wt6 and PV129-Mut5.

The second validation study involved testing the sensitivity of detection of the chosen F129 and L129allele selective primers, PV129-Wt6 and PV129-Mut5. Plasmid DNA with the L129 allele, at a concentration of 2×10⁷ molecules/ul was diluted into a background of plasmid DNA, with the F129 allele, at a constant concentration of 2×10⁷ molecules/ul, to give the following ratios: 1:1, 1:10, 1:100, 1:1,000, 1:10,000 and 1:100,000 of L129 to F129 alleles. The final plasmid concentration in the PCR is 1×10⁸ molecules/ul. These were tested, along with the wild type plasmid, mutant plasmid and water only control, with the primers PV129-Wt6, PV129-Mut5 in the assay, as described above. The results are as shown in Table 36.

TABLE 36
The results showing the sensitivity of detection
of the ARMS primers Pv129-wt6 and Pv129-mut5.

| DNA | Mutant | Wild type | | Adjusted | Observed | % mutant |
|---|---|---|---|---|---|---|
| template | allele Ct | allele Ct | ΔCt | ΔCt (2.92) | C:T ratio | allele |
| 1:1 | 17.24 | 20.02 | −2.81 | 0.11 | 1:1.08 | 48.09 |
| 1:10 | 20.16 | 20.66 | −0.5 | 2.42 | 1:5.35 | 15.74 |
| 1:100 | 23.53 | 19.06 | 4.47 | 7.23 | 1:150 | 0.66 |
| 1:1000 | 25.45 | 18.87 | 6.58 | 9.5 | 1:724 | 0.14 |
| 1:10000 | 27.75 | 18.89 | 8.86 | 11.78 | 1:3516 | 0.028 |
| 1:100000 | 29.05 | 20.01 | 9.04 | 11.96 | 1:3984 | 0.025 |

The results therefore show that the assay can detect levels of the L129 allele (mutant) in a background of the F129 allele (wild type) at 1:10000, before primer PV129-mut5 (L129 allele selective primer) binds to the inappropriate template.

### Example 15

### Further Attempt at the Design of a Scorpion Primer for use in Monitoring F129 and L129 Allele Status of P. viticola

The results from the ARMS/Scorpion assay for the detection of the F129L SNP in P. viticola all showed low fluorescence when detecting the PCR product even from high concentrations of the plasmid DNA. This may lead to unreliable detection of the PCR product in the assay from low concentrations of DNA template. The Scorpion primer was therefore redesigned as described above.

The sequence of the resultant P. viticola Scorpion primer was:

5′ FAM-CCGGCCCCCATAAAGCTTCTCTAGGTGTAAGGGCCGG MR-
HEG-CATATTTTTAGGGGTTTGTATTACGG

where: underlined regions are the hairpin forming parts (when the Scorpion primer is unreacted); FAM is the fluorescein dye; MR (methyl red) is a non-fluorigenic quencher attached to a uracil residue and HEG is the replication blocking hexethylene glycol monomer. The sequence in italics is the forward primer sequence and the sequence in bold is the Scorpion sequence that binds to the authentic P. viticola cyt b extension product of the forward primer.

The stem loop secondary structure of this Scorpion primer was visualised using the MFold program and is predicted to have an energy of −1.3 kcal/mol when not hybridised to the target cyt b gene. However in the presence of the extension product the hairpin structure is separated, as the probe sequence of the Scorpion primer binds to the extension product with a predicted energy of −4.5 kcal/mol. This separates the FAM dye from its quencher, causing emission of fluorescence detectable, for example, by an ABI Prism 7700 instrument. The annealing of the Scorpion element onto the newly synthesised strand is therefore energetically favourable compared to the Scorpion stem loop.

The Scorpion primer was synthesised by Oswel DNA Service (Lab 5005, Medical and Biological Sciences Building, Southampton). Before use, this primer were diluted to 5 μM in a total volume of 500 μl double distilled nuclease free H₂O. The primer was then further diluted to a final concentration of 500 nM in the PCRs.

### Example 16

### Further Validation of the use of ARMS and Scorpion Primers for the Detection and Quantitation of the F129L SNP in P. viticola

The Scorpion primer described in example 15 was used in combination with the previously selected ARMS primers described in example 14, in the following validation studies to understand the sensitivity of detection of the assay. First the chosen ARMS primer pair, PV129-T6 and PV129-C5, was tested through a 10 fold dilution series of wild type (F129) and mutant (L129) plasmid DNA template to see how the specificity window varies with template concentration. The wild type and mutant plasmid DNA cassettes described previously were diluted in Bovine Serum Albumin (BSA) (Fraction V Powder minimum 96%, Sigma A9647) at a concentration of 1 mg/ml through a 10 fold dilution series across a 6 orders of magnitude range covering the concentration 2×10⁸ molecules/ul to 2×10² molecules/ul. Both plasmid DNA templates and a no template (water only) control were tested in the ARMS/Scorpion assay using the chosen ARMS primers and the control primer as described above. The results are summarised in Tables 37 and 38.

TABLE 37
L129 ARMS Primer, PV129-Mut5, tested across a
dilution range of plasmid DNA template with the
Scorpion primer designed in example 15.

| | | | | Observed |
|---|---|---|---|---|
| Concentration | Mutant Ct | Wild type Ct | Delta Ct | mut:wt ratio |
| 2 × 10⁸ | 12.43 | 28.68 | 16.25 | 1:77936 |
| 2 × 10⁷ | 16.87 | 30.33 | 13.46 | 1:11268 |
| 2 × 10⁶ | 19.85 | 34.25 | 14.41 | 1:21769 |
| 2 × 10⁵ | 22.84 | 37.44 | 14.60 | 1:24834 |
| 2 × 10⁴ | 25.20 | 40.54 | 15.34 | 1:41476 |
| 2 × 10³ | 28.17 | 39.65 | 11.48 | 1:2856 |
| 2 × 10² | 31.90 | 40.01 | 8.10 | 1:274 |

TABLE 38
F129 ARMS Primer, PV129-Wt6, tested across a
dilution range of plasmid DNA template with
the Scorpion primer designed in example 15.

| | | | | Observed |
|---|---|---|---|---|
| Concentration | Mutant Ct | Wild type Ct | Delta Ct | mut:wt ratio |
| 2 × 10⁸ | 30.50 | 15.69 | 14.81 | 28725:1 |
| 2 × 10⁷ | 33.95 | 18.45 | 15.50 | 46341:1 |
| 2 × 10⁶ | 37.97 | 23.02 | 14.95 | 31652:1 |
| 2 × 10⁵ | 39.57 | 25.76 | 13.81 | 14362:1 |
| 2 × 10⁴ | 43.09 | 28.51 | 14.59 | 24662:1 |
| 2 × 10³ | 45.04 | 32.56 | 12.48 | 5173:1 |
| 2 × 10² | 45.32 | 35.40 | 9.92 | 969:1 |

The specificity range was constant across the whole template concentration range for the F129 and L129 allele selective primers PV129-Wt6 and PV129-Mut5, except for the lowest template concentrations.

The second validation study involved testing the sensitivity of detection of the chosen F129 and L129 allele selective primers, PV129-Wt6 and PV129-Mut5 with the Scorpion primer described in example 15. The experiment was carried out as described in example 14 and the results are shown in Table 39.

TABLE 39
The results showing the sensitivity of detection of the ARMS primers Pv129-wt6
and Pv129-mut5, with the Scorpion primer designed in example 15.

| DNA | Mutant | Wild type | | Adjusted | Observed | % mutant |
|---|---|---|---|---|---|---|
| template | allele Ct | allele Ct | ΔCt | ΔCt (3.3) | C:T ratio | allele |
| 1:1 | 17.04 | 20.35 | −3.32 | −0.02 | 1.01:1 | 50.31 |
| 1:10 | 20.23 | 20.37 | −0.14 | 3.16 | 1:3.74 | 10.08 |
| 1:100 | 24.29 | 18.93 | 5.37 | 8.66 | 1:404 | 0.25 |
| 1:1000 | 26.84 | 18.7 | 8.14 | 11.43 | 1:2759 | 0.036 |
| 1:10000 | 30.01 | 19.47 | 10.54 | 13.84 | 1:14664 | 0.007 |
| 1:100000 | 31.45 | 19.72 | 11.73 | 15.03 | 1:33456 | 0.003 |

The results therefore show that the assay can detect levels of the L129 allele (mutant) in a background of the F129 allele (wild type) at 1:10000, before primer PV129-mut5 (L129 allele selective primer) binds to the inappropriate template.

A third validation experiment was designed to test whether the preferred ARMS primers amplify with the same efficiency. It is important to ensure that the efficiency of amplification is approximately equal, as the difference in Ct between the two primers corresponds directly to the frequency of the resistant allele in the sample. One way to test this is to compare how the ΔCt varies with template concentration. The log DNA input is plotted against the ΔCt and resulting slope should be less than 0.1 This was carried out as described in example 8. The template dilutions, along with the wild type and mutant plasmid DNA, and the water only controls were tested with the F129 and L129 allele specific primers, PV129- and wt6 and PV129-mut5 and the Scorpion primer described in example 15.

TABLE 40
The results from the relative efficiency experiment,
using ARMS primer Pv129-wt6 and Pv129-mut5 with
the Scorpion primer designed in example 15.

| DNA | Log DNA | | Wild type | | Adjusted |
|---|---|---|---|---|---|
| template | concentration | Mutant Ct | Ct | ΔCt | ΔCt (2.54) |
| 128 | 2.107 | 17.78 | 18.58 | −0.8 | 1.74 |
| 64 | 1.806 | 18.53 | 18.42 | 0.11 | 2.65 |
| 32 | 1.505 | 19.2 | 19.66 | −0.45 | 2.09 |
| 16 | 1.204 | 20.7 | 21.23 | −0.53 | 2.01 |
| 8 | 0.903 | 21.07 | 20.82 | 0.25 | 2.79 |
| 4 | 0.602 | 21.38 | 23.57 | −2.2 | 0.34 |
| 2 | 0.301 | 23.24 | 22.63 | 0.61 | 3.15 |
| 1 | 0 | 24.21 | 24.22 | 0.0 | 2.54 |

The Ct was plotted against log DNA template concentration and the slope of the line was calculated using excel. The slope of the line was less the 0.1 so the ARMS primers PV129-mut5 and PV129-wt6 amplify with approximately the same efficiency (FIG. 18).

A fourth validation experiment was designed to investigate if host plant DNA (in this case, grape vine DNA) can influence the assay, e.g. by acting as template in the PCR. Vine DNA will be present in any samples where infected leaf material is collected and tested directly.

To carry out this study genomic DNA was extracted from a sample of vine leaves using the Qiagen DNeasy plant mini kit (100 mg of material was first ground in a 1.5 ml microcentrifuge tube containing a steel ball by agitation for 10 minutes in the Centriprep mixer mill). The resulting DNA was diluted across a 5-fold serial dilution in double distilled H₂O, giving the following concentrations: “neat” (as obtained directly from the mini kit preparation), 1 in 5, 1 in 25 and 1 in 125 (plant DNA to H₂O). Two mixtures of L129 allele: F129 allele plasmid DNA, 1:100 and 1:10000 were also made (as described above). These stock solutions were mixed with a decreasing background of plant material to give the following PCR inputs: 1:100 L129 allele: F129 allele+neat plant DNA, 1:100 L129 allele: F129 allele+1 in 5 plant DNA, 1:100 L129 allele: F129 allele+1 in 25 plant, DNA 1:100 L129 allele: F129 allele+1 in 125 plant DNA, 1:10,000 L129 allele: F129 allele+neat plant DNA, 1:10,000 L129 allele: F129 allele+1 in 5 plant DNA, 1:10,000 L129 allele: F129 allele+1 in 25 plant DNA, 1:10,000 L129 allele: F129 allele+1 in 125 plant DNA. These were all tested with the preferred ARMS primers in the assay, along with the plant dilutions, the ratios of L129 allele: F129 allele plasmid DNA alone, the wild type and mutant plasmid DNA at a concentration 2×10⁷ molecules/ul and a water only control, as described above. The results were analysed in a similar manner to that described for the validation experiments described in this example.

The final stage of the validation involved testing the ARMS primers PV129-mut5 and PV129-wt6 and the control primer on biological samples. The biological sample used in this example was a sporangial pellet. The sporangia were washed from a sample of 30 vine leaves to form a sporangial suspension; this was centrifuged, and the supernatant removed, leaving a sporangia pellet. (P. viticola infected vine leaves may also be used as starting biological material). 100 mg of the biological material was ground using the Spex CertiPrep 8000 mixer mill (Glen Creston Ltd) as described in example 11. A genomic DNA prep was then be carried out using the Qiagen DNeasy plant mini kit following the manufacturers protocol, also described in example 11.

The resulting gDNA was diluted 1:10 and 1:100 in sterile double distilled H₂O, and these template dilutions were tested in the ARMS/scorpion assay as described above. Each template dilution was tested with the ARMS primers, PV129-mut5 and Pv129-wt6, and the control primer; wild type and mutant plasmid DNA and a water only control were also be included as positive and negative controls respectively. The results were analysed in a similar manner to that described for the validation experiments described in this example.

### Example 17

### Design of ARMS/Scorpions Assays Capable of Detection F129 and L129 Alleles in any P. viticola Isolate

In order to detect other single nucleotide polymorphism capable of converting F₁₂₉ to L₂₉, a sense ARMS oligo pair/antisense Scorpion combination capable of distinguishing only position 1 of the 129 codon (i.e whether a thymine or a cytosine residue is present at position 1) may be used. Likewise an antisense ARMS oligo pair/sense Scorpion combination capable of distinguishing all possible residues at position 3 of the 129 codon, i.e. a thymine, cytosine, adenineuor a guanine residue may detect alternative position 3 substitutions which can result in L₁₂₉ mediated resistance. In combination these position 1 and 3 assays also provide a means of assessing the level of double mutations which might result in conversion of F₁₂₉ to L₁₂₉ (codons: CTA and CTG).

The antisense ARMS oligo pair/sense Scorpion combination may utilise the Scorpion primer design previously detailed in example 13 where the detection system is incorporated on the forward PCR primer, used in combination with the following SNP detection ARMS primer and control primer.

Three reverse ARMS primers based on a thymine residue at position 3 of the 129 codon:

| | |
|---|---|
| PV129-1 | TCCCCAAGGCAAAACATAACCCAAA |
| PV129-2 | TCCCCAAGGCAAAACATAACCCACA |
| PV129-3 | TCCCCAAGGCAAAACATAACCCAGA |

Three reverse ARMS primers based on an adenine residue at position 3 of the 129 codon:

| | |
|---|---|
| PV129-4 | TCCCCAAGGCAAAACATAACCCAAT |
| PV129-5 | TCCCCAAGGCAAAACATAACCCACT |
| PV129-6 | TCCCCAAGGCAAAACATAACCCAGT |

Three reverse ARMS primers based on a cytosine residue at position 3 of the 129 codon:

| | |
|---|---|
| PV129-7 | TCCCCAAGGCAAAACATAACCCAAG |
| PV129-8 | TCCCCAAGGCAAAACATAACCCACG |
| PV129-9 | TCCCCAAGGCAAAACATAACCCAGG |

Three reverse ARMS primers based on a guanine residue at position 3 of the 129 codon:

| | |
|---|---|
| PV129-10 | TCCCCAAGGCAAAACATAACCCAAC |
| PV129-11 | TCCCCAAGGCAAAACATAACCCACC |
| PV129-12 | TCCCCAAGGCAAAACATAACCCAGC |

and a control primer designed downstream from the point mutation:

| | |
|---|---|
| PV129-S2 | TTGTCCCCAAGGCAAAACATAACCC |

In each of the above ARMS primers, the −1 base (the 3′ end of the primer sequence) corresponds to the target point mutation site. Bases presented in bold differ from the wild type P. viticola cytochrome b sequence. In the PV129-1, PV129-4, PV129-7.and PV129-10 primers the −2 positions changed from an A to a T. In the PV129-2, PV129-5, PV129-8 and PV129-11 primers the −2 position is changed from an A to a G. In the PV129-3, PV129-6, PV129-9 and PV129-12 primers the −2 position is changed from an A to a C. These alterations to the sequence are made to destabilise the template/primer hybrid and render any primer extension more specific to the corresponding template. The resulting amplicon will be 126 bp long with the ARMS primers and 129 bp long with the control primer.

All primers can be synthesised by Oswel DNA Service (Lab 5005, Medical and Biological Sciences Building, Southampton). Before use, the primers will be diluted to 5 μM in a total volume of 500 μl double distilled nuclease free H₂O each. The primers will then be further diluted to a final concentration of 500 nM in the PCRs.

In order to detect a thymine or cytosine residue at position one of codon 129, using a sense ARMS oligo pair/antisense Scorpion combination, another Scorpion design is detailed below. Again utilising the sequences of wild type P. viticola and samples I112 and I116b cytochrome b genes obtained according to example 11 the Scorpion oligonucleotides will be designed with the detection system incorporated on the reverse PCR primer; the Scorpion primer may then be used with the SNP detection ARMS primers and control primers.

Specifically the Scorpion primer was designed using Oligo 5 and MFold programs (MFold predicts optimal and suboptimal secondary structures for RNA or DNA molecules using the energy minimization method of Zucker (Zucker, M. (1989) Science 244, 48-52; SantaLucia, J.Jr. (1998). Proc. Natl. Acad. Sci. USA 95, 1460-1465).

The sequence of the resultant P. viticola Scorpion primer was:

5′ FAM-CCCGCCGTAATTGTAGGGGCTGTACTAATACGGCGGG
MRHEG-GATACCTAATGGATTATTTGAACCTACCT3′

Underlined regions are the hairpin forming parts (when the Scorpion primer is unreacted); FAM is the fluorescein dye; MR (methyl red) is a non-fluorigenic quencher attached to a uracil residue and HEG is the replication blocking hexethylene glycol monomer. The sequence in italics is the reverse primer sequence and the sequence in bold is the Scorpion sequence that binds to the authentic P. viticola cyt b extension product of the reverse primer. Bases that are underlined and also in bold participate as both part of the hairpin stem and the Scorpion sequence that binds to the extension product of the reverse primer.

The stem loop secondary structure of this Scorpion primer can be visualised using the MFold program (see FIG. 19) and is predicted to have an energy of −2.2 kcal/mol when not hybridised to the target cyt b gene. However in the presence of the extension product the hairpin structure is separated, as the probe sequence of the Scorpion primer binds to the extension product with a predicted energy of −6.1 kcal/mol. This separates the FAM dye from its quencher, causing emission of fluorescence detectable, for example, by an ABI Prism 7700 instrument. The annealing of the Scorpion element onto the newly synthesised strand is therefore energetically favourable compared to the Scorpion stem loop.

The antisense Scorpion primer may be used in combination with the following sense ARMS primers for detecting the thymine or cytosine residue at position I in codon 129.

Three forward ARMS primers based on a thymine residue at position 1 of the 129 codon:

| | |
|---|---|
| PV129-13 | TTTTTATTTTAATGATGGCGACTGCTT |
| PV129-14 | TTTTTATTTTAATGATGGCGACTGCCT |
| PV129-15 | TTTTTATTTTAATGATGGCGACTGCGt |

Three forward ARMS primers based on a cytosine residue at position 1 of the 129 codon:

| | |
|---|---|
| PV129-16 | TTTTTATTTTAATGATGGCGACTGCTC |
| PV129-17 | TTTTTATTTTAATGATGGCGACTGCCC |
| PV129-18 | TTTTTATTTTAATGATGGCGACTGCGC |

and a control primer designed upstream from the point mutation:

| | |
|---|---|
| PV129-S3 | TATTTTTATTTTAATGATGGCGACTGC |

In each of the above ARMS primers, the −1 base (the 3′end base of the primer sequence) correspond to the target point mutation site. Bases presented in bold differ from the wild type P. viticola cytochrome b sequence. In the PV129-13 and PV129-16 primers the −2 position is changed from an A to a T. In the PV129-14 and PV129-17 primers the −2 position is changed from an A to a C. In the PV129-15 and PV129-18 primers the −2 position is changed from an A to a G. These alterations to the sequence are made to destabilise the template/primer hybrid and render any primer extension more specific to the corresponding template. The resulting amplicon will be 278 bp long with the ARMS primer and 280 bp long with the control primer.

All primers may be synthesised by Oswel DNA Service (Lb 5005, Medical and Biological Sciences Building, Southampton). Before use the primers will be diluted to 5 uM in a total volume of 500 ul double distilled nuclease free H2O each. The primers will then further diluted to a final concentration of 500 nm in the PCR's.

### Example 18

### The design of a MGB (Minor Groove Binder) Hybridisation Assay for the Detection and Quantitation of the F129 and L129 alleles in P. viticola

Utilising the sequences of wild type P. viticola and sample I1112 and I116b cytochrome b genes obtained according to Example 11 an MGB hybridisation assay was designed to detect the T to C point mutation (SNP—single nucleotide polymorphism) at the first position of codon 129 that encodes the phenylalanine to leucine amino acid change. The assay utilises a common forward primer upstream from the point mutation (SNP), a common reverse primer downstream from the point mutation (SNP) and two MGB probes that cover the point mutation (SNP), one that matches the wild type sequence and one that matches the mutant sequence. The assay was designed using Primer Express vs 1.5 software following the design criteria.

The sequence of the primers and probes is as follows:

| | |
|---|---|
| Forward Primer: | 5′ CGGATCTTATATTACACCTAGAGAAGCTTT 3′ |
| Reverse Primer: | 5′ TTGTCCCCAAGGCAAAACAT 3′ |
| Mutant Probe | 5′ AACCCATAAGTGCAGTC 3′ |
| (L129 allele specific): | |
| Wild type Probe | 5′ ACCCATAAATGCAGTCG 3′ |
| (F129 allele specific): | |

The probes are designed to the complementary sequence. The base highlighted in bold and underlined is the point mutation (SNP). The mutant probe is labelled with the fluorophore FAM at the 5′ end, and the wild type probe is labelled with the fluorophore VIC at the 5′ end. Both probes are modified at the 3′ end with the attachment of an MGB unit. The primers and probes were synthesised by Applied Biosystems (Kelvin Close, Birchwood Science Park North, Warrington, Cheshire, WA3 7PB).

### Example 19

### The Validation of the MGB Hybridisation Assay for the Detection and Quantitation of the F129L SNP in P. viticola

A first validation experiment involved testing the specificity of hybridisation of the wild type and the mutant specific MGB probes to their correct DNA template compared with their incorrect DNA template. All MGB hybridisation assays are performed using the following reaction conditions: The forward and reverse primer are at a final concentration of 900 nM in the reaction, the wild type or mutant MGB hybridisation probe are at a final concentration of 200 nM in the reaction, the taqman universal PCR master mix is supplied at 2× concentration, 5 ul of DNA is used and the reaction is made up to a total volume of 25 ul with nuclease free H₂O. The PCR cycling conditions are as follows: One cycle at 50° C. for 2 minutes, followed by one cycle at 95° C. for 10 minutes, followed by 95° C. for 15 seconds and 60° C. for 1 minute for 50 cycles. Fluorescence is monitored during the annealing/extension stage throughout all cycles.

The specificity of the wild type and mutant MGB probes was tested using plasmid DNA constructs containing the partial cyt b gene either encoding the wild type (F129 allele) or mutant (L129 allele) sequence at position 129. These were prepared as described in example 14. The plasmid DNA containing the wild type (F129 allele) cyt b gene sequence and that containing the mutant (L129 allele) cyt b gene sequence were diluted through a 10 fold dilution series from 2×10⁸-2×10² molecules/ul. These were all tested in both the assay with the wild type MGB probe and that with the mutant MGB probe as described above. A water only control was also included. The results are given in Tables 41 and 42.

TABLE 41
Mutant MGB probe assay tested across a
dilution range of plasmid DNA template.

| Plasmid DNA | Mutant template | Wild type | |
|---|---|---|---|
| template dilution | Ct | template Ct | ΔCt |
| 2 × 10⁸ | 10.56 | no amplification | n/a |
| 2 × 10⁷ | 13.73 | no amplification | n/a |
| 2 × 10⁶ | 16.76 | no amplification | n/a |
| 2 × 10⁵ | 20.32 | no amplification | n/a |
| 2 × 10⁴ | 24.03 | no amplification | n/a |
| 2 × 10³ | 27.08 | no amplification | n/a |
| 2 × 10² | 30.9 | no amplification | n/a |

TABLE 42
Wild type MGB probe assay tested across a
dilution range of plasmid DNA template.

| Plasmid DNA | Mutant template | Wild type | |
|---|---|---|---|
| template dilution | Ct | template Ct | ΔCt |
| 2 × 10⁸ | no amplification | 10.05 | n/a |
| 2 × 10⁷ | no amplification | 14.22 | n/a |
| 2 × 10⁶ | no amplification | 17.25 | n/a |
| 2 × 10⁵ | no amplification | 20.21 | n/a |
| 2 × 10⁴ | no amplification | 23.24 | n/a |
| 2 × 10³ | no amplification | 27.06 | n/a |
| 2 × 10² | no amplification | 29.99 | n/a |

These results show that neither the wild type or the mutant MGB hybiridisation probe bind to their incorrect template across the dilution series in template DNA concentration.

A second validation experiment investigated the sensitivity of detection of the mutant MGB hybridisation probe and the wild type MGB hybridisation probe. Plasmid DNA with the L129 allele (mutant), at a concentration of 2×10⁷ molecules/ul was diluted into a background of plasmid DNA, with the F129 allele (wild type), at a constant concentration of 2×10⁷ molecules/ul, to give the following ratios: 1:1, 1:10, 1:100, 1:1,000, 1:10,000 and 1:100,000 of L129 to F129 alleles. The final plasmid concentration in the PCR was 1×10⁸ molecules/ul. These were tested, along with the wild type plasmid, mutant plasmid and water only control in both the assay with the wild type MGB probe and the mutant MGB probe. The results are shown in Table 43.

TABLE 43
The results showing the sensitivity of detection
of the wild type and the mutant MGB probe assays.

| DNA | Mutant | Wild type | | Mut:wt | % mutant |
|---|---|---|---|---|---|
| template | allele Ct | allele Ct | ΔCt | ratio | allele |
| 1:1 | 11.47 | 12.26 | −0.79 | 1.72:1 | 63.4% |
| 1:10 | 14.47 | 12.77 | 1.7 | 1:3.25 | 23.5% |
| 1:100 | no amplification | 13.01 | n/a | n/a | n/a |
| 1:1000 | no amplification | 13.05 | n/a | n/a | n/a |
| 1:10000 | no amplification | 12.97 | n/a | n/a | n/a |
| 1:100000 | no amplification | 13.03 | n/a | n/a | n/a |

The sensitivity of detection of this assay is only 1 mutant (L129) allele in a background of 10 wild type (F129) alleles. The hybridisation assays using a TaqMan probe or TaqMan MGB probe for the detection of the mutation, in combination with a common forward and reverse primer pair, are able to detect the mutant allele at the level of 5-10% within a fungal sample. When lower levels of detection of the mutant allele are required, it is more preferable to use an ARMS primer in combination with a suitable detection system ie Scorpion primer.

### Example 20

### Characterisation of Cytochrome b Gene from Alternaria solani Isolates Showing Reduced Sensitivity to QoI Site Inhibitor Fungicides

Cytochrome b genes have also been characterised from two isolates of A. solani showing reduced sensitivity to QoI inhibitor fungicides when tested in a bioassay. The DNA was extracted from these isolates, and the resulting gDNA (genomic DNA) was used as template in PCRs to amplify the cytochrome b gene. PCRs were performed as described previously using the following primers:

| | |
|---|---|
| Forward | 5′CTG TTA TCT TTA TCT TAA TGA TGG 3′ |
| primer: | |
| Reverse | 5′GGA ATA GAT CTT AAT ATA GCA TAG 3′ |
| primer: | |

under the following condtions: one cycle of 94° C. for 3 mins, followed by 30 cycles of 45 secs at 94° C., 45 secs at 58° C. and 1 min 30 secs at 72° C., followed by one 1 cycle of 10 minutes at 72° C.

PCR products were visualised by gel electrophoresis and those of the correct predicted size were cloned using TOPO TA cloning kit from Invitrogen and transformed into E. coli. Transformant E. coli colonies were selected, sub-cultured and plasmid DNA prepared by miniprepe as described previously. Plasmid DNAs were sequenced (see example 11) and the sequence data was analysed using appropriate bioinformatics software (e.g. Seqman, Editseq and Macaw). The deduced cytochrome b gene sequence from the two isolates was compared to the known wild type cytochrome b sequence (determined previously). The nucleotide and amino acid alignments are shown in FIGS. 20 and 21, with the amino acid sequences having been predicted using the ‘Mold, Protozoan and Coelenterate Mitochondrial Code—Number 4’ as described in the Genetic Codes (NCPI taxonomy):- http://www3.ncbi.nlm.nih.gov/htbin-post/Taxonomy/wprintgc?mode=t.

The presence of a T to a C mutation (SNP) was observed at the first position of codon 129 in the cytochrome b gene of the first A. solani sample. This mutation, when compared with a baseline/parental sample, results in a phenylalanine to leucine amino acid change at position 129 according to the S. cerrivisae numbering system. The second sample (sample 2) also showed the presence of a mutation (SNP) in codon 129, however this was from C to A at the third position of codon 129. This also results in a phenyalanine to leucine change at position 129 according to the S.cerrivisae numbering system. The reduced sensitivity to QoI site inhibitor compounds in each of these two samples hat was observed in the bioassay is likely to be associated with the amino acid change of phenylalanine (F) to leucine (L) at position 129. This is the first time that single nucleotide polymorphisms (SNP) conferring a F to L amino acid change has been seen in A. solani. It is also the first time in any fungal species that two different SNPs in cytochrome b codon 129 have been detected with eah encoding a F to L amino acid change.

1. A method for the detection of one or more mutations in a fungal cytochrome b gene resulting in an amino acid replacement at the position corresponding to S. cerevisiae cytochrome b residue 129 in the encoded protein wherein the presence of said mutation(s) gives rise to fungal resistance to a strobilurin analogue or any other compound in the same cross resistance group said method comprising identifying the presence or absence of said mutation(s) in fungal nucleic acid using any (or a) single nucleotide polymorphism detection technique.

2. The method according to claim 1 for the detection of one or more mutations in a fungal cytochrome b gene resulting in a phenyalanine to leucine replacement at the position corresponding to S. cerevisiae cytochrome b residue 129 (F129L) in the encoded protein wherein the presence of said mutation(s) gives rise to fungal resistance to a strobilurin analogue or any other compound in the same cross resistance group said method comprising identifying the presence or absence of said mutation(s) in fungal nucleic acid using any (or a) single nucleotide polymorphism detection technique.

3. The method according to claim 1 wherein said method comprises detecting the presence of an amplicon generated during a PCR reaction wherein said PCR reaction comprises contacting a test sample comprising fungal nucleic acid with a diagnostic primer in the presence of appropriate nucleotide triphosphates and an agent for polymerisation wherein the detection of said amplicon is directly related to presence or absence of said mutation(s) in said nucleic acid.

4. The method according to claim 1 wherein said method uses an allele selective hybridisation probe technique.

5. A method for the diagnosis of one or more nucleotide polymorphisms in a fungal cytochrome b gene which method comprises determining the sequence of a fungal nucleic acid that encodes a fungal cytochrome b protein at a position corresponding to one or more of the bases in the triplet coding for the amino acid at the position corresponding to S. cerevisiae cytochrome b residue 129 in the fungal cytochrome b protein and determining the resistance status of the said fungi to a strobilurin analogue or a compound in the same cross resistance group by reference to one or more polymorphisms in the cytochrome b gene.

6. A method for detecting fungal resistance to a strobilurin analogue or any other compound in the same cross resistance group said method comprising identifying the presence or absence of one or more mutation(s) in a fungal nucleic acid encoding a fungal cytochrome b gene wherein the presence of said mutation(s) gives rise to resistance to a strobilurin analogue or any other compound in the same cross resistance group said method comprising identifying the presence or absence of a single nucleotide polymorphism occurring at a position corresponding to one or more of the bases in the triplet coding for the amino acid at the position corresponding to S. cerevisiae cytochrome b residue 129 in the fungal cytochrome b protein.

7. The method according to claim 1 wherein a single nucleotide polymorphism mutation occurs at the first and/or third base in the triplet coding for the amino acid at the position corresponding to S. cerevisiae cytochrome b residue 129.

8. The method according to claim 1 wherein a single nucleotide polymorphism mutation occurs at the first or third base in the triplet coding for the amino acid at the position corresponding to S. cerevisiae cytochrome b residue 129.

9. The method according to claim 1 wherein the single nucleotide polymorphism mutation occurs at the third base in the triplet coding for the amino acid at the position corresponding to S. cerevisiae cytochrome b residue 129.

10. The method according to claim 1 wherein the single nucleotide polymorphism mutation occurs at the first base in the triplet coding for the amino acid at position corresponding to S. cerevisiae cytochrome b residue 129.

11. A fungal DNA sequence encoding all or part of a cytochrome b protein which, when said sequence is lined up against the corresponding wild type DNA sequence encoding a cytochrome b protein, is seen to contain a single nucleotide polymorphism mutation at a position in the DNA corresponding to one or more of the bases in the triplet coding for the amino acid at the position corresponding to S. cerevisiae cytochrome b residue 129 in the protein which results in the replacement of the normal phenylalanine residue with an alternative amino acid.

12. The fungal DNA sequence according to claim 11 wherein said alternative amino acid is a leucine residue.

13. The fungal DNA sequence according to claim 11 obtained or obtainable from a fungus selected from the group consisting of Plasmopara viticola, Erysiphe graminis f.sp. tritici, Erysiphe graminis f.sp. hordei, Rhynchosporium secails, Pyrenophora teres, Mycosphaerella graminicola, Venturia inaequalis, Mycosphaerella fijiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Mycosphaerella musicola, Cercospora arachidola, Colletotrichum acutatum, Wilsonomyces carpophillum, Didymella bryoniae, Peronospora tabacina, Puccinia recondita and Puccinia horiana.

14. The fungal DNA sequence according to claim 13 obtained or obtainable from a fungus selected from the group consisting of Plasmopara viticola, Erysiphe graminis f.sp. tritici, Erysiphe graminis f.sp. hordei, Rhynchosporium secalis, Pyrenophora teres, Mycosphaerella graminicola, Venturia inaequalis, Mycosphaerelia fjiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Mycosphaerella musicola and Cercospora arachidola.

15. A method for the detection of the presence of absence of one or more mutation(s) in a fungal cytochrome b gene, said mutation(s) resulting in replacement in the encoded protein of a phenyalanine residue at the position corresponding to S. cerevisiae cytochrome b residue 129 said method comprising identifying the presence or absence of said mutation(s) in a sample of fungal nucleic acid wherein any (or a) single nucleotide polymorphism detection method is based on the sequence information from around 30 to 90 nucleotides upstream and/or downstream of the position corresponding to one or more of the bases in the triplet coding for the amino acid at the position corresponding to S. cerevisiae cytochrome b residue 129 in either the wild type or mutant protein.

16. The method according to claim 15 wherein said detection method is based on the sequence information from around 30 to 90 nucleotides upstream and/or downstream of the position corresponding to the first or third base in the triplet coding for the amino acid at the position corresponding to S. cerevisiae cytochrome b residue 129 in either the wild type or mutant protein.

17. The method according to claim 16 wherein said detection method is based on the sequence infornation from around 30 to 90 nucleotides upstream and/or downstream of the position corresponding to the third base in the triplet coding for the amino acid at the position corresponding to S. cerevisiae cytochrome b residue 129 in either the wild type or mutant protein.

18. The method according to claim 16 wherein said detection method is based on the sequence information from around 30 to 90 nucleotides upstream and/or downstream of the position corresponding to the first base in the triplet coding for the amino acid at the position corresponding to S. cerevisiae cytochrome b residue 129 in either the wild type or mutant protein.

19. An allele specific oligonucleotide capable of binding to a fungal nucleic acid sequence encoding a wild type cytochrome b protein wherein said oligonucleotide comprises a sequence which recognises a nucleic acid sequence encoding a phenylalanine residue at the position corresponding to S. cerevisiae cytochrome b residue 129.

20. An allele specific oligonucleotide capable of binding to a fungal nucleic acid sequence encoding a wild type cytochrome b protein selected from the group consisting of Plasmopara viticola, Erysiphe graminis f.sp. triticii, Erysiphe graminis f.sp. hordei, Rhynchosporium secalis, Pyrenophora teres, Mycosphaerella graminicola, Venturia inaequalis, Mycosphaerella rjiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Mycosphaerella musicola, Cercospora arachidola, Colletotrichum acutatum, Wilsonomyces carpophillum, Didymella bryoniae, Peronospora tabacina, Puccinia recondita and Puccinia horiana wherein said oligonucleotide comprises a sequence which recognises a nucleic acid sequence encoding a phenylalanine residue at the position corresponding to S. cerevisiae cytochrome b residue 129.

21. An allele specific oligonucleotide capable of binding to a fungal nucleic acid sequence encoding a mutant cytochrome b protein wherein said oligonucleotide comprises a sequence which recognises a nucleic acid sequence encoding an amino acid selected from the group isoleucine, leucine, serine, cysteine, valine, tyrosine, and most preferably leucine at the position corresponding to S. cerevisiae cytochrome b residue 129.

22. The allele specific oligonucleotide according to claim 21 capable of binding to a fungal nucleic acid sequence encoding a mutant cytochrome b protein selected from the group consisting of Plasmopara viticola, Erysiphe graminis f.sp. tritici, Erysiphe graminis f.sp. hordei, Rhynchosporium secalis, Pyrenophora teres, Mycosphaerella graminicola, Venturia inaequalis, Mycosphaerella filiensis var. difformis, Sphaerotheca fuliginea, Uncinula necator, Colletotrichum graminicola, Pythium aphanidermatum, Colletotrichum gloeosporioides, Oidium lycopersicum, Phytophthora infestans, Leveillula taurica, Pseudoperonospora cubensis, Alternaria solani, Rhizoctonia solani, Mycosphaerella musicol, Cercospora arachidola, Colletotrichum acutatum, wilsonomyces carpophillum, Didymella bryoniae, Peronospora tabacina, Puccinia recondita and Puccinia horiana wherein said oligonucleotide comprises a sequence which recognises a nucleic acid sequence encoding an amino acid selected from the group isoleucine, leucine, serine, cysteine, valine, tyrosine, and most preferably leucine at the position corresponding to S. cerevisiae cytochrome b residue 129.

23. An allele specific oligonucleotide probe capable of detecting a wild type cytochrome b gene sequence at a position in the DNA corresponding to one or more of the bases in the triplet coding for the amino acid at the position corresponding to S. cerevisiae cytochrome b residue 129 in the protein.

24. An allele specific oligonucleotide probe capable of detecting a fungal cytochrome b gene polymorphism at a position in the DNA corresponding to one or more of the bases in the triplet coding for the amino acid at the position corresponding to S. cerevisiae cytochrome b residue 129 in the protein.

25. The allele specific oligonucleotide probe according to claim 24 capable of detecting a fungal cytochrome b gene polymorphism at positions in the DNA corresponding to the first and/or third base in the triplet coding for the amino acid at the position corresponding to S. cerevisiae cytochrome b residue 129.

26. The allele specific oligonucleotide probe according to claim 24 capable of detecting a fungal cytochrome b gene polymorphism at positions in the DNA corresponding to the first or third base in the triplet coding for the amino acid at the position corresponding to S. cerevisiae cytochrome b residue 129.

27. The allele specific oligonucleotide probe according to claim 24 capable of detecting a fungal cytochrome b gene polymorphism at positions in the DNA corresponding to the third base in the triplet coding for the amino acid at the position corresponding to S. cerevisiae cytochrome b residue 129.

28. The allele specific oligonucleotide probe according to claim 24, capable of detecting a fungal cytochrome b gene polymorphism at positions in the DNA corresponding to the first base in the triplet coding for the amino acid at the position corresponding to S. cerevisiae cytochrome b residue 129.

29. An allele specific primer capable of detecting a cytochrome b gene polymorphism at a position in the DNA corresponding to one or more of the bases in the triplet coding for the amino acid at the position corresponding to S. cerevisiae cytochrome b residue 129 in the protein.

30. The allele specific primer according to claim 29 capable of detecting a cytochrome b gene polymorphism at a position in the DNA corresponding to the first and/or third base in the triplet coding for the amino acid at the position corresponding to S. cerevisiae cytochrome b residue 129.

31. The allele specific primer according to claim 29 capable of detecting a fungal cytochrome b gene polymorphism at positions in the DNA corresponding to the first or third base in the triplet coding for the amino acid at the position corresponding to S. cerevisiae cytochrome b residue 129.

32. The allele specific primer according to claim 29 capable of detecting a fungal cytochrome b gene polymorphism at positions in the DNA corresponding to the third base in the triplet coding for the amino acid at the position corresponding to S. cerevisiae cytochromewsidue 129.

33. The allele specific primer according to claim 29 capable of detecting a fungal cytochrome b gene polymorphism at positions in the DNA corresponding to the first base in the triplet coding for the amino acid at the position corresponding to S. cerevisiae cytochrome b residue 129.

34. A diagnostic primer capable of binding to a template comprising a mutant type fungal cytochrome b nucleotide sequence at a position corresponding to first and/or the third base in the triplet coding for the amino acid at the position corresponding to S. cerevisiae cytochrome b residue 129 in the cytochrome b protein wherein the final 3′ nucleotide of the primer corresponds to a nucleotide present in said mutant form of a fungal cytochrome b gene and the presence of said nucleotide gives rise to fungal resistance to a strobilurin analogue or any other compound in the same cross resistance group.

35. A diagnostic primer capable of binding to a template comprising a mutant type fungal cytochrome b nucleotide sequence at a position corresponding to first or the third base in the triplet coding for the amino acid at the position corresponding to S. cerevisiae cytochrome b residue 129 in the cytochrome b protein wherein the final 3′ nucleotide of the primer corresponds to a nucleotide present in said mutant form of a fungal cytochrome b gene and the presence of said nucleotide gives rise to fungal resistance to a strobilurin analogue or any other compound in the same cross resistance group.

36. A kit for use in the method of any of claim 1.

37. The diagnostic kit according to claim 36 comprising one or more of the following: diagnostic, wild type, control and common oligonucleotide primers: appropriate nucleotide triphosphates, for example dATP, dCTP, dGTP, dTTP, a suitable polymerase and a buffer solution.

38. The kit according to claim 36 comprising an allele selective hybridisation probe and one or more of the following: oligonucleotide primers which allow the selective amplification of a segment of DNA comprising the region of the target pathogen cytochrome b gene including codon 129 from both wild type and isolates resistant to strobilurin analogue or any other compound in the same cross resistance group diagnostic wild type (F129) and resistant (A129) selective hybridisation probes, appropriate nucleotide triphosphates, for example dATP, dCTP, dGTP, dTTP, a suitable polymerase as previously described, and a buffer solution.
